(19) United States
(12) Patent Application Publication
(10) Pub. No.: US 2024/0108752 A1
(43) Pub. Date: Apr. 4, 2024

(54) ADENOVIRAL GENE THERAPY VECTORS

(71) Applicant: Ensoma, Inc., Boston, MA (US)

(72) Inventors: Soumitra Roy, Townsend, DE (US); Ashvin Reddy Bashyam, Cambridge, MA (US)

(21) Appl. No.: 18/268,392

(22) PCT Filed: Dec. 22, 2021
(86) PCT No.: PCT/US21/64979

§ 371 Date: Jun. 20, 2023

Related U.S. Application Data
(60) Provisional application No. 63/129,233, filed on Dec. 22, 2020.

(51) Int. Cl. A61K 48/00; A61K 31/7088; A61K 31/7105; A61K 38/17; A61K 38/46; C12N 15/86

(52) U.S. Cl. CPC A61K 48/0041; A61K 31/7088; A61K 31/7105; A61K 38/1774; A61K 38/465; C12N 15/86; C12N 2710/10022; C12N 2710/10043; C12N 2710/10071

(57) ABSTRACT

The present disclosure includes adenoviral vectors characterized by efficient transduction of HSCs, e.g., for in vivo gene therapy. The present disclosure includes, among other things, Ad3, Ad7, Ad11, Ad14, Adpatentdocket@choate.com16, Ad21, Ad34, Ad37, and Ad50 vectors and genomes. Ad3, Ad7, Ad11, Ad14, Ad16, Ad21, Ad34, Ad37, and Ad50 vectors and genomes of the present disclosure can include therapeutic payloads.

### PRIORITY APPLICATION

The present application claims the benefit of U.S. Provisional Patent Application No. 63/129,233, filed Dec. 22, 2020, the content of which is hereby incorporated by reference herein in its entirety.

### BACKGROUND

Many medical conditions are caused by genetic mutation and/or are treatable, at least in part, by gene therapy. Some conditions are particularly treatable by modification of hematopoietic stem cells (HSCs). Compositions and methods for HSC gene therapy are therefore needed.

### SUMMARY

Gene therapy can treat many conditions that have a genetic component, including without limitation hemoglobinopathies, immune deficiencies, and cancers. In various gene therapies, hematopoietic stem cells (HSCs) are an important target. However, current methods and compositions for modifying HSCs are limited. For instance, some vectors for gene therapy such as lentiviruses have a relatively limited payload capacity. Others, such as adenoviral serotype 5, are characterized by substantial payload capacity but are sufficiently prevalent that a significant fraction of humans have antibodies directed against vector proteins, some of which antibodies may be neutralizing. Moreover, different viral vectors are characterized by distinct transduction efficiencies for various cell types, such as HSCs. The present disclosure identified adenoviral serotypes characterized by high payload capacity and high transduction efficiency for HSCs.

The present disclosure includes, among other things, Ad3, 7, 11, 14, 16, 21, 34, 37, or 50 vectors and Ad3, 7, 11, 14, 16, 21, 34, 37, or 50 genomes (e.g., “recombinant” or “engineered” adenoviral vectors and adenoviral genomes). Ad3, 7, 11, 14, 16, 21, 34, 37, or 50 vectors and genomes of the present disclosure can include various payloads. In various embodiments, a payload can include one or more of a nucleic acid sequence encoding a CRISPR system, base editing system, prime editing system, or other expression product. The present disclosure includes, among other things, combination adenoviral vectors and adenoviral genomes that include nucleic acid sequences encoding a plurality of expression products that together contribute to treatment of a disease or condition. The present disclosure includes, among other things, Ad3, 7, 11, 14, 16, 21, 34, 37, or 50 adenoviral vectors and Ad3, 7, 11, 14, 16, 21, 34, 37, or 50 adenoviral genomes for integration of a nucleic acid payload into a target cell genome. The present disclosure includes, among other things, Ad3, 7, 11, 14, 16, 21, 34, 37, or 50 donor vectors, Ad3, 7, 11, 14, 16, 21, 34, 37, or 50 adenoviral donor genomes, helper dependent Ad3, 7, 11, 14, 16, 21, 34, 37, or 50 adenoviral donor vectors, helper dependent Ad3, 7, 11, 14, 16, 21, 34, 37, or 50 adenoviral donor genomes, support vectors, support genomes, Ad3, 7, 11, 14, 16, 21, 34, 37, or 50 helper vectors, and Ad3, 7, 11, 14, 16, 21, 34, 37, or 50 helper genomes. For avoidance of doubt, a list of serotypes such as “Ad3, 7, 11, 14, 16, 21, 34, 37, or 50” can alternatively be written as “Ad3, Ad7, Ad11, Ad14, Ad16, Ad21, Ad34, Ad37, or Ad50.”

In at least one aspect, the present disclosure provides a method of in vivo gene therapy in a mammalian subject, the method including administering to the subject an adenoviral vector, where the adenoviral vector includes: (a) a capsid including one or more viral polypeptides of an Ad3, Ad7, Ad11, Ad14, Ad16, Ad21, Ad34, Ad37, or Ad50 serotype (e.g., having at least 80% sequence identity to a reference polypeptide of the serotype), where the one or more viral polypeptides include one or more of a: (i) fiber knob; (ii) fiber shaft; (iii) fiber tail; (iv) penton; and (v) hexon; and (b) a double-stranded DNA genome including a heterologous nucleic acid payload. In various embodiments, the genome further includes: (a) a 3′ ITR and a 5′ ITR, where each of the 3′ ITR and the 5′ ITR are of the viral polypeptide serotype (e.g., having at least 80% sequence identity to a reference sequence of the same serotype as the serotype of the viral polypeptides) (b) a packaging sequence, where the packing sequence is of the viral polypeptide serotype. In various embodiments, the method includes mobilization of hematopoietic stem cells of the subject prior to administration of the adenoviral vector. In various embodiments, the heterologous nucleic acid payload includes a selectable marker, optionally where the selectable marker is MGMTP140K. In various embodiments, the method includes administering a selecting agent to the subject, optionally where the selecting agent includes O6BG and/or BCNU. In various embodiments, the method includes administering one or more immunosuppression agents to the subject, optionally where the administration of the one or more immunosuppression agents is prior to the administration of the adenoviral vector.

In at least one aspect, the present disclosure provides an adenoviral donor vector including: (a) a capsid including one or more viral polypeptides of an Ad3, Ad7, Ad11, Ad14, Ad16, Ad21, Ad34, Ad37, or Ad50 serotype, where the one or more viral polypeptides include one or more of a: (i) fiber knob; (ii) fiber shaft; (iii) fiber tail; (iv) penton; and (v) hexon; and (b) a double-stranded DNA genome including a heterologous nucleic acid payload. In various embodiments, the genome further includes: (a) a 3′ ITR and a 5′ ITR, where each of the 3′ ITR and the 5′ ITR are of the viral polypeptide serotype; and (b) a packaging sequence, where the packing sequence is of the viral polypeptide serotype. In various embodiments, the heterologous nucleic acid payload includes a selectable marker, optionally where the selectable marker is MGMTP140K.

In various embodiments of aspects provided by the present disclosure, the one or more viral polypeptides include the: (a) fiber knob and fiber shaft; (b) fiber knob and fiber tail; (c) fiber knob and penton; (d) fiber knob and hexon; (e) fiber knob, hexon, and penton; (f) fiber shaft and fiber tail; (g) fiber shaft and penton; (h) fiber shaft and hexon; (i) fiber shaft, hexon, and penton; (j) fiber tail and penton; (k) fiber tail and hexon; (1) fiber tail, hexon, and penton; (m) fiber knob, fiber shaft, and fiber tail; (n) fiber knob, fiber shaft, and penton; (o) fiber knob, fiber shaft, and hexon; (p) fiber knob, fiber shaft, hexon, and penton; (q) fiber knob, fiber shaft, fiber tail, and penton; (r) fiber knob, fiber shaft, fiber tail, penton, and hexon; or (s) penton and hexon.

In various embodiments of aspects provided by the present disclosure, the fiber knob has a sequence that has at least 80% identity to a sequence selected from SEQ ID NOs: 14, 30, 46, 62, 78, 94, 110, 126, and 142. In various embodiments of aspects provided by the present disclosure, the fiber shaft has a sequence that has at least 80% identity to a sequence selected from SEQ ID NOs: 13, 29, 45, 61, 77, 93, 109, 125, and 141. In various embodiments of aspects provided by the present disclosure, the fiber tail has a sequence that has at least 80% identity to a sequence selected from SEQ ID NOs: 157, 158, 159, 160, 161, 162, 163, 164, and 165. In various embodiments of aspects provided by the present disclosure, the penton has a sequence that has at least 80% identity to a sequence selected from SEQ ID NOs: 15, 31, 47, 63, 79, 95, 111, 127, and 143. In various embodiments of aspects provided by the present disclosure, the hexon has a sequence that has at least 80% identity to a sequence selected from SEQ ID NOs: 16, 32, 48, 64, 80, 96, 112, 128, and 144. In various embodiments of aspects provided by the present disclosure, the adenoviral vector includes a fiber of the serotype of the viral peptides. In various embodiments of aspects provided by the present disclosure, the fiber has a sequence that has at least 80% identity to a sequence selected from SEQ ID NOs: 12, 28, 44, 60, 76, 92, 108, 124, and 140.

In various embodiments of aspects provided by the present disclosure, the adenoviral vector is a chimeric vector characterized in that the capsid includes at least one of a fiber knob, fiber shaft, fiber tail, hexon, or penton that is not of the serotype of the viral peptides.

In various embodiments of aspects provided by the present disclosure, the adenoviral vector is a helper dependent vector.

In at least one aspect, the present disclosure provides an adenoviral donor vector genome including: (a) a 3′ ITR and a 5′ ITR, where the 3′ ITR and the 5′ ITR are each of the same serotype selected from an Ad3, Ad7, Ad11, Ad14, Ad16, Ad21, Ad34, Ad37, or Ad50 serotype; (b) a packaging sequence, where the packing sequence is of the ITR serotype; and (c) a heterologous nucleic acid payload. In certain embodiments, the heterologous nucleic acid payload includes a selectable marker, optionally where the selectable marker is MGMTP140K.

In various embodiments of aspects provided by the present disclosure, the heterologous nucleic acid payload encodes a protein. In various embodiments of aspects provided by the present disclosure, the heterologous nucleic acid payload encodes a small RNA, optionally where the small RNA is an shRNA. In various embodiments of aspects provided by the present disclosure, the heterologous nucleic acid payload encodes a gene editing enzyme or system, where the gene editing is selected from CRISPR editing, base editing, prime editing, or zinc finger nuclease editing.

In various embodiments of aspects provided by the present disclosure, the heterologous nucleic acid payload encodes an agent for treatment of a condition selected from hemoglobinopathies, platelet disorders, Fanconi anemia, alpha-1 antitrypsin deficiency, sickle cell anemia, thalassemia, thalassemia intermedia, von Willebrand Disease, hemophilia A, hemophilia B, Factor V Deficiency, Factor VII Deficiency, Factor X Deficiency, Factor XI Deficiency, Factor XII Deficiency, Factor XIII Deficiency, Bernard-Soulier Syndrome, Gray Platelet Syndrome, mucopolysaccharidosis, cystic fibrosis, Tay-Sachs disease, and phenylketonuria. In various embodiments of aspects provided by the present disclosure, the heterologous nucleic acid payload encodes an agent for treatment of a condition selected from Grave's Disease, rheumatoid arthritis, pernicious anemia, Multiple Sclerosis (MS), inflammatory bowel disease, systemic lupus erythematosus (SLE), adenosine deaminase deficiency (ADA-SCID) or severe combined immunodeficiency disease (SCID), Wiskott-Aldrich syndrome (WAS), chronic granulomatous disease (CGD), Deficiency of Adenosine Deaminase 2, Fanconi anemia (FA), Battens disease, adrenoleukodystrophy (ALD) or metachromatic leukodystrophy (MLD), muscular dystrophy, pulmonary alveolar proteinosis (PAP), pyruvate kinase deficiency, Schwachman-Diamond-Blackfan anemia, dyskeratosis congenita, cystic fibrosis, Parkinson's disease, Alzheimer's disease, and amyotrophic lateral sclerosis (Lou Gehrig's disease).

In various embodiments of aspects provided by the present disclosure, the serotype of the viral polypeptides is Ad34. In various embodiments of aspects provided by the present disclosure, the serotype of the viral polypeptides is Ad3. In various embodiments of aspects provided by the present disclosure, the serotype of the viral polypeptides is Ad7. In various embodiments of aspects provided by the present disclosure, the serotype of the viral polypeptides is Ad11. In various embodiments of aspects provided by the present disclosure, the serotype of the viral polypeptides is Ad14. In various embodiments of aspects provided by the present disclosure, the serotype of the viral polypeptides is Ad16. In various embodiments of aspects provided by the present disclosure, the serotype of the viral polypeptides is Ad21. In various embodiments of aspects provided by the present disclosure, the serotype of the viral polypeptides is Ad37. In various embodiments of aspects provided by the present disclosure, the serotype of the viral polypeptides is Ad50

In various embodiments, the present disclosure provides a pharmaceutical composition including an adenoviral vector of the present disclosure, where the pharmaceutical composition is formulated for injection to a subject in need thereof.

In various embodiments, the present disclosure provides a method, vector, genome, or pharmaceutical composition in which an adenoviral vector infects and/or transduces CD34+ cells, CD34+high cells, CD34+/CD90+ cells, and/or CD34+high/CD90+ cells, optionally wherein the cells are hematopoietic cells.

### Definitions

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Adoptive cell therapy: As used herein, “adoptive cell therapy” or “ACT” involves transfer of cells with a therapeutic activity into a subject, e.g., a subject in need of treatment for a condition, disorder, or disease. In some embodiments, ACT includes transfer into a subject of cells after ex vivo and/or in vitro engineering and/or expansion of the cells.

Affinity: As used herein, “affinity” refers to the strength of the sum total of non-covalent interactions between a particular binding agent (e.g., a viral vector), and/or a binding moiety thereof, with a binding target (e.g., a cell). Unless indicated otherwise, as used herein, “binding affinity” refers to a 1:1 interaction between a binding agent and a binding target thereof (e.g., a viral vector with a target cell of the viral vector). Those of skill in the art appreciate that a change in affinity can be described by comparison to a reference (e.g., increased or decreased relative to a reference), or can be described numerically. Affinity can be measured and/or expressed in a number of ways known in the art, including, but not limited to, equilibrium dissociation constant (K_{D}) and/or equilibrium association constant (K_{A}). K_{D} is the quotient of k_{off}/kₒₙ, whereas K_{A} is the quotient of kₒₙ/k_{off}, where kₒₙ refers to the association rate constant of, e.g., viral vector with target cell, and k_{off} refers to the dissociation of, e.g., viral vector from target cell. The kₒₙ and k_{off} can be determined by techniques known to those of skill in the art.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Allogeneic: As used herein, term “allogeneic” refers to any material derived from one subject which is then introduced to another subject, e.g., allogeneic HSC transplantation.

Between or From: As used herein, the term “between” refers to content that falls between indicated upper and lower, or first and second, boundaries, inclusive of the boundaries. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents; indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Cancer: As used herein, the term “cancer” refers to a condition, disorder, or disease in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer can include one or more tumors. In some embodiments, a cancer can be or include cells that are precancerous (e.g., benign), malignant, pre-metastatic, metastatic, and/or non-metastatic. In some embodiments, a cancer can be or include a solid tumor. In some embodiments, a cancer can be or include a hematologic tumor.

Chimeric antigen receptor: As used herein, “Chimeric antigen receptor” or “CAR” refers to an engineered protein that includes (i) an extracellular domain that includes a moiety that binds a target antigen; (ii) a transmembrane domain; and (iii) an intracellular signaling domain that sends activating signals when the CAR is stimulated by binding of the extracellular binding moiety with a target antigen. CARs are also known as chimeric T cell receptors or chimeric immunoreceptors.

Combination therapy: As used herein, the term “combination therapy” refers to administration to a subject of to two or more agents or regimens such that the two or more agents or regimens together treat a condition, disorder, or disease of the subject. In some embodiments, the two or more therapeutic agents or regimens can be administered simultaneously, sequentially, or in overlapping dosing regimens. Those of skill in the art will appreciate that combination therapy includes but does not require that the two agents or regimens be administered together in a single composition, nor at the same time.

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE.

Dosing regimen: As used herein, the term “dosing regimen” can refer to a set of one or more same or different unit doses administered to a subject, typically including a plurality of unit doses administration of each of which is separated from administration of the others by a period of time. In various embodiments, one or more or all unit doses of a dosing regimen may be the same or can vary (e.g., increase over time, decrease over time, or be adjusted in accordance with the subject and/or with a medical practitioner's determination). In various embodiments, one or more or all of the periods of time between each dose may be the same or can vary (e.g., increase over time, decrease over time, or be adjusted in accordance with the subject and/or with a medical practitioner's determination). In some embodiments, a given therapeutic agent has a recommended dosing regimen, which can involve one or more doses. Typically, at least one recommended dosing regimen of a marketed drug is known to those of skill in the art. In some embodiments, a dosing regimen is correlated with a desired or beneficial outcome when administered across a relevant population (i.e., is a therapeutic dosing regimen).

Downstream and Upstream: As used herein, the term “downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

Effective amount: An “effective amount” is the amount of a formulation necessary to result in a desired physiological change in a subject. Effective amounts are often administered for research purposes.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

Expression: As used herein, “expression” refers individually and/or cumulatively to one or more biological process that result in production from a nucleic acid sequence of an encoded agent, such as a protein. Expression specifically includes either or both of transcription and translation.

Flank: As used herein, a first element (e.g., a nucleic acid sequence or amino acid sequence) present in a contiguous sequence with a second element and a third element is “flanked” by the second element and third element if it is positioned in the contiguous sequence between the second element and the third element. Accordingly, in such arrangement, the second element and third element can be referred to as “flanking” the first element. Flanking elements can be immediately adjacent to a flanked element or separated from the flanked element by one or more relevant units. In various examples in which the contiguous sequence is a nucleic acid or amino acid sequence, and the relevant units are bases or amino acid residues, respectively, the number of units in the contiguous sequence that are between a flanked element and, independently, first and/or second flanking elements can be, e.g., 50 units or less, e.g., no more than 50, 45, 40, 35, 30, 25, 20, 15, 10, 5, 4, 3, 2, 1, or 0 units.

Fragment: As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment of a polymer includes or consists of at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means.

Gene, Transgene: As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Host cell, target cell: As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, an intended or potential host cell can be referred to as a target cell.

In various embodiments, a host cell or target cell is identified by the presence, absence, or expression level of various surface markers.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Identity: As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. The term “% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. For instance, calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally accounting for the number of gaps, and the length of each gap, which may need to be introduced for optimal alignment of the two sequences. The comparison of sequences and determination of percent identity between two sequences can be accomplished using a computational algorithm, such as BLAST (basic local alignment search tool). Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated. In some embodiments, isolated agents are 80%, 85%, 90%, 91%, 92%, 93%, 94%, 95%, 96%, 97%, 98%, 99%, or more than 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Operably linked: As used herein, “operably linked” or “operatively linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to a composition in which an active agent is formulated together with one or more pharmaceutically acceptable carriers.

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, N Y, 1989).

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represents measure under comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof. Without wishing to be bound by any particular embodiment(s), in various embodiments a reference sequence can be a sequence associated with a sequence accession number provided herein, certain of which sequences associated with sequence accession numbers are provided in FIG. 40.

Regulatory sequence: As used herein in the context of expression of a nucleic acid coding sequence, a regulatory sequence is a nucleic acid sequence that controls expression of a coding sequence. In some embodiments, a regulatory sequence can control or impact one or more aspects of gene expression (e.g., cell-type-specific expression, inducible expression, etc.).

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.”

Therapeutic agent: As used herein, the term “therapeutic agent” refers to any agent that elicits a desired pharmacological effect when administered to a subject. In some embodiments, an agent is considered to be a therapeutic agent if it demonstrates a statistically significant effect across an appropriate population. In some embodiments, the appropriate population can be a population of model organisms or a human population. In some embodiments, an appropriate population can be defined by various criteria, such as a certain age group, gender, genetic background, preexisting clinical conditions, etc. In some embodiments, a therapeutic agent is a substance that can be used for treatment of a disease, disorder, or condition. In some embodiments, a therapeutic agent is an agent that has been or is required to be approved by a government agency before it can be marketed for administration to humans. In some embodiments, a therapeutic agent is an agent for which a medical prescription is required for administration to humans.

Therapeutically effective amount: As used herein, “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition. A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition and is administered to the subject for the purpose of reducing the severity or progression of the condition.

Unit dose: As used herein, the term “unit dose” refers to an amount administered as a single dose and/or in a physically discrete unit of a pharmaceutical composition. In many embodiments, a unit dose contains a predetermined quantity of an active agent, for instance a predetermined viral titer (the number of viruses, virions, or viral particles in a given volume). In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic moiety(s). For example, acceptable carriers (e.g., pharmaceutically acceptable carriers), diluents, stabilizers, buffers, preservatives, etc., can be included. It will be appreciated by those skilled in the art, in many embodiments, a total appropriate daily dosage of a particular therapeutic agent can include a portion, or a plurality, of unit doses, and can be decided, for example, by a medical practitioner within the scope of sound medical judgment. In some embodiments, the specific effective dose level for any particular subject or organism can depend upon a variety of factors including the disorder being treated and the severity of the disorder; activity of specific active compound employed; specific composition employed; age, body weight, general health, sex, and diet of the subject; time of administration, and rate of excretion of the specific active compound employed; duration of the treatment; drugs and/or additional therapies used in combination or coincidental with specific compound(s) employed, and like factors well known in the medical arts.

### BRIEF DESCRIPTION OF THE DRAWING

FIG. 1 is a chart showing results of anti-hexon staining of CD34+ cells three hours after infection of the cells with indicated adenoviral serotypes. Cells were infected at 5,000 viral particles per cell or 2,000 viral particles per cell. For each tested serotype, the chart includes two replicates of data, each replicate including, in the order shown, results of analysis at 5,000 viral particles per cell and 2,000 viral particles per cell. Data represent infection efficiency.

FIG. 2 is a chart showing results of qPCR analysis of adenoviral DNA in CD34+ cells infected with the indicated adenoviral serotypes. Cells were infected at 5,000 viral particles per cell or 2,000 viral particles per cell. For each tested serotype, the chart includes two replicates of data, each replicate including, in the order shown, results of analysis at 5,000 viral particles per cell and 2,000 viral particles per cell. Data represent relative infection efficiency.

FIG. 3 is a chart showing results of anti-hexon staining of CD34+ cells from Donor 1 three hours after infection of the cells with indicated adenoviral serotypes. Cells were infected at 2,000 viral particles per cell. For each tested serotype, the chart includes two replicates of data. Data represent infection efficiency.

FIG. 4 is a chart showing results of anti-hexon staining of CD34+ cells from Donor 1 six hours after infection of the cells with indicated adenoviral serotypes. Cells were infected at 2,000 viral particles per cell. For each tested serotype, the chart includes two replicates of data. Data represent infection efficiency.

FIG. 5 is a chart showing results of anti-hexon staining of CD34+ cells from Donor 1 three hours after infection of the cells with indicated adenoviral serotypes. Cells were infected at 5,000 viral particles per cell. For each tested serotype, the chart includes two replicates of data. Data represent infection efficiency.

FIG. 6 is a chart showing results of anti-hexon staining of CD34+ cells from Donor 1 six hours after infection of the cells with indicated adenoviral serotypes. Cells were infected at 5,000 viral particles per cell. For each tested serotype, the chart includes two replicates of data. Data represent infection efficiency.

FIG. 7 is a chart showing results of anti-hexon staining of CD34+ cells from Donor 2 three hours after infection of the cells with indicated adenoviral serotypes. Cells were infected at 2,000 viral particles per cell. For each tested serotype, the chart includes two replicates of data. Data represent infection efficiency. Two replicate preparations of serotype F35 were tested. GLN indicates that the indicated adenoviral vector included an expression cassette encoding a GFP luminescence reporter.

FIG. 8 is a chart showing results of anti-hexon staining of CD34+ cells from Donor 2 six hours after infection of the cells with indicated adenoviral serotypes. Cells were infected at 2,000 viral particles per cell. For each tested serotype, the chart includes three replicates of data. Data represent infection efficiency.

FIG. 9 is a chart showing results of anti-hexon staining of CD34+ cells from Donor 2 three hours after infection of the cells with indicated adenoviral serotypes. Cells were infected at 5,000 viral particles per cell. For each tested serotype, the chart includes three replicates of data. Data represent infection efficiency.

FIG. 10 is a chart showing results of anti-hexon staining of CD34+ cells from Donor 2 six hours after infection of the cells with indicated adenoviral serotypes. Cells were infected at 5,000 viral particles per cell. For each tested serotype, the chart includes three replicates of data. Data represent infection efficiency.

FIG. 11 is a chart showing results of anti-hexon staining of CD34+ cells from Donor 3 three hours after infection of the cells with indicated adenoviral serotypes. Cells were infected at 2,000 viral particles per cell. For each tested serotype, the chart includes three replicates of data. Data represent infection efficiency.

FIG. 12 is a chart showing results of anti-hexon staining of CD34+ cells from Donor 3 six hours after infection of the cells with indicated adenoviral serotypes. Cells were infected at 2,000 viral particles per cell. For each tested serotype, the chart includes two or three replicates of data. Data represent infection efficiency.

FIG. 13 is a chart showing results of anti-hexon staining of CD34+ cells from Donor 3 three or six hours after infection of the cells with indicated adenoviral serotypes. Cells were infected at 5,000 viral particles per cell. For each tested serotype, the chart includes two replicates of data, each replicate including, in the order shown, results of analysis at three hours and six hours after infection. Data represent infection efficiency.

FIG. 14 is a chart showing results of qPCR analysis of adenoviral DNA in CD34+ cells from Donor 1 three hours after infection of the cells with indicated adenoviral serotypes. Cells were infected at 2,000 viral particles per cell. For each tested serotype, the chart includes two replicates of data. Error bars represent technical replicates. Data represent relative infection efficiency.

FIG. 15 is a chart showing results of qPCR analysis of adenoviral DNA in CD34+ cells from Donor 1 six hours after infection of the cells with indicated adenoviral serotypes. Cells were infected at 2,000 viral particles per cell. For each tested serotype, the chart includes two replicates of data. Data represent relative infection efficiency.

FIG. 16 is a chart showing results of qPCR analysis of adenoviral DNA in CD34+ cells from Donor 1 three hours after infection of the cells with indicated adenoviral serotypes. Cells were infected at 5,000 viral particles per cell. For each tested serotype, the chart includes two replicates of data. Error bars represent technical replicates. Data represent relative infection efficiency.

FIG. 17 is a chart showing results of qPCR analysis of adenoviral DNA in CD34+ cells from Donor 1 six hours after infection of the cells with indicated adenoviral serotypes. Cells were infected at 5,000 viral particles per cell. For each tested serotype, the chart includes two replicates of data. Data represent relative infection efficiency.

FIG. 18 is a chart showing results of qPCR analysis of adenoviral DNA in CD34+ cells from Donor 2 three hours after infection of the cells with indicated adenoviral serotypes. Cells were infected at 2,000 viral particles per cell. For each tested serotype, the chart includes two replicates of data. Error bars represent technical replicates. Data represent relative infection efficiency.

FIG. 19 is a chart showing results of qPCR analysis of adenoviral DNA in CD34+ cells from Donor 2 six hours after infection of the cells with indicated adenoviral serotypes. Cells were infected at 2,000 viral particles per cell. For each tested serotype, the chart includes three replicates of data. Error bars represent technical replicates. Data represent relative infection efficiency.

FIG. 20 is a chart showing results of qPCR analysis of adenoviral DNA in CD34+ cells from Donor 2 three hours after infection of the cells with indicated adenoviral serotypes. Cells were infected at 5,000 viral particles per cell. For each tested serotype, the chart includes three replicates of data. Error bars represent technical replicates. Data represent relative infection efficiency.

FIG. 21 is a chart showing results of qPCR analysis of adenoviral DNA in CD34+ cells from Donor 2 six hours after infection of the cells with indicated adenoviral serotypes. Cells were infected at 5,000 viral particles per cell. For each tested serotype, the chart includes three replicates of data. Error bars represent technical replicates. Data represent relative infection efficiency.

FIG. 22 is a chart showing results of qPCR analysis of adenoviral DNA in CD34+ cells from Donor 3 three hours after infection of the cells with indicated adenoviral serotypes. Cells were infected at 2,000 viral particles per cell. For each tested serotype, the chart includes three replicates of data. Error bars represent technical replicates. Data represent relative infection efficiency.

FIG. 23 is a chart showing results of qPCR analysis of adenoviral DNA in CD34+ cells from Donor 3 six hours after infection of the cells with indicated adenoviral serotypes. Cells were infected at 2,000 viral particles per cell. For each tested serotype, the chart includes two or three replicates of data. Error bars represent technical replicates. Data represent relative infection efficiency.

FIG. 24 is a chart showing results of qPCR analysis of adenoviral DNA in CD34+ cells from Donor 2 three or six hours after infection of the cells with indicated adenoviral serotypes. Cells were infected at 5,000 viral particles per cell. For each tested serotype, the chart includes two replicates of data, each replicate including, in the order shown, results of analysis at three hours and six hours after infection. Error bars represent technical replicates. Data represent relative infection efficiency.

FIG. 25 is an image of a gel showing digestion of first generation adenoviral genomes of serotype Ad11 obtained from an Ad preparation produced from HEK293 cells transfected with a plasmid including a first generation adenoviral genome of serotype Ad11. As indicated in the table included in the figure, the purified adenoviral genomes were digested with either BspHI (lane 3) or SmaI (lane 5), while parental plasmids were also digested for comparison (lanes 2 and 4, respectively). A representation of the predicted digestion fragments based on the sequences of the Ad genome and plasmid is also shown.

FIG. 26 is an image of a gel showing digestion of first generation adenoviral genomes of serotype Ad34 obtained from an Ad preparation produced from HEK293 cells transfected with a plasmid including a first generation adenoviral genome of serotype Ad34. As indicated in the table included in the figure, the purified adenoviral genomes were digested with either SmaI (lane 2) or SspI (lane 3). A representation of the predicted digestion fragments based on the sequence of the Ad genome is also shown.

FIG. 27 is an image of a gel showing digestion of first generation Ad35++ genomes obtained from an Ad preparation produced from HEK293 cells transfected with a plasmid including a first generation Ad35++ genome. As indicated in the table included in the figure, the purified adenoviral genomes were digested with BspHI (lane 2), while parental plasmid was also digested for comparison (lane 3). A representation of the predicted digestion fragments based on the sequences of the Ad genome and plasmid is also shown. * indicates a lane with a repeated sample.

FIG. 28 are images of gels showing digestion of first generation Ad35++ genomes obtained from an Ad preparation produced from HEK293 cells transfected with a plasmid including a first generation Ad35++ genome. The gel labelled Observed #1 was electrophoresed for a longer duration to resolve large DNA fragments, while the gel labelled Observed #2 was electrophoresed for a shorter duration to resolve shorter DNA fragments. As indicated in the table included in the figure, the purified adenoviral genomes were digested with SmaI (lane 2), while parental plasmid was also digested for comparison (lane 3). A representation of the predicted digestion fragments based on the sequences of the Ad genome and plasmid is also shown.

FIG. 29 is a chart showing results of GFP analysis of HEK293 cells 25 hours after infection of the cells with first generation adenoviral vectors of the indicated adenoviral serotypes. Cells were infected at 100, 200, 500, and 1,000 viral particles per cell. Data represent infection efficiency. NTC indicates non-treated control.

FIG. 30 is a chart showing results of GFP analysis of HEK293 cells 24 hours after infection of the cells with first generation adenoviral vectors of the indicated adenoviral serotypes. Cells were infected at 100, 200, 500, 1,000, and 2,000 viral particles per cell. Data represent infection efficiency.

FIG. 31 is a chart showing results of GFP analysis of K562 cells 24 hours after infection of the cells with first generation adenoviral vectors of the indicated adenoviral serotypes. Cells were infected at 100, 200, 500, 1,000, and 2,000 viral particles per cell. Data represent infection efficiency.

FIG. 32 is a chart showing results of GFP analysis of CD34+ cells from Donor 2 48 hours after infection of the cells with first generation adenoviral vectors of the indicated adenoviral serotypes. Cells were infected at 500, 2,000, and 5,000 viral particles per cell. For conditions using 2,000 and 5,000 viral particles per cell, the chart includes two replicates of data. Data represent infection efficiency.

FIG. 33 depicts the gating used for analysis CD34+ and CD34+/CD90+ populations using flow cytometry. Purified CD34+ cells were stained with anti-CD34 and anti-CD90 antibodies and transduction efficiency was measured in CD34+high/CD90+ cells. Boxes indicate gates used to define a population of cells. Arrows from one plot to another indicate that the gated population in the first plot is being displayed in the second plot. Percentages indicate the percent of cells contained with each indicated gate. The data shown in this figure corresponds to CD34+ cells from Donor 1, 46 hours after infection of the cells with first generation adenoviral vector of serotype Ad34 at 5,000 viral particles per cell.

FIG. 34 is a chart showing results of GFP analysis of CD34+ cells and CD34+/CD90+ cells from Donor 1 46 hours after infection of the cells with first generation adenoviral vectors of the indicated adenoviral serotypes. Percent of cells that are GFP positive is shown. Cells were infected at 2,000 and 5,000 viral particles per cell. Data represent infection efficiency. * indicates an absence of data collected for the indicated condition.

FIG. 35 is a chart showing results of GFP analysis of CD34+ cells and CD34+/CD90+ cells from Donor 3 46 hours after infection of the cells with first generation adenoviral vectors of the indicated adenoviral serotypes. Percent of cells that are GFP positive is shown. Cells were infected at 500, 2,000, and 5,000 viral particles per cell. Data represent infection efficiency.

FIG. 36 is a chart showing results of GFP analysis of CD34+ cells and CD34+/CD90+ cells from Donor 1 46 hours after infection of the cells with first generation adenoviral vectors of the indicated adenoviral serotypes. Geometric mean fluorescence intensity (MFI) of GFP for GFP positive cells is shown. Cells were infected at 2,000 and 5,000 viral particles per cell. Data represent infection efficiency. * indicates an absence of data collected for the indicated condition.

FIG. 37 is a chart showing results of GFP analysis of CD34+ cells and CD34+/CD90+ cells from Donor 3 46 hours after infection of the cells with first generation adenoviral vectors of the indicated adenoviral serotypes. Geometric mean fluorescence intensity (MFI) of GFP for GFP positive cells is shown. Cells were infected at 500, 2,000, and 5,000 viral particles per cell. Data represent infection efficiency.

FIG. 38 is a chart showing results of qPCR analysis of adenoviral DNA in HEK293 cells three hours after infection of the cells with indicated adenoviral serotypes. Cells were infected at 100 and 500 viral particles per cell. Data represent relative infection efficiency.

FIG. 39 is a chart showing results of qPCR analysis of adenoviral DNA in CD34+ cells from Donor 2 three hours after infection of the cells with indicated adenoviral serotypes. Cells were infected at 500, 2,000, and 5,000 viral particles per cell. For conditions using 2,000 and 5,000 viral particles per cell, the chart includes two replicates of data. Data represent relative infection efficiency.

FIG. 40 is a listing of nucleic acid sequences and amino acid sequences corresponding to publicly available sequence accession numbers, certain of which sequences and/or sequence accession numbers are included and/or utilized, in whole and/or in part, in the present disclosure, and/or certain of which sequences and/or sequence accession numbers are included herein as references.

### DETAILED DESCRIPTION

The present disclosure provides methods and compositions that include adenoviral vectors advantageous for gene therapy targeting HSCs. Methods and compositions of the present disclosure are based at least in part on the observation that adenoviral vectors of serotypes 3, 7, 11, 14, 16, 21, 34, 37, and 50 demonstrate certain advantageous properties for gene therapy targeting HSCs, at least as compared to one or more reference adenoviral vectors (e.g., an Ad5 vector or an Ad5/35 vector). Adenovirus (or, interchangeably, “adenoviral”) vectors include virus particles characterized by one or more adenoviral protein sequences and optionally include an adenoviral genome. Adenoviral genomes include nucleic acid sequences that include adenoviral sequences sufficient to (a) support packaging of the nucleic acid sequence (including conditional packaging) into an adenoviral vector and to (b) express a coding sequence. Adenoviral genomes can be linear, double-stranded DNA sequences and/or molecules. As those of skill in the art will appreciate, a linear genome such as an adenoviral genome can be present in a circular plasmid, e.g., for viral production purposes. Natural adenoviral genomes range from 26 kb to 45 kb in length, depending on the serotype.

The present disclosure includes methods and compositions that include engineered adenoviral vectors and adenoviral genomes. Adenoviral vectors include engineered adenoviral vectors that include an engineered adenoviral protein or engineered adenoviral genome. Engineered adenoviral genomes can be engineered to add or remove adenoviral genome sequences, e.g., as compared to a reference sequence.

Among adenoviruses, there are 57 known human serotypes. One in particular, Adenovirus serotype 5 (Ad5), has historically been widely used in gene therapy research and adenoviral vector constructs. Certain research has been conducted using HDAd5/35 vectors that include Ad5 capsid proteins except that the fibers are chimeric in that they include an Ad5 fiber tail, an Ad35 fiber shaft, and an Ad35 fiber knob (see, e.g., Shayakhmetov et al. 2000 J. Virol 74(6):2567-2583), optionally wherein the Ad35 fiber knob is mutated for increased affinity to CD46 (e.g., Ad5/35++). In particular embodiments, an Ad5/35++ vector is a chimeric Ad5/35 vector with a mutant Ad35++ fiber knob (see, e.g., Wang et al. 2008 J. Virol. 82(21):10567-79, which is incorporated herein by reference in its entirety and particularly with respect to fiber knob mutations). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593(24): 3623-3648, 2019). In certain embodiments, an adenoviral vector is a chimeric “F35” vector in which all proteins are Ad5 proteins except that the fibers are chimeric in that they include an Ad5 fiber tail, Ad35 fiber shaft, and an Ad35 fiber knob (e.g., as described in Shayakhmetov 2000 J Virol. 74(6): 2567-2583), where the Ad35 fiber knob is a mutant Ad35 fiber knob including mutations D207G and T245A causing increased affinity to CD46 (see, e.g., Wang 2008 J Virol. 82(21):10567-79), and optionally where the genome encoding the Ad5/35 vector includes an E1 deletion. The majority of humans, however, have neutralizing serum antibodies directed against Ad5 capsid proteins, which can block in vivo transduction with adenoviral vectors that include an Ad5 capsid, such as HDAd5/35 vectors. While the existence of neutralizing serum antibodies directed against Ad5 capsid proteins does not negate the therapeutic value of adenoviral vectors that include Ad5 capsids, adenoviral vectors that do not include Ad5 capsids would provide an additional benefit. At least one reason for this benefit is that Ad5 vectors can cause a clinically significant immune response in subjects that have serum antibodies directed against Ad5 capsid proteins (see, e.g., Somanathan et al. 2020 Mol. Ther. 28(3): 784-793), where serotypes without Ad5 capsid proteins may be less likely to cause such an immune response. At least a second reason is that neutralizing serum antibodies directed against Ad5 capsid proteins can reduce therapeutic efficacy of an Ad5 gene therapy vector by inactivating vector particles, where serotypes without Ad5 capsid proteins may be less likely to be inactivated.

The present disclosure includes adenoviral serotypes that demonstrate infection of target HSCs, including in various embodiments increased infection of HSCs as compared to reference adenoviral serotypes, e.g., Ad5 and/or Ad5/35, and are therefore useful for the production of adenoviral vectors for transduction of HSCs. Methods and compositions of the present disclosure included adenoviral vectors of serotypes 3, 7, 11, 14, 16, 21, 34, 37, and 50.

### I. Gene Therapy Vectors

### 1(A). Adenoviral Vectors

The present disclosure includes adenoviral vectors and adenoviral genomes useful in gene therapy. Adenoviruses are large, icosahedral-shaped, non-enveloped viruses. Natural adenoviral capsids include three types of proteins: fiber, penton, and hexon. The hexon makes up the majority of the viral capsid, forming 20 triangular faces. A penton base is located at each of the 12 vertices of the capsid, and a fiber (also referred to as knobbed fiber) protrudes from each penton base. Penton and fiber, and in particular the fiber knob, are of particular importance in receptor binding and internalization as they facilitate the attachment of the capsid to host cells.

Adenoviral genomes include Adenoviral DNA flanked on both ends by serotype-specific inverted terminal repeats (ITRs), which are understood to be cis elements that contribute to or are necessary for viral genome replication and packaging. Depending on the serotype, ITRs can be approximately 100-200 base pairs (e.g., about 160 base pairs) in length, with highest conservation at nucleotide positions (e.g., −50 base pairs) closest to the adenoviral genome terminii. Adenoviral genomes also include a packaging sequence (e.g., a conditional or non-conditional packaging sequence), which can facilitate packaging of the viral genome into viral vectors. Packaging sequences are located in the left portion of the genome.

Natural adenoviral genomes encode several proteins including early transcriptional units, E1, E2, E3, and E4 and late transcriptional units which encode structural protein components of the adenoviral vector. Early (E) and late (L) transcription are divided by the onset of viral genome replication. The E1 region (E1A and E1B) encodes proteins responsible for the regulation of transcription of the viral genome. The expression of the E2 region (E2A and E2B) results in the synthesis of the proteins for viral genome replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection. mRNAs transcribed using this promoter can include a 5′-tripartite leader (TPL) sequence that facilitates translation.

### 1(B). Ad3, 7, 11, 14, 16, 21, 34, 37, and 50 Gene Therapy Vectors

The present disclosure includes Ad3, 7, 11, 14, 16, 21, 34, 37, or 50 genomes. In various embodiments, an Ad3, 7, 11, 14, 16, 21, 34, 37, or 50 genome is a single-stranded or double-stranded DNA sequence that includes ITRs of an Ad3, 7, 11, 14, 16, 21, 34, 37, or 50 vector (e.g., a 5′ ITR according to SEQ ID NO: 1, 17, 33, 49, 65, 81, 97, 113, or 129 and a 3′ ITR according to SEQ ID NO: 2, 18, 34, 50, 66, 82, 98, 114, or 130), or ITRs that individually and/or together have at least 75% sequence identity (e.g., at least 75%, 80%, 85%, 90%, 95%, 96%, 97%, 98%, 99%, or 100% identity) thereto. In various embodiments, an Ad3, 7, 11, 14, 16, 21, 34, 37, or 50 genome is a single-stranded or double-stranded DNA sequence that includes a packaging sequence of an Ad3, 7, 11, 14, 16, 21, 34, 37, or 50 vector (e.g., a packaging sequence according to SEQ ID NO: 3, 19, 35, 51, 67, 83, 99, 115, or 131), or a packaging sequence having at least 75% sequence identity (e.g., at least 75%, 80%, 85%, 90%, 95%, 96%, 97%, 98%, 99%, or 100% identity) thereto. In various embodiments, an Ad3, 7, 11, 14, 16, 21, 34, 37, or 50 genome is a single-stranded or double-stranded DNA sequence that includes a sequence with at least 75% sequence identity (e.g., at least 75%, 80%, 85%, 90%, 95%, 96%, 97%, 98%, 99%, or 100% identity) to all, a portion of, or a contiguous corresponding portion of, or a discontiguous corresponding portion of a reference Ad3, 7, 11, 14, 16, 21, 34, 37, or 50 genome (e.g., SEQ ID NO: 145, 146, 147, 148, 149, 150, 151, 152, or 153).

In various embodiments, an Ad3, 7, 11, 14, 16, 21, 34, 37, or 50 genome is any nucleotide sequence that includes at least ITRs of an Ad3, 7, 11, 14, 16, 21, 34, 37, or 50 vector (e.g., a 5′ ITR according to SEQ ID NO: 1, 17, 33, 49, 65, 81, 97, 113, or 129 and a 3′ ITR according to SEQ ID NO: 2, 18, 34, 50, 66, 82, 98, 114, or 130), or ITRs that individually and/or together have at least 75% sequence identity (e.g., at least 75%, 80%, 85%, 90%, 95%, 96%, 97%, 98%, 99%, or 100% identity) thereto. In various embodiments, an Ad3, 7, 11, 14, 16, 21, 34, 37, or 50 genome is an Ad3, 7, 11, 14, 16, 21, 34, 37, or 50 genome from which one or more nucleotides, coding sequences, and/or genes are completely or partially deleted as compared to a reference sequence. For example, in some embodiments, an Ad3, 7, 11, 14, 16, 21, 34, 37, or 50 genome can be a genome that does not include one or more of E1, E2, E3, and E4. In certain embodiments, an Ad3, 7, 11, 14, 16, 21, 34, 37, or 50 genome is a genome that does not include any coding sequences of an Ad3, 7, 11, 14, 16, 21, 34, 37, or 50 genome (e.g., a “gutless” vector that includes ITRs having at least 75% sequence identity to Ad3, 7, 11, 14, 16, 21, 34, 37, or 50 genome ITRs but includes none of the coding sequences present in a reference Ad3, 7, 11, 14, 16, 21, 34, 37, or 50 genome).

In various embodiments, an Ad3, 7, 11, 14, 16, 21, 34, 37, or 50 genome includes, does not include, or includes a deletion of, all or a portion of an E1 sequence according to SEQ ID NO: 4, 20, 36, 52, 68, 84, 100, 116, or 132, or a sequence having at least 75% sequence identity (e.g., at least 75%, 80%, 85%, 90%, 95%, 96%, 97%, 98%, 99%, or 100% identity) thereto.

In various embodiments, an Ad3, 7, 11, 14, 16, 21, 34, 37, or 50 genome includes, does not include, or includes a deletion of, all or a portion of an E2 sequence according to SEQ ID NO: 5, 21, 37, 53, 69, 85, 101, 117, or 133, or a sequence having at least 75% sequence identity (e.g., at least 75%, 80%, 85%, 90%, 95%, 96%, 97%, 98%, 99%, or 100% identity) thereto.

In various embodiments, an Ad3, 7, 11, 14, 16, 21, 34, 37, or 50 genome includes, does not include, or includes a deletion of, all or a portion of an E3 sequence according to SEQ ID NO: 4 6, 22, 38, 54, 70, 86, 102, 118, or 134, or a sequence having at least 75% sequence identity (e.g., at least 75%, 80%, 85%, 90%, 95%, 96%, 97%, 98%, 99%, or 100% identity) thereto.

In various embodiments, an Ad3, 7, 11, 14, 16, 21, 34, 37, or 50 genome includes, or does not include, a sequence that encodes a fiber, wherein the sequence has at least 75% sequence identity (e.g., at least 75%, 80%, 85%, 90%, 95%, 96%, 97%, 98%, 99%, or 100% identity) to SEQ ID NO: 7, 23, 39, 55, 71, 87, 103, 119, or 135.

In various embodiments, an Ad3, 7, 11, 14, 16, 21, 34, 37, or 50 genome includes, or does not include, a sequence that encodes a fiber shaft, wherein the sequence has at least 75% sequence identity (e.g., at least 75%, 80%, 85%, 90%, 95%, 96%, 97%, 98%, 99%, or 100% identity) to SEQ ID NO: 8, 24, 40, 56, 72, 88, 104, 120, or 136.

In various embodiments, an Ad3, 7, 11, 14, 16, 21, 34, 37, or 50 genome includes, or does not include, a sequence that encodes a fiber knob, wherein the sequence has at least 75% sequence identity (e.g., at least 75%, 80%, 85%, 90%, 95%, 96%, 97%, 98%, 99%, or 100% identity) to SEQ ID NO: 9, 25, 41, 57, 73, 89, 105, 121, or 137.

In various embodiments, an Ad3, 7, 11, 14, 16, 21, 34, 37, or 50 genome includes, or does not include, a sequence that encodes a fiber tail, wherein the sequence has at least 75% sequence identity (e.g., at least 75%, 80%, 85%, 90%, 95%, 96%, 97%, 98%, 99%, or 100% identity) to a fiber tail of SEQ ID NO: 7, 23, 39, 55, 71, 87, 103, 119, or 135 (e.g., to the portion of the fiber sequence that includes all nucleotides 5′ of the sequence encoding the fiber shaft and/or that includes all nucleotides encoding the portion of the fiber N-terminal to the fiber shaft).

In various embodiments, an Ad3, 7, 11, 14, 16, 21, 34, 37, or 50 genome includes, or does not include, a sequence that encodes a penton, wherein the sequence has at least 75% sequence identity (e.g., at least 75%, 80%, 85%, 90%, 95%, 96%, 97%, 98%, 99%, or 100% identity) to SEQ ID NO: 10, 26, 42, 58, 74, 90, 106, 122, or 138.

In various embodiments, an Ad3, 7, 11, 14, 16, 21, 34, 37, or 50 genome includes, or does not include, a sequence that encodes a hexon, wherein the sequence has at least 75% sequence identity (e.g., at least 75%, 80%, 85%, 90%, 95%, 96%, 97%, 98%, 99%, or 100% identity) to SEQ ID NO: 11, 27, 43, 59, 75, 91, 107, 123, or 139.

The present disclosure includes Ad3, 7, 11, 14, 16, 21, 34, 37, or 50 vectors that include a fiber having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber (e.g., a fiber according to SEQ ID NO: 12, 28, 44, 60, 76, 92, 108, 124, or 140).

The present disclosure includes Ad3, 7, 11, 14, 16, 21, 34, 37, or 50 vectors that include a fiber tail having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber tail (e.g., a fiber tail of a fiber according to SEQ ID NO: 12, 28, 44, 60, 76, 92, 108, 124, or 140, e.g., where the fiber tail is the portion of the fiber including all amino acids N-terminal to the fiber shaft).

The present disclosure includes Ad3, 7, 11, 14, 16, 21, 34, 37, or 50 vectors that include a fiber shaft having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber shaft (e.g., a fiber shaft according to SEQ ID NO: 13, 29, 45, 61, 77, 93, 109, 125, or 141).

The present disclosure includes Ad3, 7, 11, 14, 16, 21, 34, 37, or 50 vectors that include a fiber knob having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber knob (e.g., a fiber knob according to SEQ ID NO: 14, 30, 46, 62, 78, 94, 110, 126, or 142).

The present disclosure includes Ad3, 7, 11, 14, 16, 21, 34, 37, or 50 vectors that include a penton having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 penton (e.g., a penton according to SEQ ID NO: 15, 31, 47, 63, 79, 95, 111, 127, or 143).

The present disclosure includes Ad3, 7, 11, 14, 16, 21, 34, 37, or 50 vectors that include a hexon having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 hexon (e.g., a hexon according to SEQ ID NO: 16, 32, 48, 64, 80, 96, 112, 128, or 144).

In various embodiments, an Ad3, 7, 11, 14, 16, 21, 34, 37, or 50 vector is any adenoviral vector that includes at least a fiber having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber (e.g., a fiber according to SEQ ID NO: 12, 28, 44, 60, 76, 92, 108, 124, or 140).

In various embodiments, an Ad3, 7, 11, 14, 16, 21, 34, 37, or 50 vector is any adenoviral vector that includes at least a fiber tail having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber tail (e.g., a fiber tail of a fiber according to SEQ ID NO: 12, 28, 44, 60, 76, 92, 108, 124, or 140, e.g., where the fiber tail is the portion of the fiber including all amino acids N-terminal to the fiber shaft).

In various embodiments, an Ad3, 7, 11, 14, 16, 21, 34, 37, or 50 vector is any adenoviral vector that includes at least a fiber shaft having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber shaft (e.g., a fiber shaft according to SEQ ID NO: 13, 29, 45, 61, 77, 93, 109, 125, or 141).

In various embodiments, an Ad3, 7, 11, 14, 16, 21, 34, 37, or 50 vector is any adenoviral vector that includes at least a fiber knob having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber knob (e.g., a fiber knob according to SEQ ID NO: 14, 30, 46, 62, 78, 94, 110, 126, or 142).

In various embodiments, an Ad3, 7, 11, 14, 16, 21, 34, 37, or 50 vector is any adenoviral vector that includes at least a penton having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 penton (e.g., a penton according to SEQ ID NO: 15, 31, 47, 63, 79, 95, 111, 127, or 143).

In various embodiments, an Ad3, 7, 11, 14, 16, 21, 34, 37, or 50 vector is any adenoviral vector that includes at least a hexon having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 hexon (e.g., a hexon according to SEQ ID NO: 16, 32, 48, 64, 80, 96, 112, 128, 144).

Thus, an Ad3, 7, 11, 14, 16, 21, 34, 37, or 50 vector can be a chimeric adenoviral vector that includes at least a fiber knob having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber knob and at least one protein or portion thereof (such as a fiber shaft, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 7, 11, 14, 16, 21, 34, 37, or 50 vector can be a chimeric adenoviral vector that includes at least a fiber shaft having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber shaft and at least one protein or portion thereof (such as a fiber knob, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 7, 11, 14, 16, 21, 34, 37, or 50 vector can be a chimeric adenoviral vector that includes at least a fiber tail having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 fiber tail and at least one protein or portion thereof (such as a fiber knob, fiber shaft, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 7, 11, 14, 16, 21, 34, 37, or 50 vector can be a chimeric adenoviral vector that includes at least a penton having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 penton and at least one protein or portion thereof (such as a fiber knob, fiber shaft, fiber tail, or hexon) that has at least 75% sequence identity (e.g., at least 75%, 80%, 85%, 90%, 95%, 96%, 97%, 98%, 99%, or 100% identity) to a different adenoviral serotype.

An Ad3, 7, 11, 14, 16, 21, 34, 37, or 50 vector can be a chimeric adenoviral vector that includes at least a hexon having at least 75% sequence identity (e.g., at least 75%, 80%, 85%, 90%, 95%, 96%, 97%, 98%, 99%, or 100% identity) to an Ad3, 7, 11, 14, 16, 21, 34, 37, or 50 hexon and at least one protein or portion thereof (such as a fiber knob, fiber shaft, fiber tail, or penton) that has at least 75% sequence identity (e.g., at least 75%, 80%, 85%, 90%, 95%, 96%, 97%, 98%, 99%, or 100% identity) to a different adenoviral serotype.

Exemplary sequences of Ad3, 7, 11, 14, 16, 21, 34, 37, or 50 components (e.g., ITRs, packaging sequences, genes, and proteins) are provided in the following tables. Viral polypeptides include proteins that are components of viral vectors and portions or fragments thereof, including for example a fiber, fiber knob, fiber shaft, fiber tail, penton, or hexon.

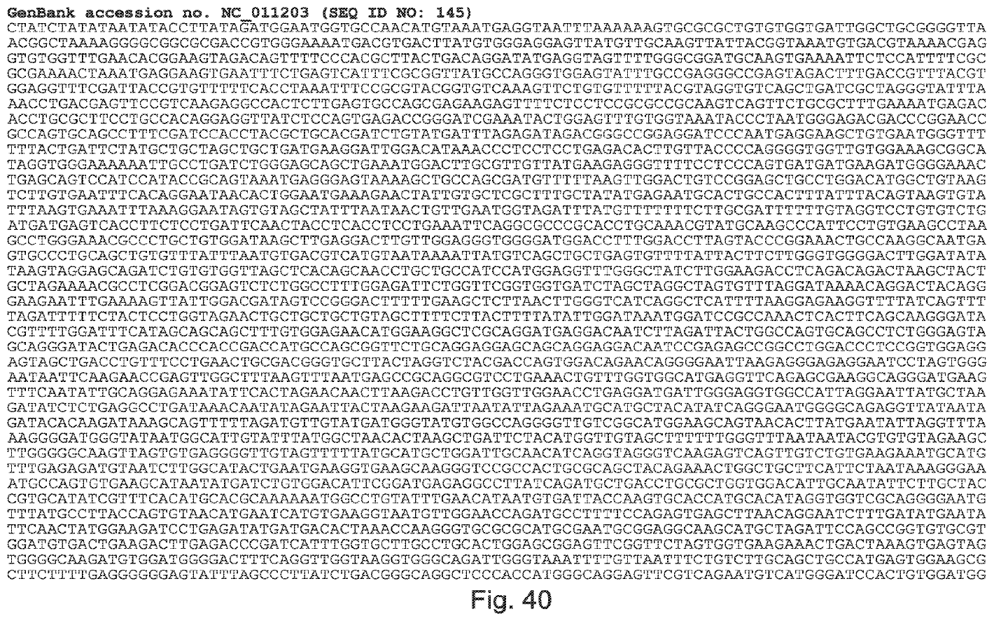

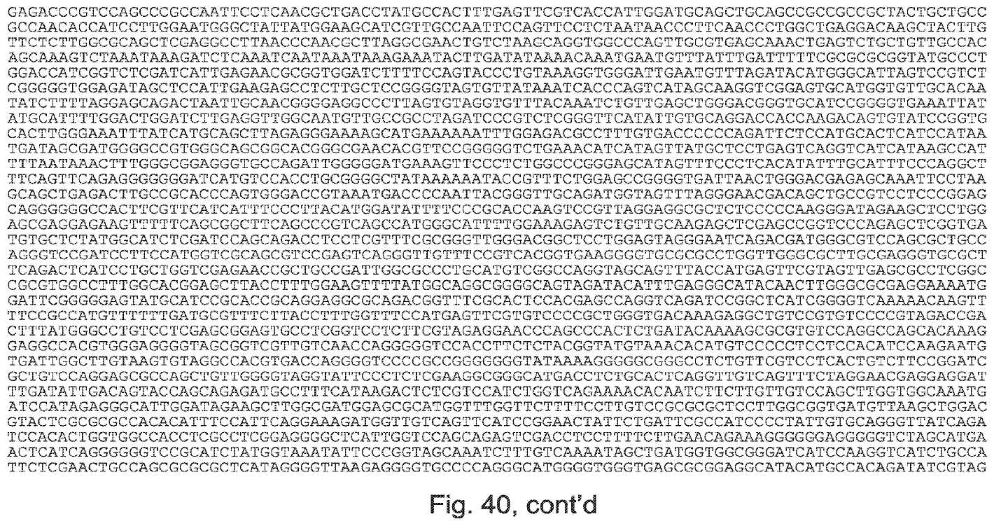

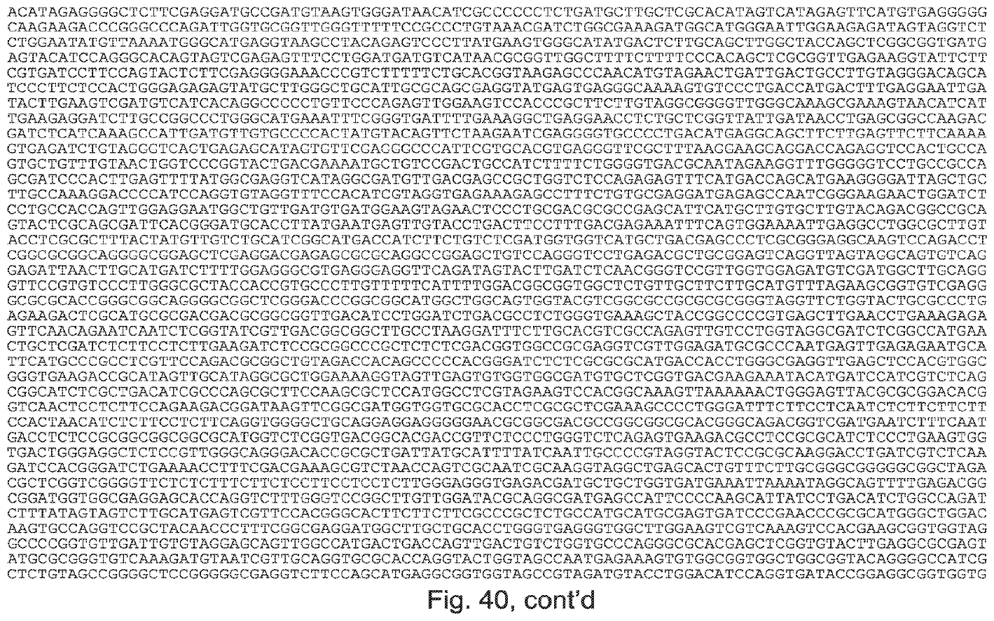

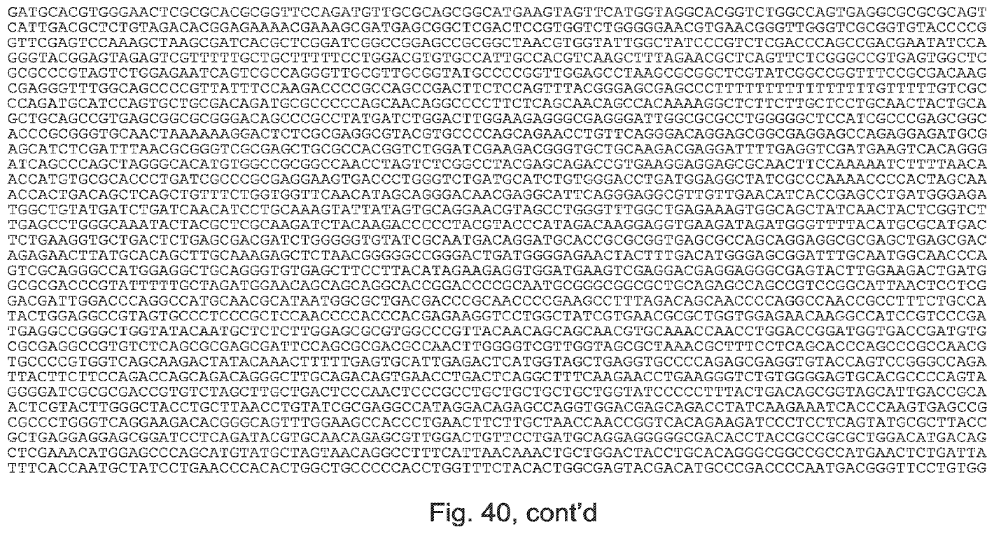

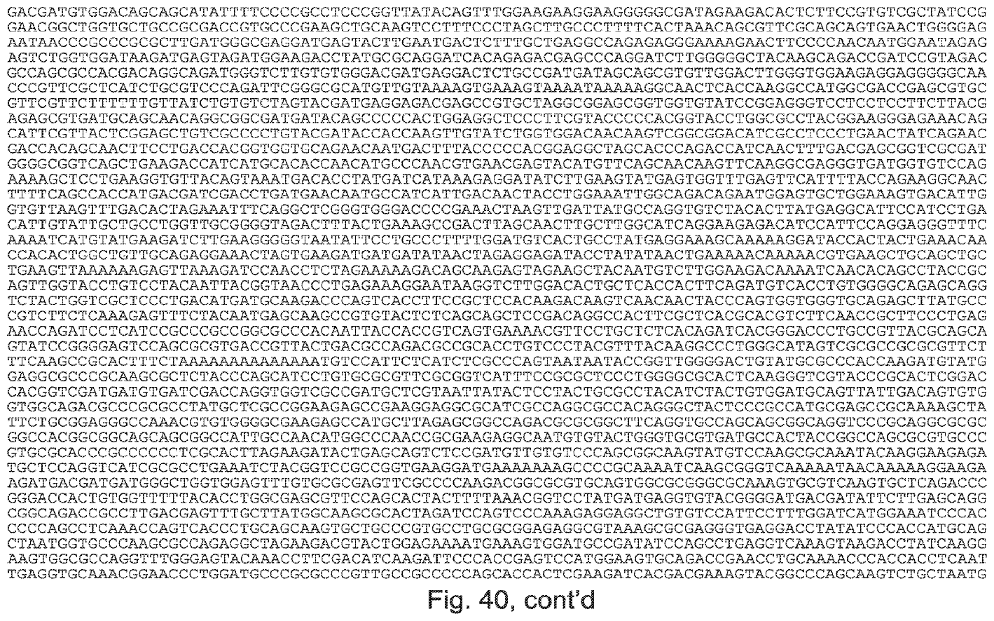

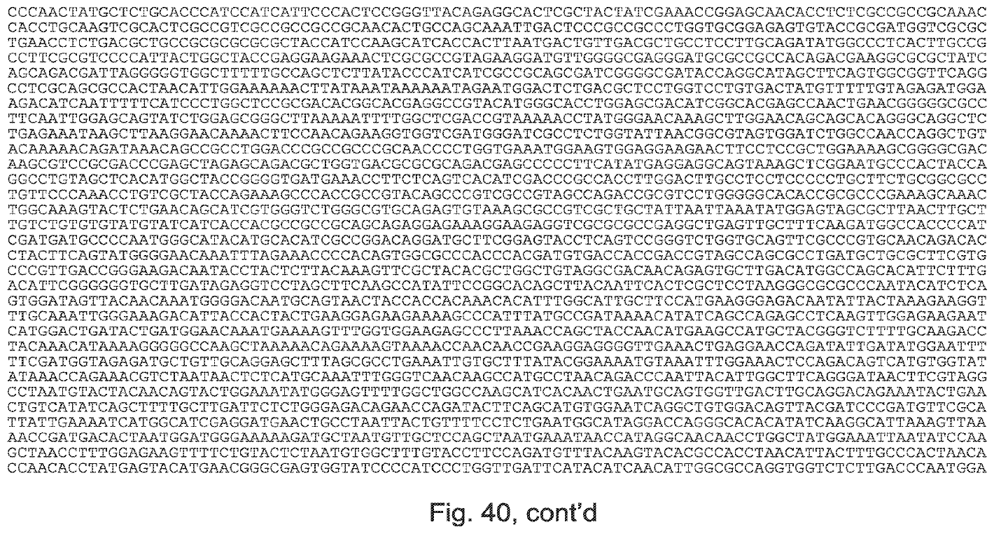

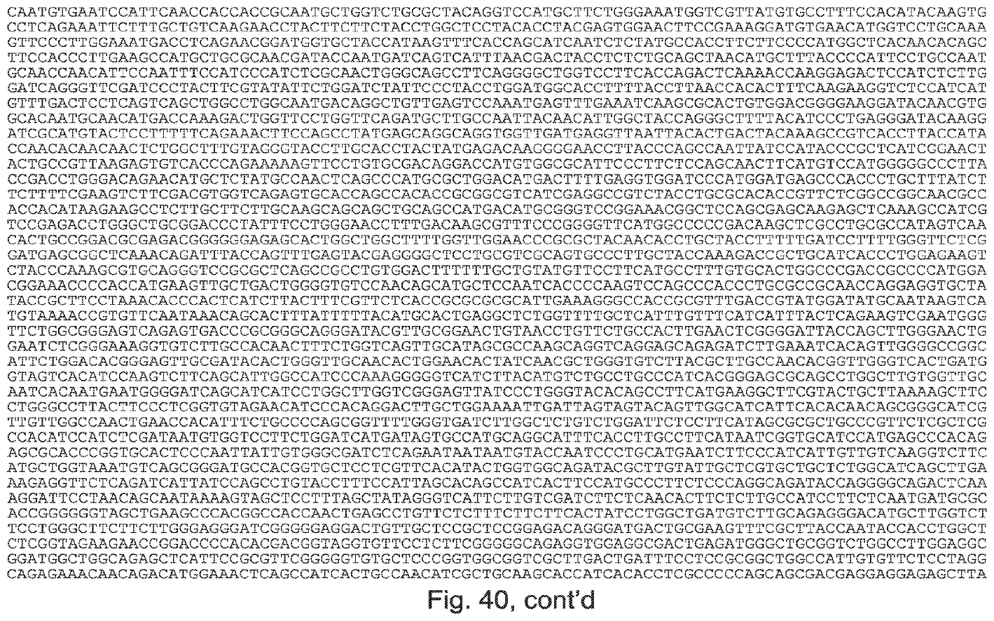

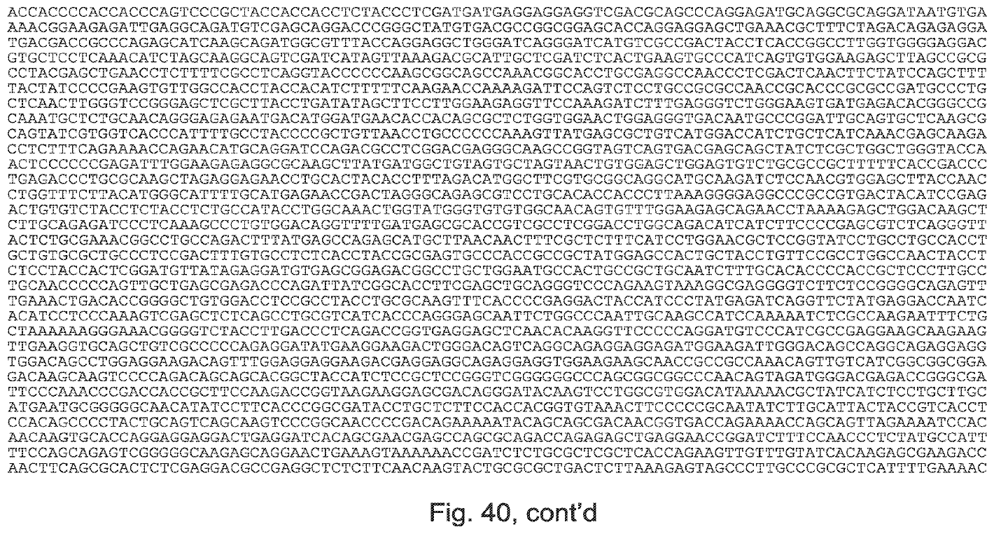

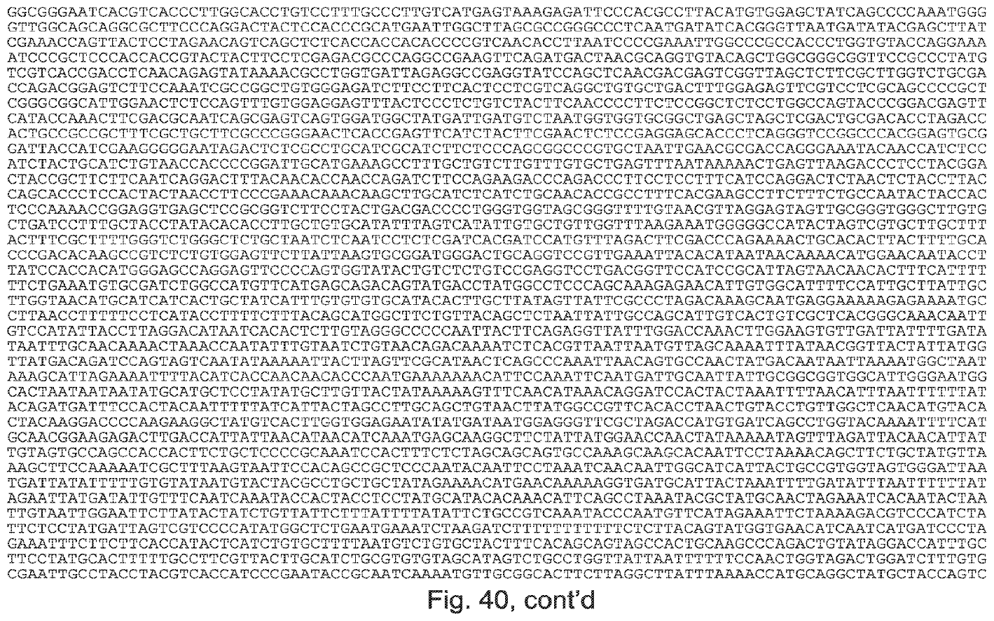

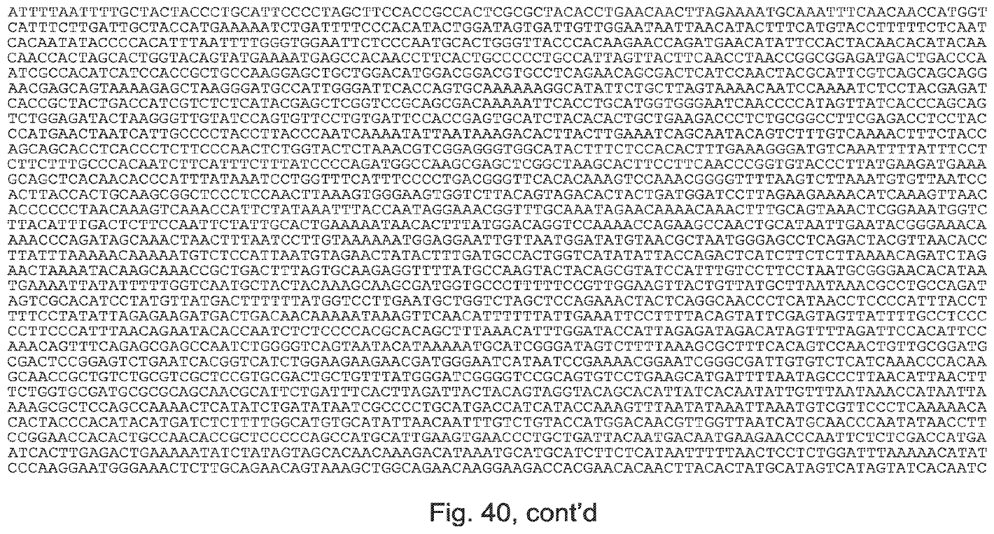

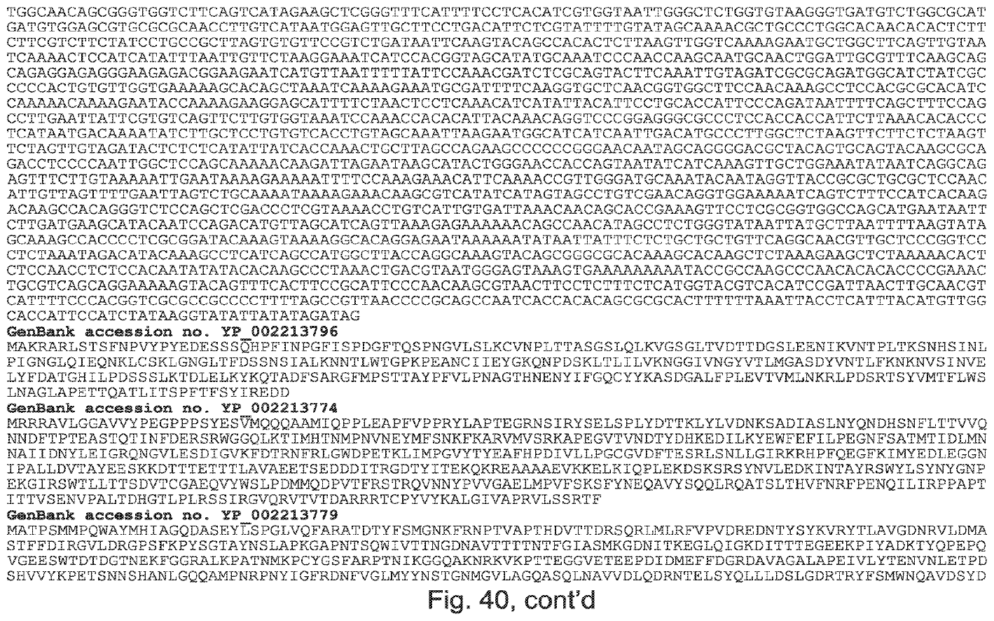

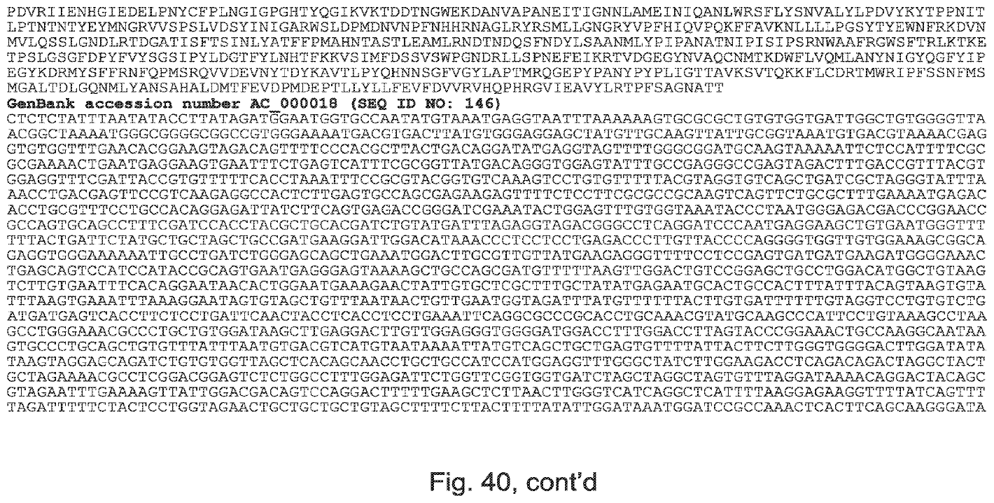

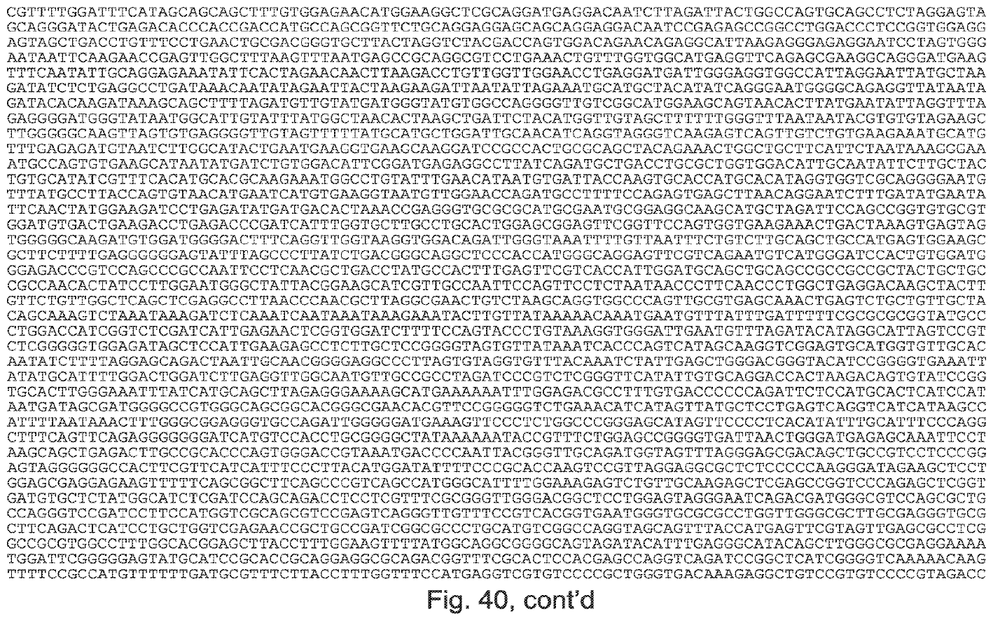

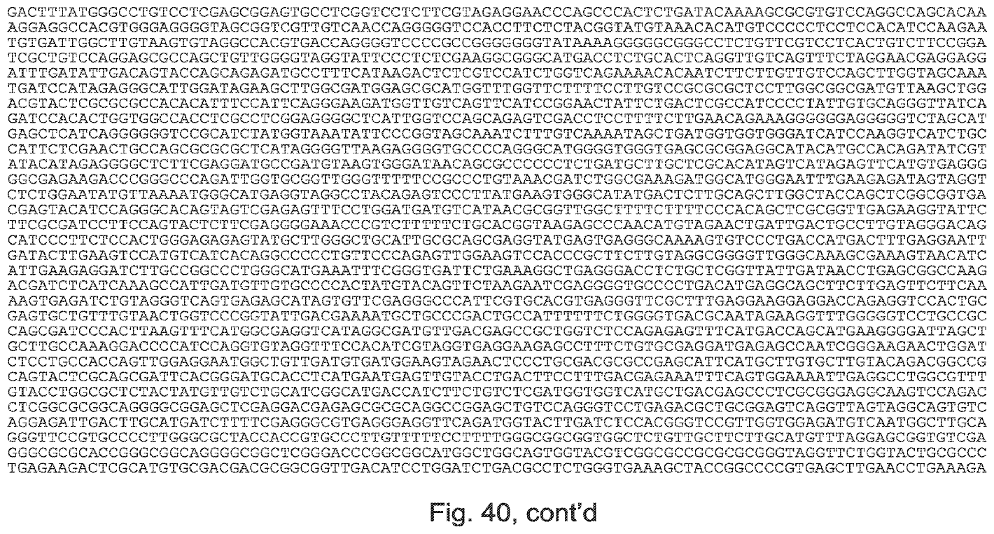

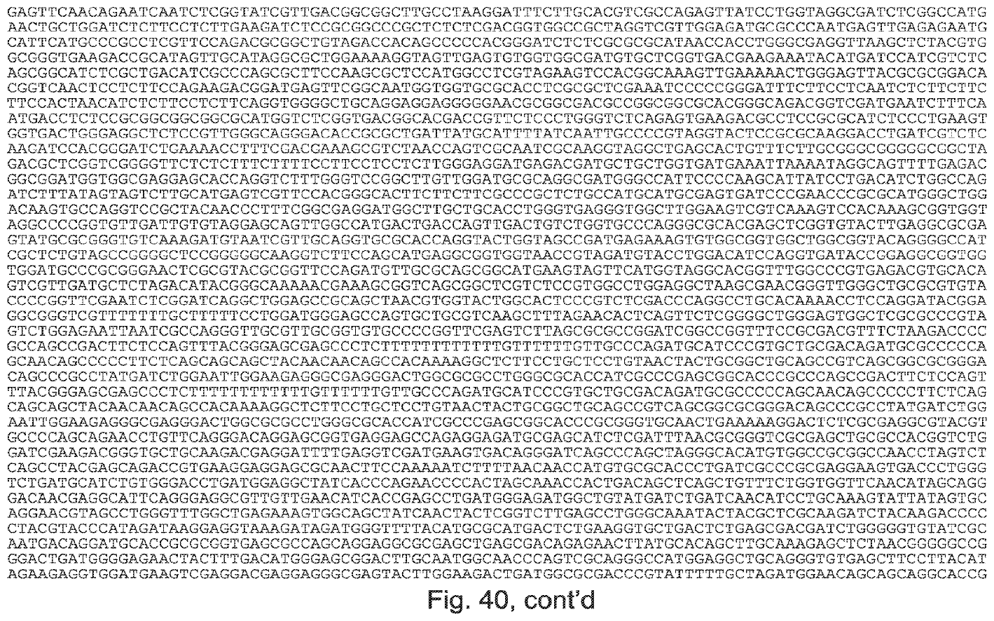

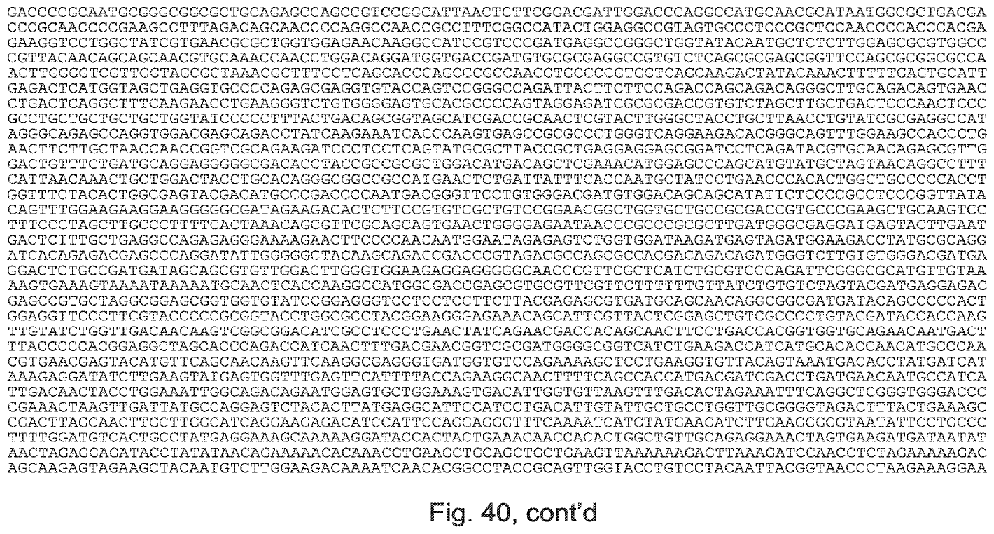

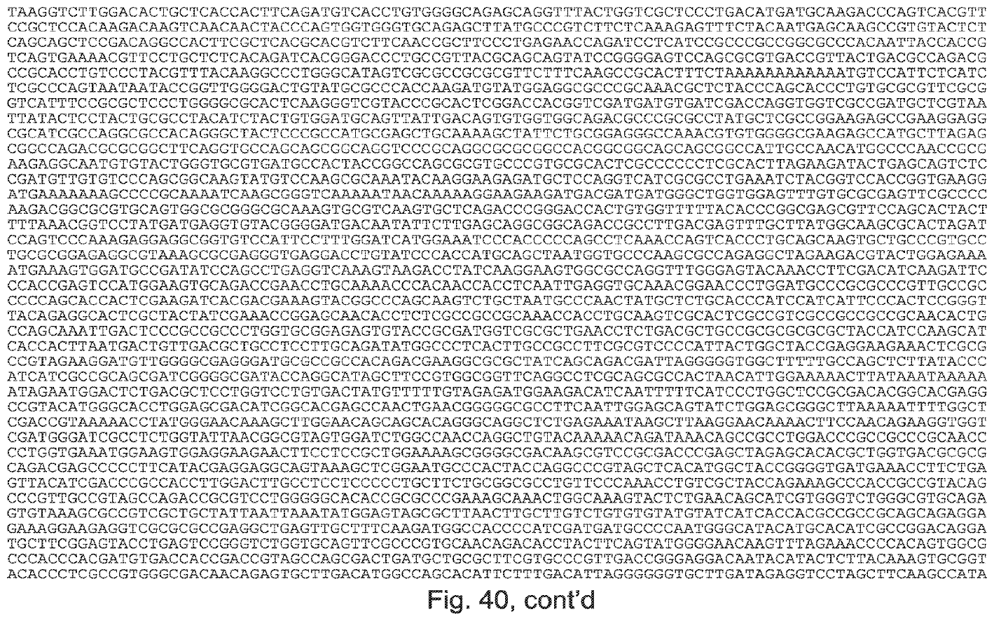

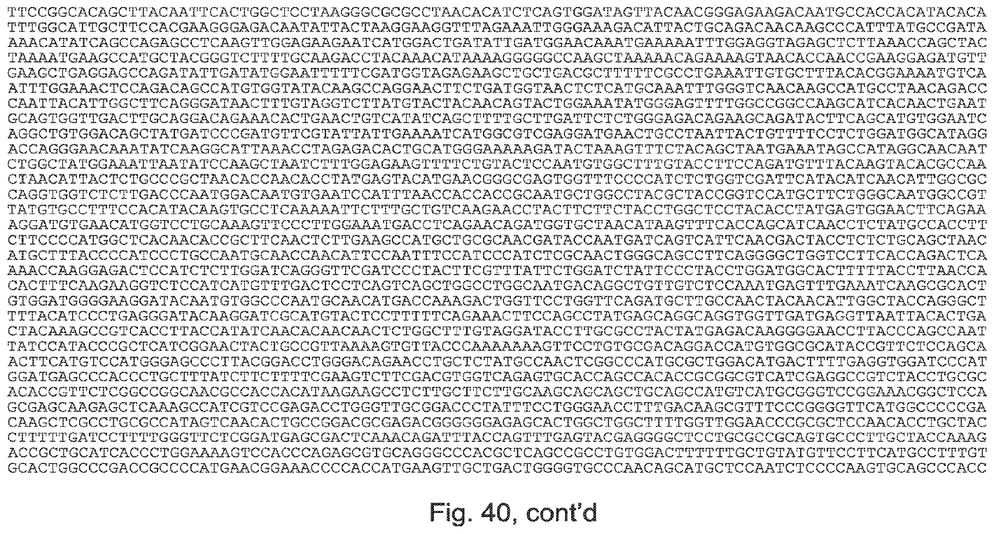

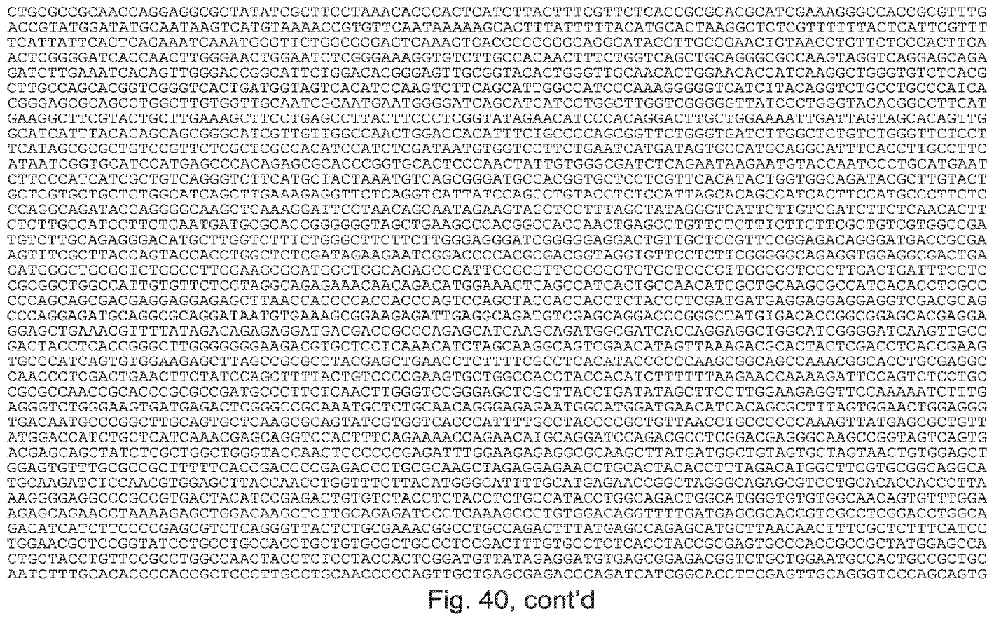

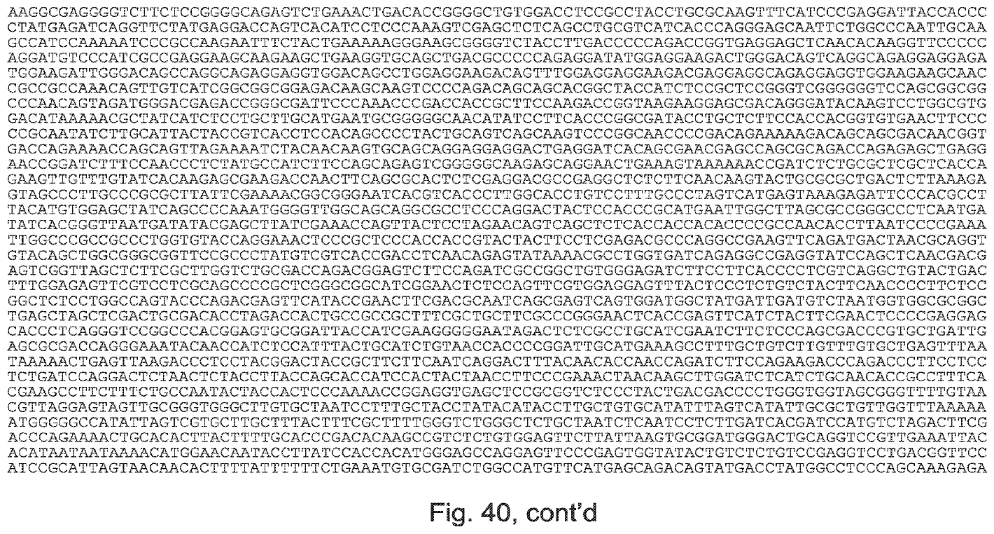

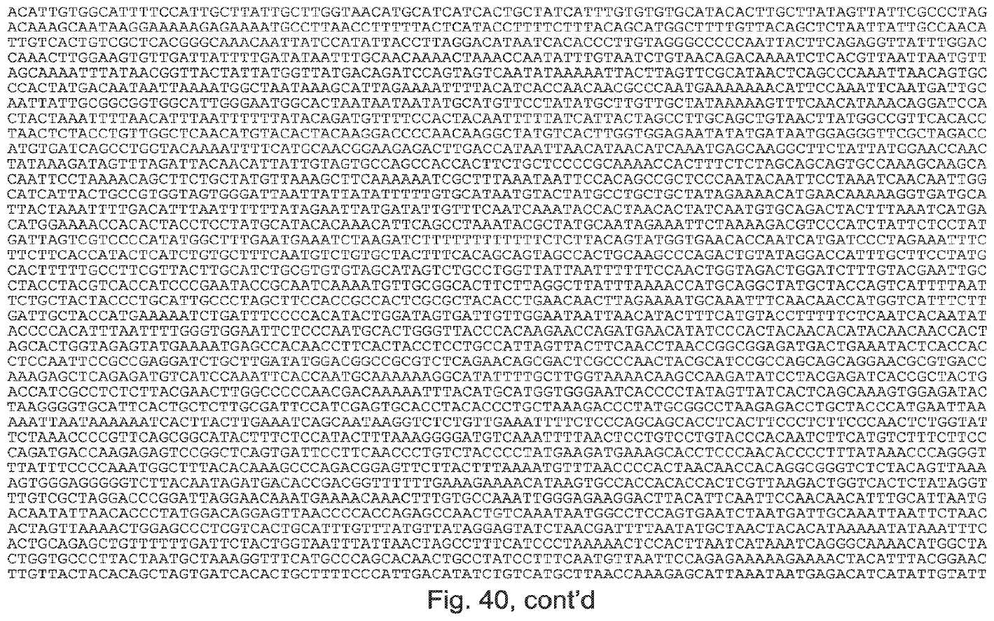

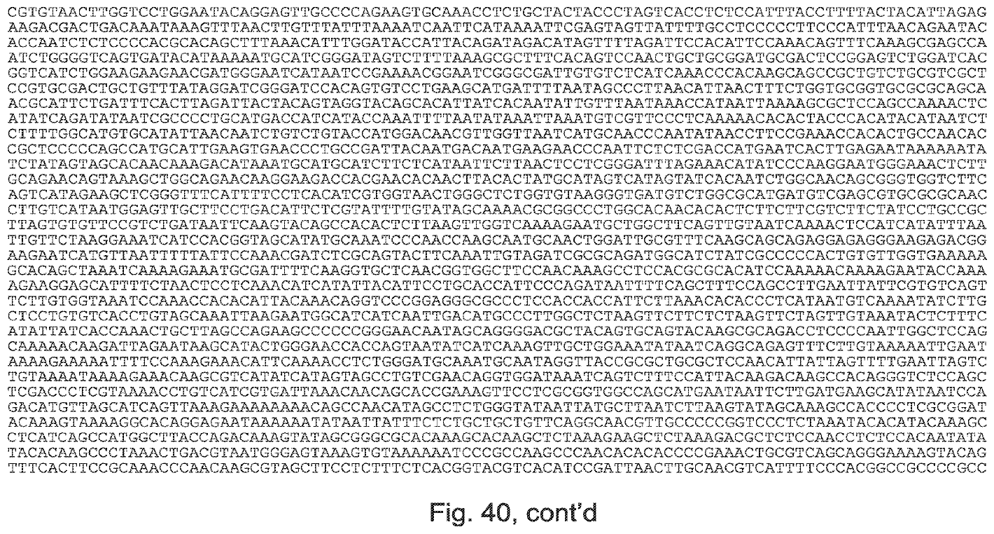

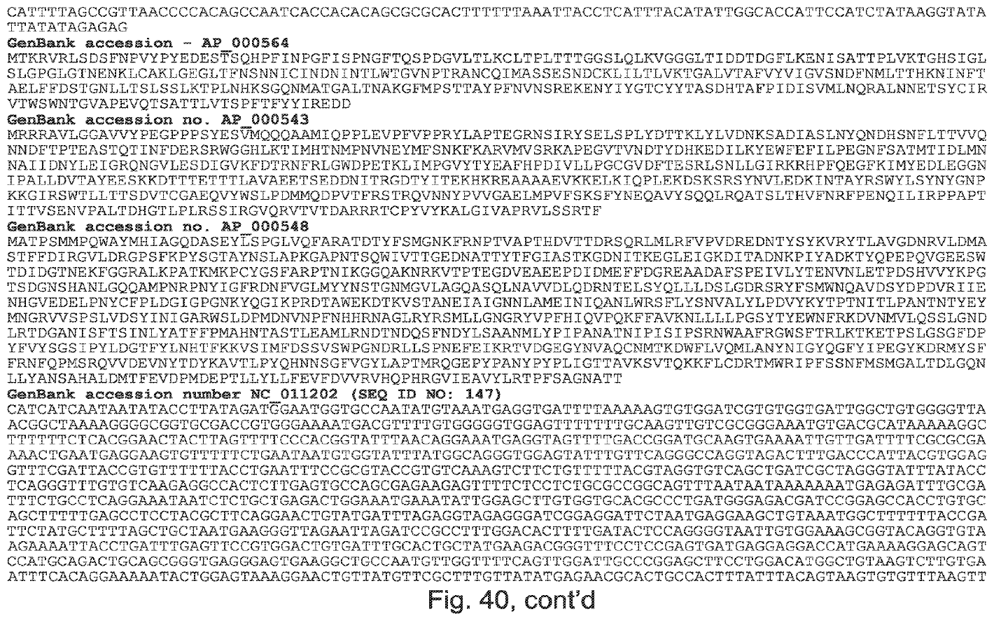

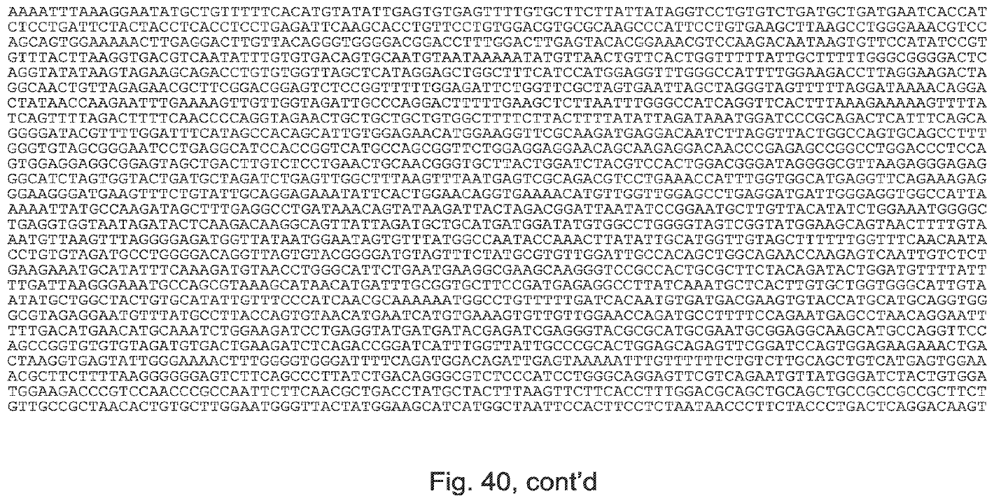

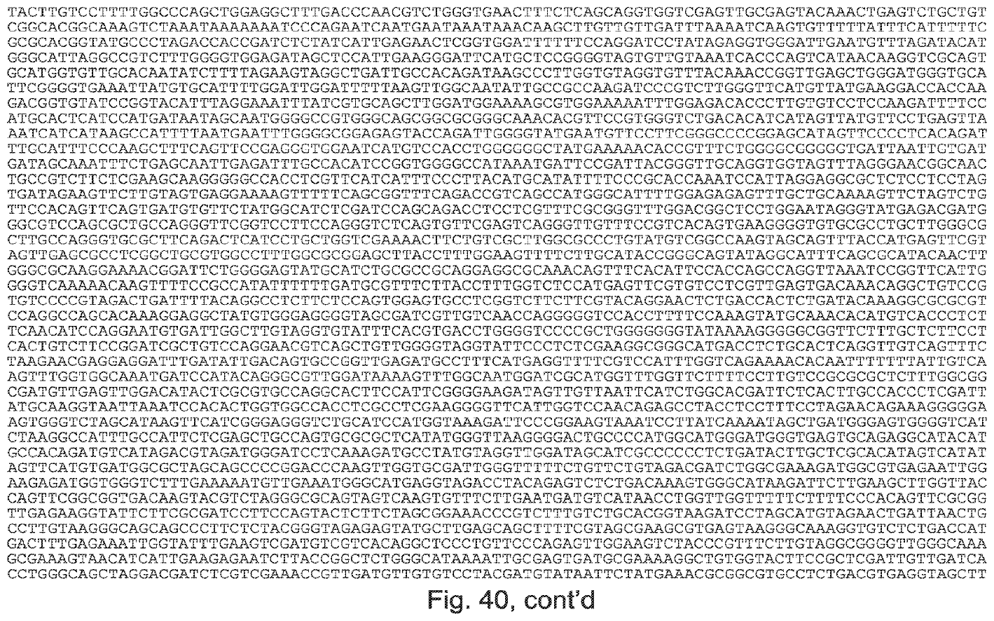

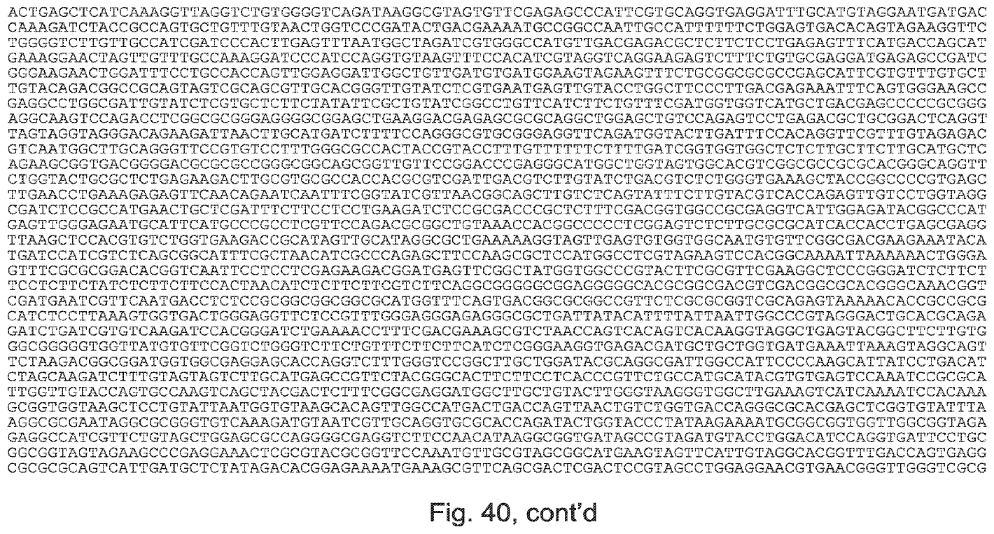

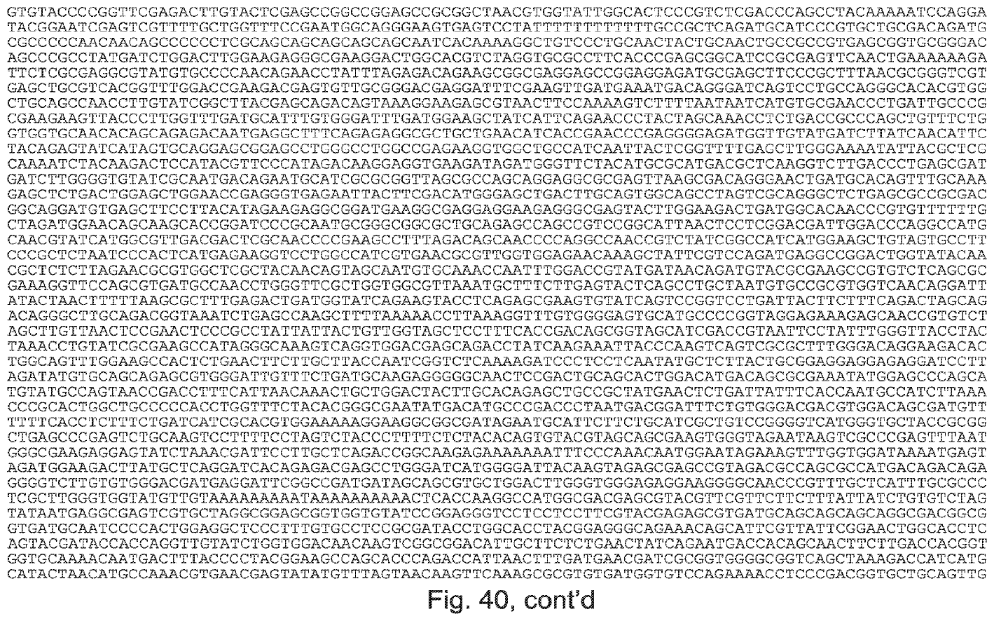

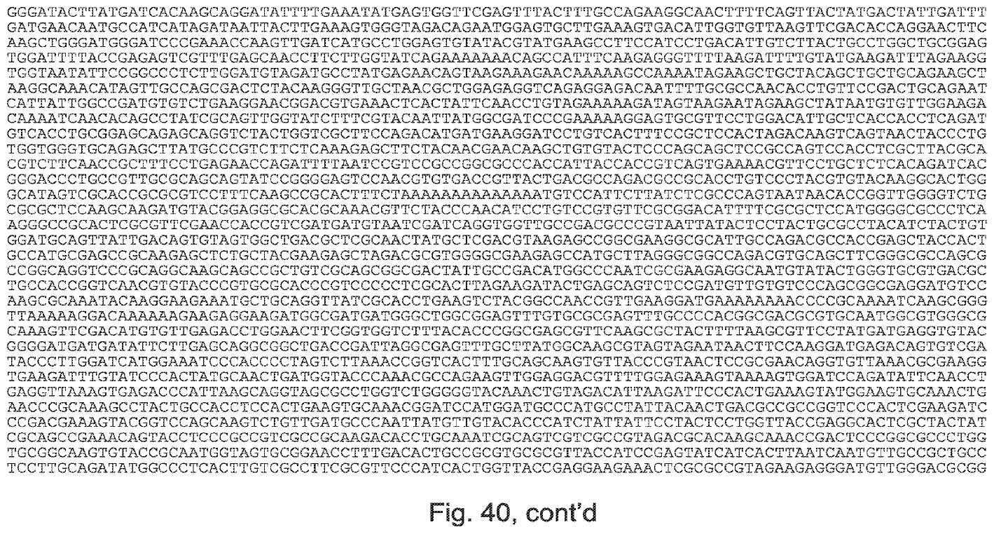

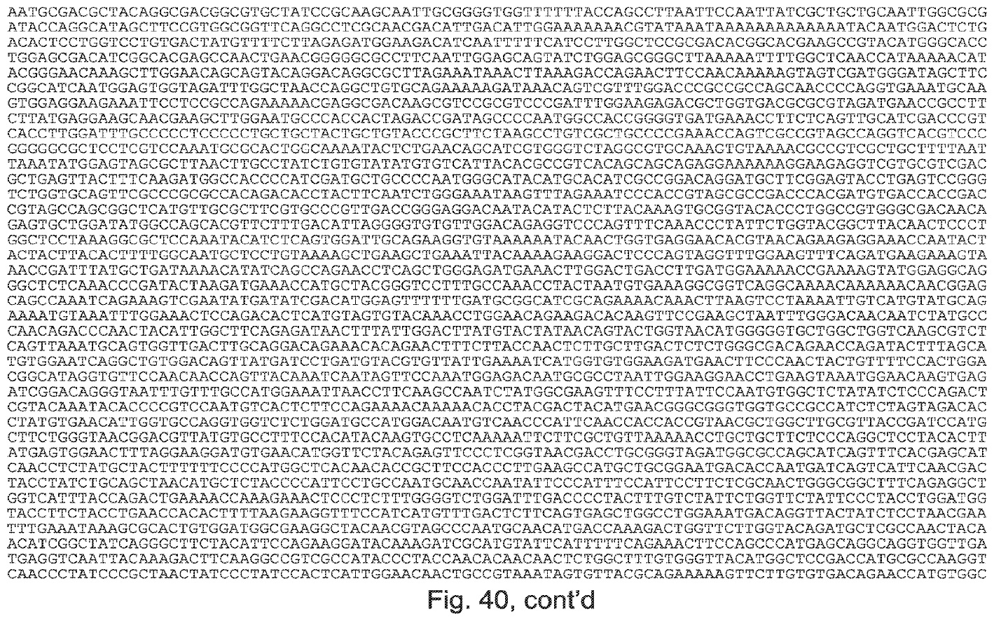

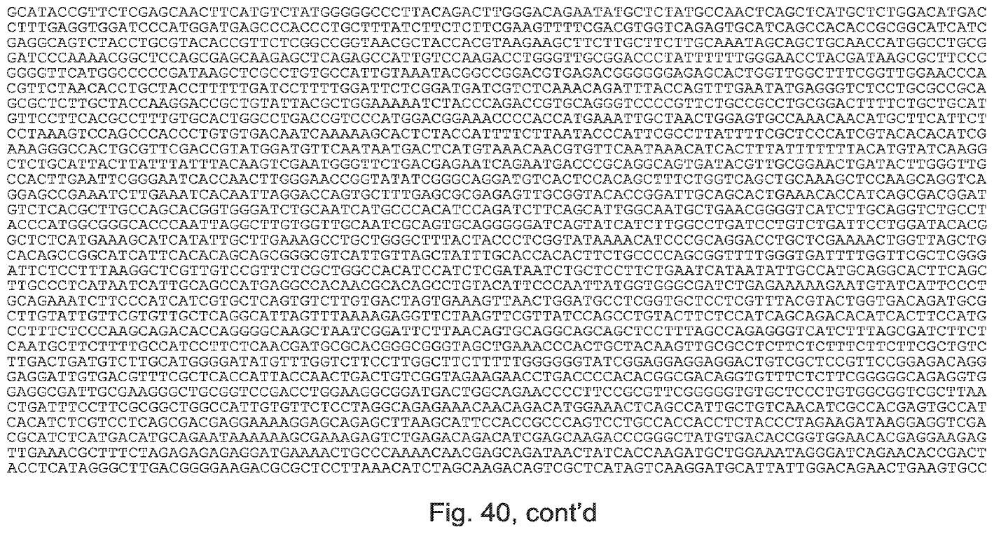

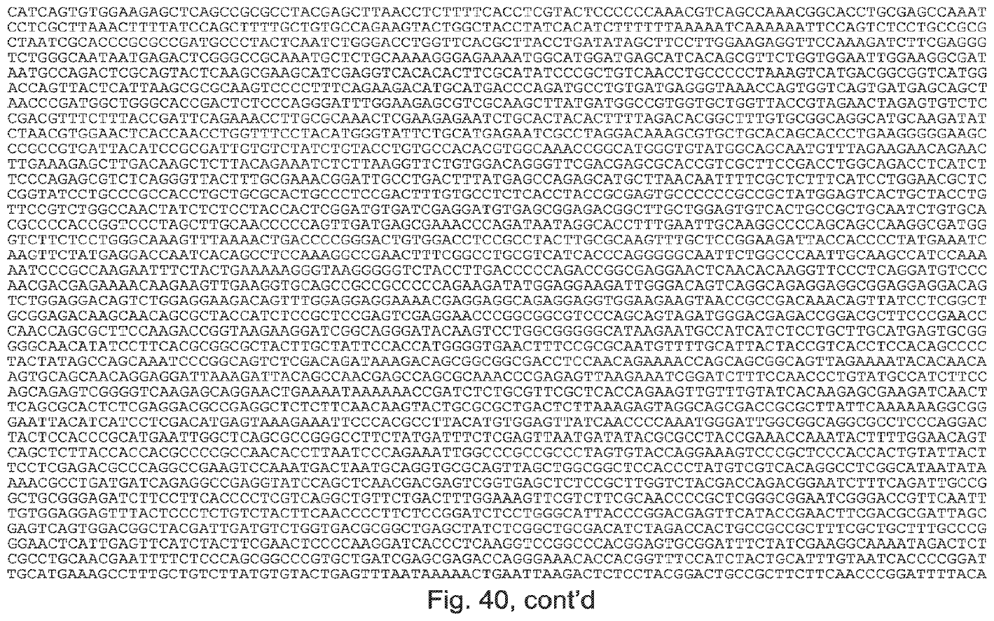

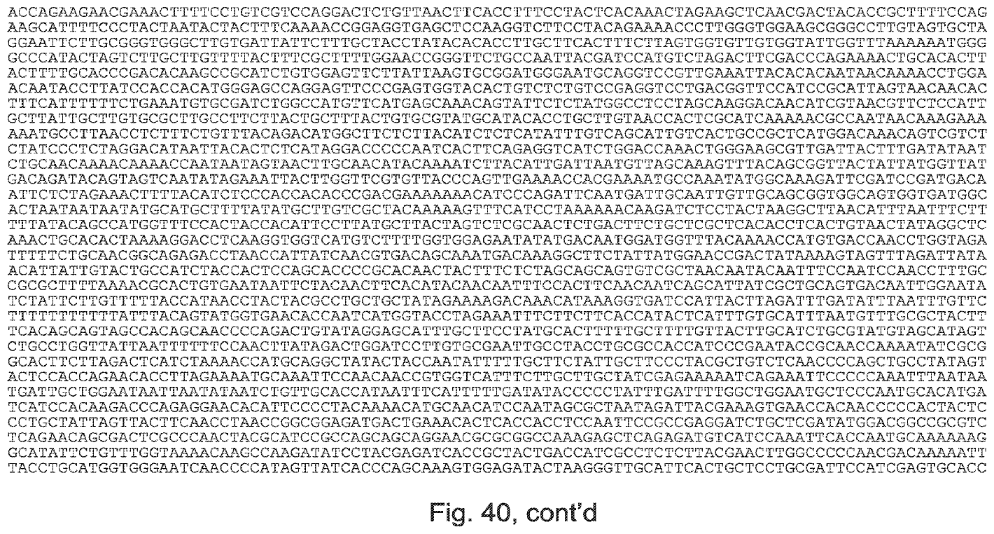

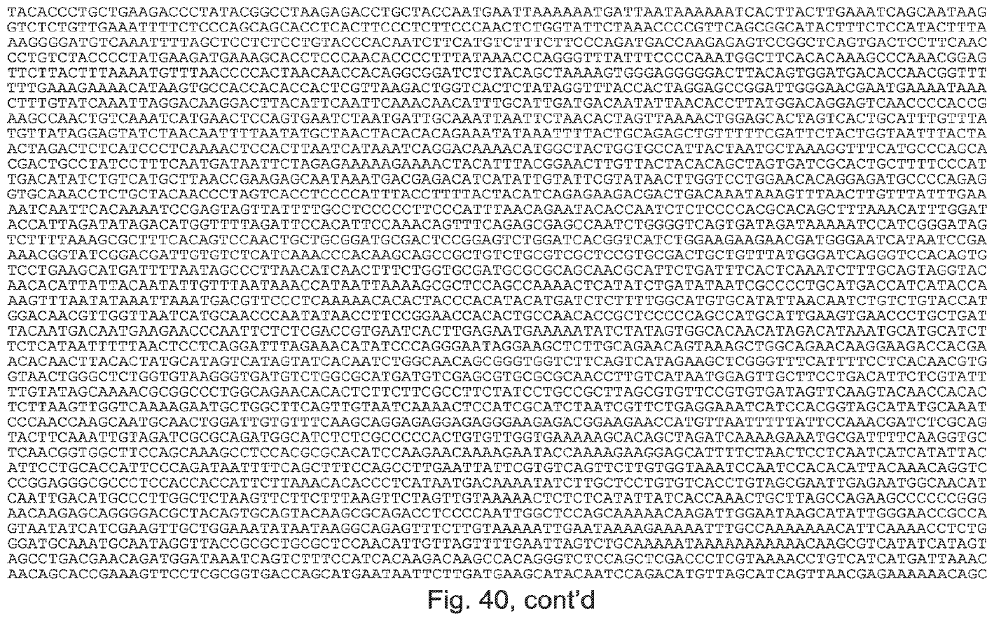

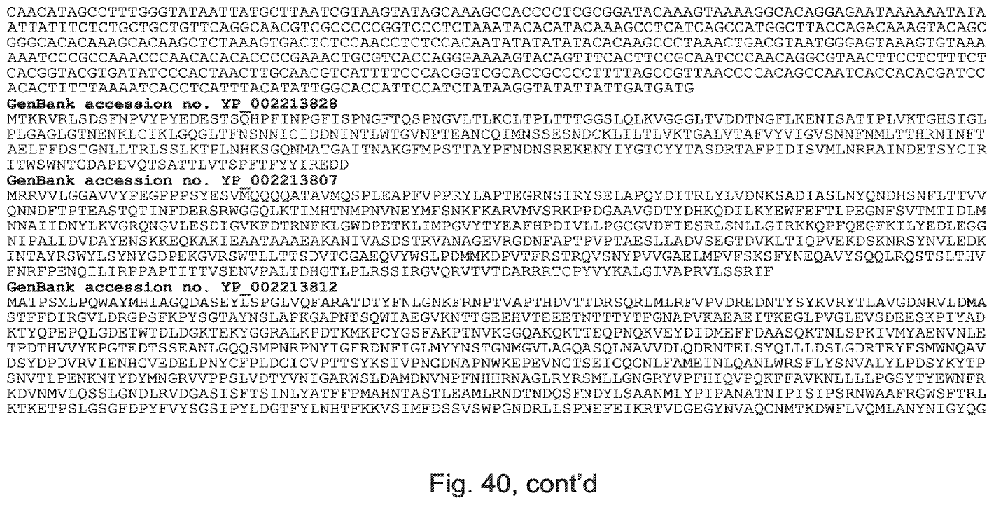

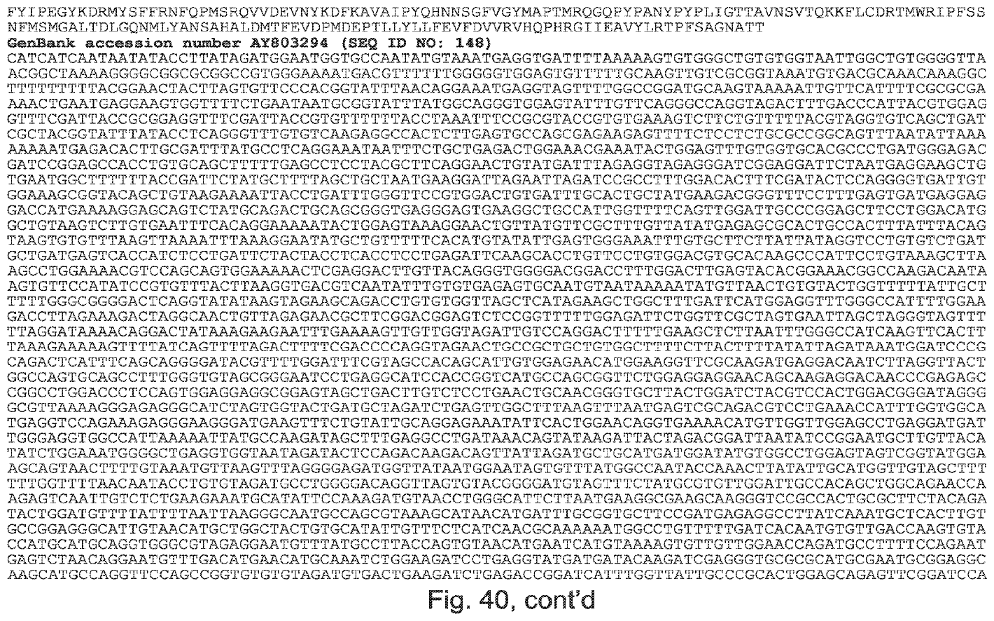

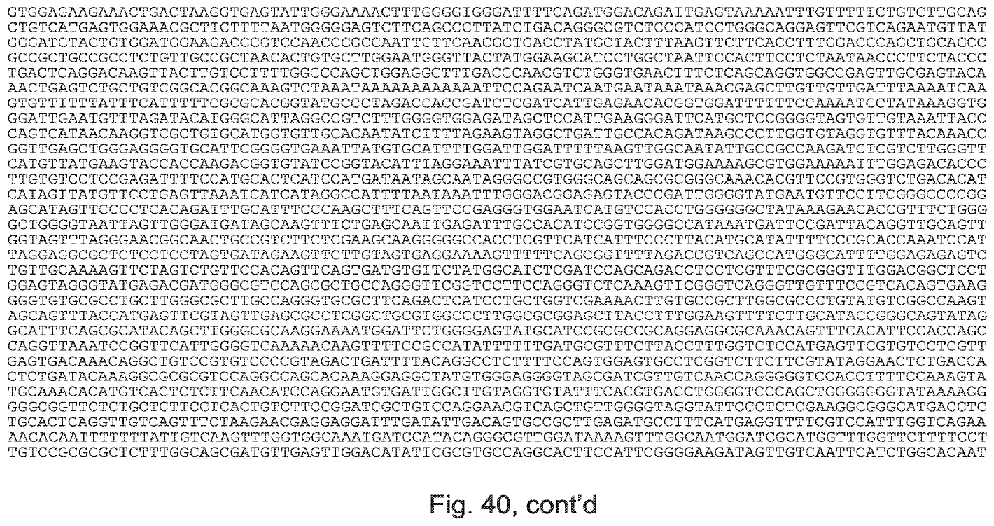

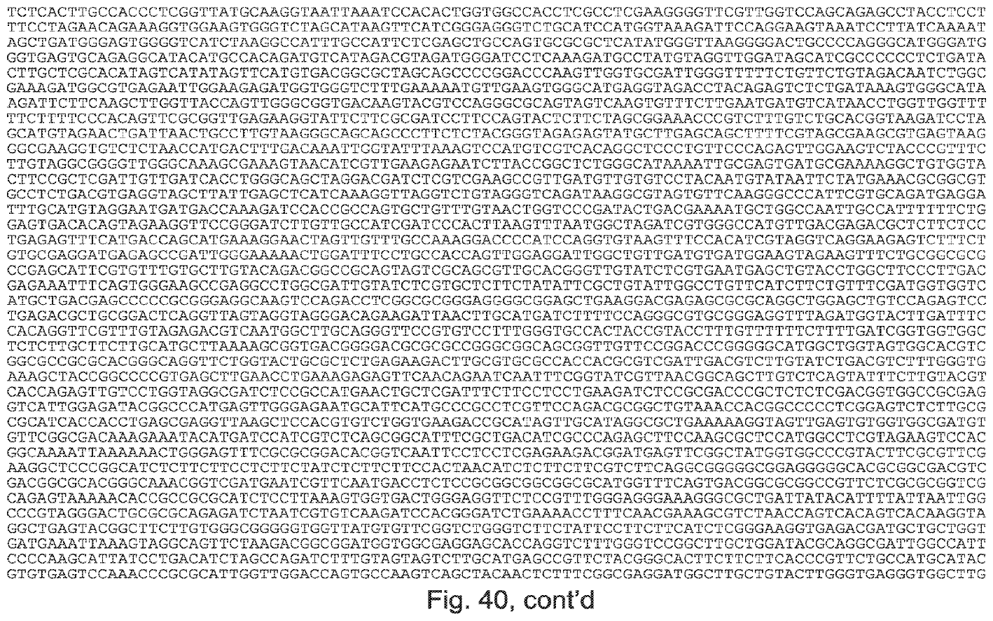

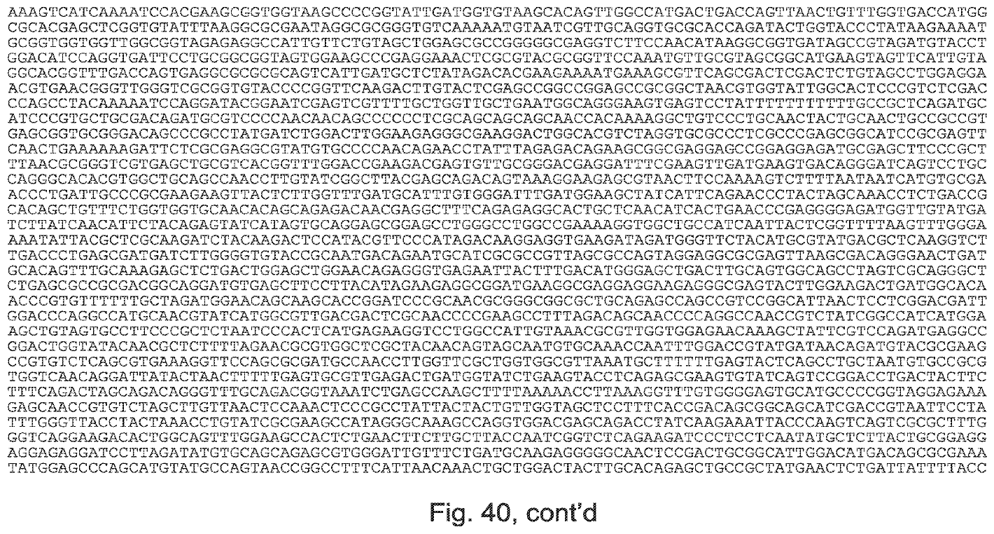

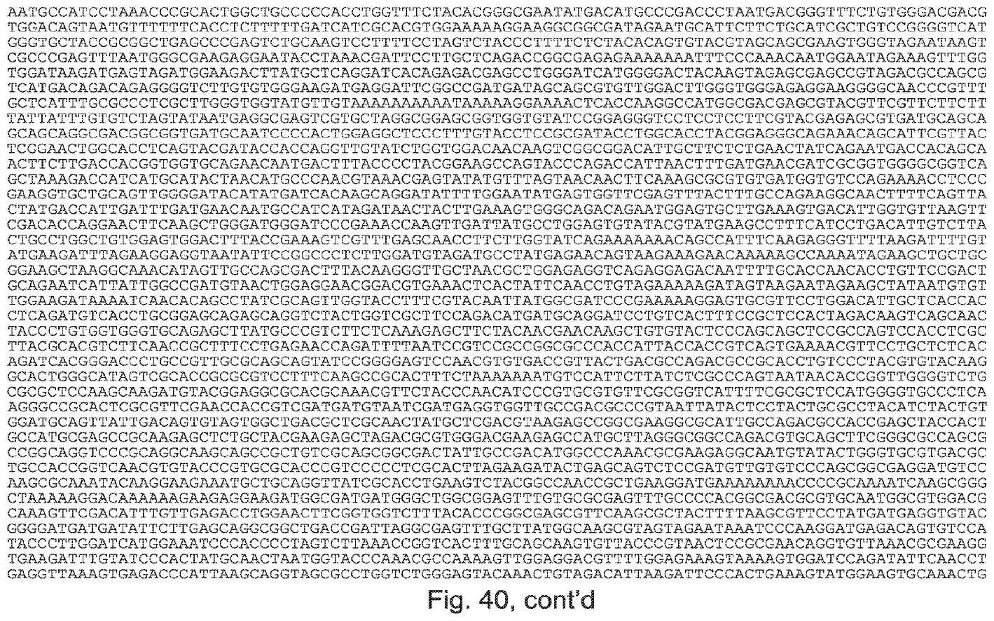

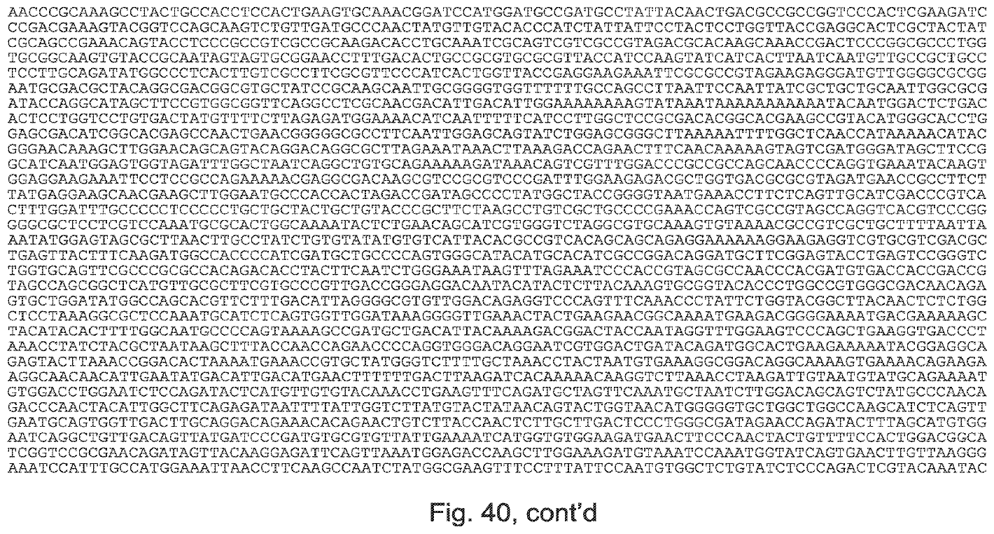

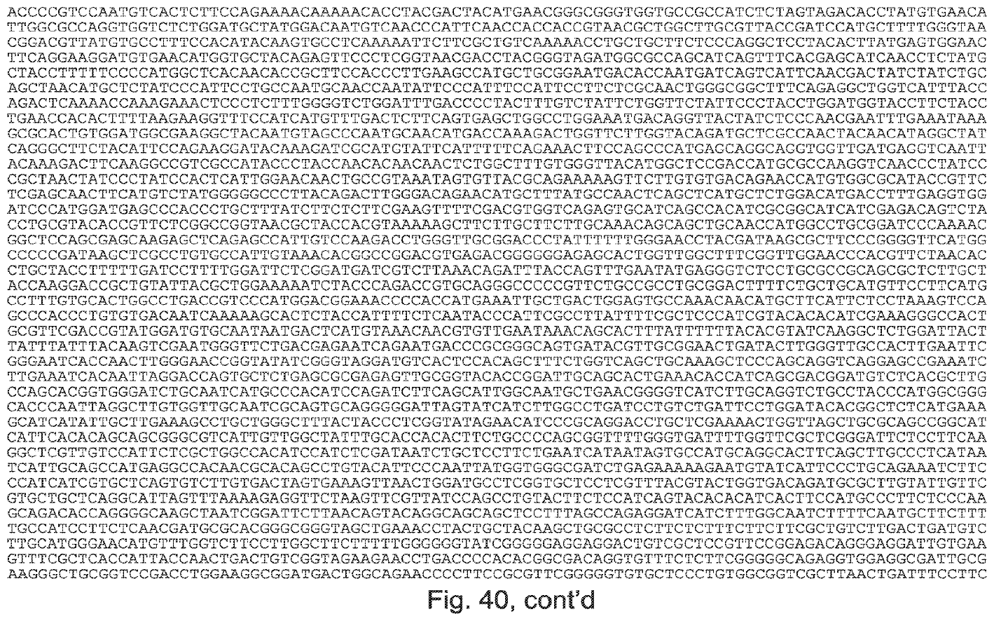

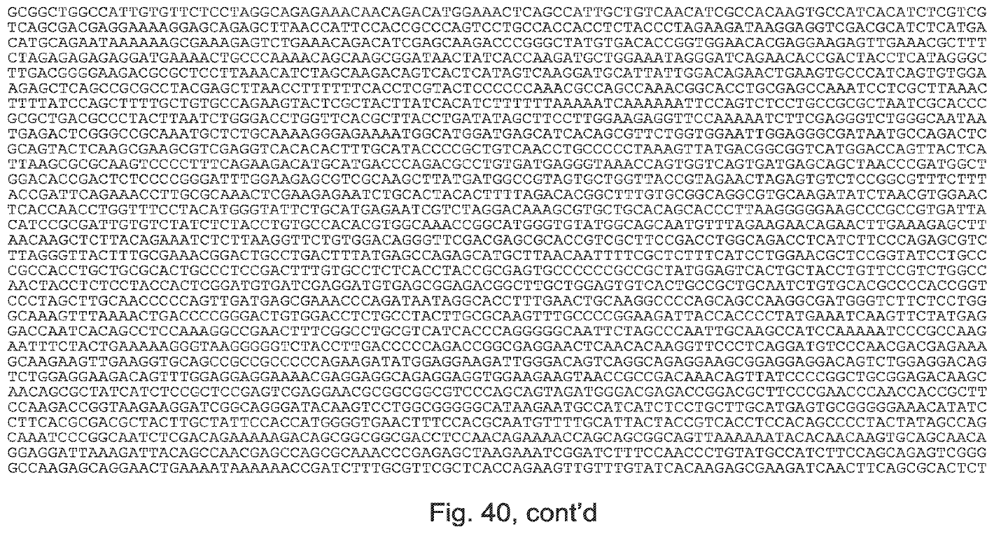

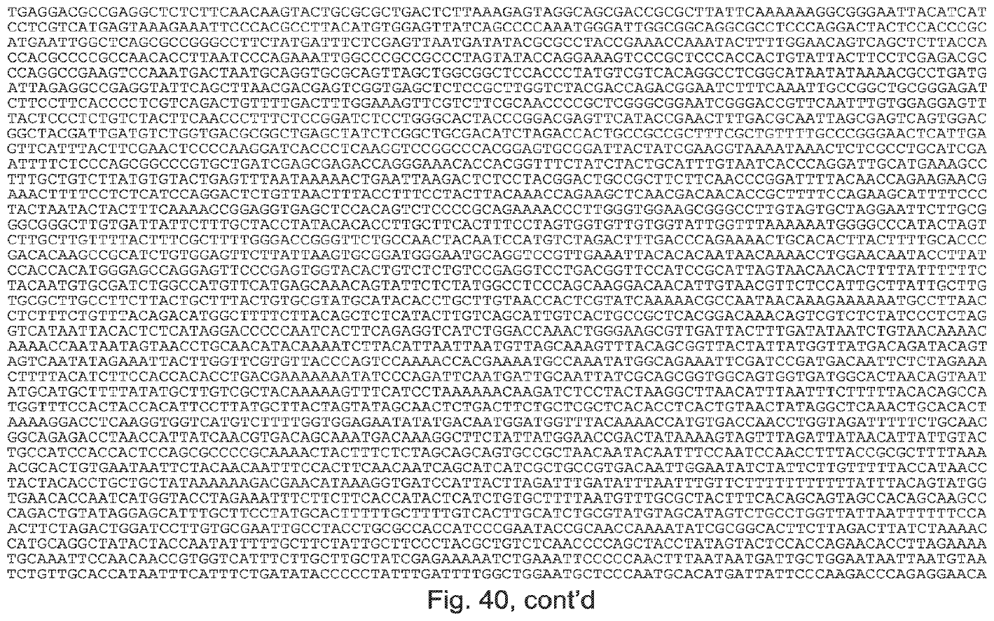

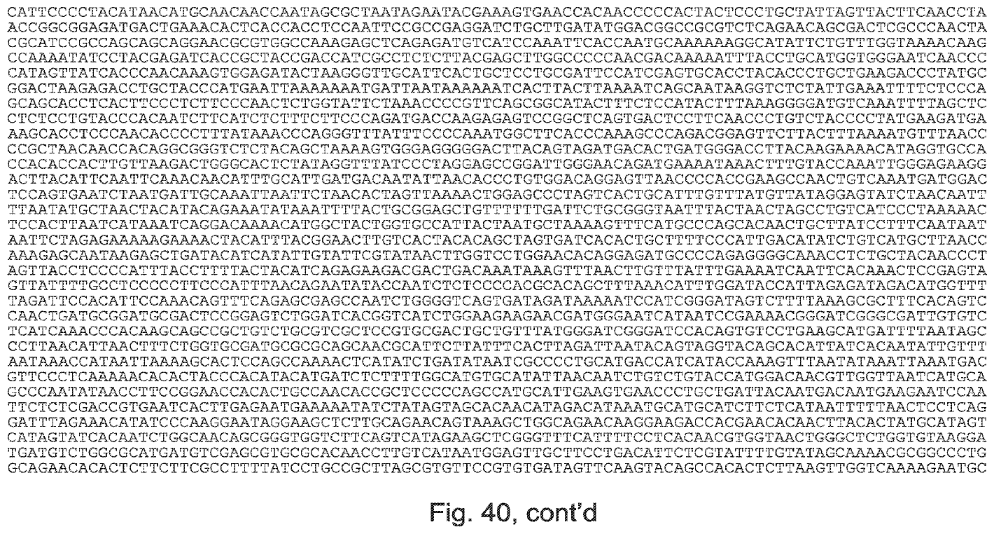

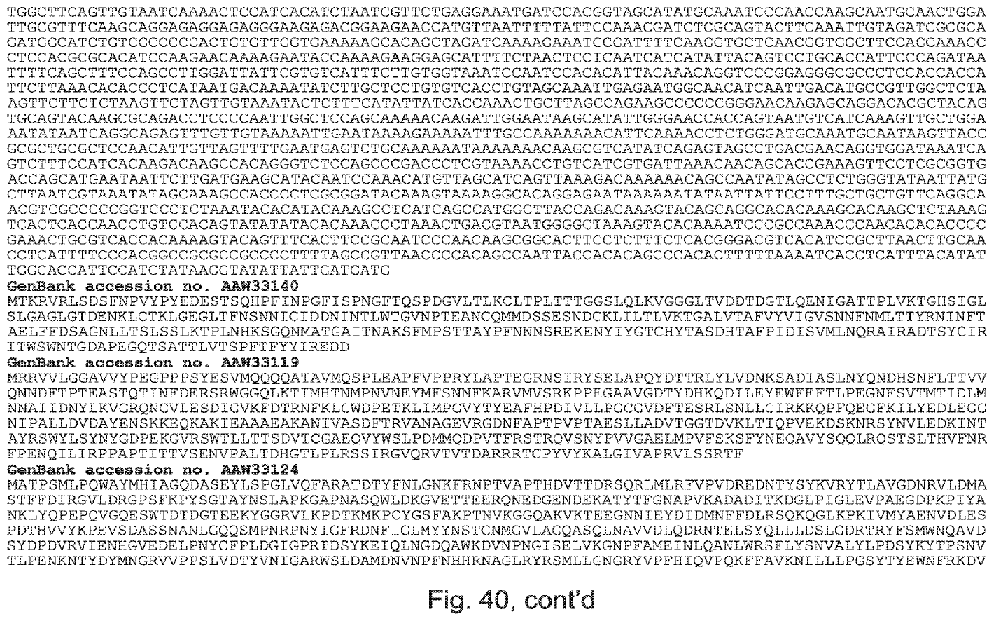

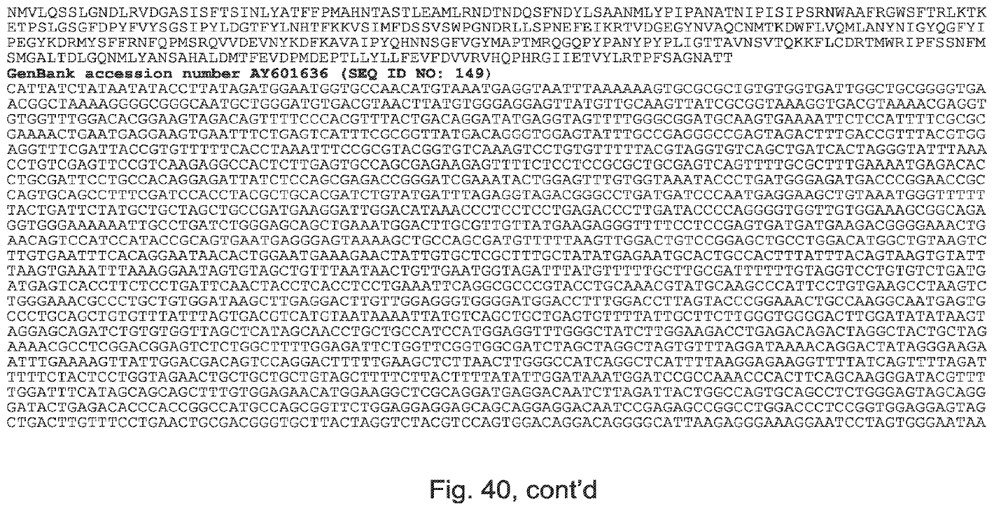

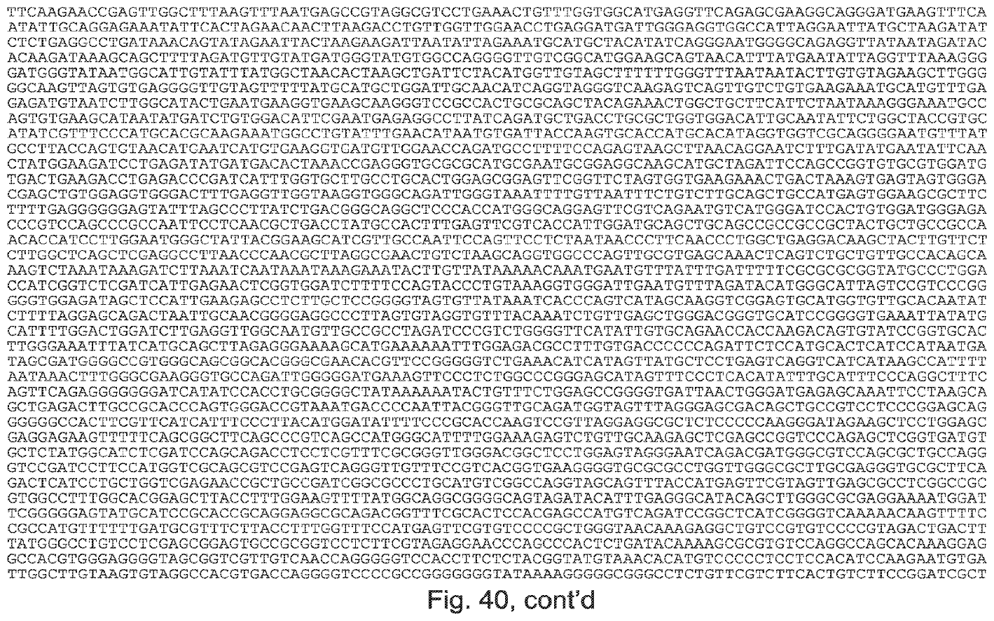

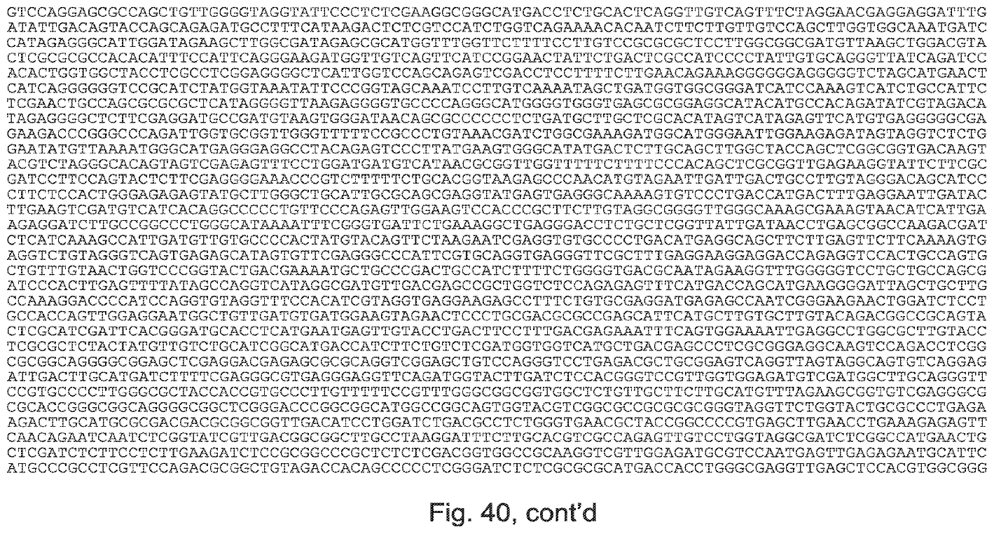

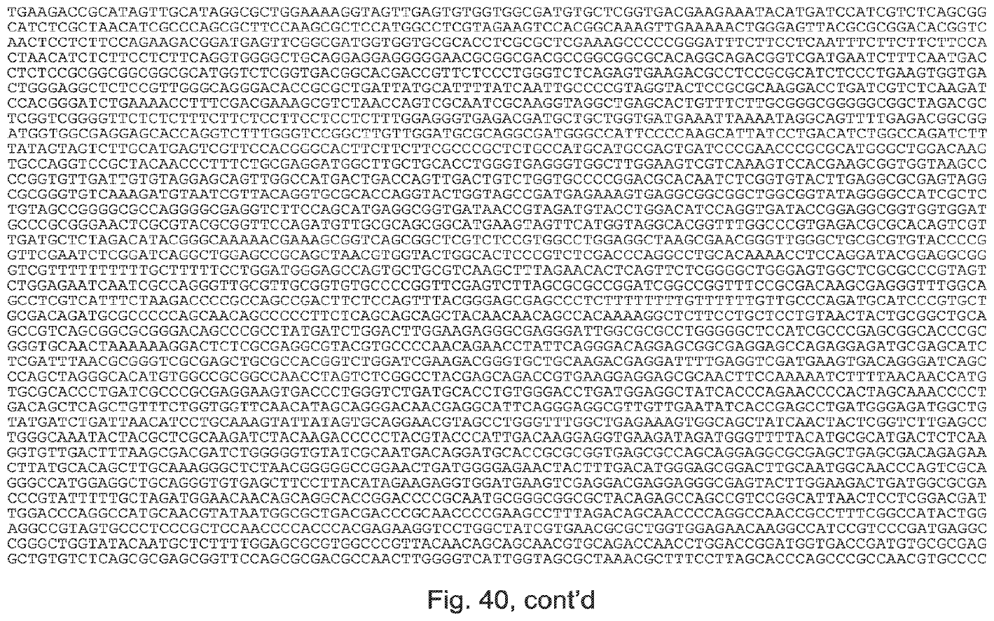

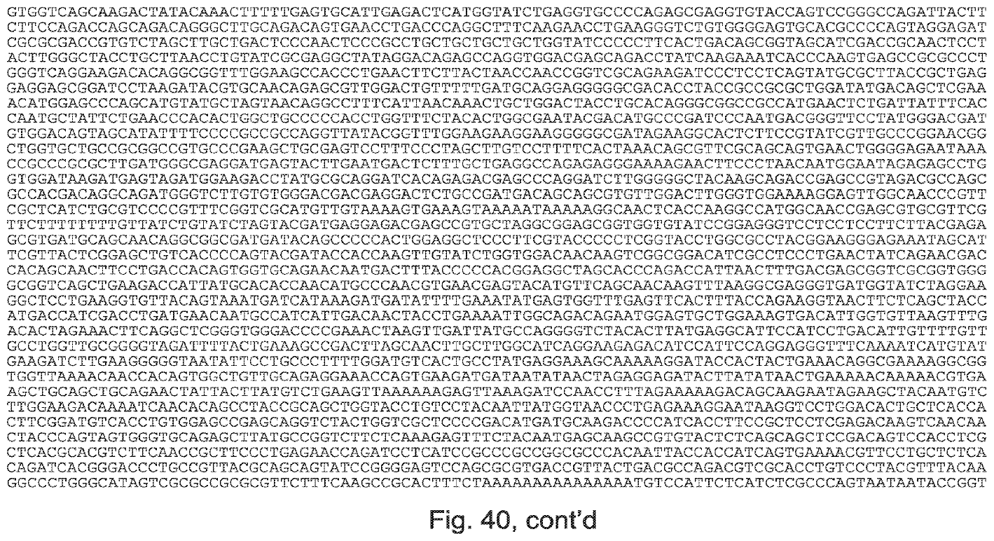

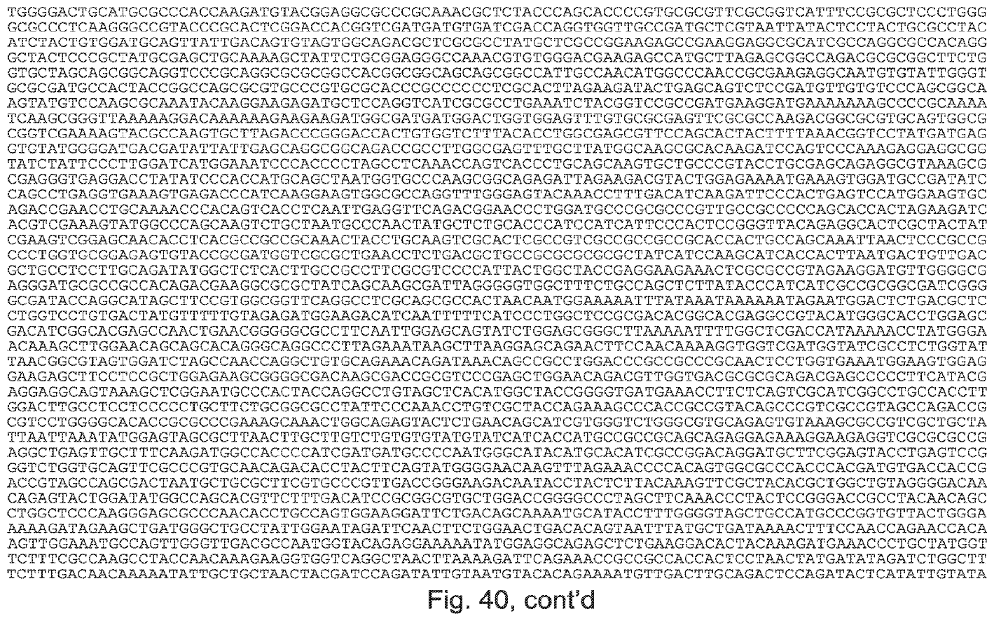

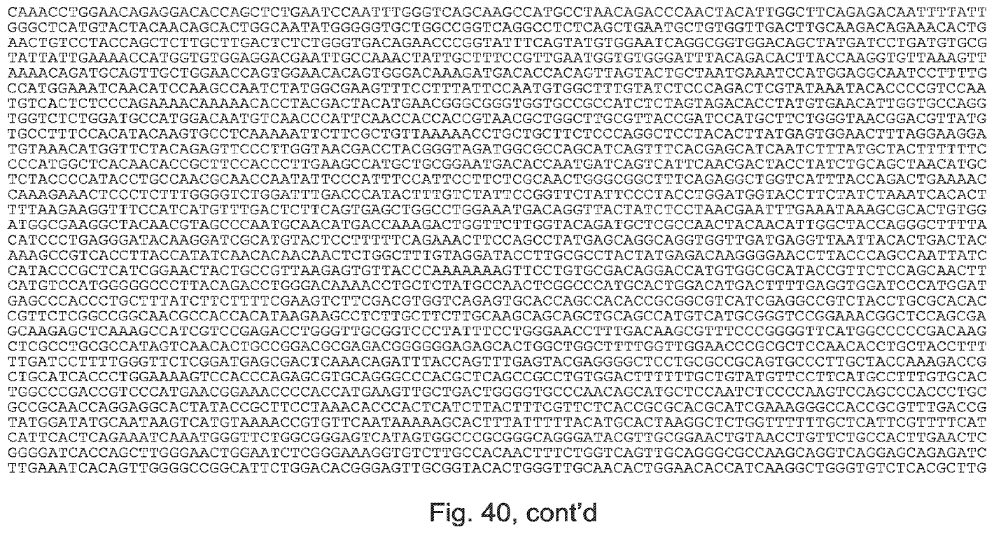

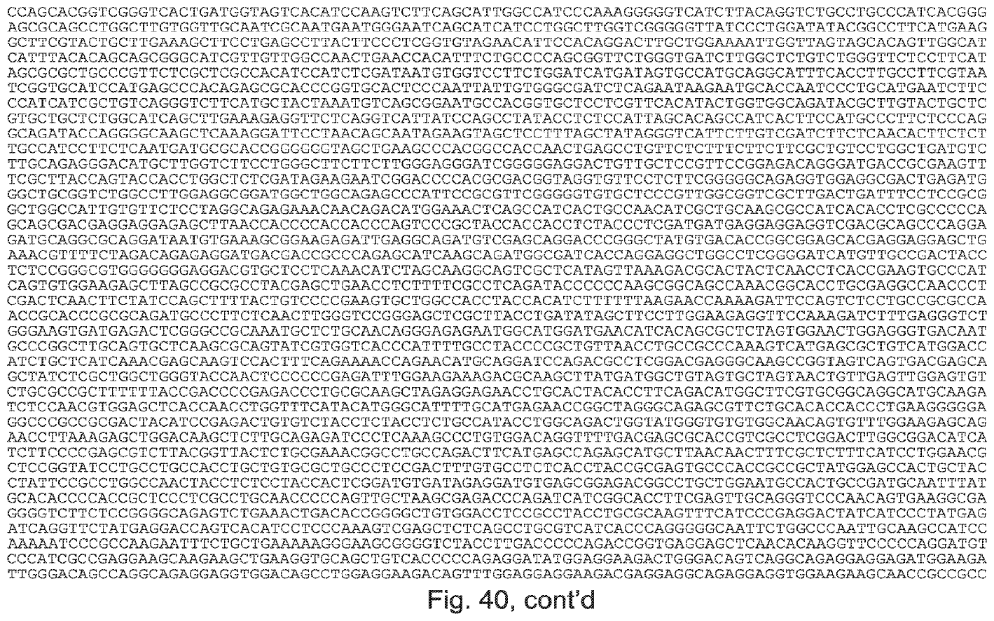

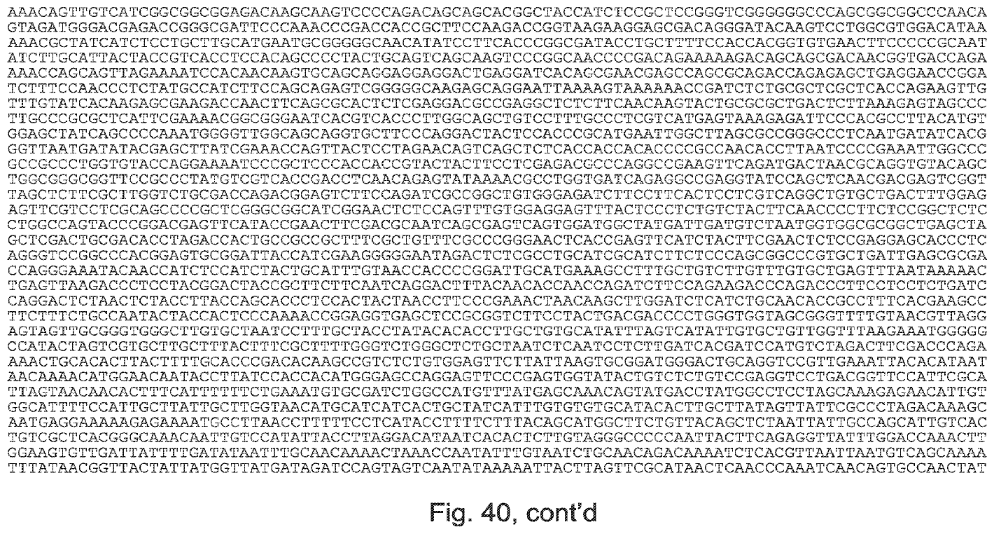

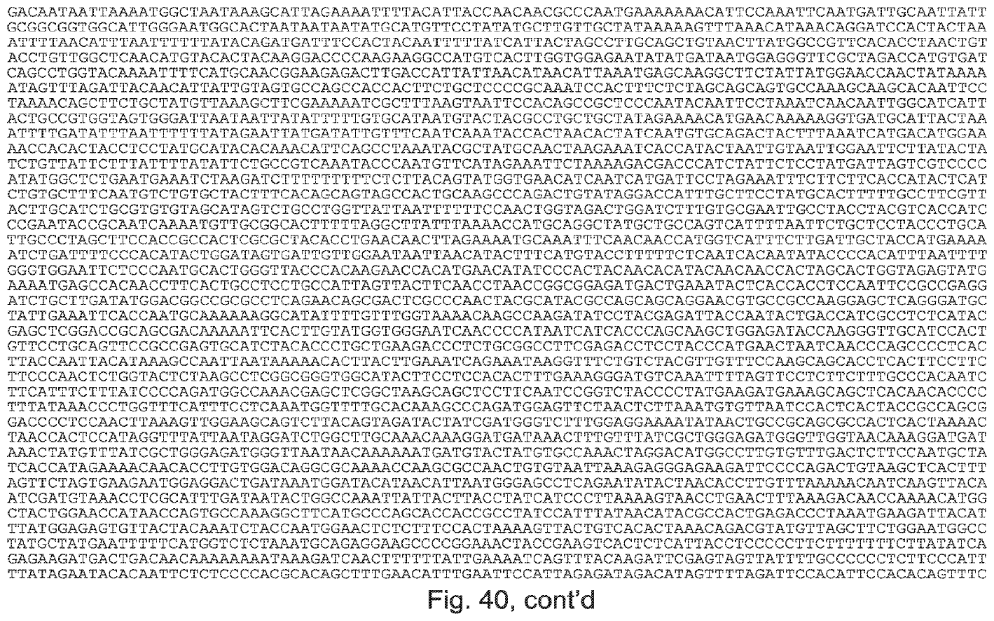

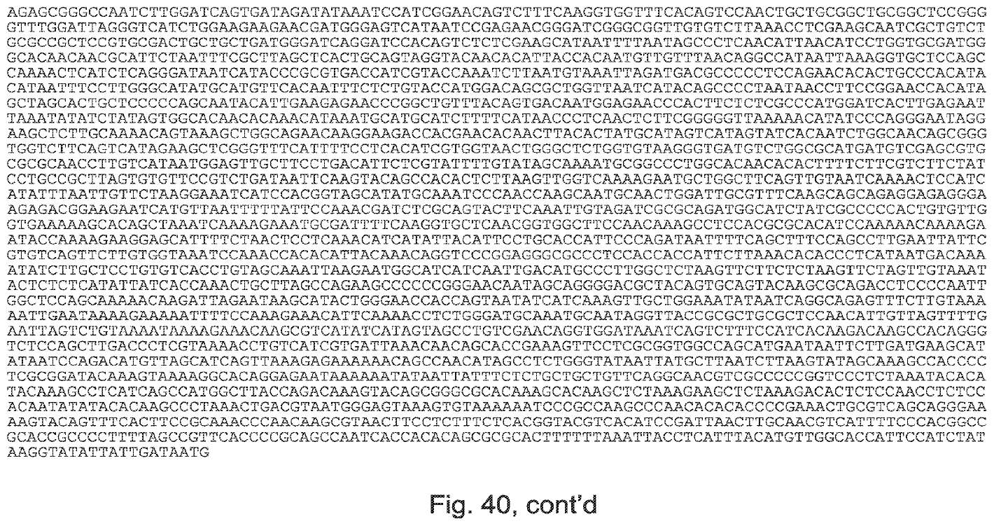

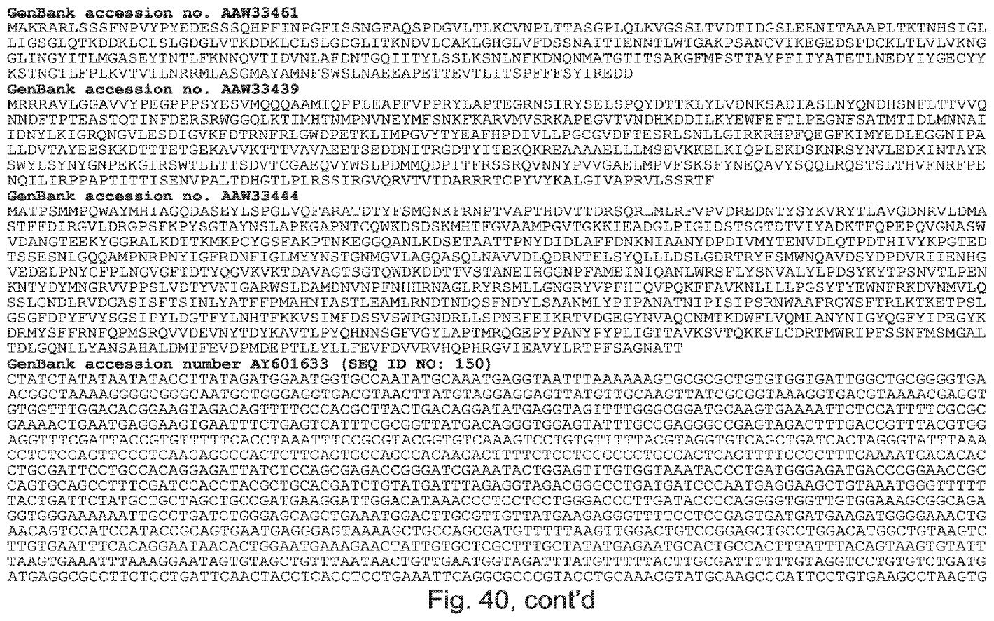

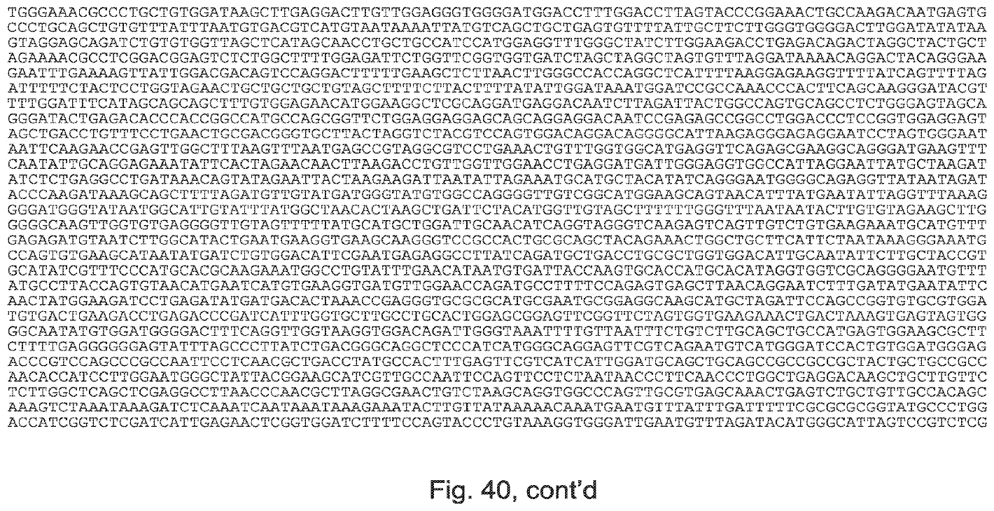

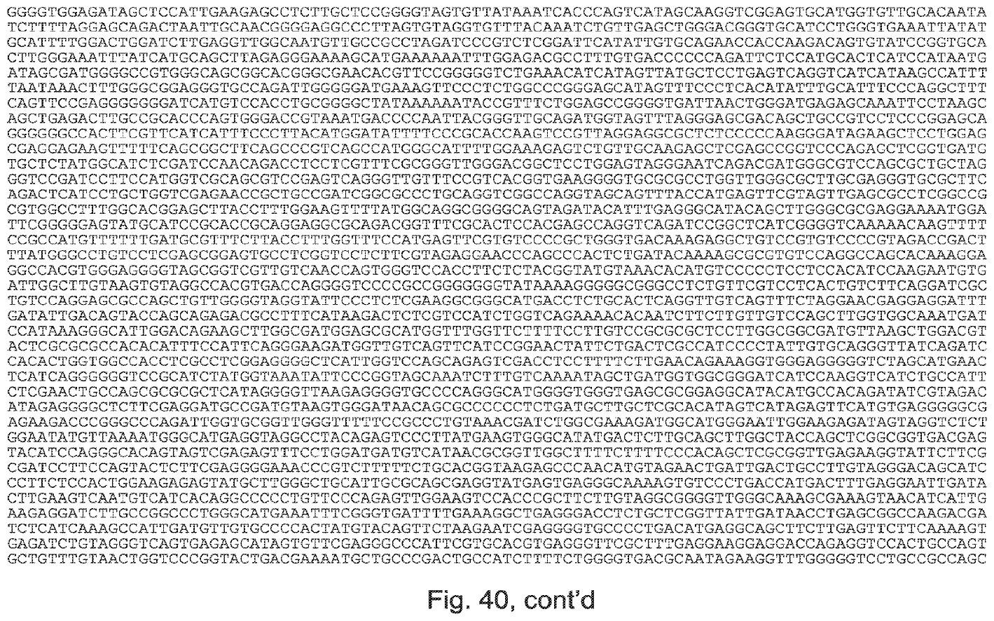

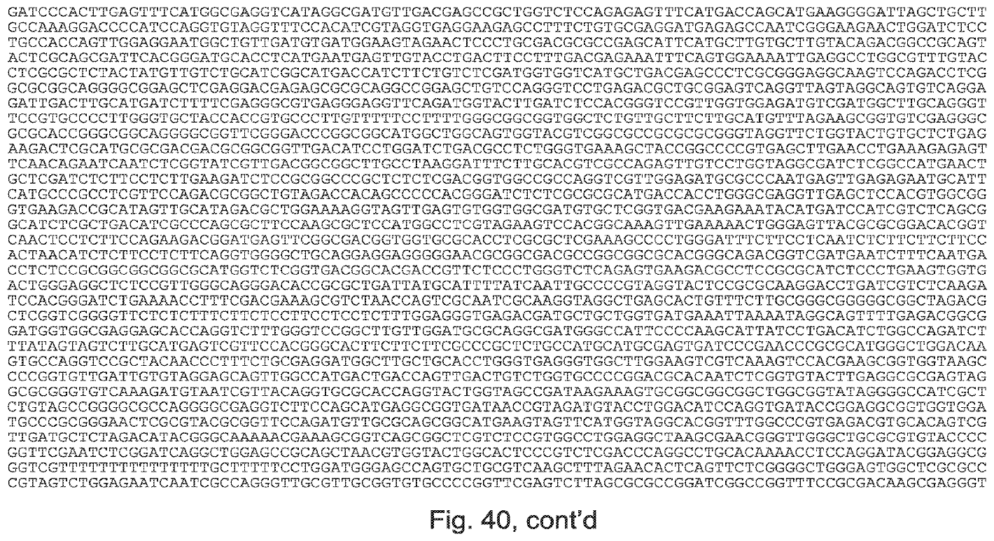

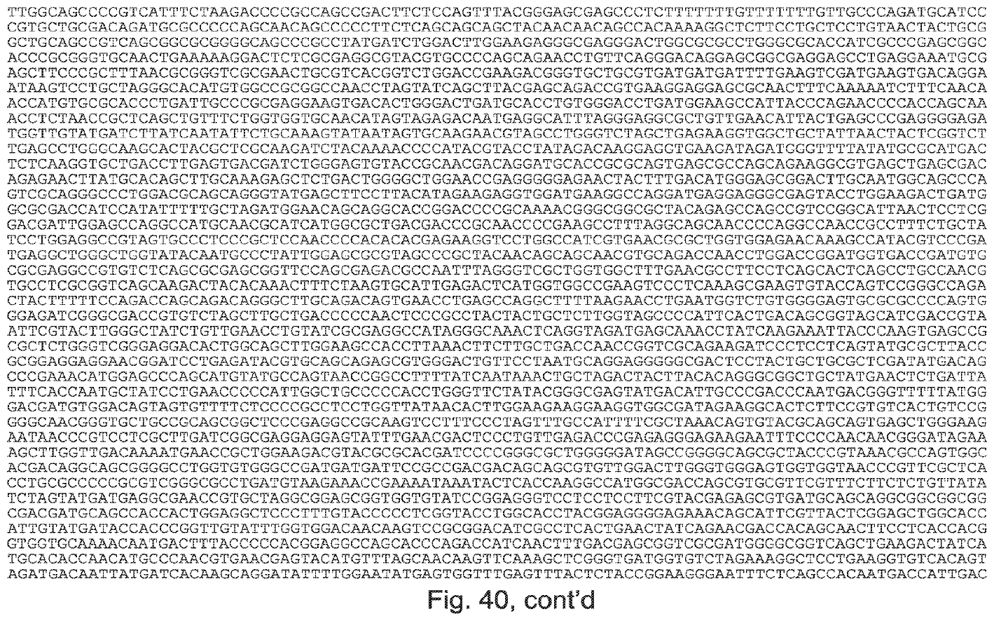

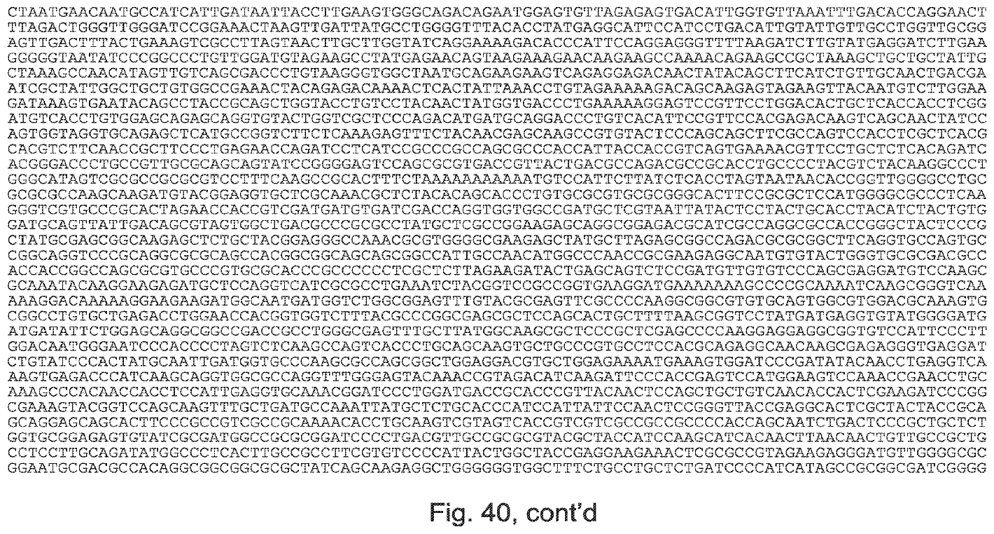

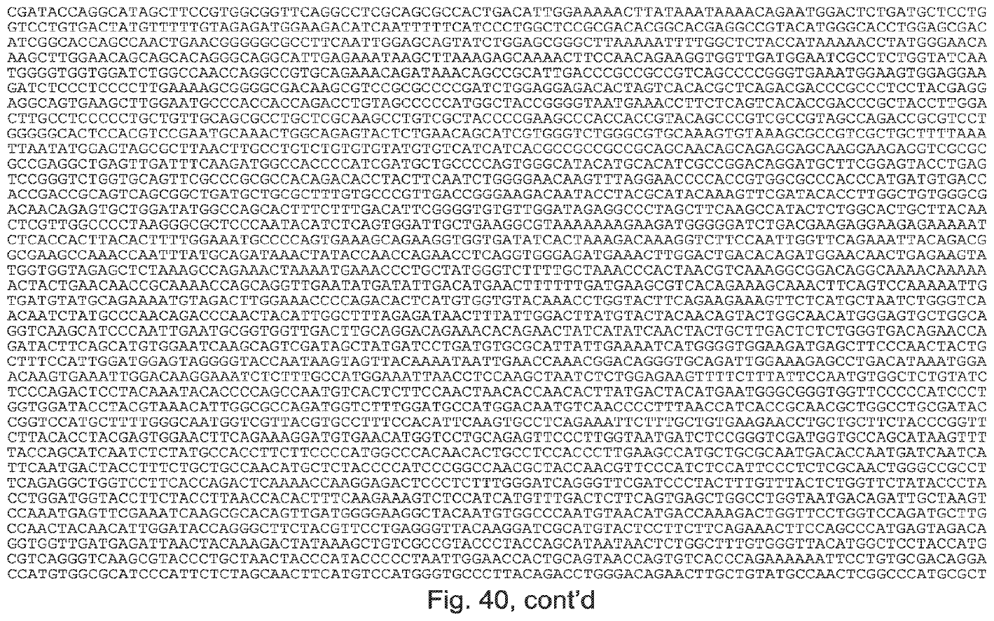

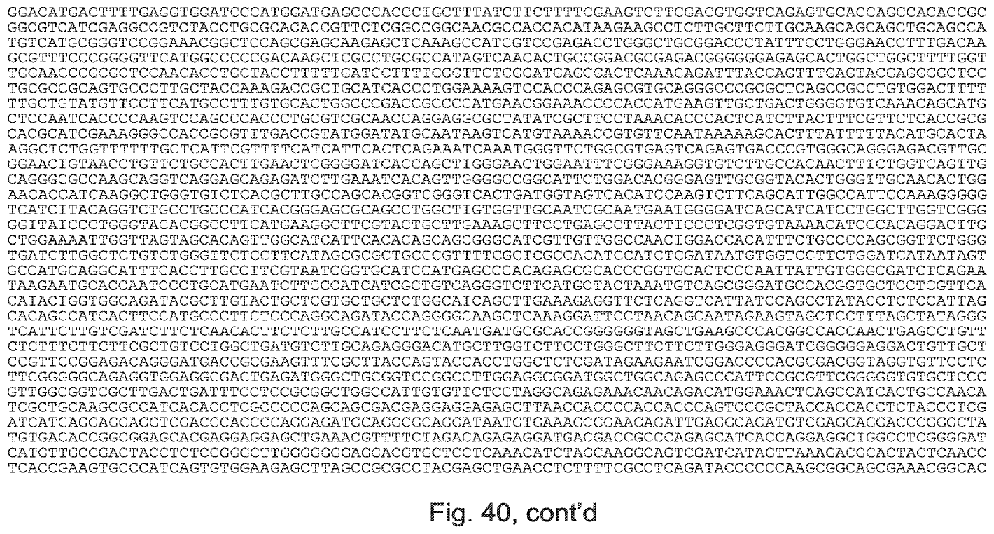

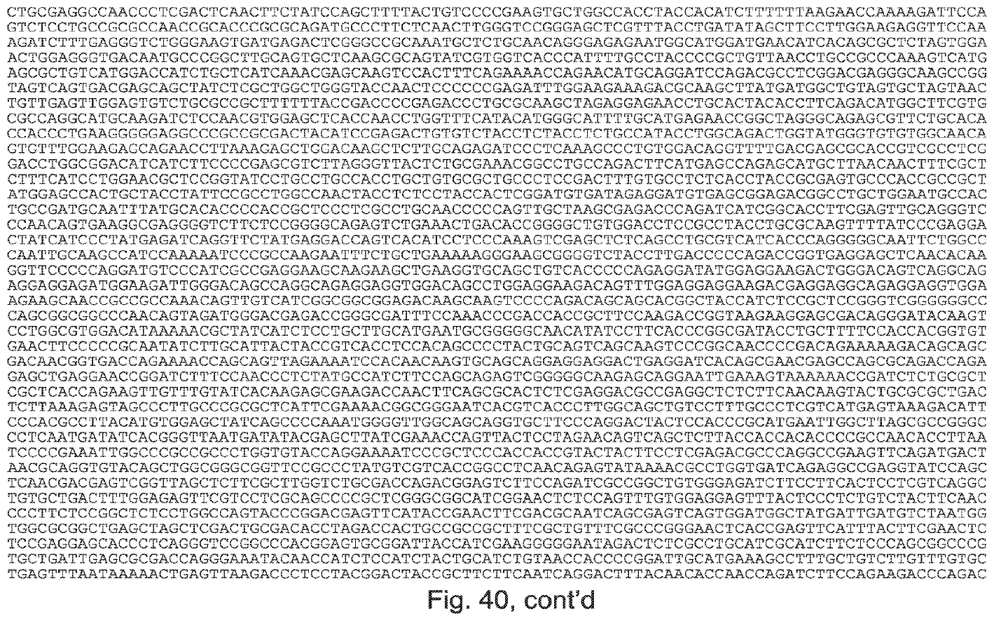

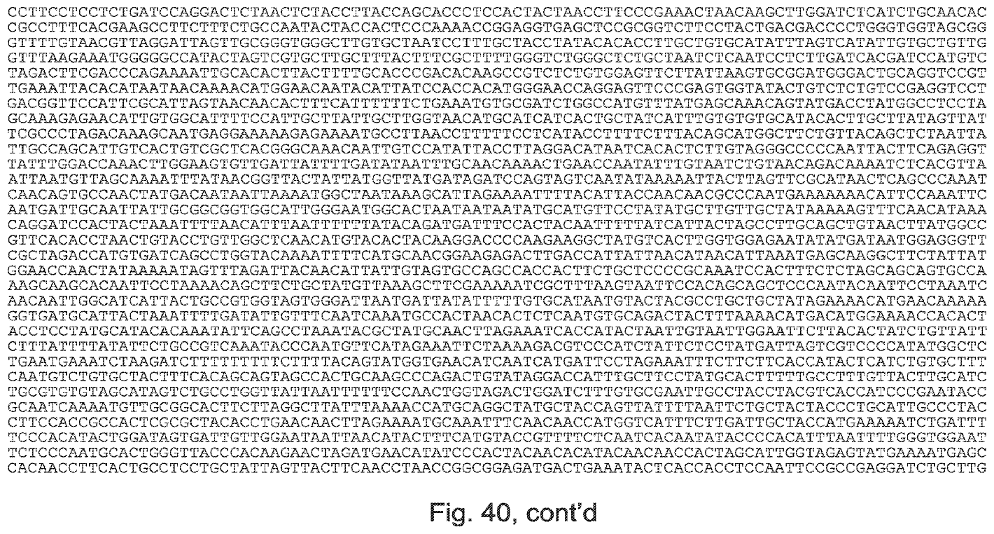

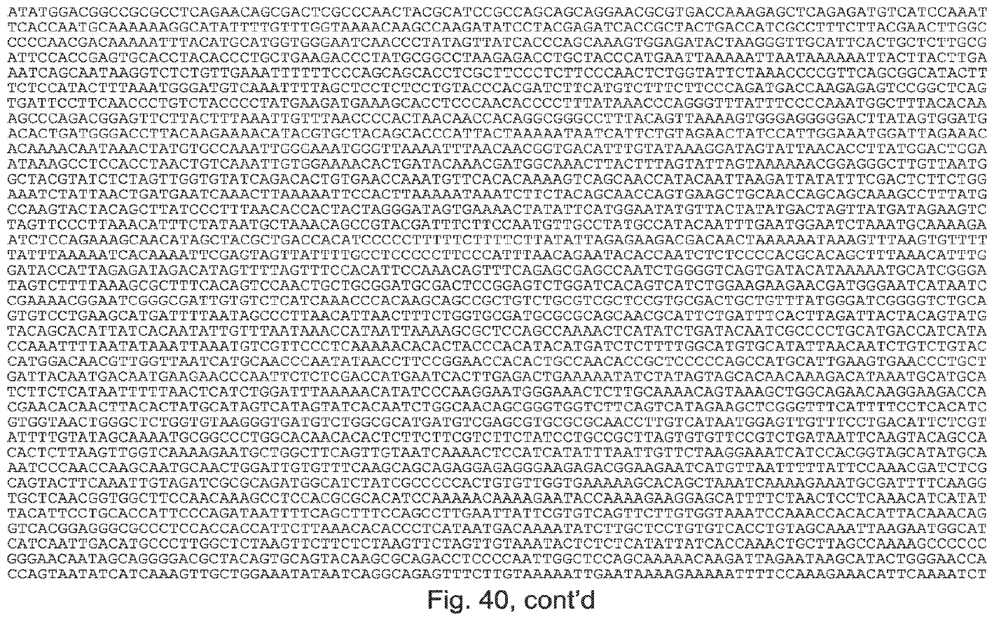

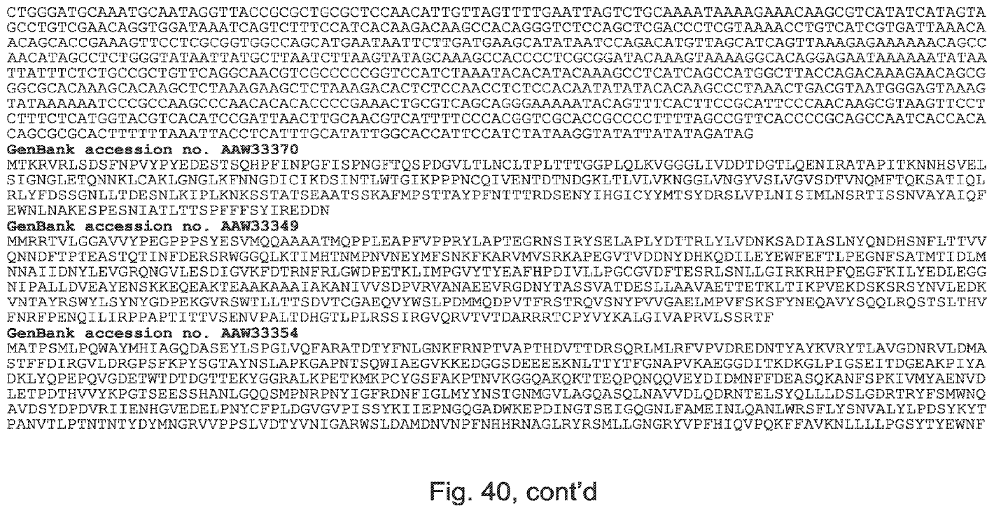

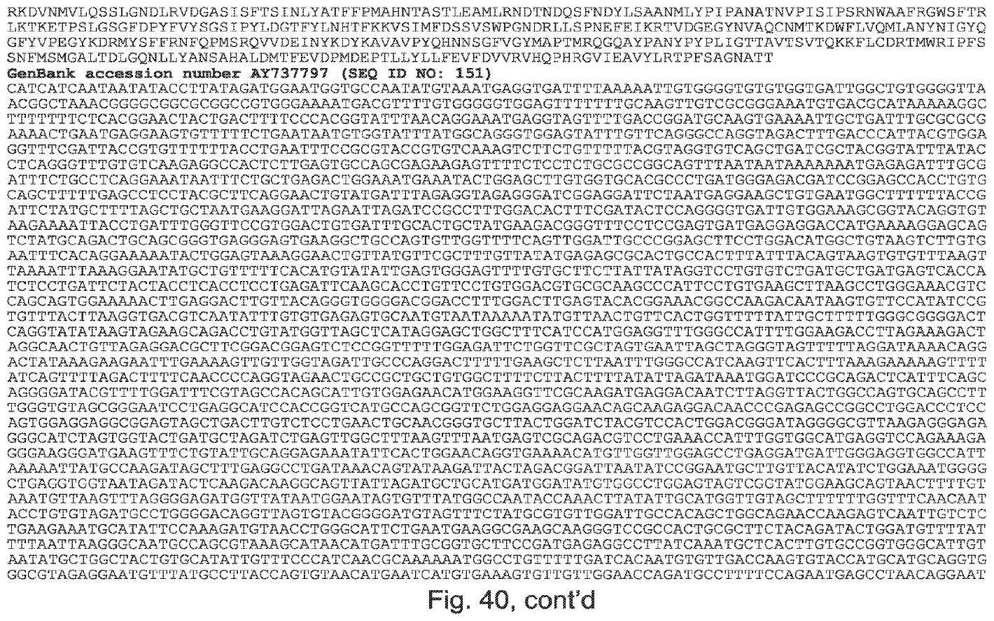

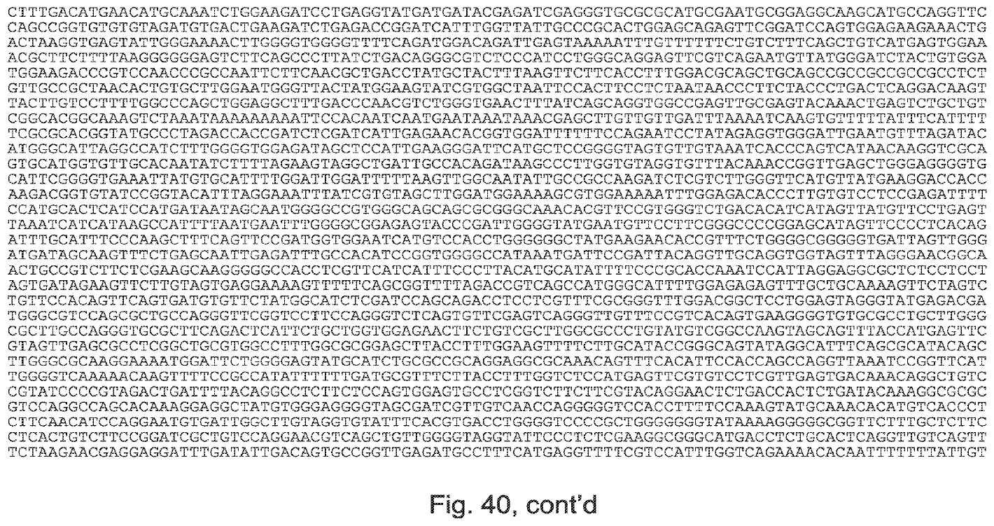

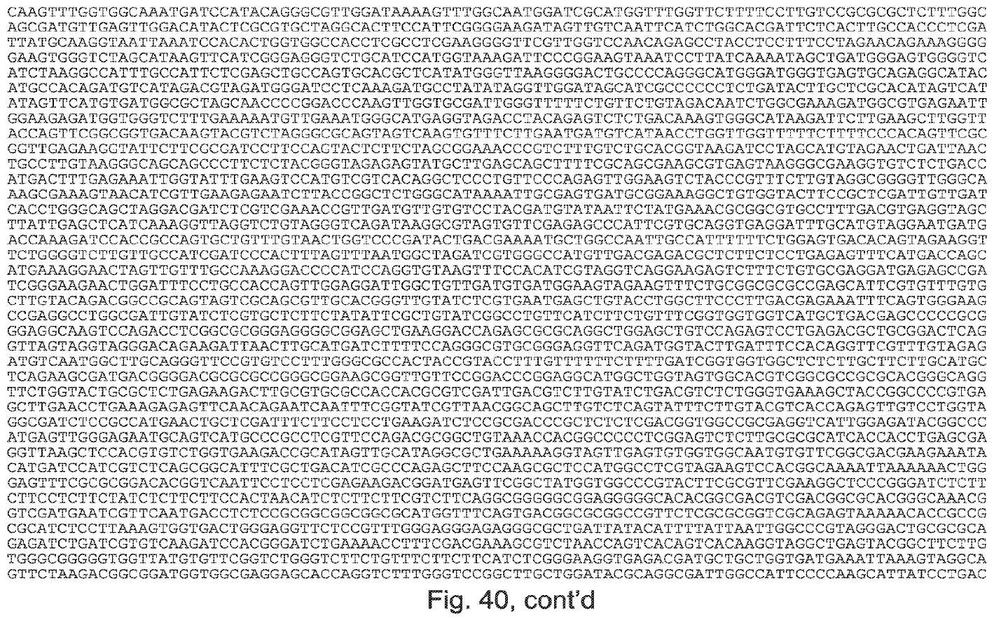

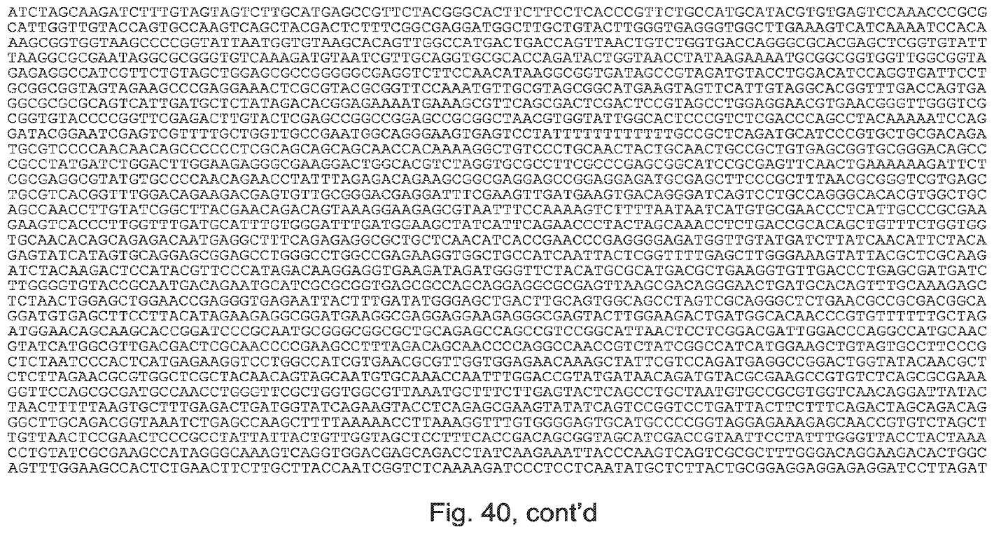

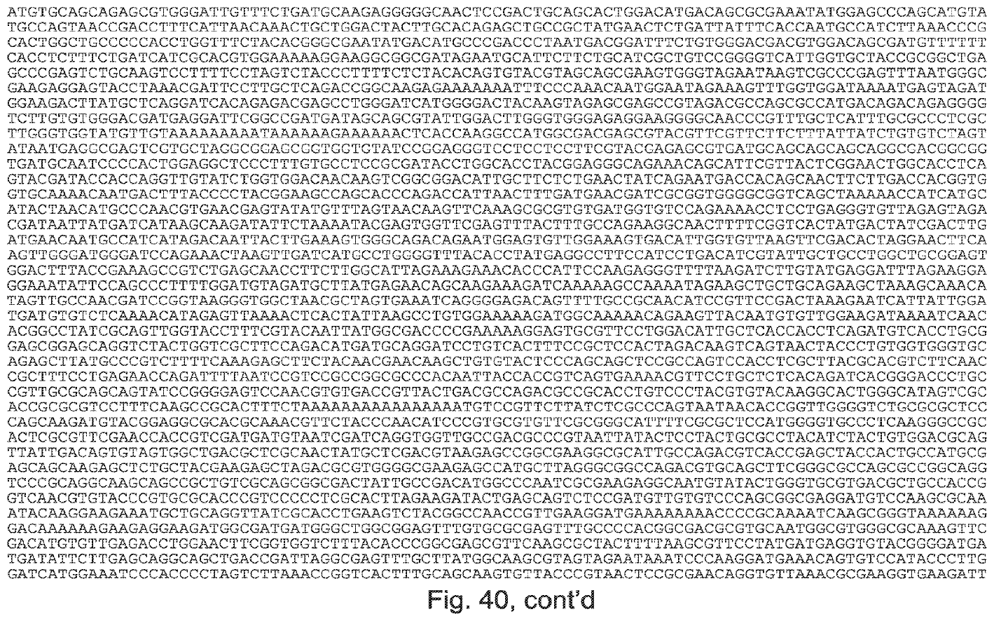

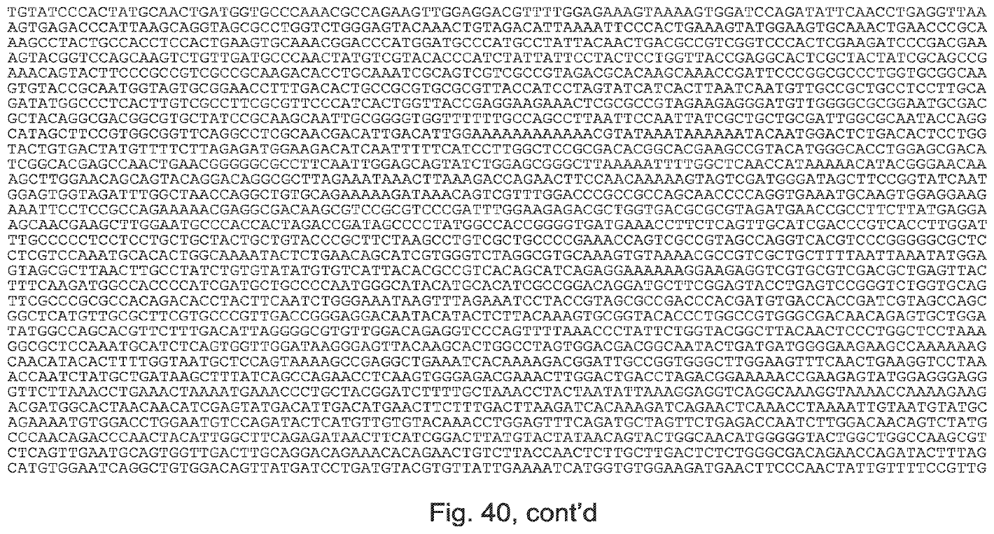

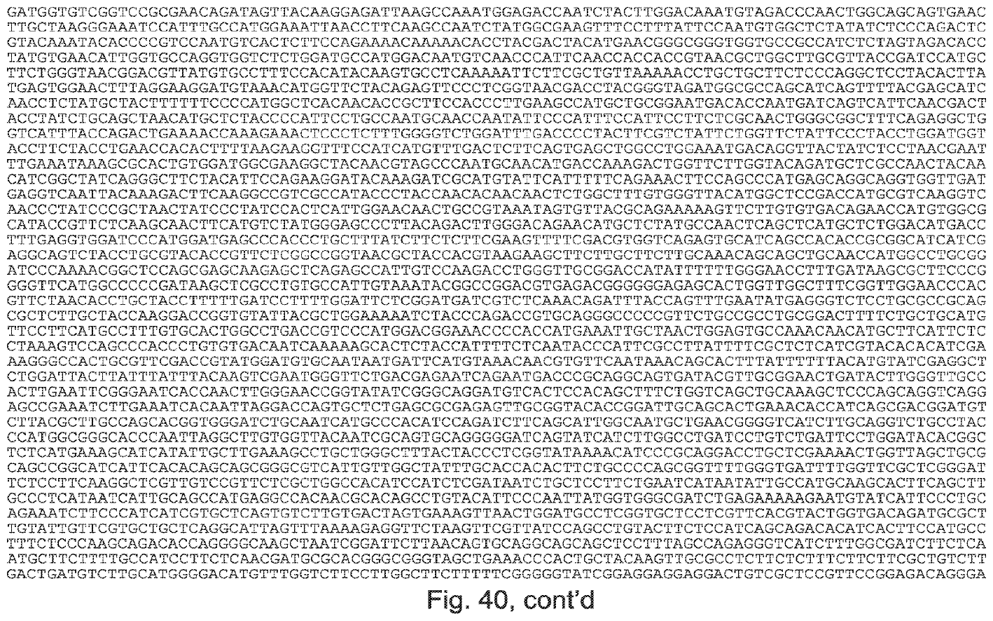

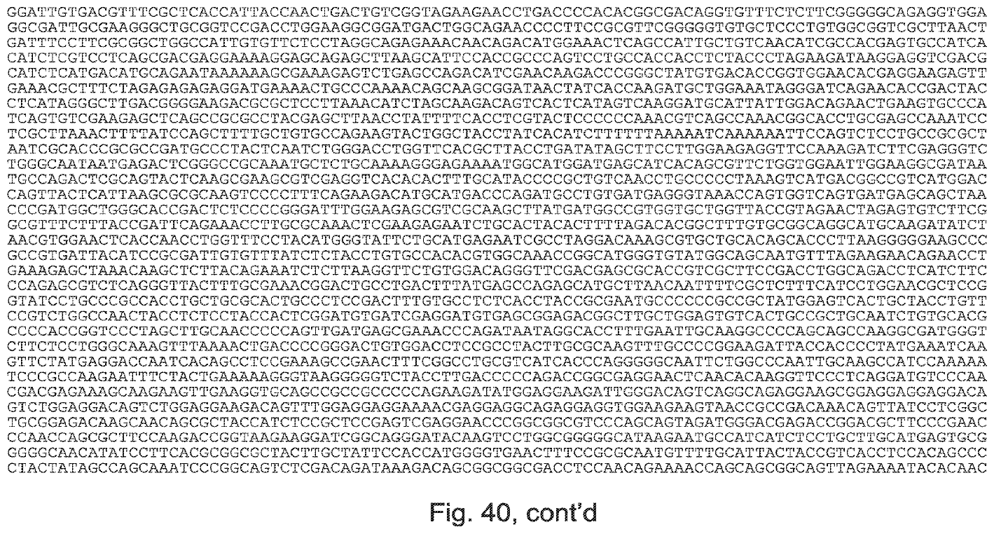

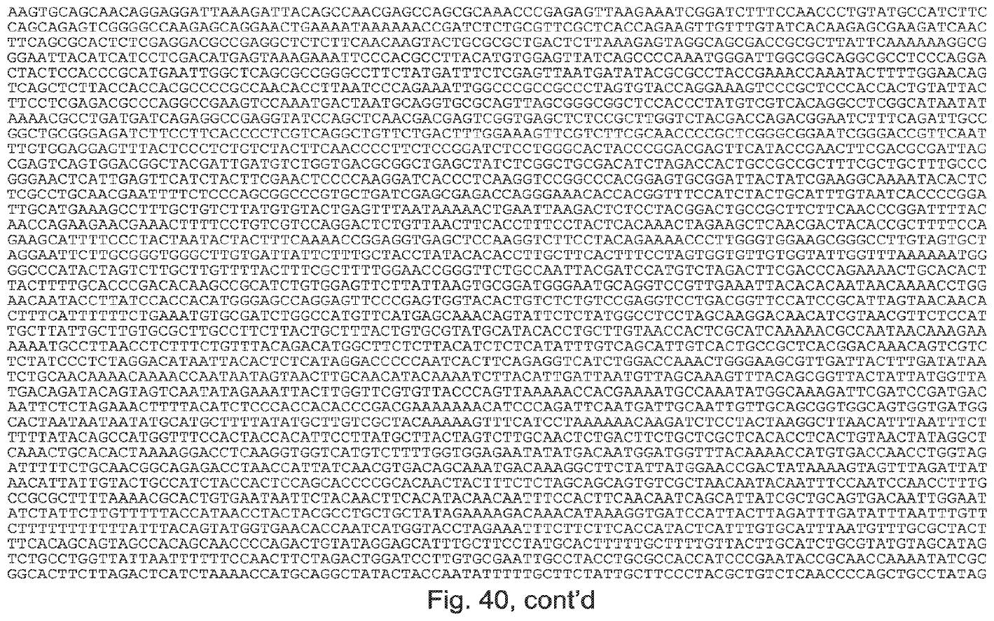

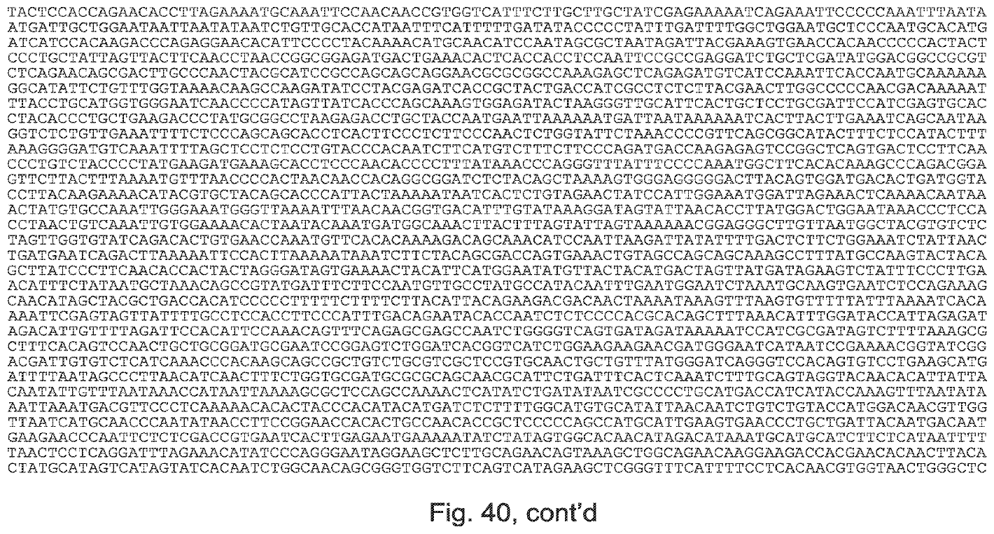

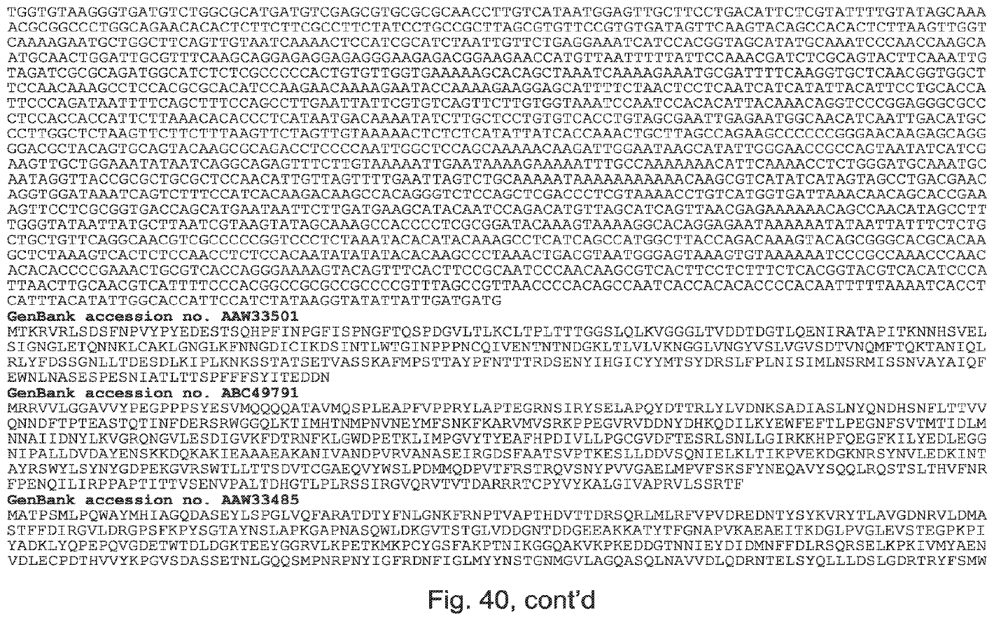

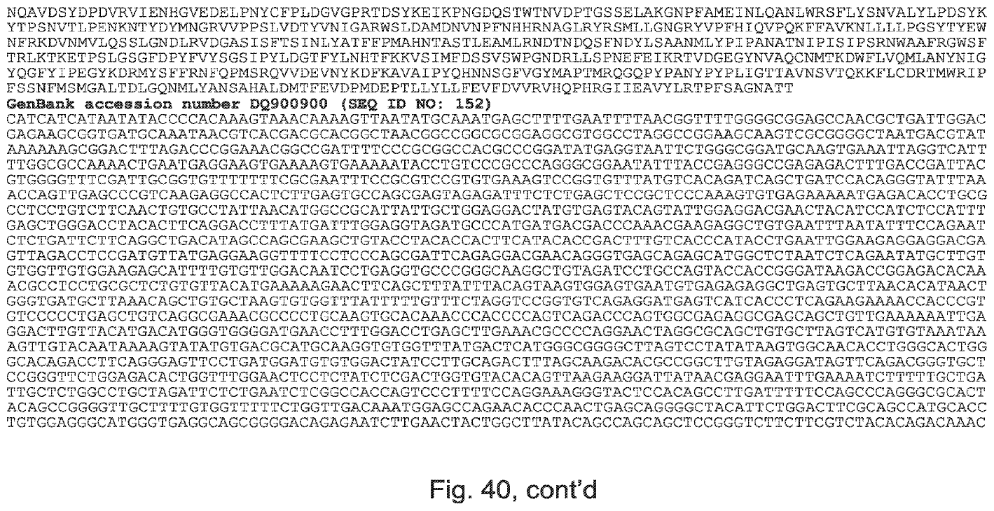

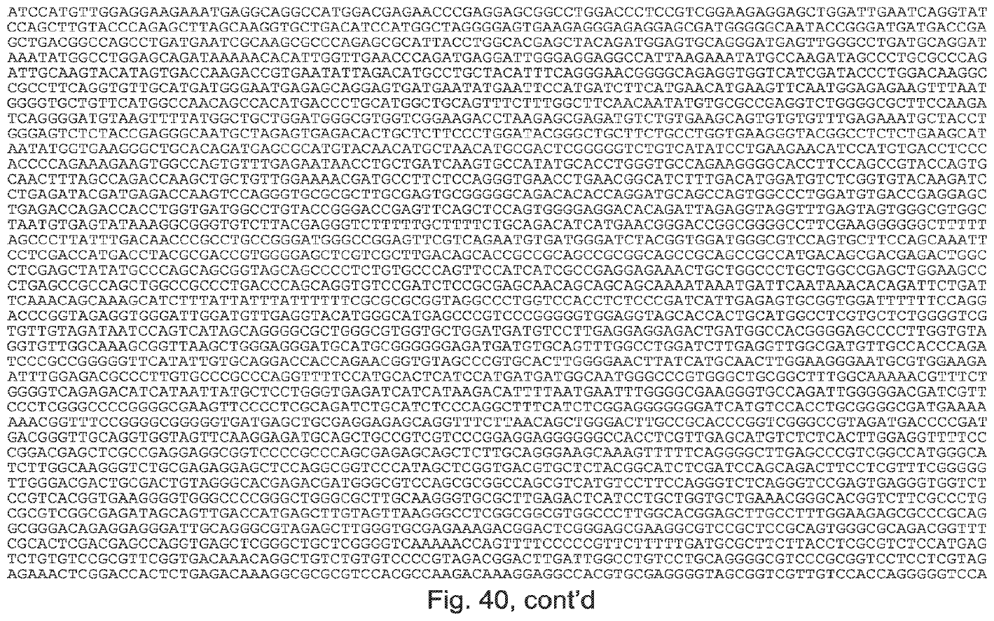

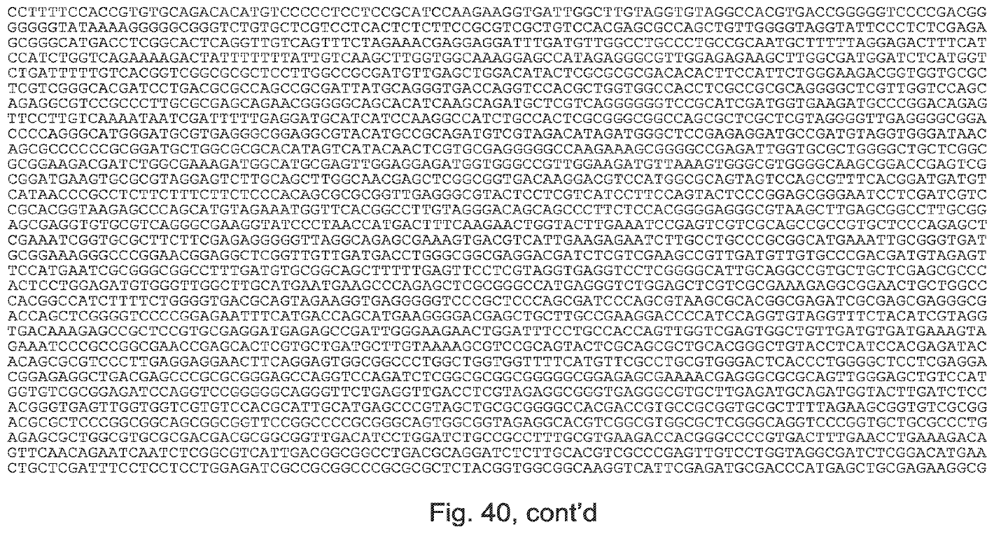

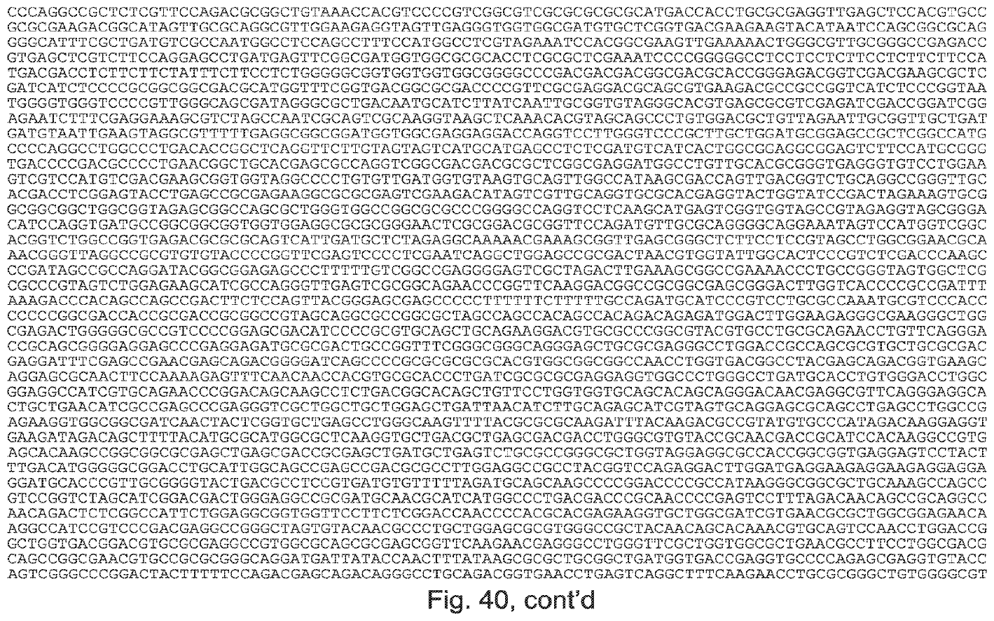

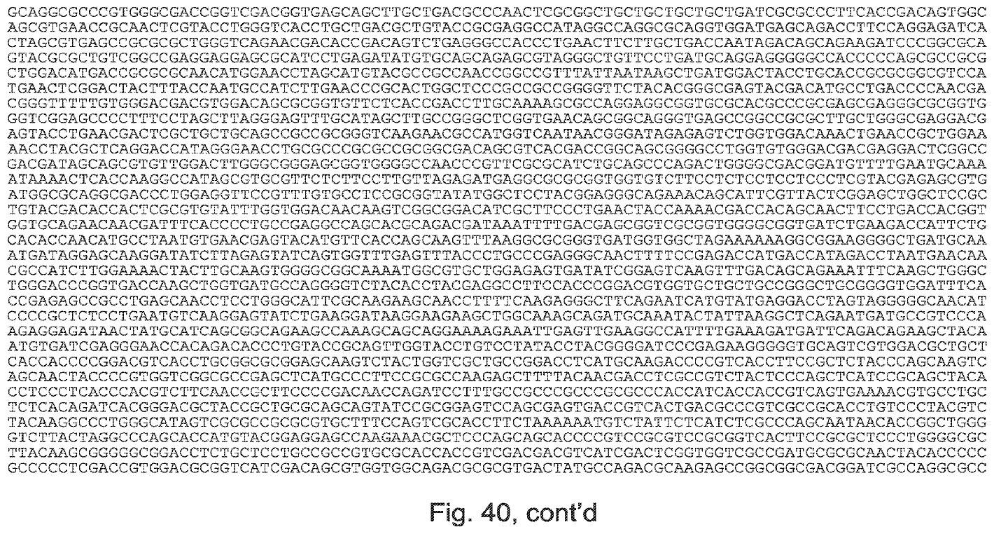

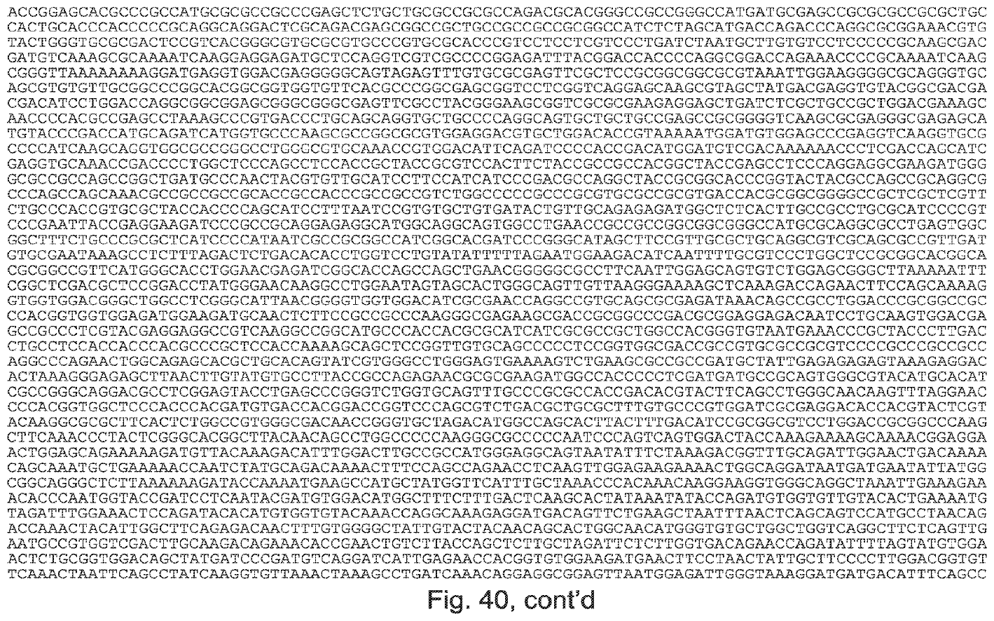

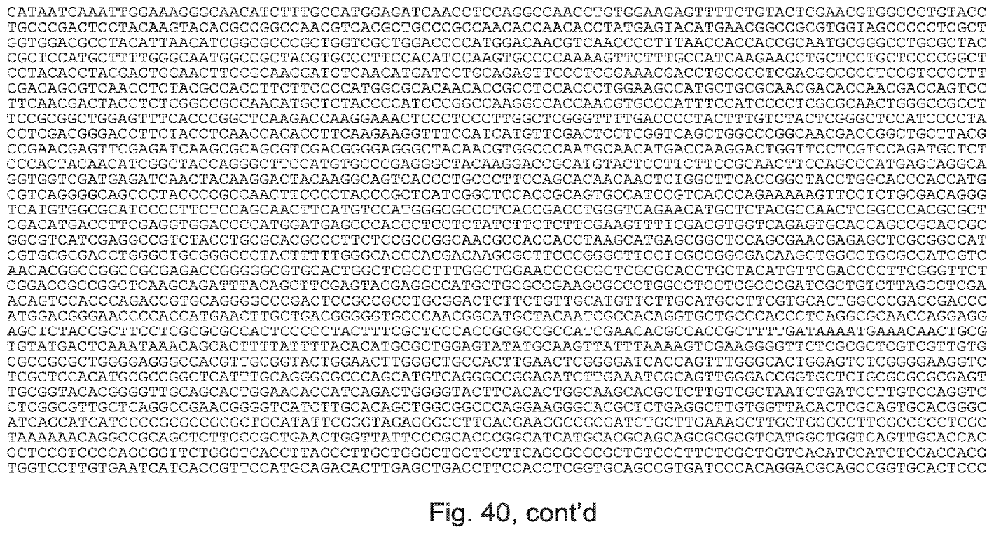

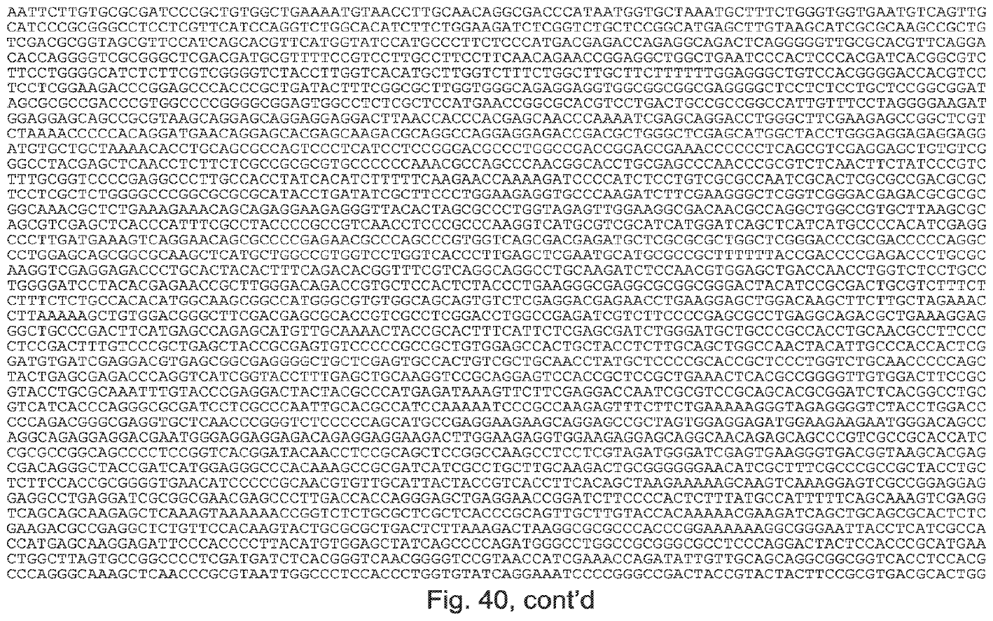

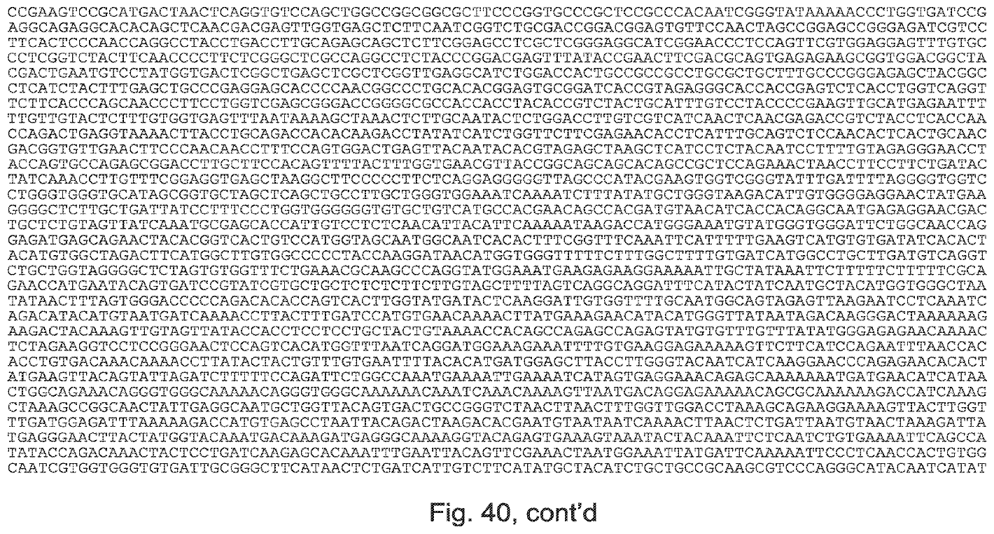

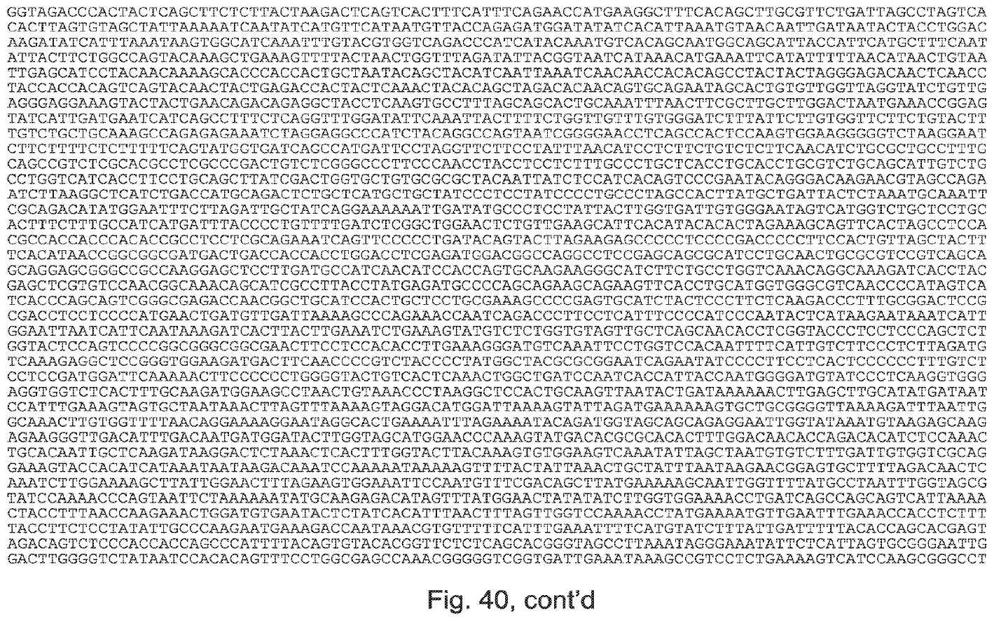

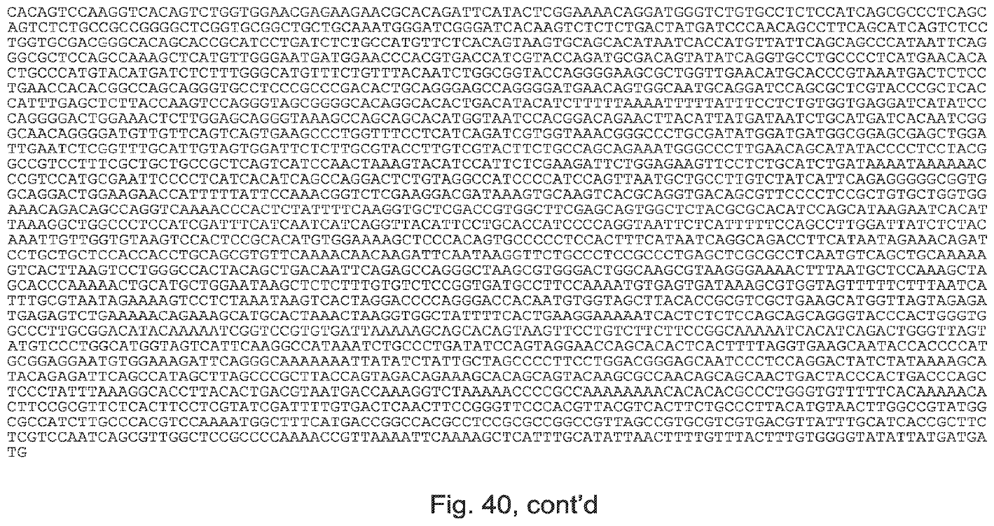

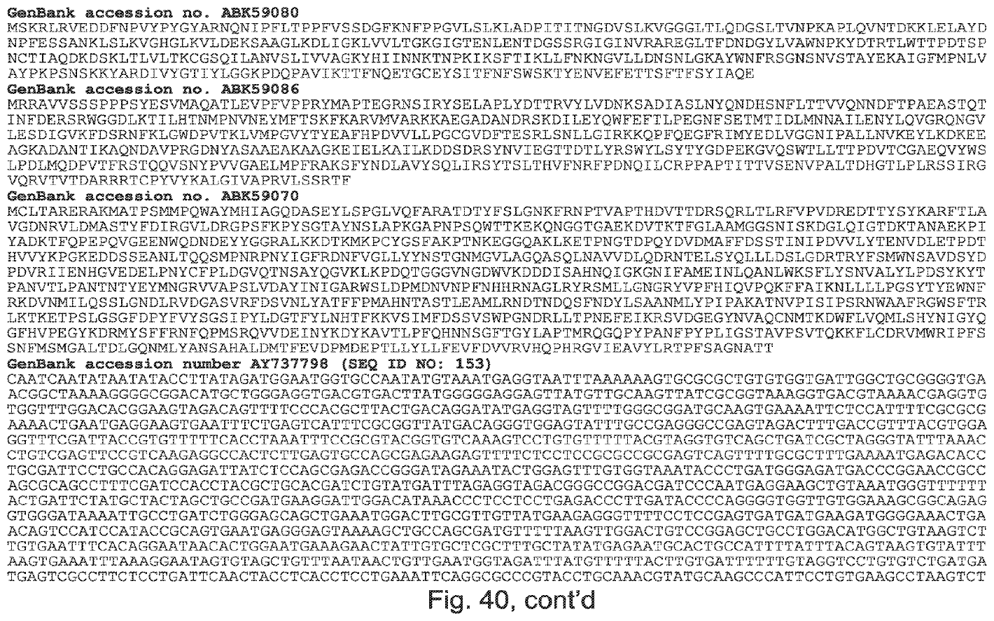

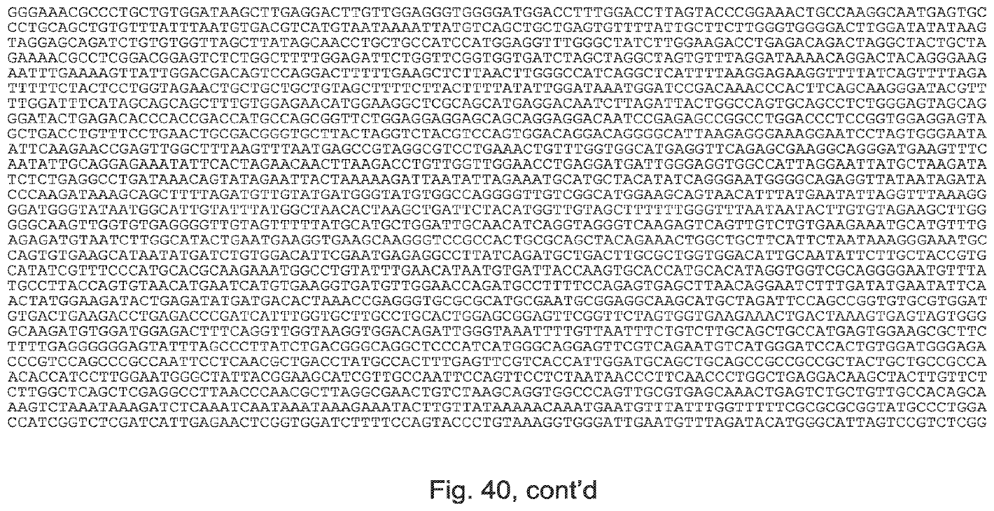

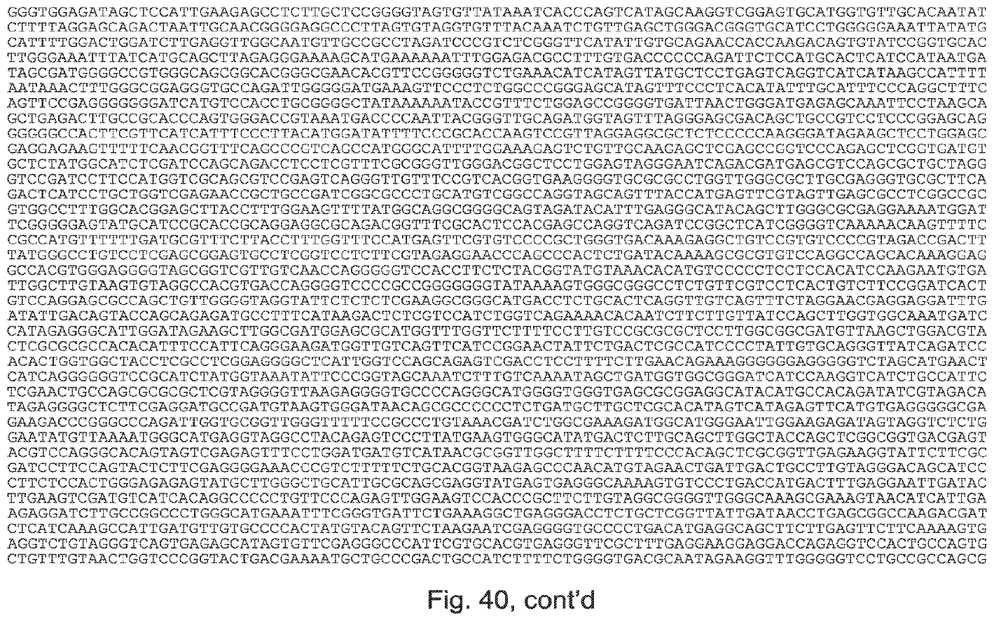

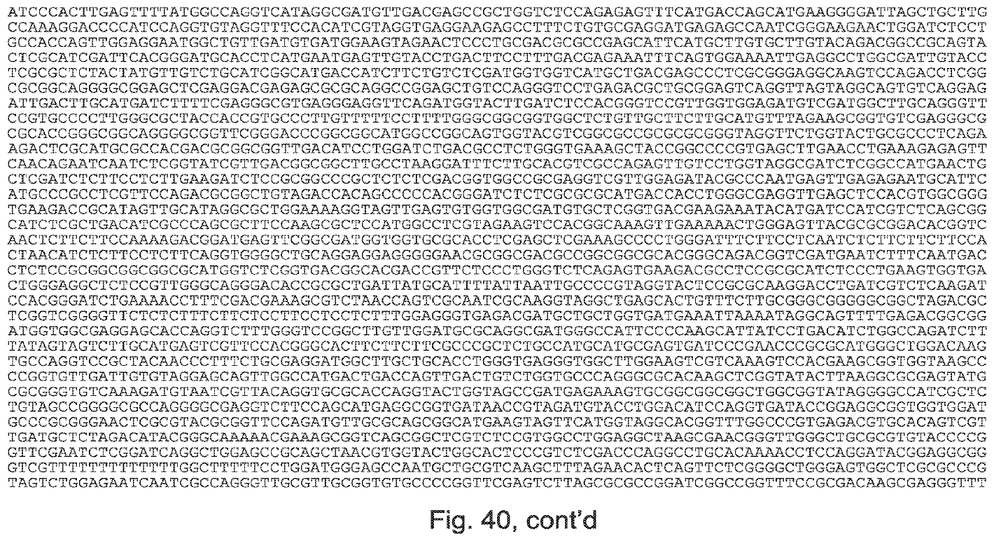

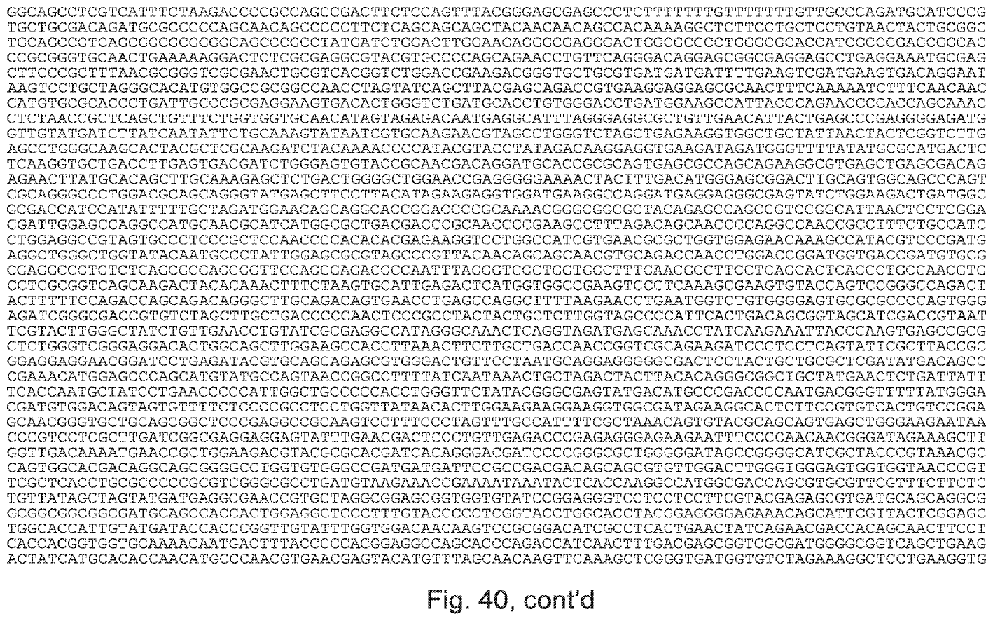

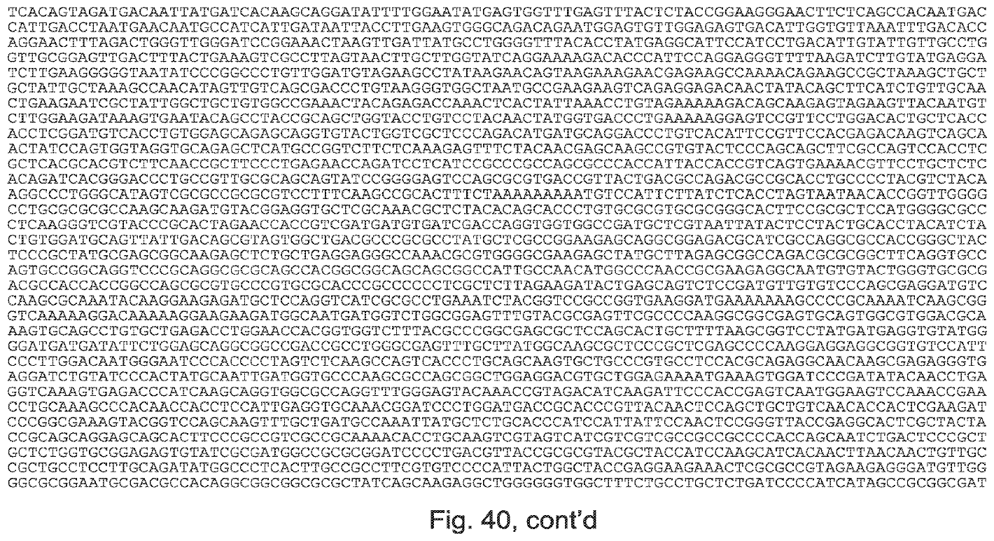

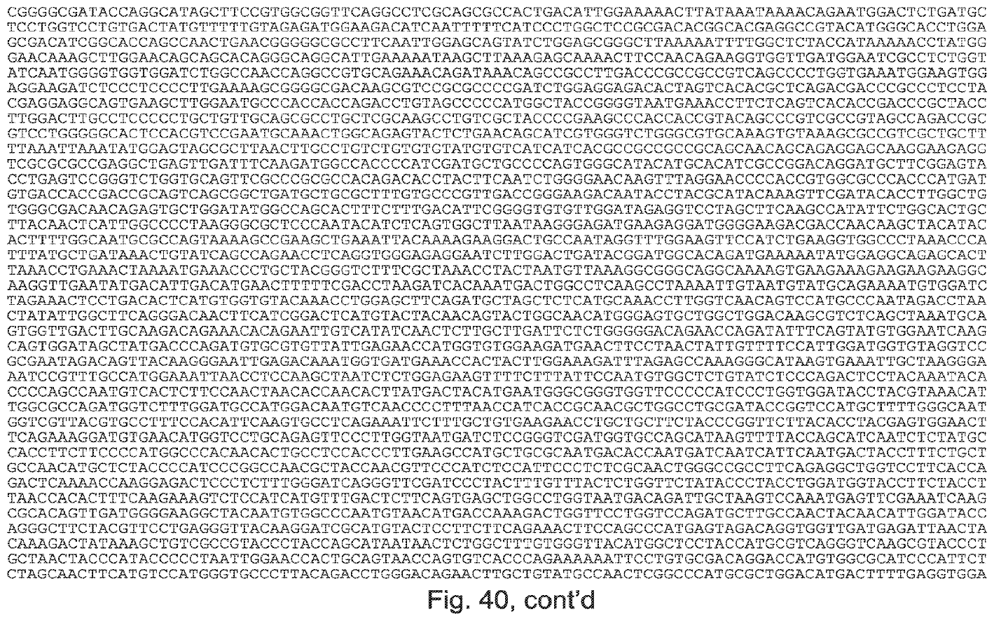

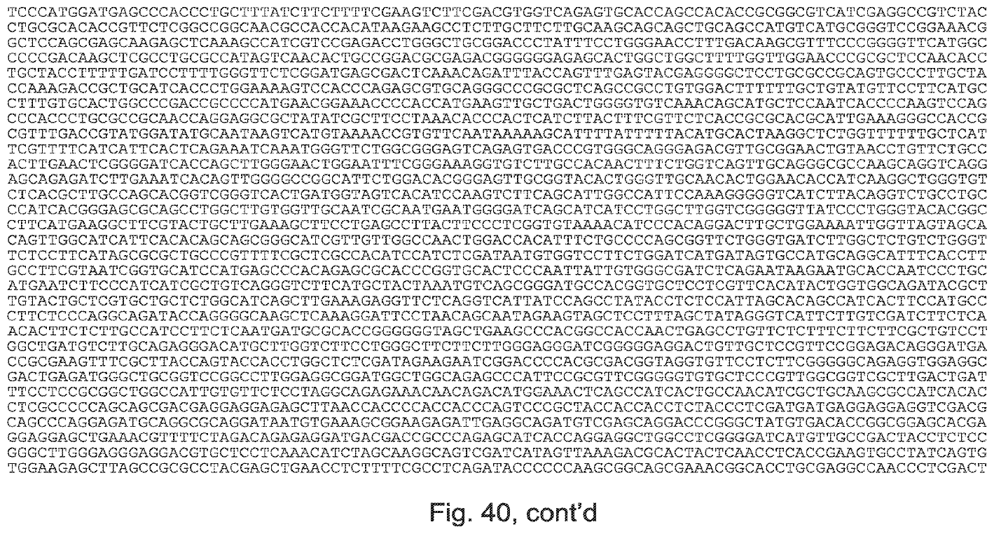

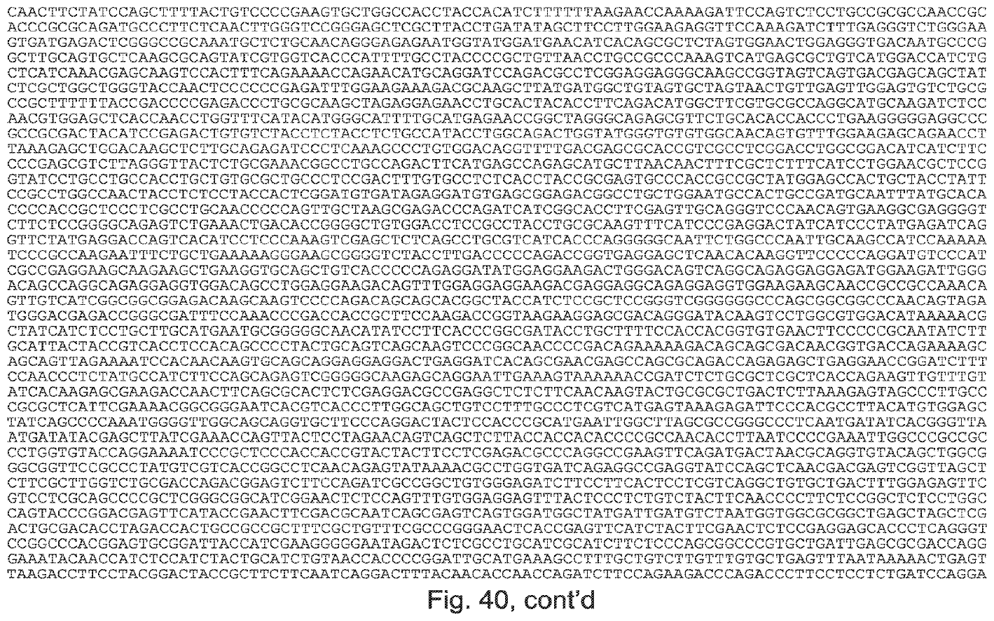

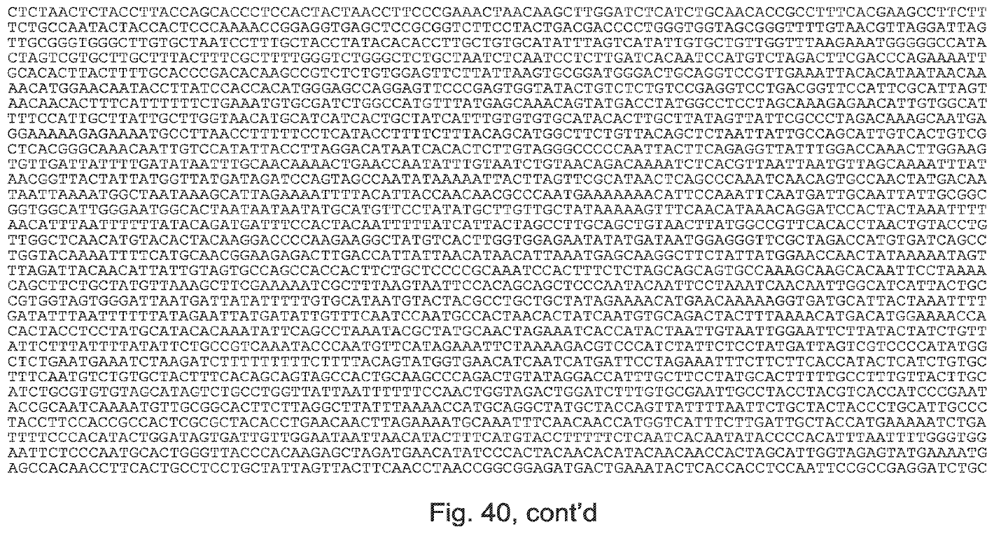

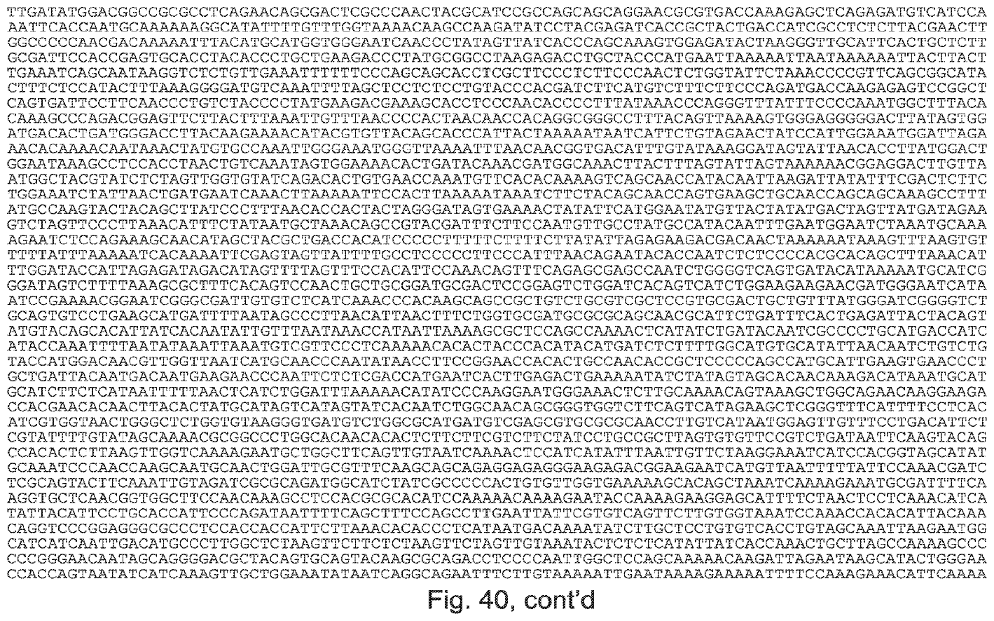

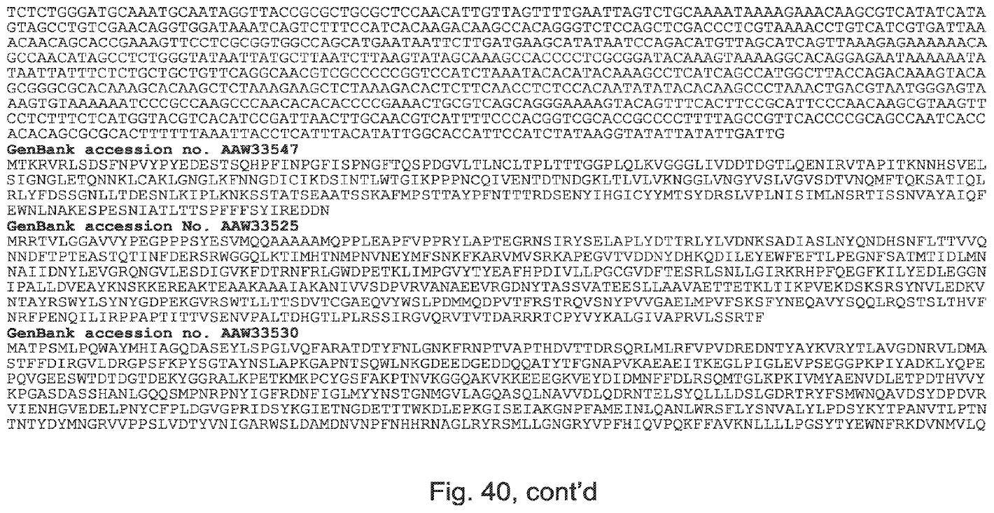

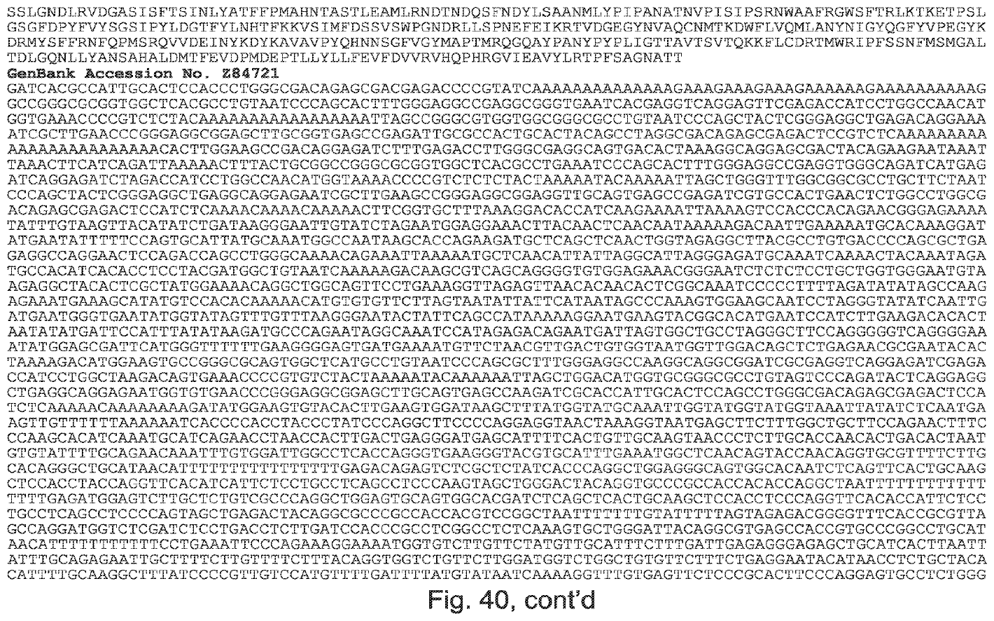

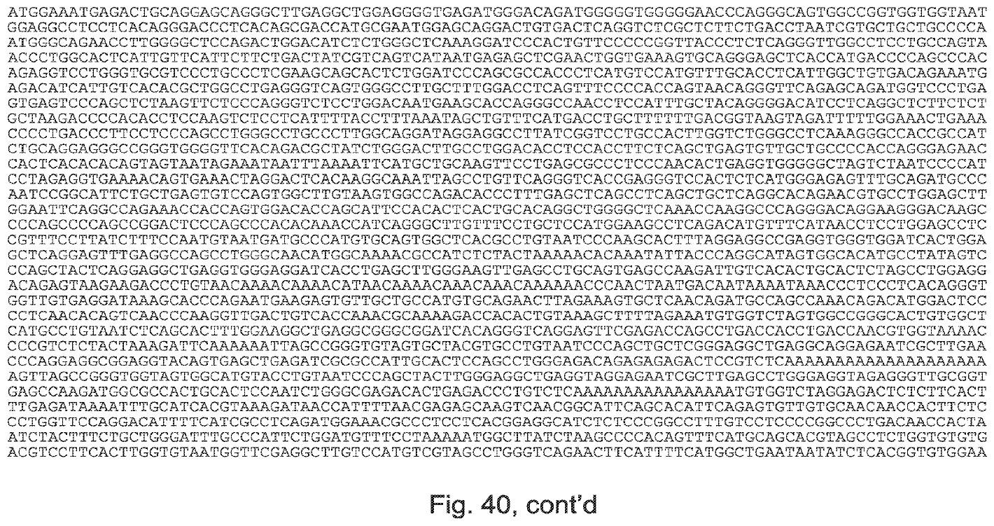

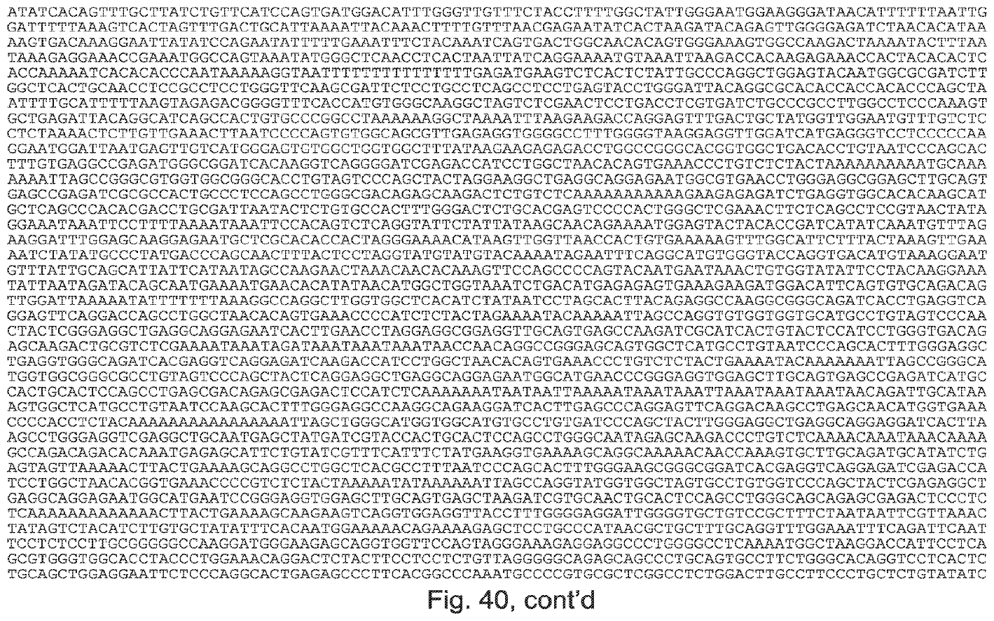

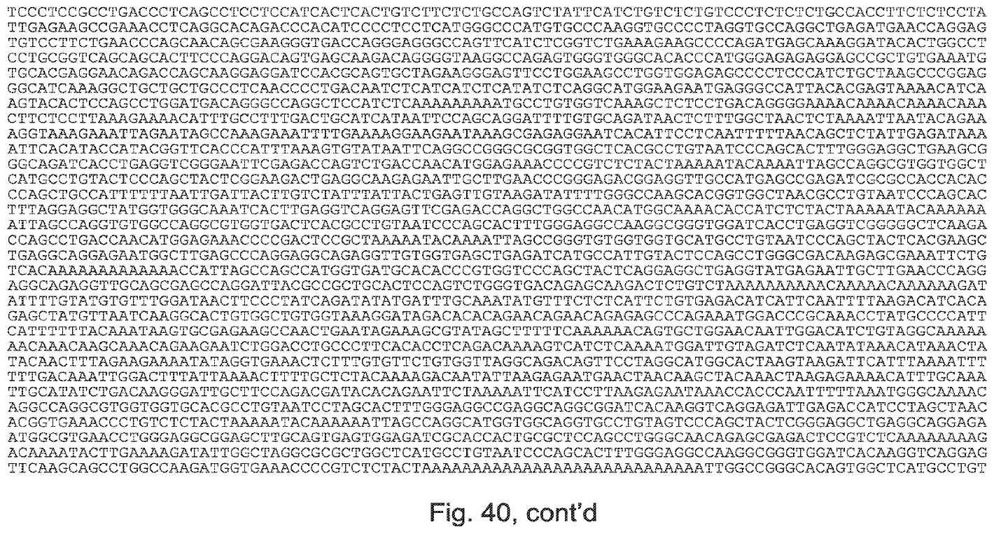

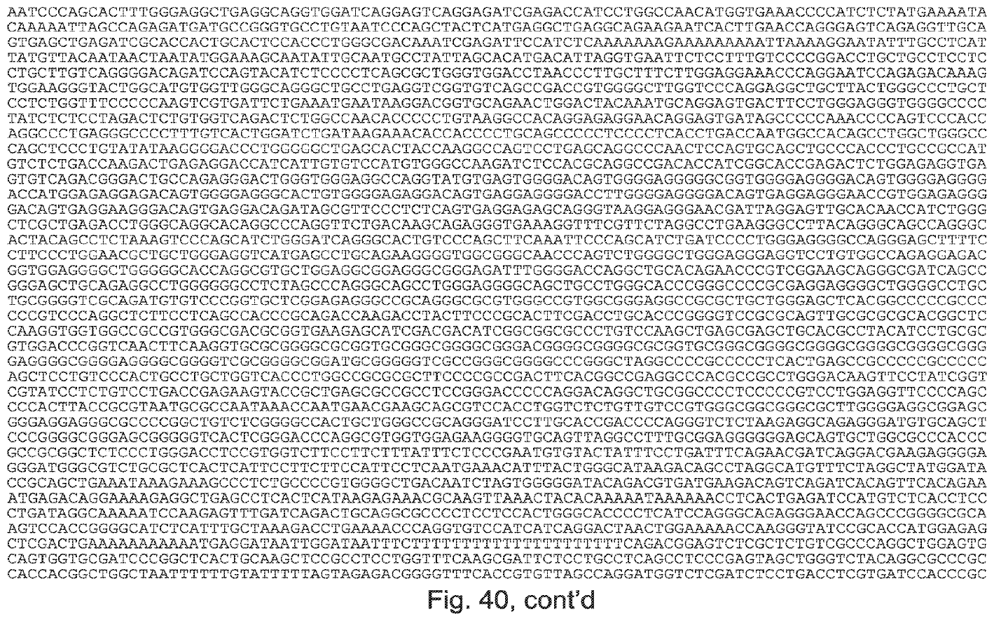

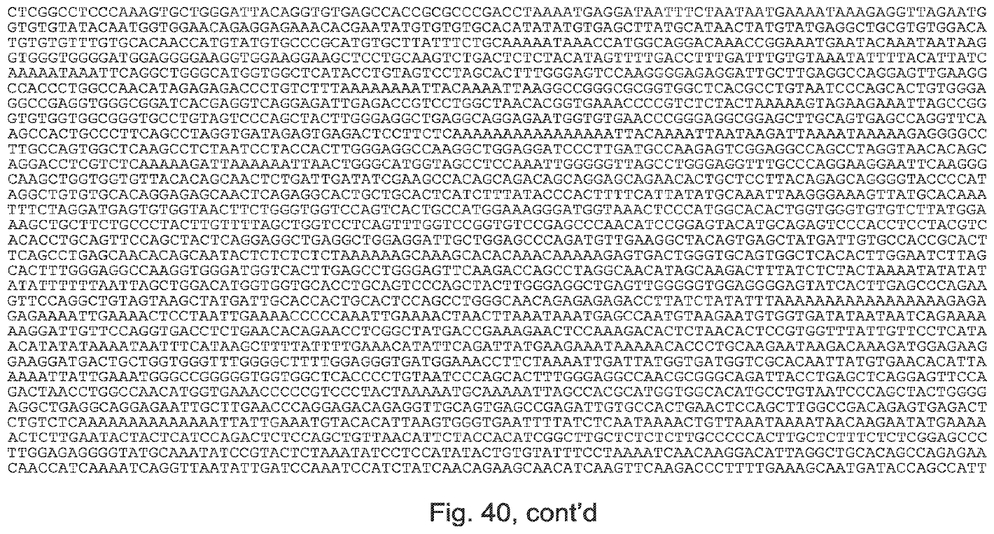

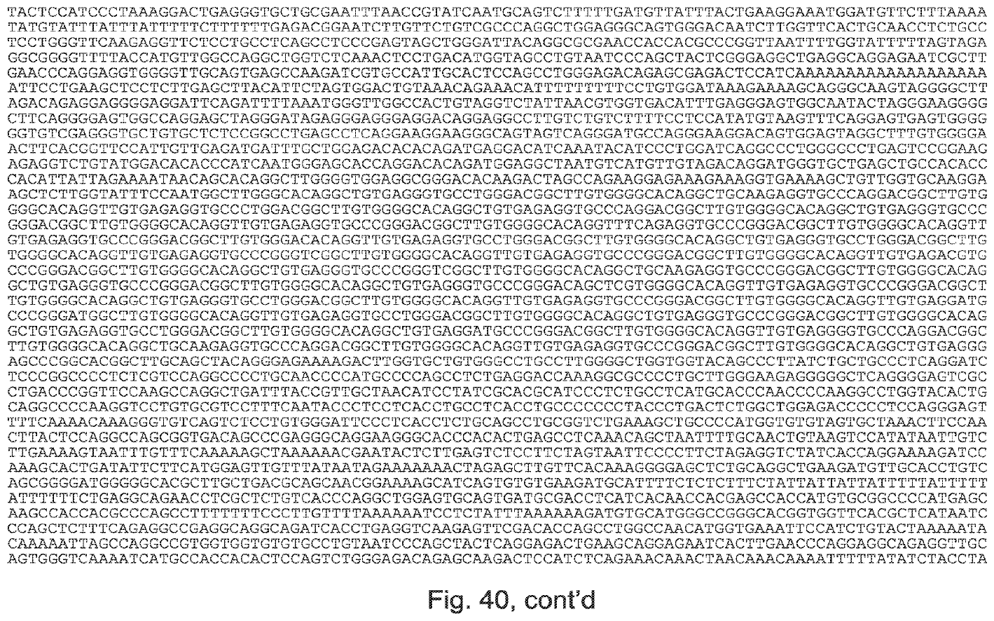

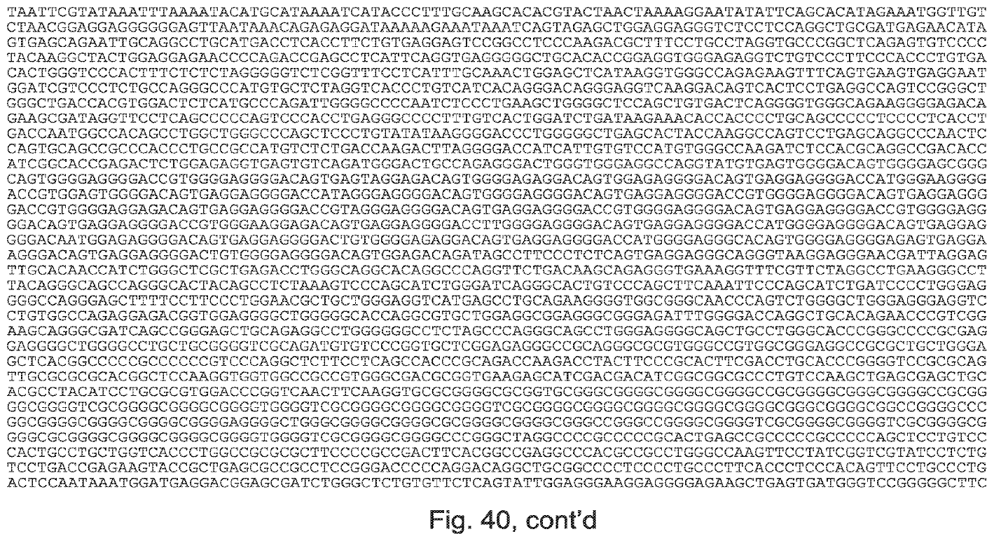

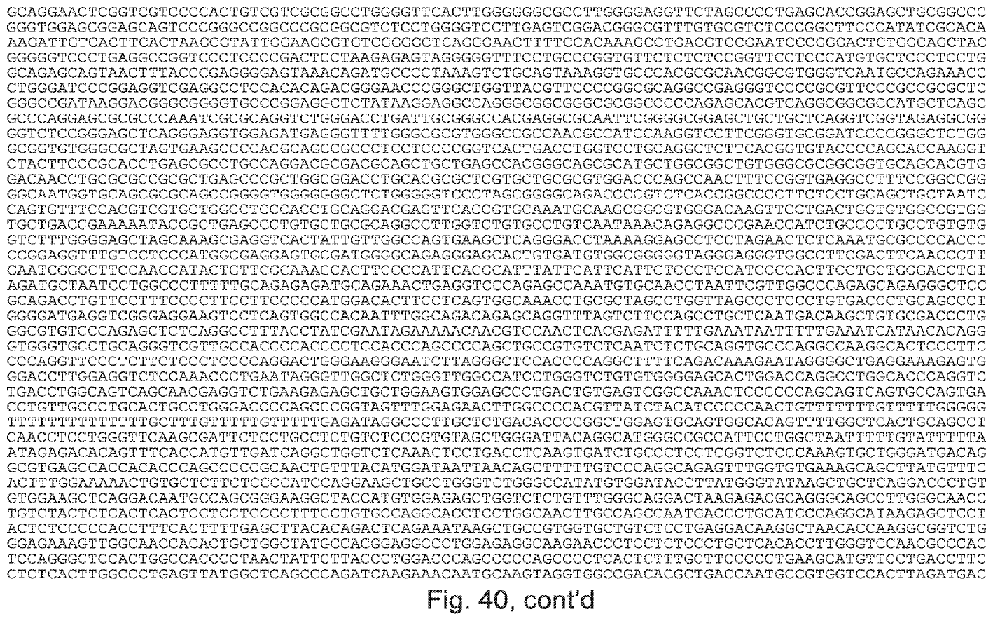

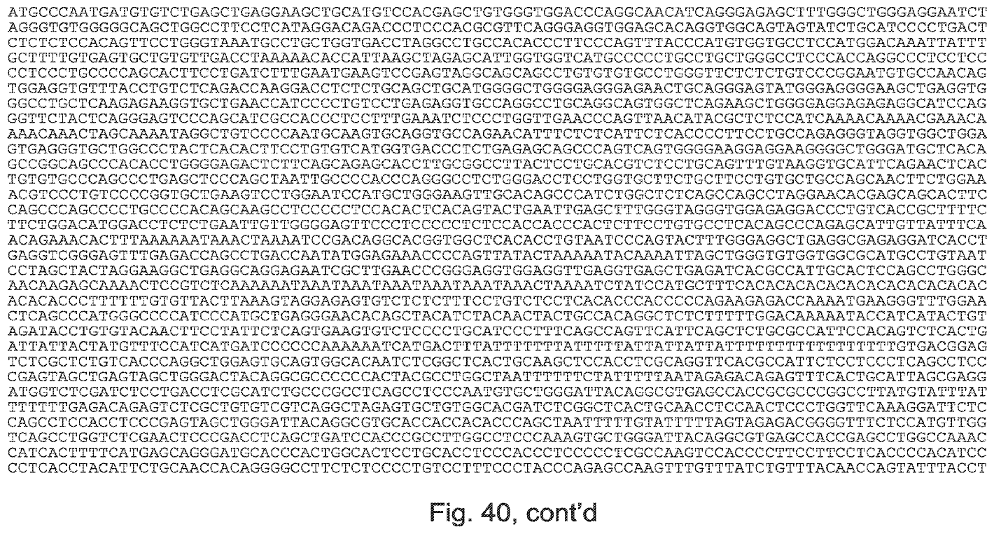

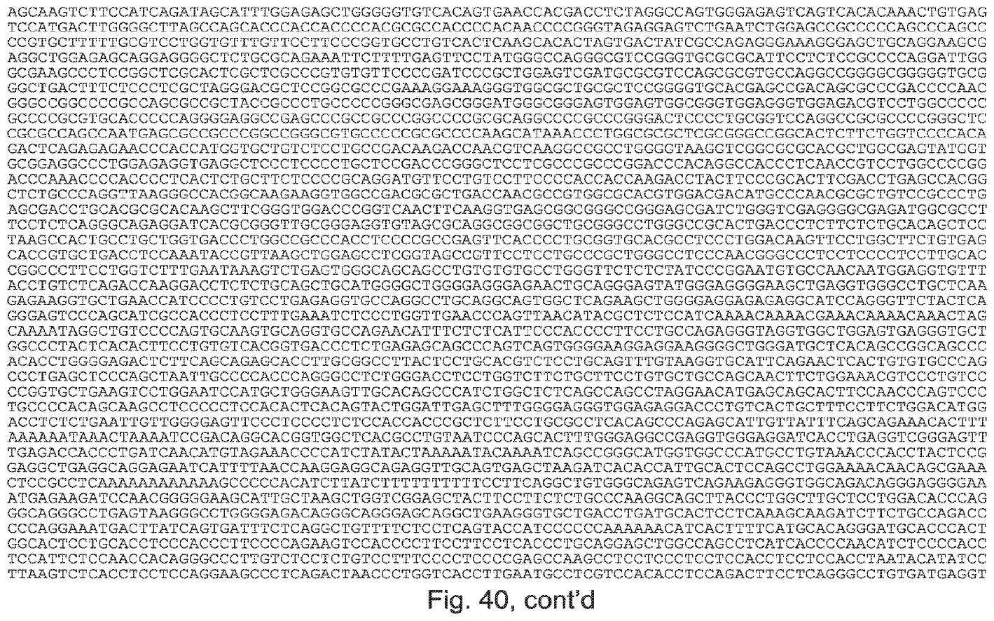

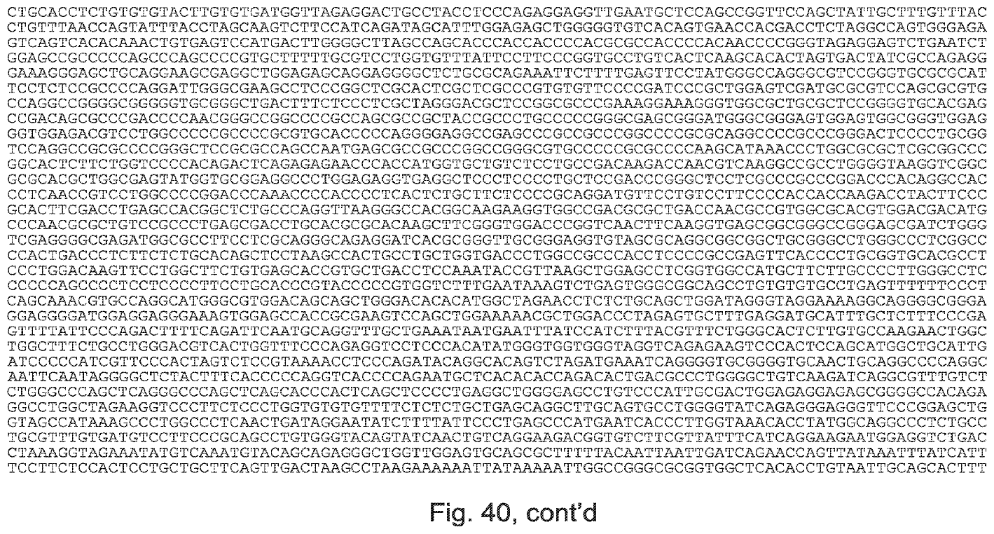

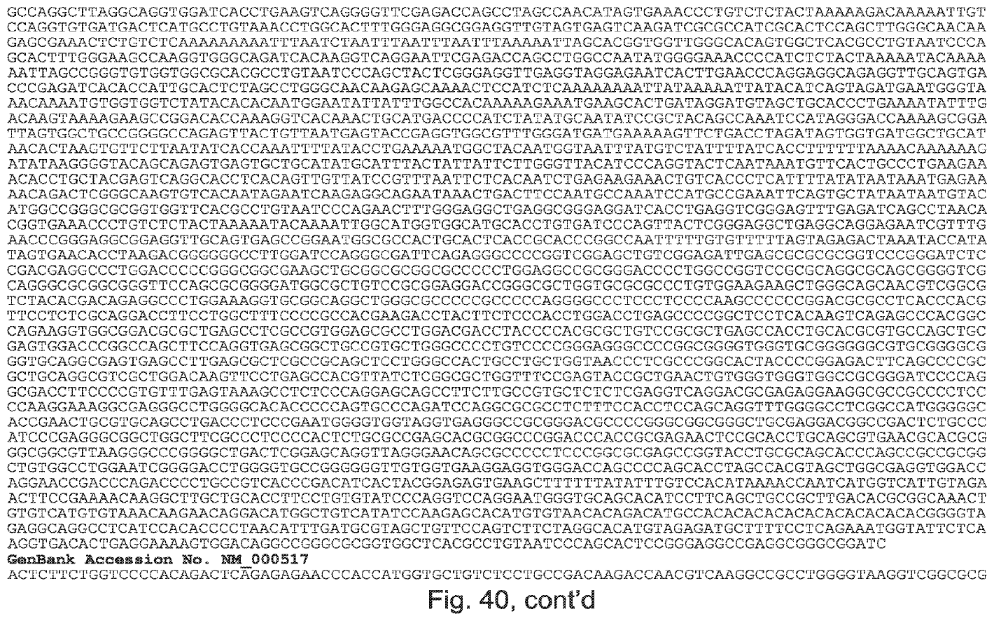

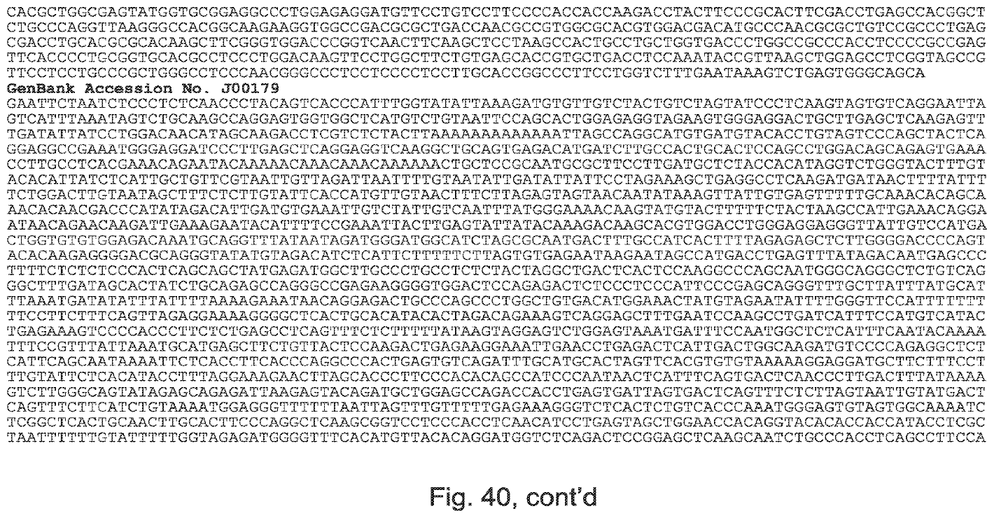

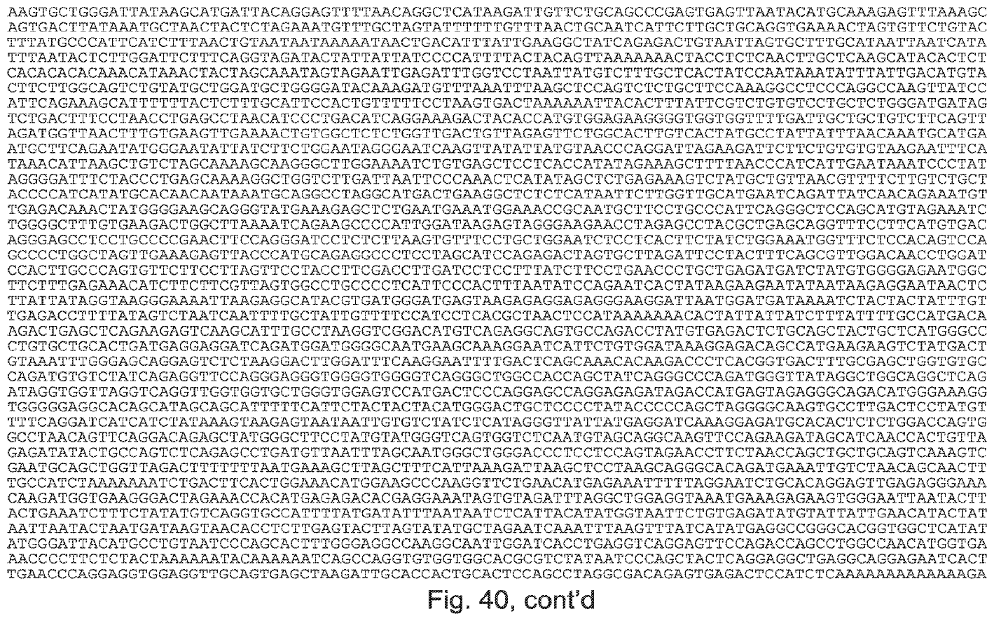

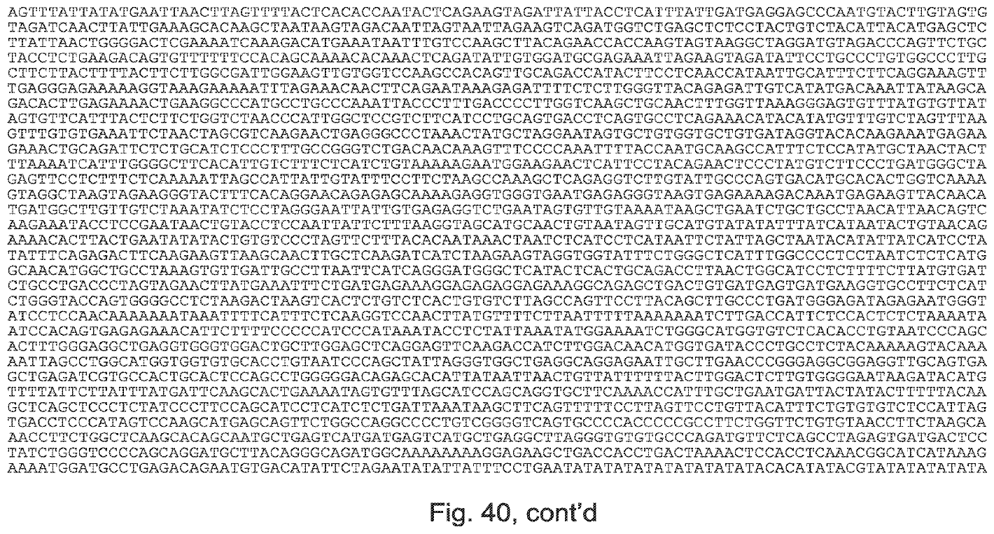

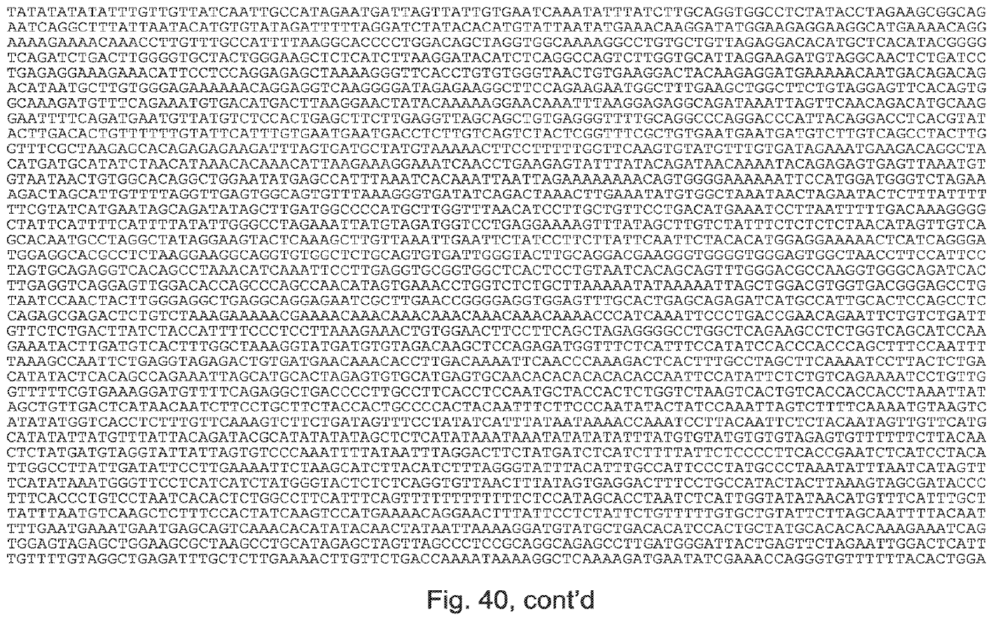

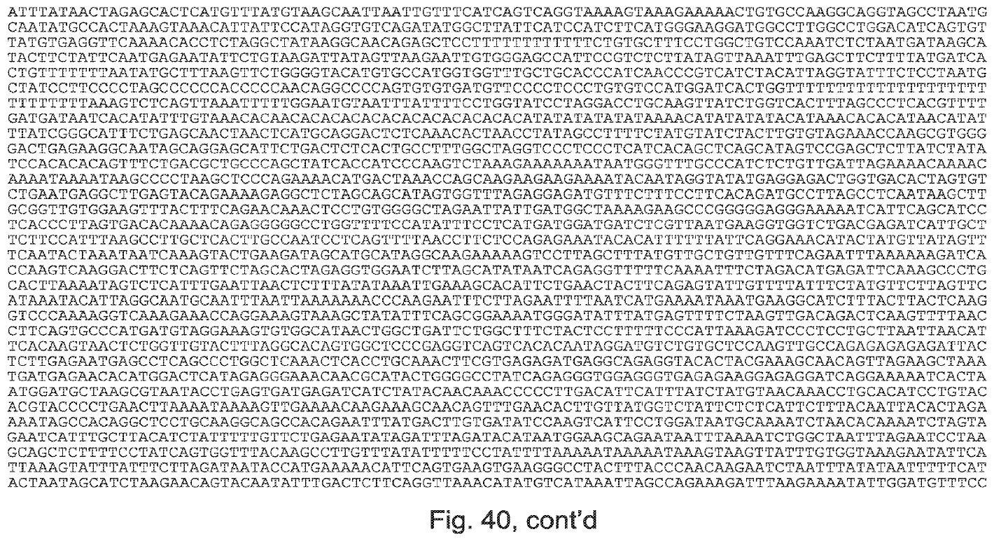

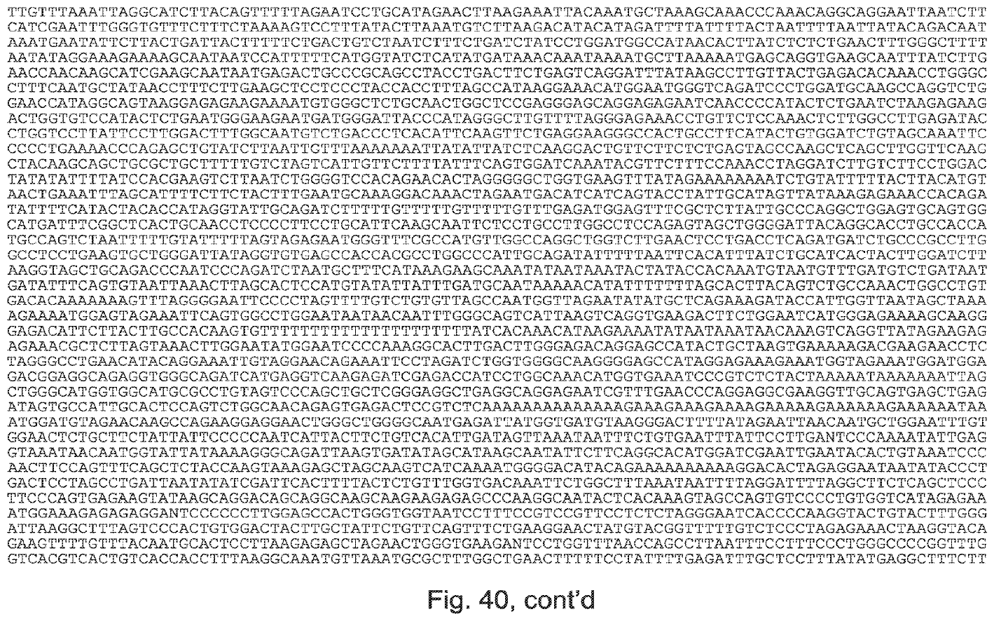

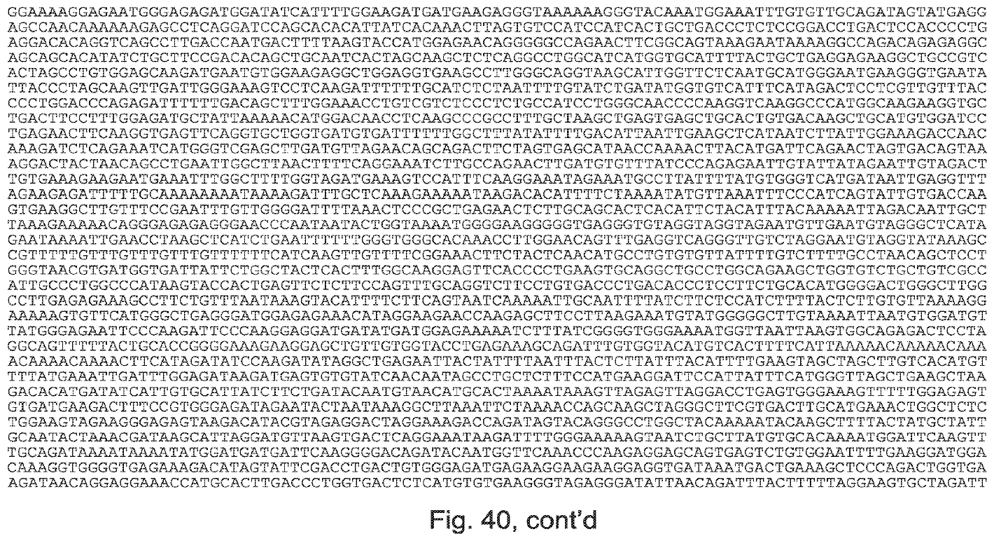

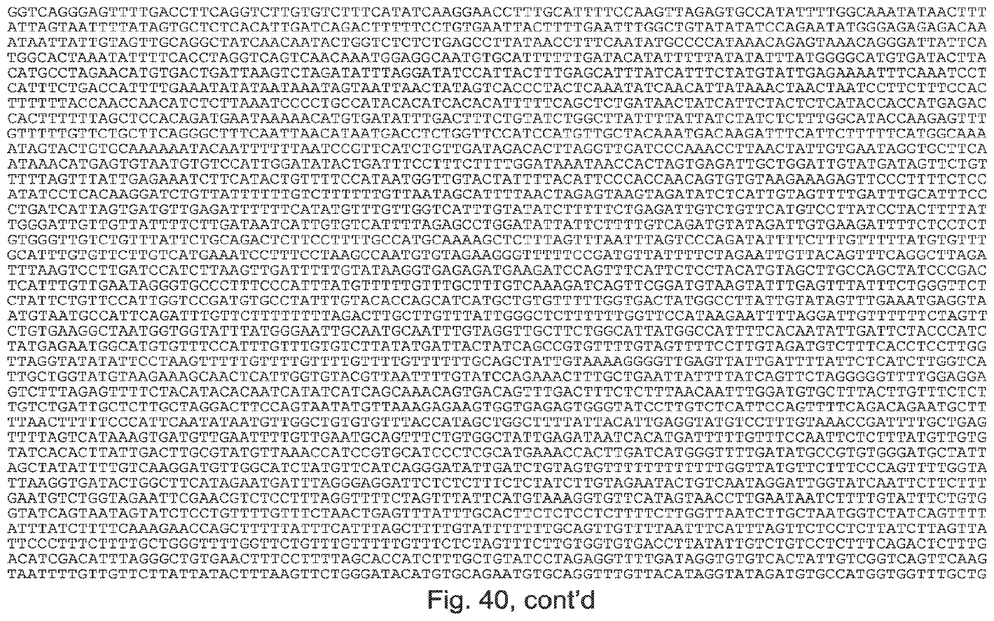

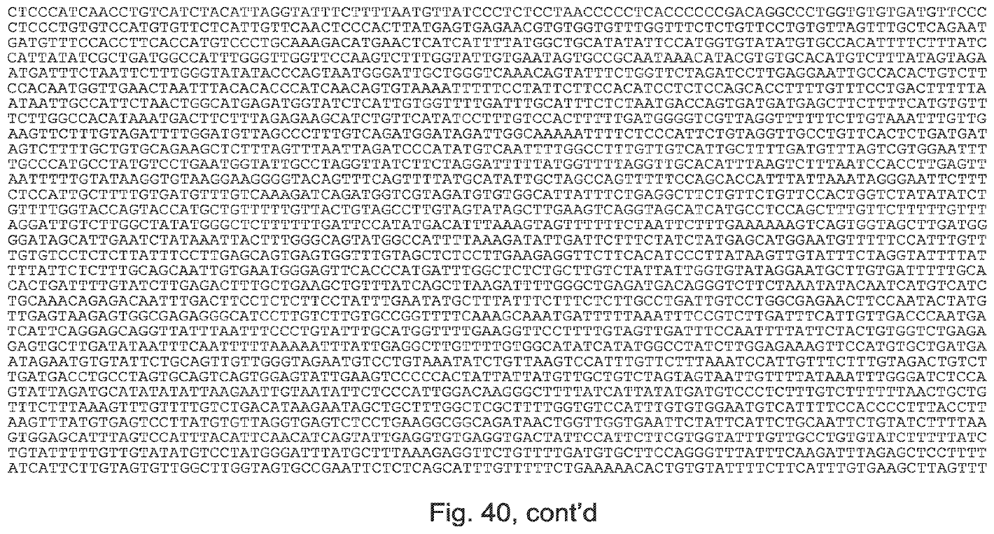

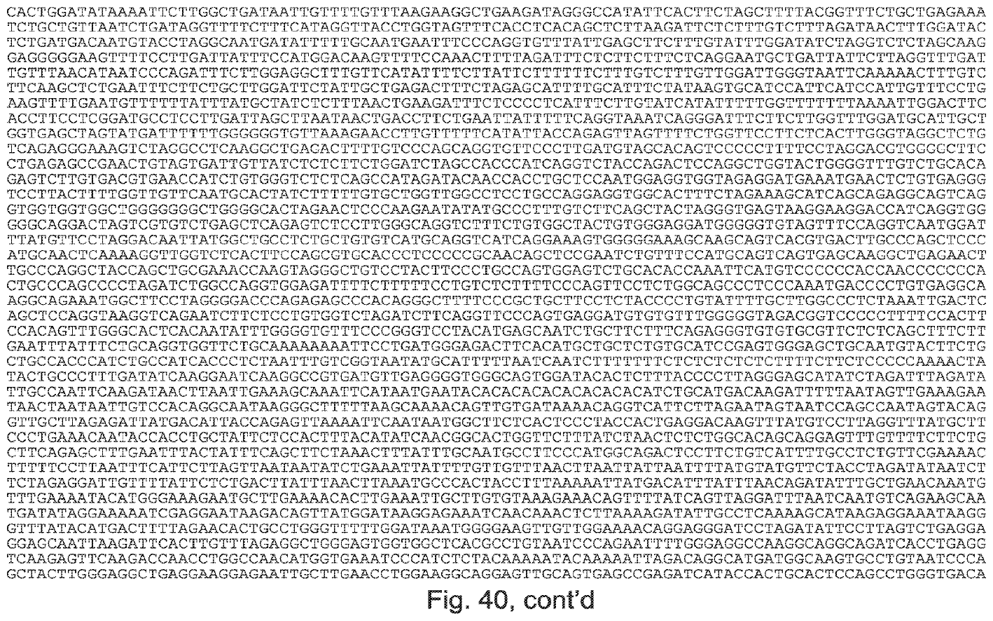

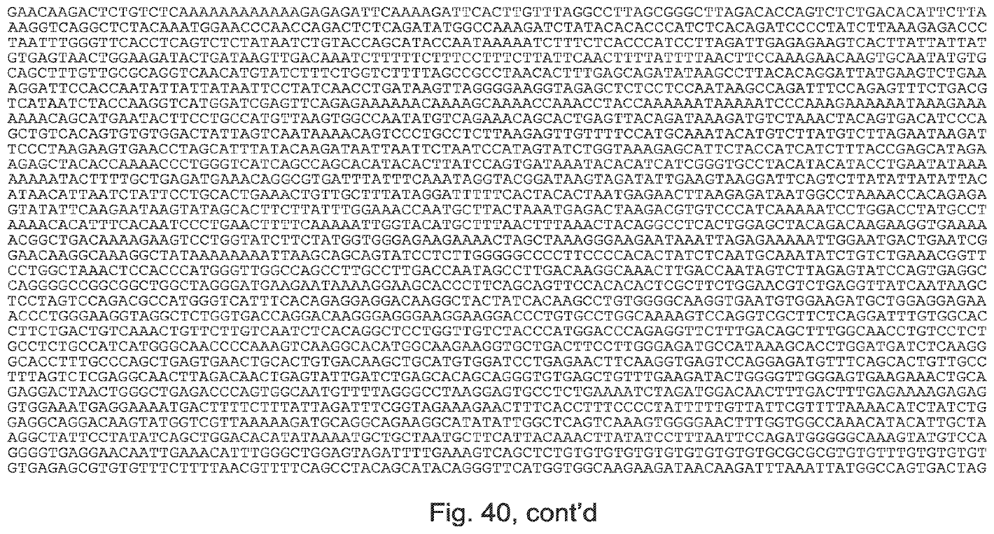

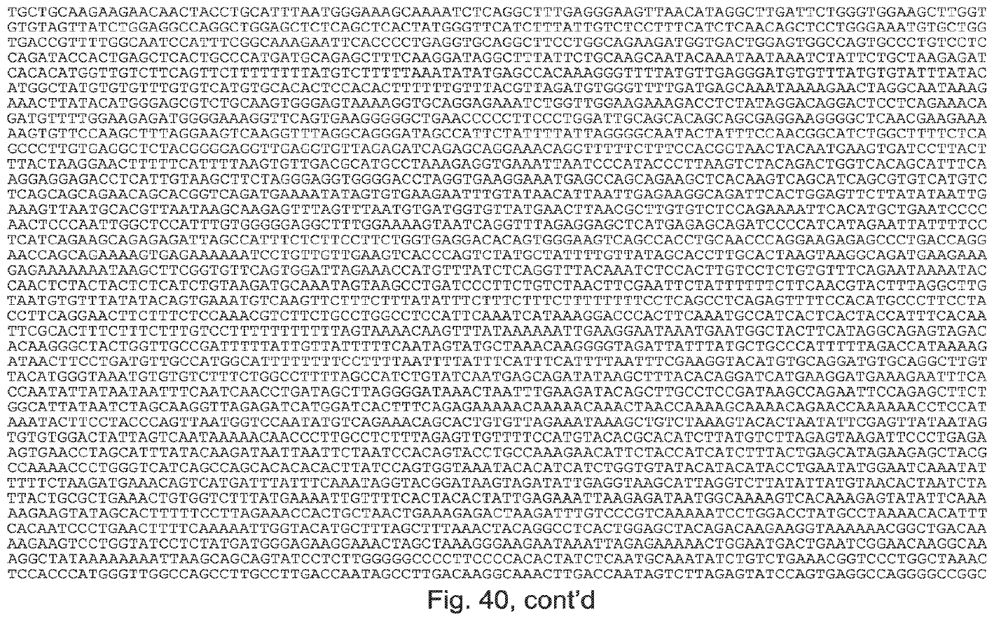

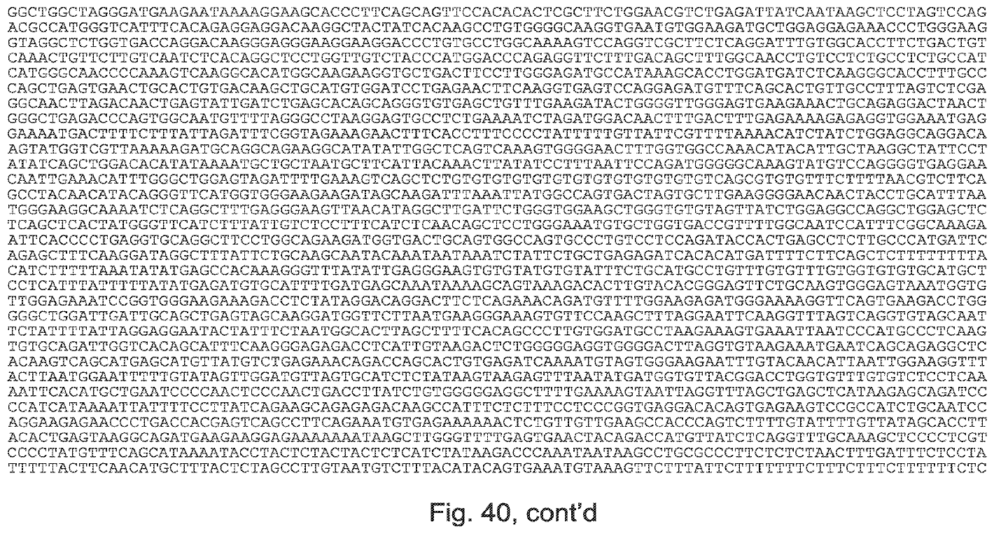

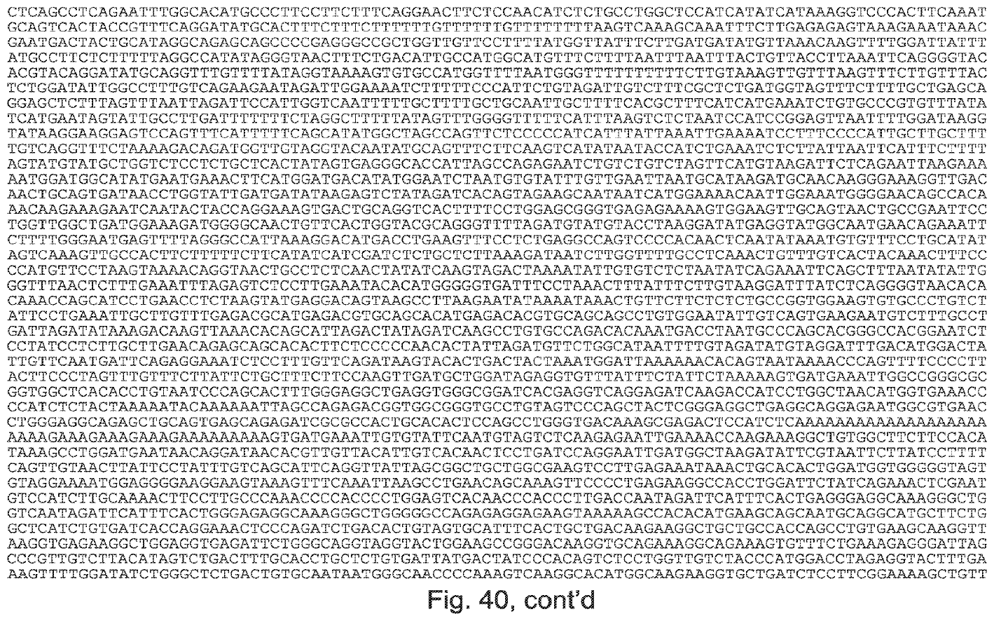

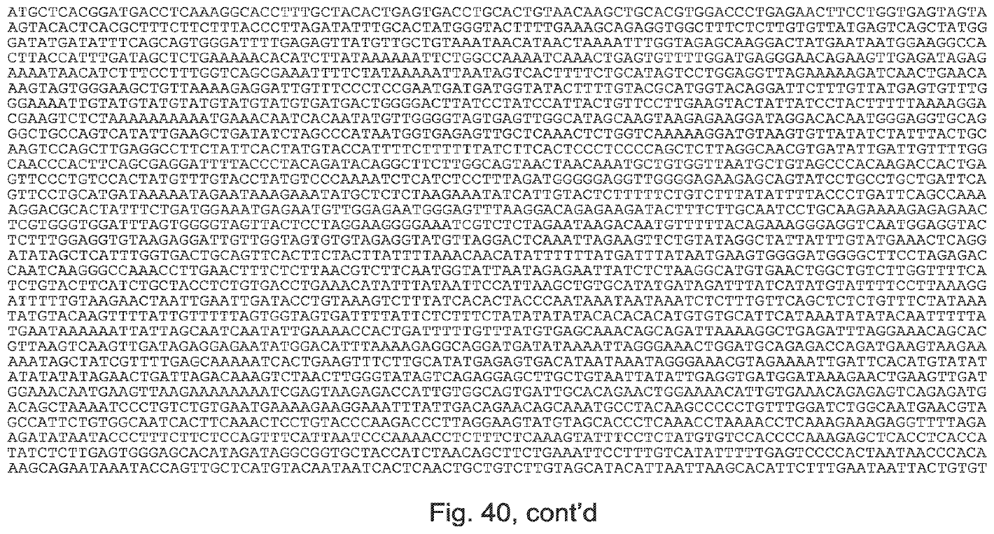

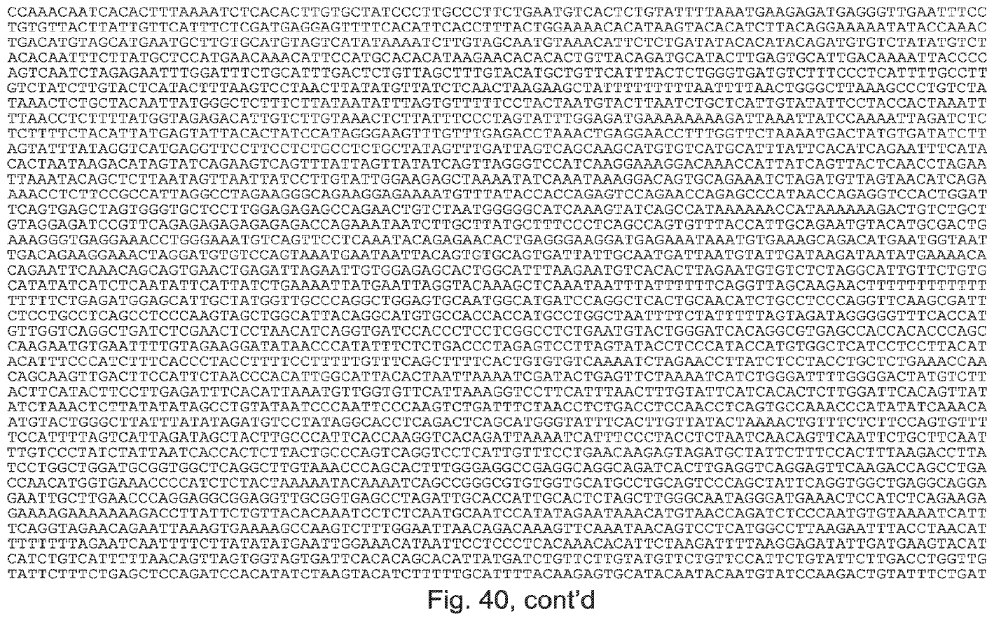

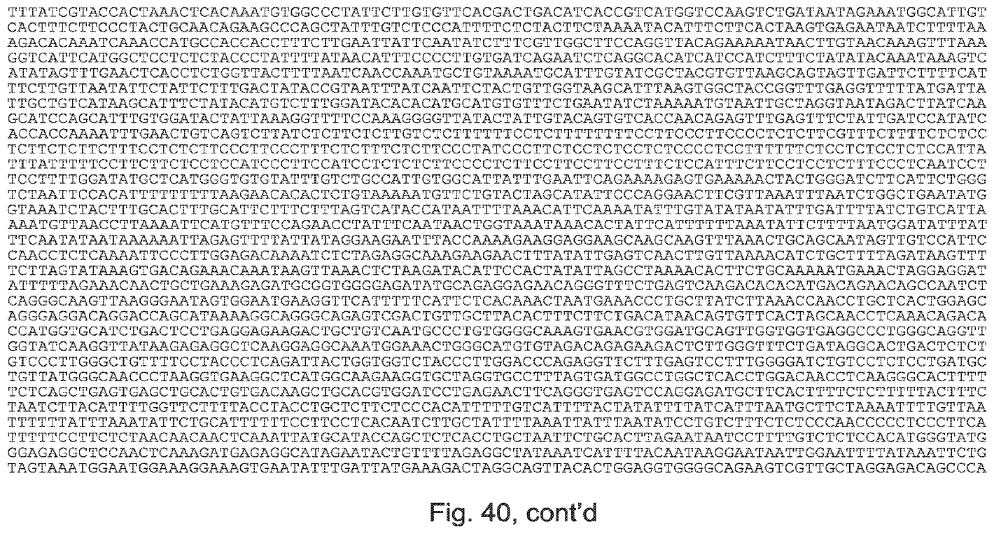

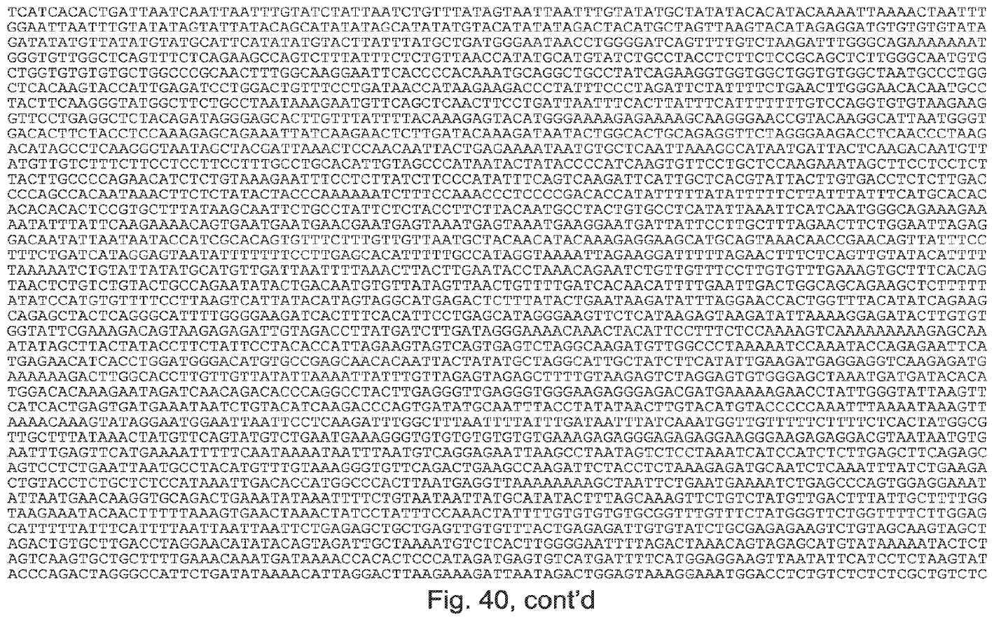

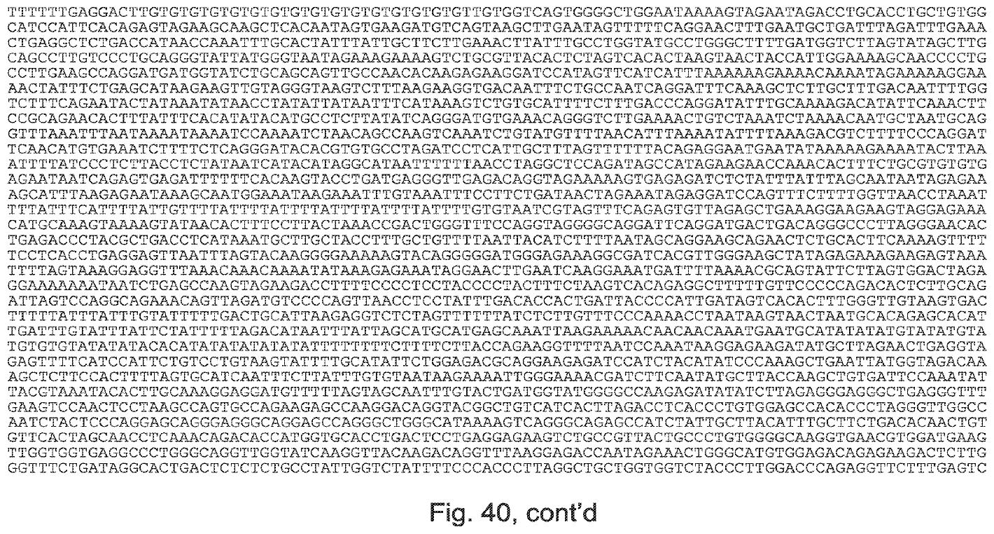

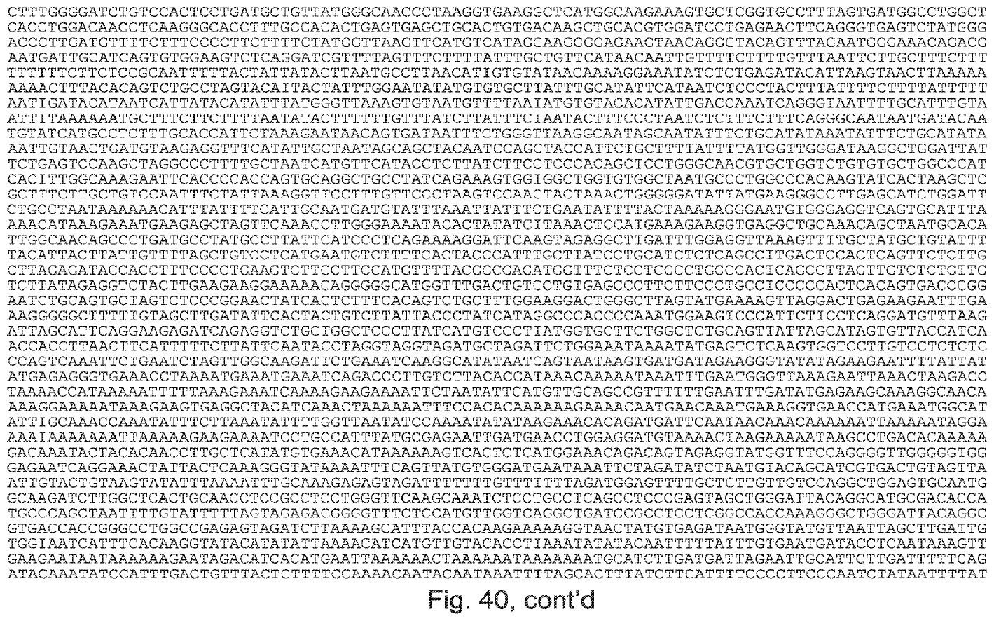

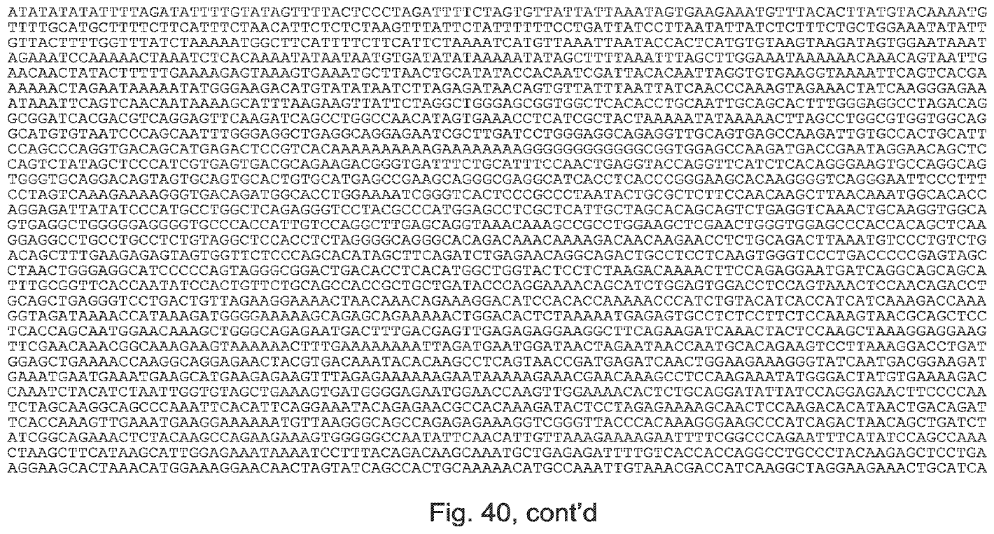

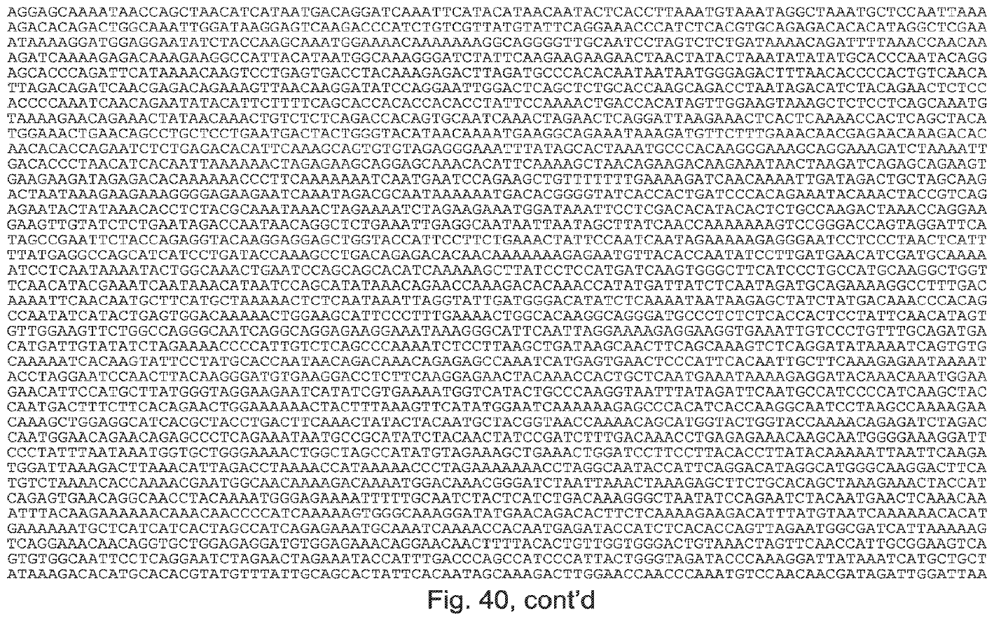

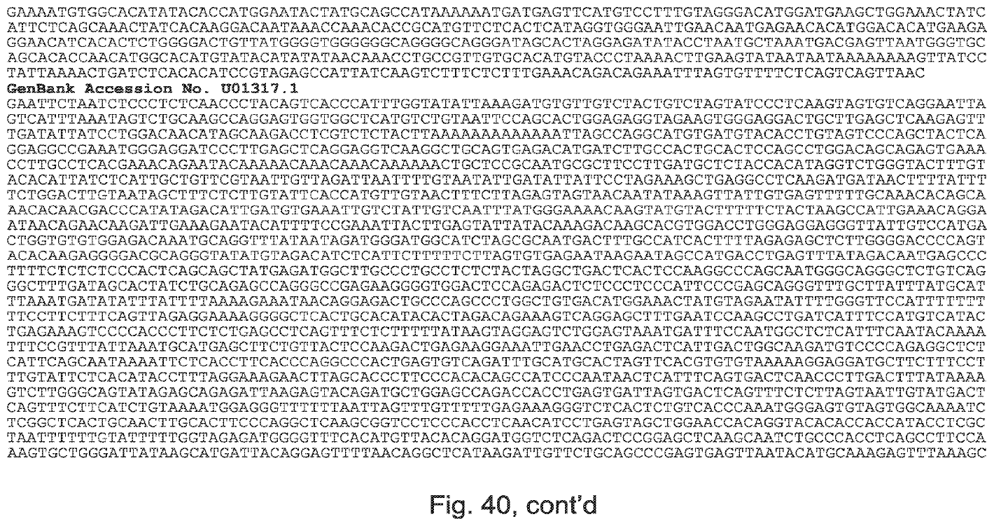

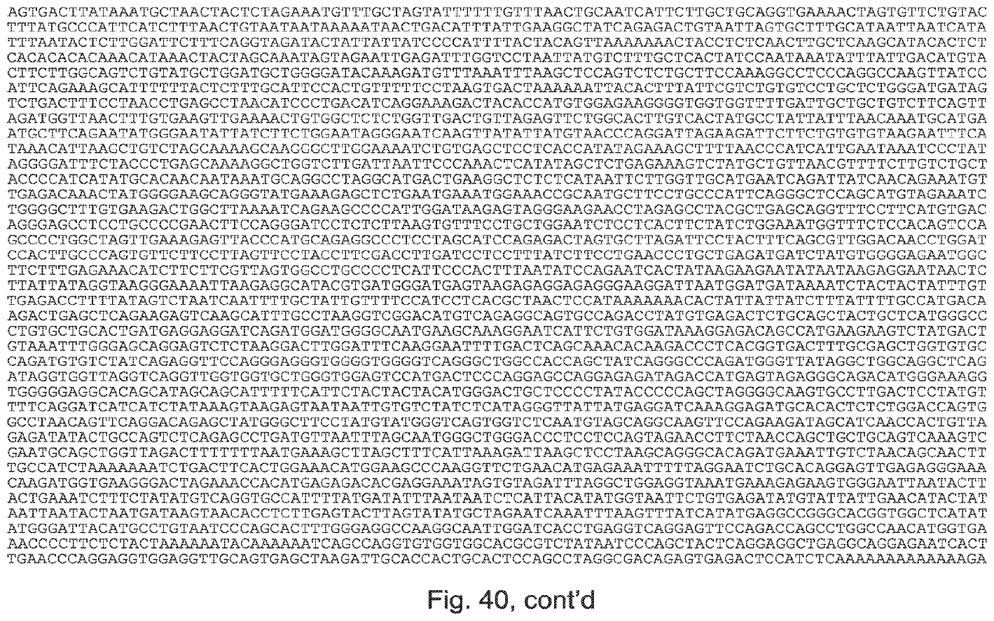

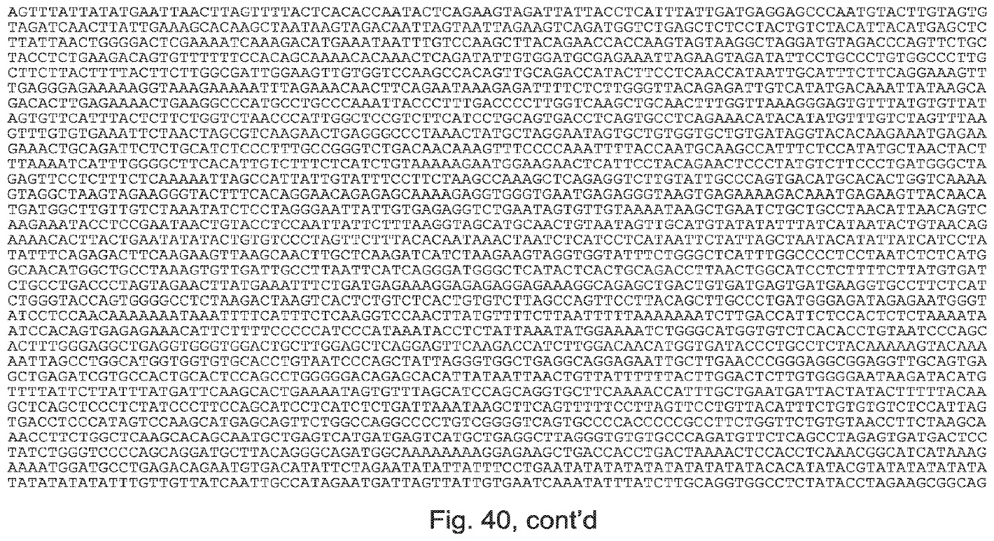

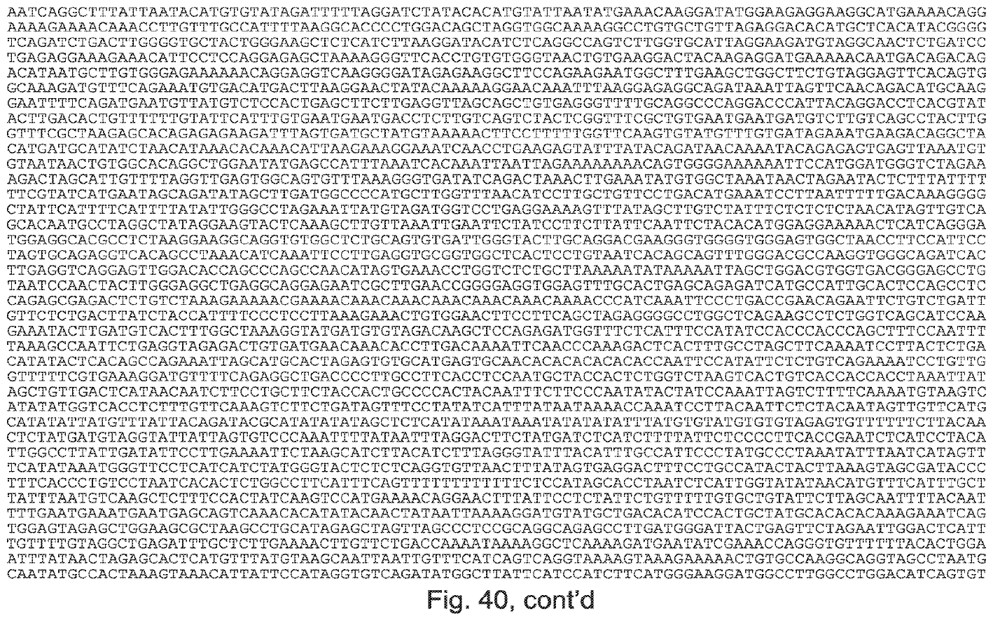

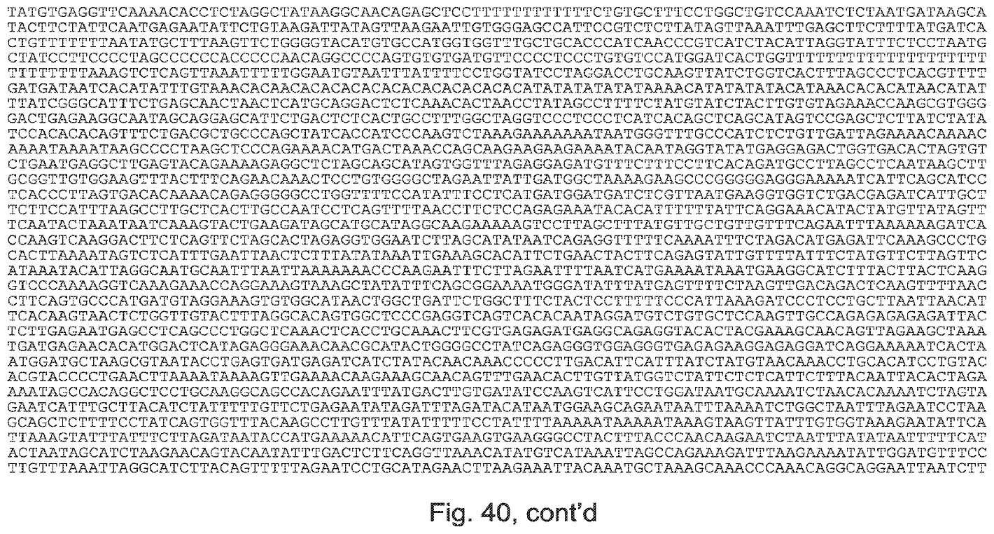

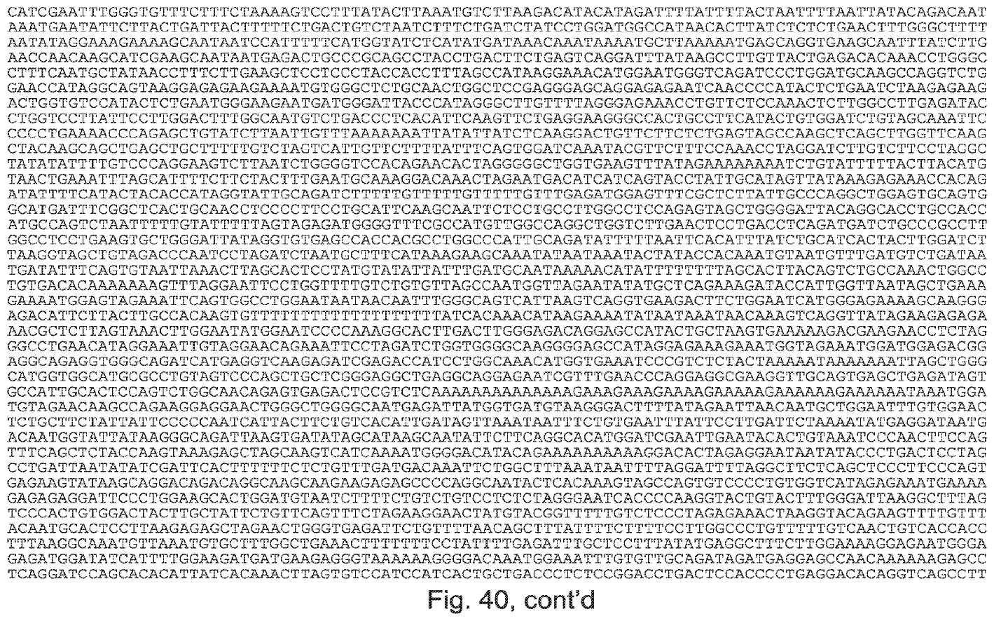

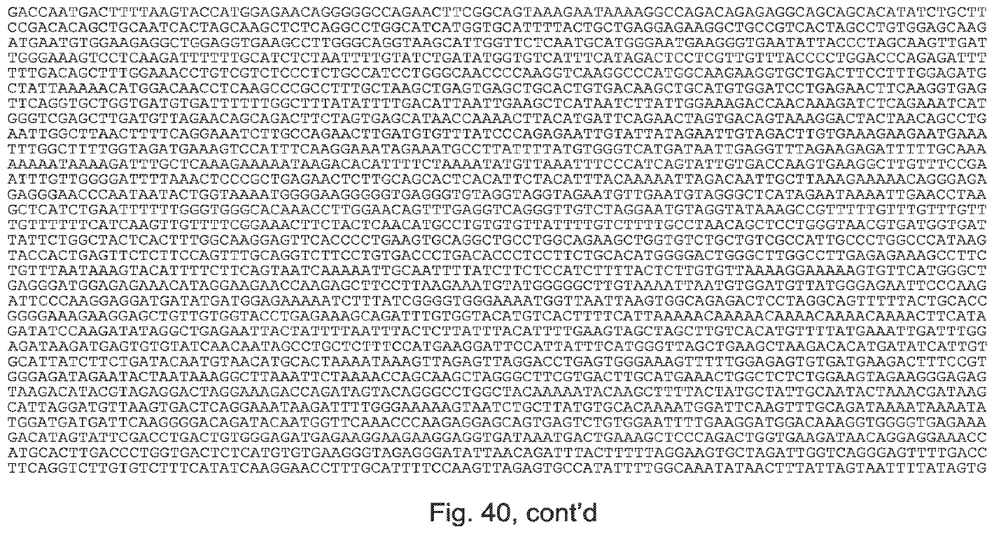

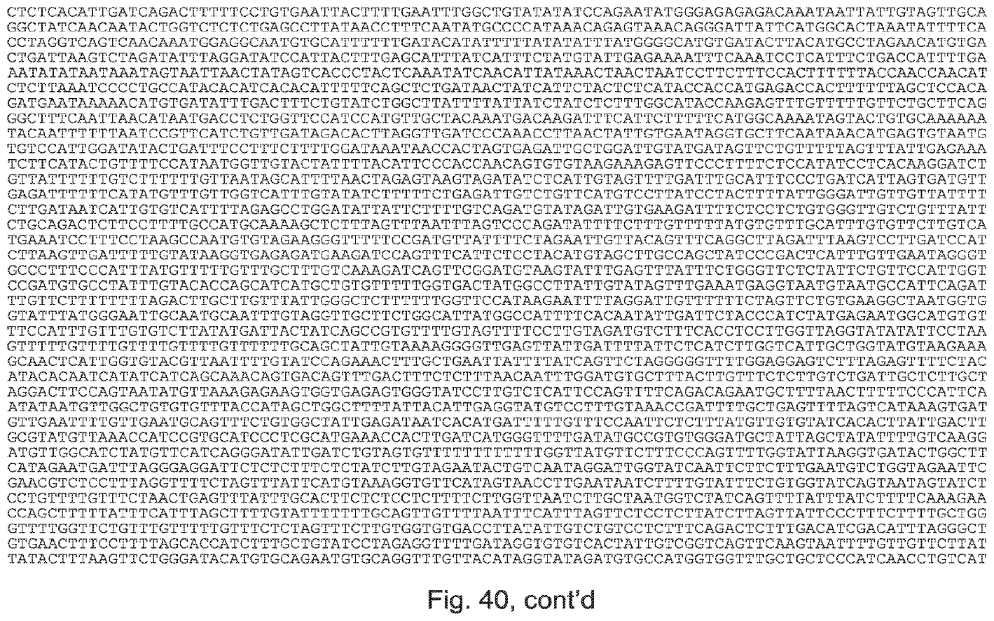

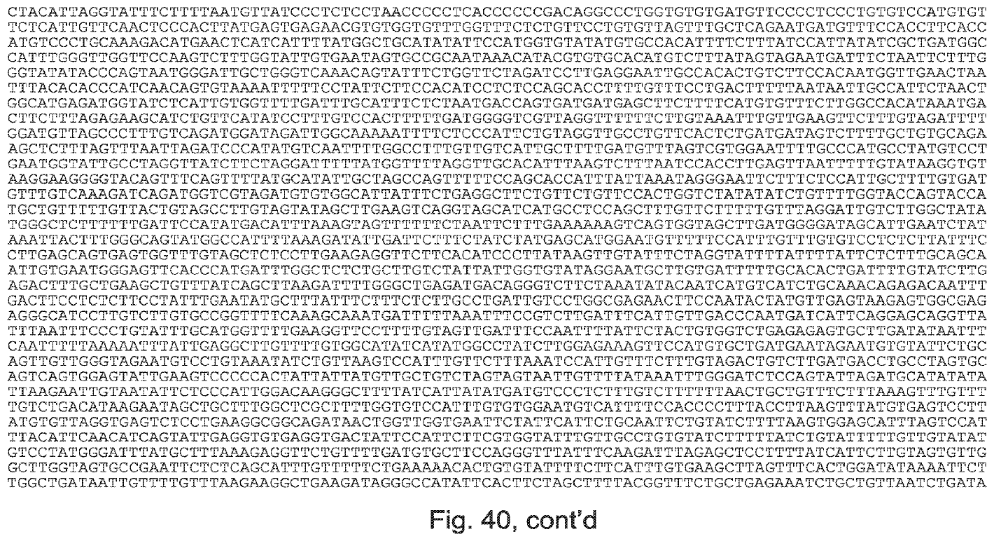

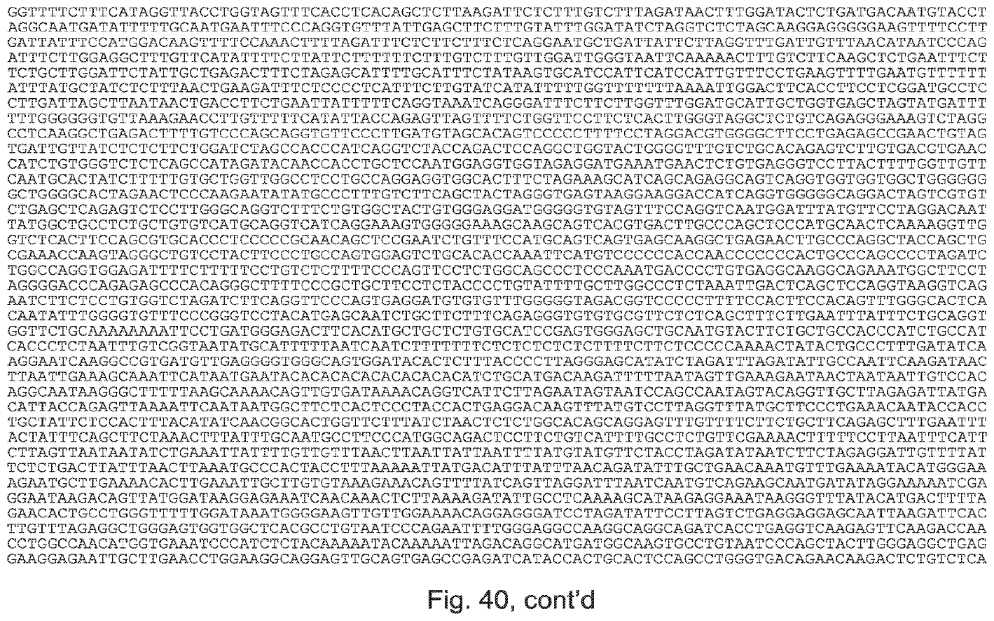

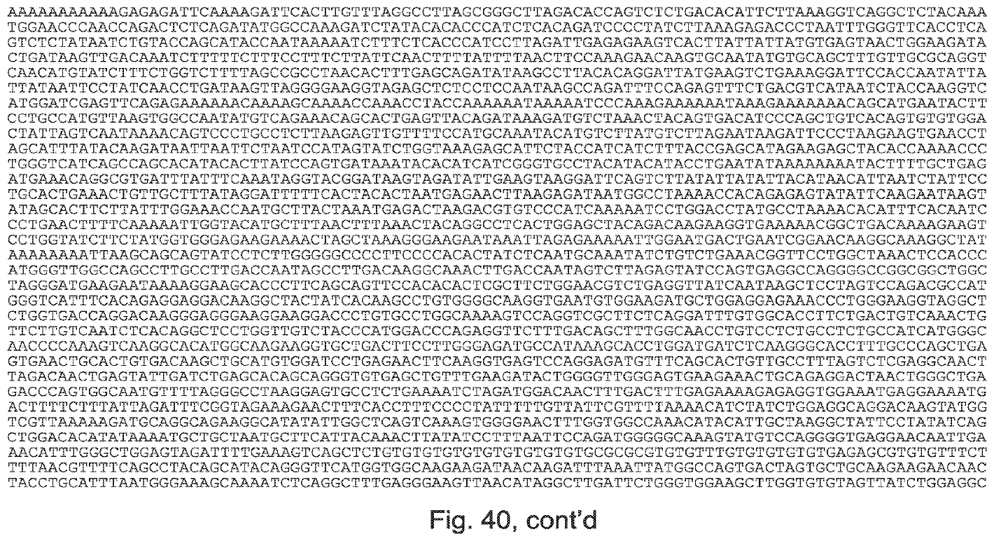

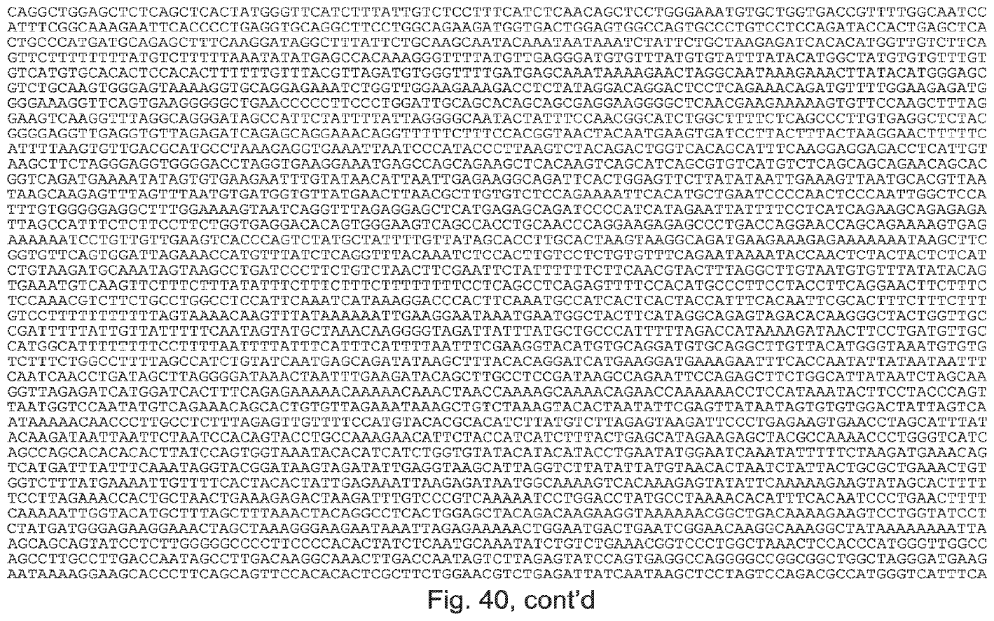

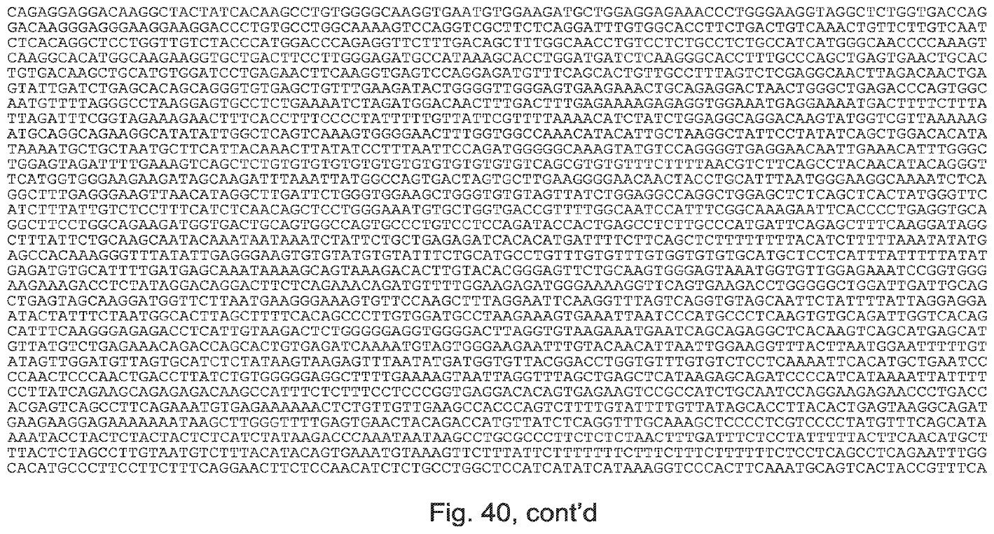

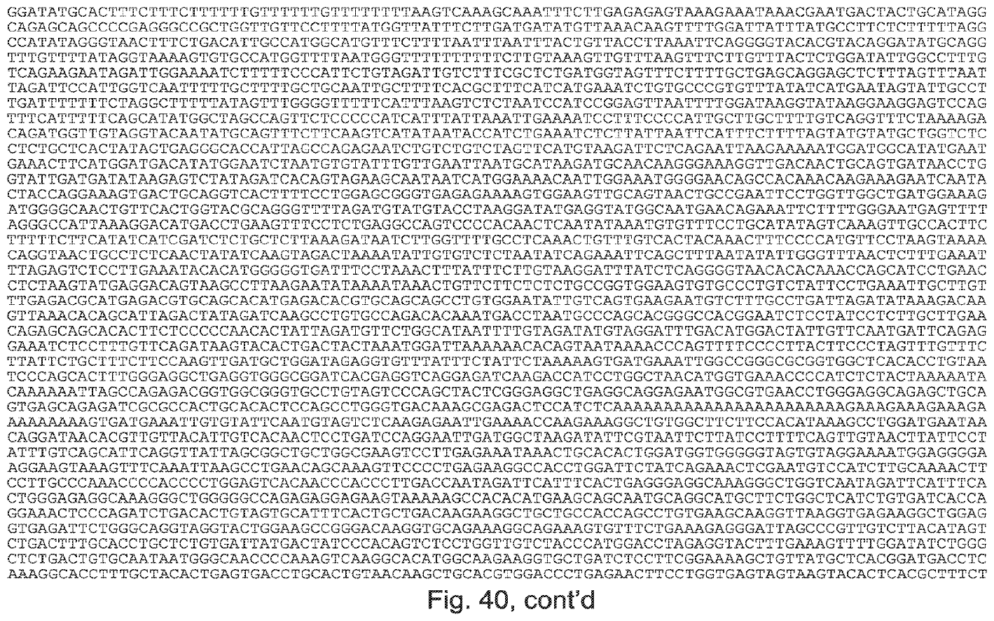

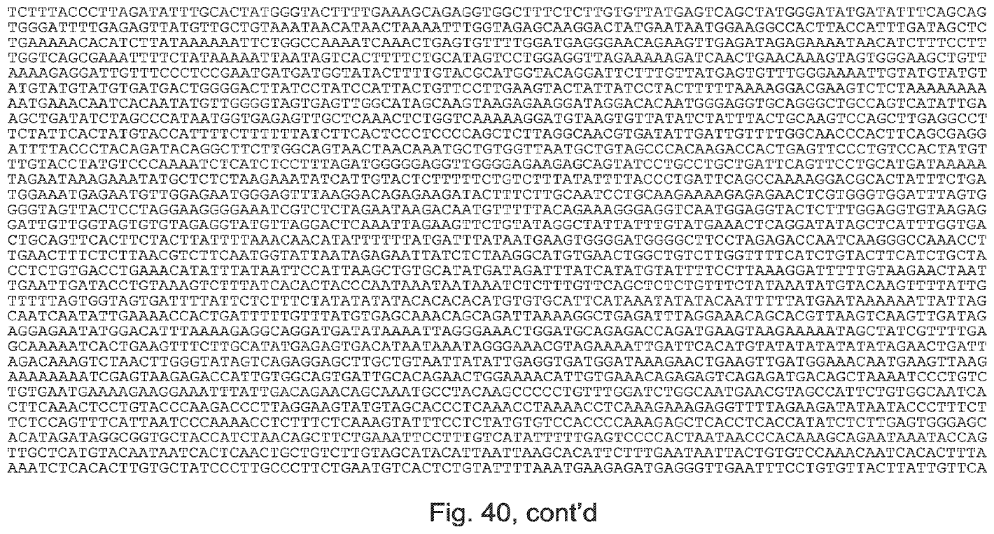

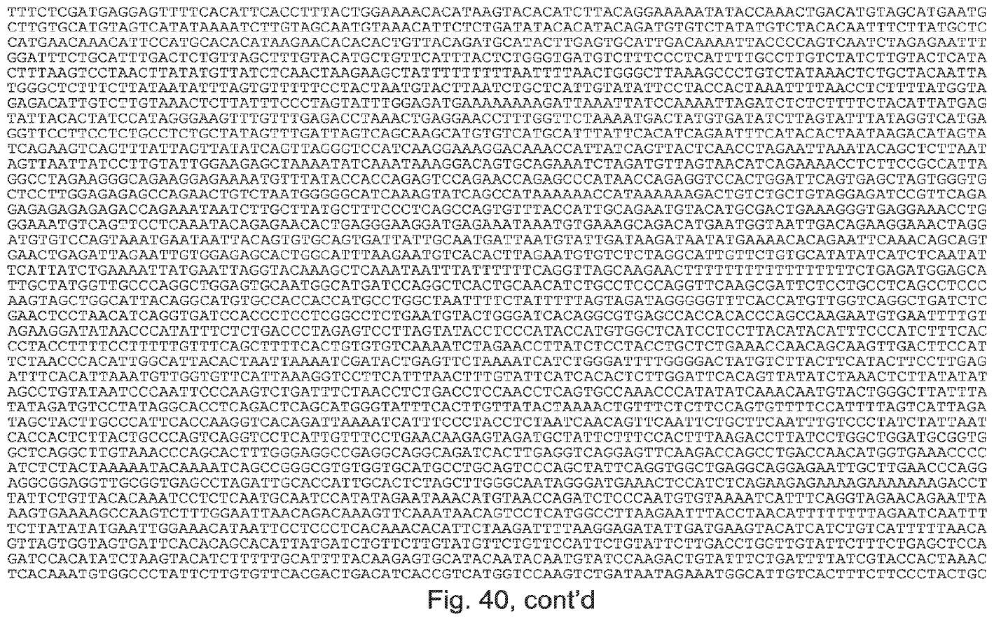

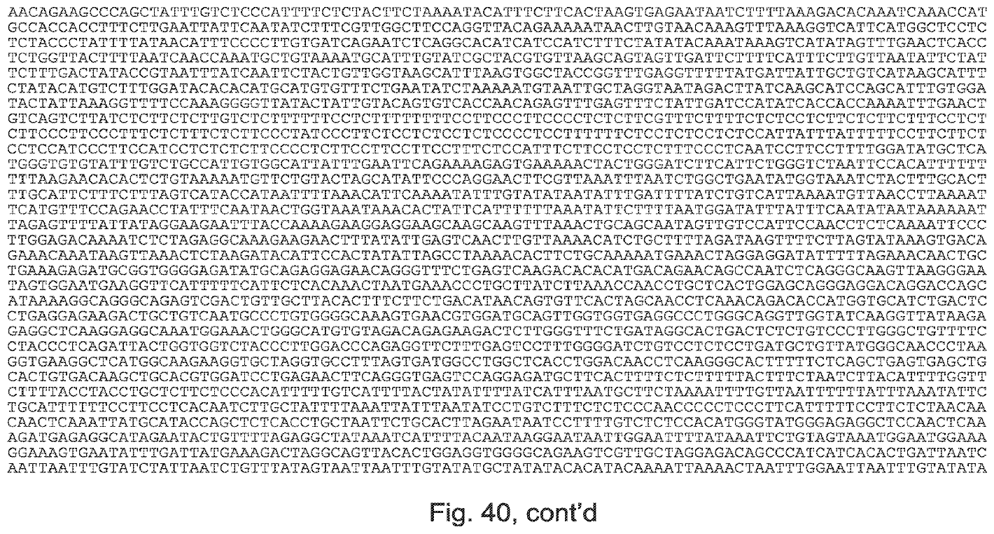

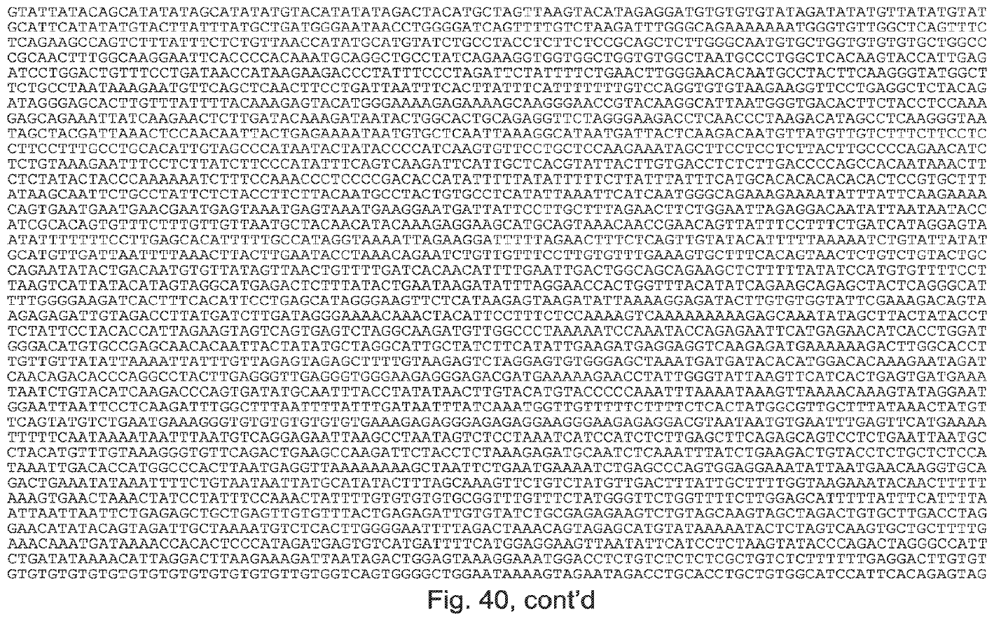

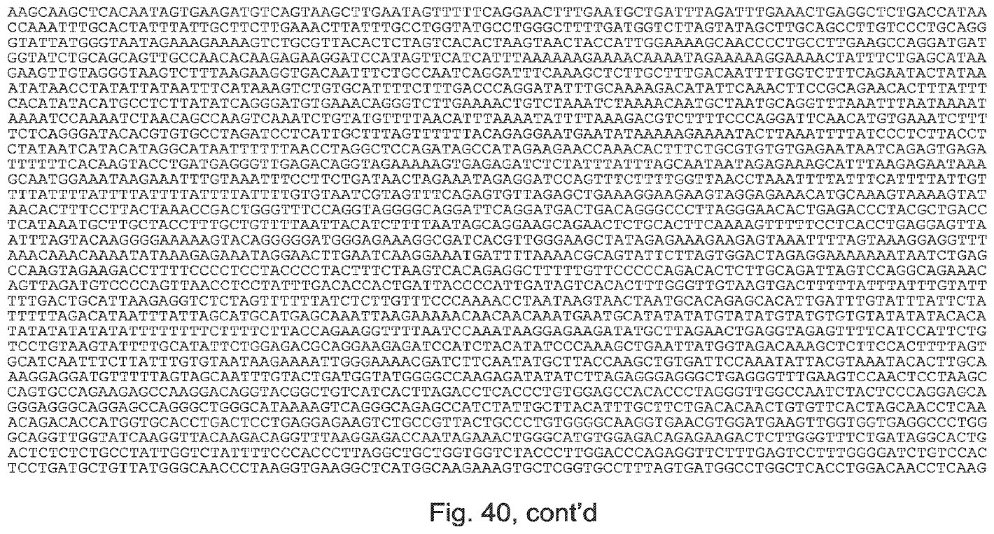

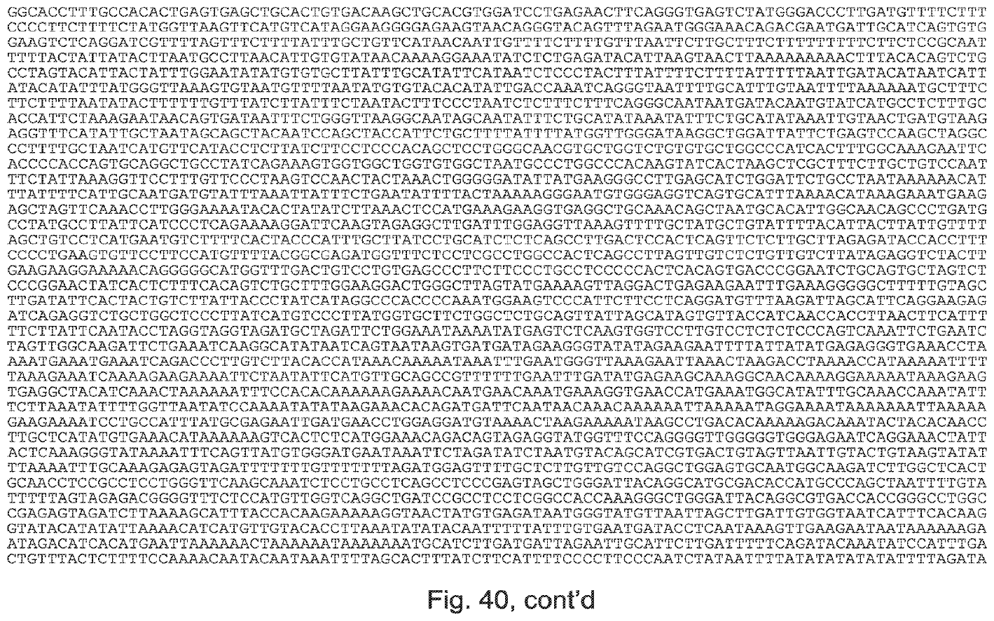

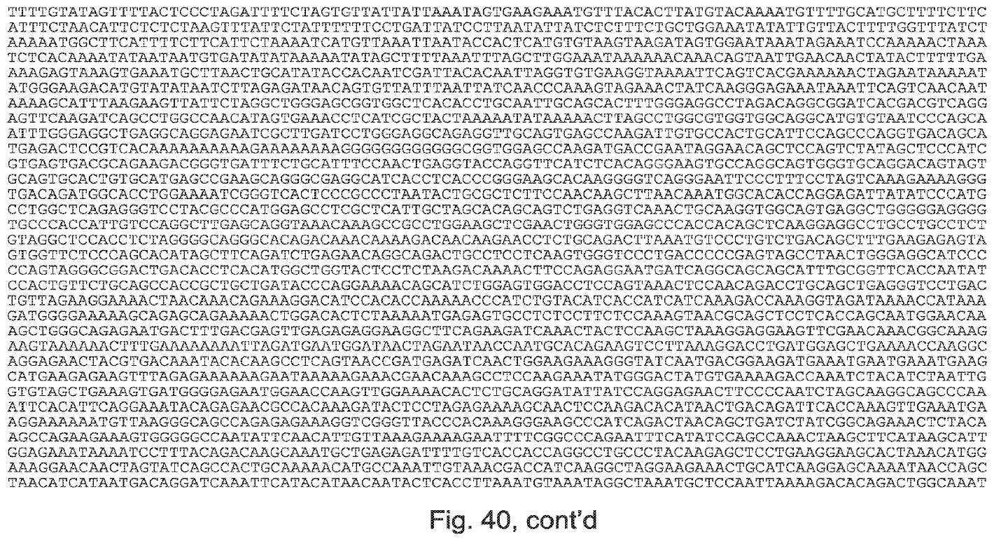

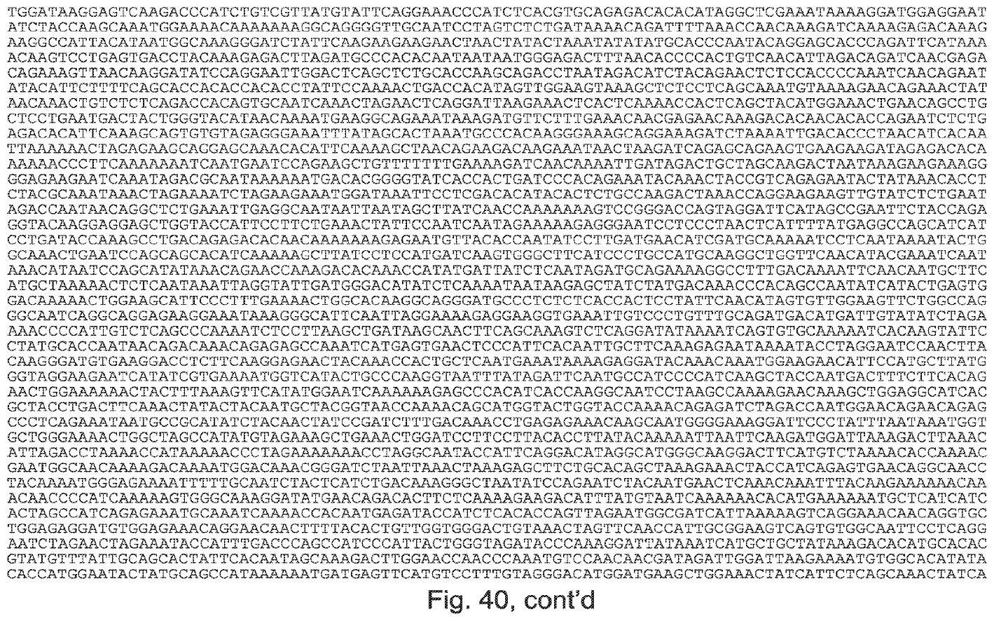

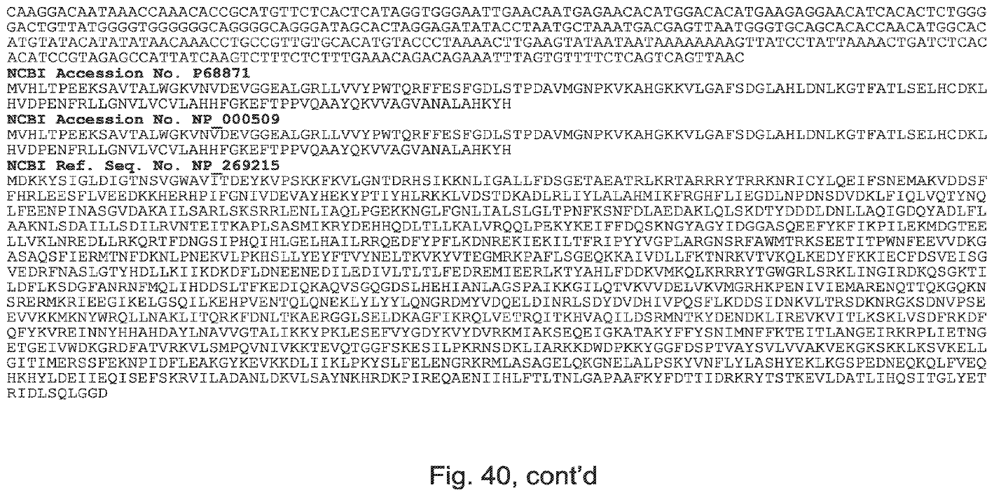

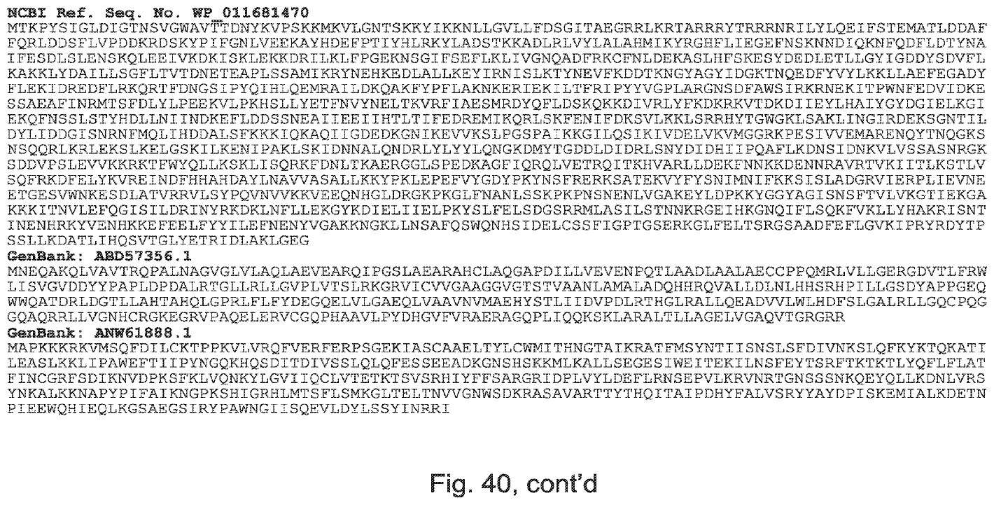

Various sequences corresponding to accession numbers disclosed herein, including e.g., accession sequences referred to herein as SEQ ID NOs: 145, 146, 147, 148, 149, 150, 151, 152, and/or 153 as indicated in Tables 1-18, are provided herein in FIG. 40. Those of skill in the art will appreciate that such sequences, including sequences disclosed in FIG. 40, can be referenced in whole (e.g., by an accession number), or in part (e.g., by reference to a nucleotide position and/or a set or range of nucleotide positions of a sequence and/or accession number).

TABLE 1
Ad3 Genomic Sequences
Ad3 Genomic Sequences
Reference Ad3 Genome Sequence: GenBank
accession no. NC_011203 (SEQ ID NO: 145)

| | Exemplary Sequence | |
|---|---|---|
| Component | (position in reference) | SEQ ID NO: |
| Ad3 5′ (left) ITR | 1-136 | 1 |
| Ad3 3′ (right) ITR | 35208-35343 | 2 |
| Ad3 Packaging | 137-479 | 3 |
| Sequence | | |
| Ad3 E1 | 480-3918 | 4 |
| Ad3 E2 | 26643-3947 | 5 |
| Ad3 E3 | 27085-31186 | 6 |
| Ad3 fiber | 31368-32327 | 7 |
| Ad3 fiber tail | 31368-31493 | 166 |
| Ad3 fiber shaft | 31494-31763 | 8 |
| Ad3 fiber knob | 31764-32324 | 9 |
| Ad3 penton | 13905-15539 | 10 |
| Ad3 hexon | 18418-21252 | 11 |

TABLE 2
Ad3 Amino Acid Sequences
Ad3 Amino Acid Sequences

| | Exemplary Sequence | SEQ ID |
|---|---|---|
| Component | (position in reference) | NO: |
| Ad3 fiber | 1-319 (GenBank accession no. YP_002213796) | 12 |
| Ad3 fiber shaft | 43-132 (GenBank accession no. YP_002213796) | 13 |
| Ad3 fiber knob | 134-319 (GenBank accession no. YP_002213796) | 14 |
| Ad3 penton | 1-544 (GenBank accession no. YP_002213774) | 15 |
| Ad3 hexon | 1-944 (GenBank accession no. YP_002213779) | 16 |
| Ad3 fiber tail | 1-42 (GenBank accession no. YP_002213796) | 157 |

TABLE 3
Ad7 Genomic Sequences
Ad7 Genomic Sequences
Reference Ad7 Genome Sequence: GenBank accession
number AC_000018 (SEQ ID NO: 146)

| | Exemplary Sequence | SEQ ID |
|---|---|---|
| Component | (position in reference) | NO: |
| Ad7 5′ (left) ITR | 1-136 | 17 |
| Ad7 3′ (right) ITR | 35379-35514 | 18 |
| Ad7 Packaging | 137-479 | 19 |
| Sequence | | |
| Ad7 E1 | 480-3919 | 20 |
| Ad7 E2 | 26867-3947 | 21 |
| Ad7 E3 | 27308-31345 | 22 |
| Ad7 fiber | 31529-32506 | 23 |
| Ad7 fiber tail | 31529 -31654 | 167 |
| Ad7 fiber shaft | 31655-31927 | 24 |
| Ad7 fiber knob | 31928-32503 | 25 |
| Ad7 penton | 14153-15787 | 26 |
| Ad7 hexon | 18666-21470 | 27 |

TABLE 4
Ad7 Amino Acid Sequences
Ad7 Amino Acid Sequences

| | Exemplary Sequence | SEQ ID |
|---|---|---|
| Component | (position in reference) | NO: |
| Ad7 fiber | 1-325 (GenBank accession - AP_000564) | 28 |
| Ad7 fiber shaft | 43-133 (GenBank accession no. AP_000564) | 29 |
| Ad7 fiber knob | 134-325 (GenBank accession no. AP_000564) | 30 |
| Ad7 penton | 1-544 (GenBank accession no. AP_000543) | 31 |
| Ad7 hexon | 1-934 (GenBank accession no. AP_000548) | 32 |
| Ad7 fiber tail | 1-42 (GenBank accession - AP_000564) | 158 |

TABLE 5
Ad11 Genomic Sequences
Ad11 Genomic Sequences
Reference Ad11 Genome Sequence: GenBank accession
number NC_011202 (SEQ ID NO: 147)

| | Exemplary Sequence | SEQ ID |
|---|---|---|
| Component | (position in reference) | NO: |
| Ad11 5′ (left) ITR | 1-137 | 33 |
| Ad11 3′ (right) ITR | 34658-34794 | 34 |
| Ad11 Packaging | 138-479 | 35 |
| Sequence | | |
| Ad11 E1 | 480-3931 | 36 |
| Ad11 E2 | 25445-3963 | 37 |
| Ad11 E3 | 26866-30624 | 38 |
| Ad11 fiber | 30811-31788 | 39 |
| Ad11 fiber tail | 30811-30936 | 168 |
| Ad11 fiber shaft | 30937-31209 | 40 |
| Ad11 fiber knob | 31210-31785 | 41 |
| Ad11 penton | 13682-15367 | 42 |
| Ad11 hexon | 18254-21100 | 43 |

TABLE 6
Ad11 Amino Acid Sequences
Ad11 Amino Acid Sequences

| | Exemplary Sequence | SEQ ID |
|---|---|---|
| Component | (position in reference) | NO: |
| Ad11 fiber | 1-325 (GenBank accession no. YP_002213828) | 44 |
| Ad11 fiber shaft | 43-133 (GenBank accession no. YP_002213828) | 45 |
| Ad11 fiber knob | 134-325 (GenBank accession no. YP_002213828) | 46 |
| Ad11 penton | 1-561 (GenBank accession no. YP_002213807) | 47 |
| Ad11 hexon | 1-948 (GenBank accession no. YP_002213812) | 48 |
| Ad11 fiber tail | 1-42 (GenBank accession no. YP_002213828) | 159 |

TABLE 7
Ad14 Genomic Sequences
Ad14 Genomic Sequences
Reference Ad14 Genome Sequence: GenBank accession
number AY803294 (SEQ ID NO: 148)

| | Exemplary Sequence | SEQ ID |
|---|---|---|
| Component | (position in reference) | NO: |
| Ad14 5′ (left) ITR | 1-137 | 49 |
| Ad14 3′ (right) ITR | 34628-34764 | 50 |
| Ad14 Packaging | 138-479 | 51 |
| Sequence | | |
| Ad14 E1 | 480-3947 | 52 |
| Ad14 E2 | 23389-3963 | 53 |
| Ad14 E3 | 26854-30601 | 54 |
| Ad14 fiber | 30788-31765 | 55 |
| Ad14 fiber tail | 30788-30913 | 169 |
| Ad14 fiber shaft | 30914-31186 | 56 |
| Ad14 fiber knob | 31187-31762 | 57 |
| Ad14 penton | 13698-15374 | 58 |
| Ad14 hexon | 18252-21089 | 59 |

TABLE 8
Ad14 Amino Acid Sequences
Ad14 Amino Acid Sequences

| | | SEQ |
|---|---|---|
| | Exemplary Sequence | ID |
| Component | (position in reference) | NO: |
| Ad14 fiber | 1-325 (GenBank accession no. AAW33140) | 60 |
| Ad14 fiber shaft | 43-133 (GenBank accession no. AAW33140) | 61 |
| Ad14 fiber knob | 134-325 (GenBank accession no. AAW33140) | 62 |
| Ad14 penton | 1-558 (GenBank accession no. AAW33119) | 63 |
| Ad14 hexon | 1-945 (GenBank accession no. AAW33124) | 64 |
| Ad14 fiber tail | 1-42 (GenBank accession no. AAW33140) | 160 |

TABLE 9
Ad16 Genomic Sequences
Ad16 Genomic Sequences
Reference Ad16 Genome Sequence: GenBank accession
number AY601636 (SEQ ID NO: 149)

| | Exemplary Sequence | SEQ ID |
|---|---|---|
| Component | (position in reference) | NO: |
| Ad16 5′ (left) ITR | 1-114 | 65 |
| Ad16 3′ (right) ITR | 35409-35522 | 66 |
| Ad16 Packaging | 115-479 | 67 |
| Sequence | | |
| Ad16 E1 | 480-3910 | 68 |
| Ad16 E2 | 23580-3954 | 69 |
| Ad16 E3 | 27107-31263 | 70 |
| Ad16 fiber | 31448-32509 | 71 |
| Ad16 fiber tail | 31448-31573 | 170 |
| Ad16 fiber shaft | 31574-31933 | 72 |
| Ad16 fiber knob | 31934-32506 | 73 |
| Ad16 penton | 13902-17534 | 74 |
| Ad16 hexon | 18450-21272 | 75 |

TABLE 10
Ad16 Amino Acid Sequences
Ad16 Amino Acid Sequences

| | | SEQ |
|---|---|---|
| | Exemplary Sequence | ID |
| Component | (position in reference) | NO: |
| Ad16 fiber | 1-353 (GenBank accession no. AAW33461) | 76 |
| Ad16 fiber shaft | 43-172 (GenBank accession no. AAW33461) | 77 |
| Ad16 fiber knob | 173-353 (GenBank accession no. AAW33461) | 78 |
| Ad16 penton | 1-555 (GenBank accession no. AAW33439) | 79 |
| Ad16 hexon | 1-940 (GenBank accession no. AAW33444) | 80 |
| Ad16 fiber tail | 1-42 (GenBank accession no. AAW33461) | 161 |

TABLE 11
Ad21 Genomic Sequences
Ad21 Genomic Sequences
Reference Ad21 Genome Sequence: GenBank accession
number AY601633 (SEQ ID NO: 150)

| | Exemplary Sequence | SEQ ID |
|---|---|---|
| Component | (position in reference) | NO: |
| Ad21 5′ (left) ITR | 1-114 | 81 |
| Ad21 3′ (right) ITR | 35269-35382 | 82 |
| Ad21 Packaging | 115-479 | 83 |
| Sequence | | |
| Ad21 E1 | 480-3911 | 84 |
| Ad21 E2 | 23611-3924 | 85 |
| Ad21 E3 | 27441-31208 | 86 |
| Ad21 fiber | 31406-32377 | 87 |
| Ad21 fiber tail | 31406-31531 | 171 |
| Ad21 fiber shaft | 31532-31804 | 88 |
| Ad21 fiber knob | 31805-32374 | 89 |
| Ad21 penton | 13878-15563 | 90 |
| Ad21 hexon | 18454-21303 | 91 |

TABLE 12
Ad21 Amino Acid Sequences
Ad21 Amino Acid Sequences

| | | SEQ |
|---|---|---|
| | Exemplary Sequence | ID |
| Component | (position in reference) | NO: |
| Ad21 fiber | 1-323 (GenBank accession no. AAW33370) | 92 |
| Ad21 fiber shaft | 43-133 (GenBank accession no. AAW33370) | 93 |
| Ad21 fiber knob | 134-323 (GenBank accession no. AAW33370) | 94 |
| Ad21 penton | 1-561 (GenBank accession no. AAW33349) | 95 |
| Ad21 hexon | 1-949 (GenBank accession no. AAW33354) | 96 |
| Ad21 fiber tail | 1-42 (GenBank accession no. AAW33370) | 162 |

TABLE 13
Ad34 Genomic Sequences
Ad34 Genomic Sequences
Reference Ad34 Genome Sequence: GenBank accession
number AY737797 (SEQ ID NO: 151)

| | Exemplary Sequence | SEQ ID |
|---|---|---|
| Component | (position in reference) | NO: |
| Ad34 5′ (left) ITR | 1-137 | 97 |
| Ad34 3′ (right) ITR | 34639-34775 | 98 |
| Ad34 Packaging | 138-479 | 99 |
| Sequence | | |
| Ad34 E1 | 480-3929 | 100 |
| Ad34 E2 | 23399-3945 | 101 |
| Ad34 E3 | 27185-30625 | 102 |
| Ad34 fiber | 30812-31783 | 103 |
| Ad34 fiber tail | 30812-30937 | 172 |
| Ad34 fiber shaft | 30938-31210 | 104 |
| Ad34 fiber knob | 31211-31780 | 105 |
| Ad34 penton | 13681-15357 | 106 |
| Ad34 hexon | 18244-21099 | 107 |

TABLE 14
Ad34 Amino Acid Sequences
Ad34 Amino Acid Sequences

| | | SEQ |
|---|---|---|
| | Exemplary Sequence | ID |
| Component | (position in reference) | NO: |
| Ad34 fiber | 1-323 (GenBank accession no. AAW33501) | 108 |
| Ad34 fiber shaft | 43-133 (GenBank accession no. AAW33501) | 109 |
| Ad34 fiber knob | 134-323 (GenBank accession no. AAW33501) | 110 |
| Ad34 penton | 1-558 (GenBank accession no. ABC49791) | 111 |
| Ad34 hexon | 1-951 (GenBank accession no. AAW33485) | 112 |
| Ad34 fiber tail | 1-42 (GenBank accession no. AAW33501) | 163 |

TABLE 15
Ad37 Genomic Sequences
Ad37 Genomic Sequences
Reference Ad37 Genome Sequence: GenBank accession
number DQ900900 (SEQ ID NO: 152)

| | Exemplary Sequence | SEQ ID |
|---|---|---|
| Component | (position in reference) | NO: |
| Ad37 5′ (left) ITR | 1-159 | 113 |
| Ad37 3′ (right) ITR | 35055-35213 | 114 |
| Ad37 Packaging | 160-479 | 115 |
| Sequence | | |
| Ad37 E1 | 480-3867 | 116 |
| Ad37 E2 | 22777-3902 | 117 |
| Ad37 E3 | 26198-30771 | 118 |
| Ad37 fiber | 31038-32135 | 119 |
| Ad37 fiber tail | 31038-31163 | 173 |
| Ad37 fiber shaft | 31164-31592 | 120 |
| Ad37 fiber knob | 31593-32132 | 121 |
| Ad37 penton | 13530-15089 | 122 |
| Ad37 hexon | 17775-20624 | 123 |

TABLE 16
Ad37 Amino Acid Sequences
Ad37 Amino Acid Sequences

| | | SEQ |
|---|---|---|
| | Exemplary Sequence | ID |
| Component | (position in reference) | NO: |
| Ad37 fiber | 1-361 (GenBank accession no. ABK59080) | 124 |
| Ad37 fiber shaft | 43-185 (GenBank accession no. ABK59080) | 125 |
| Ad37 fiber knob | 186-361 (GenBank accession no. ABK59080) | 126 |
| Ad37 penton | 1-519 (GenBank accession no. ABK59086) | 127 |
| Ad37 hexon | 1-949 (GenBank accession no. ABK59070) | 128 |
| Ad37 fiber tail | 1-42 (GenBank accession no. ABK59080) | 164 |

TABLE 17
Ad50 Genomic Sequences
Ad50 Genomic Sequences
Reference Ad50 Genome Sequence: GenBank accession
number AY737798 (SEQ ID NO: 153)

| | Exemplary Sequence | SEQ ID |
|---|---|---|
| Component | (position in reference) | NO: |
| Ad50 5′ (left) ITR | 1-114 | 129 |
| Ad50 3′ (right) ITR | 35272-35385 | 130 |
| Ad50 Packaging | 115-479 | 131 |
| Sequence | | |
| Ad50 E1 | 480-3910 | 132 |
| Ad50 E2 | 23590-3923 | 133 |
| Ad50 E3 | 27102-31222 | 134 |
| Ad50 fiber | 31409-32380 | 135 |
| Ad50 fiber tail | 31409-31534 | 174 |
| Ad50 fiber shaft | 31535-31807 | 136 |
| Ad50 fiber knob | 31808-32377 | 137 |
| Ad50 penton | 13888-15570 | 138 |
| Ad50 hexon | 18460-21282 | 139 |

TABLE 18
Ad50 Amino Acid Sequences
Ad50 Amino Acid Sequences

| | | SEQ |
|---|---|---|
| | Exemplary Sequence | ID |
| Component | (position in reference) | NO: |
| Ad50 fiber | 1-323 (GenBank accession no. AAW33547) | 140 |
| Ad50 fiber shaft | 43-133 (GenBank accession no. AAW33547) | 141 |
| Ad50 fiber knob | 134-323 (GenBank accession no. AAW33547) | 142 |
| Ad50 penton | 1-560 (GenBank accession No. AAW33525) | 143 |
| Ad50 hexon | 1-940 (GenBank accession no. AAW33530) | 144 |
| Ad50 fiber tail | 1-42 (GenBank accession no. AAW33547) | 165 |

In various embodiments, an Ad3, 7, 11, 14, 16, 21, 34, 37, or 50 vector or genome includes modifications that reduce and/or eliminate replication of the virus in recipients. Broadly, there are three recognized “generations” of adenoviral vectors and genomes engineered to reduce and/or eliminate replication of the virus in recipients. Adenoviral vectors of the present disclosure can include vectors according to any of these three generations.

In various embodiments, an Ad3, 7, 11, 14, 16, 21, 34, 37, or 50 genome differs from a reference Ad sequence (e.g., one or more canonical, representative, exemplary, or wild-type sequence of an adenovirus of a serotype of interest) at least in that the regulatory E1 gene (E1a and E1b) is removed from the Ad genome (“first generation” vector modifications). E1a and E1b are the first transcriptional regulatory factors produced during the adenoviral replication cycle. E1 deletion reduces or eliminates expression of certain viral genes controlled by E1, and E1-deleted helper viruses are replication-defective. Thus, first generation Ad vectors are deficient for replication in a recipient. In some embodiments, first-generation adenoviral vectors are engineered to remove E1 and E3 genes. Retained portions of the reference genome can be identical in sequence to a reference genome or can have less than 100% identity with a reference genome, e.g., at least 99%, 98%, 97%, 96%, 95%, 90%, 85%, 80%, or 75% identity. Without these E1 (or E1 and E3) genes, adenoviral vectors cannot replicate on their own but can be produced in mammalian cell lines that express E1 (e.g., of the same serotype) or another protein sufficient to restore expression of the certain viral genes. For illustration, where an E1-deficient Ad5 vector encodes an Ad5 E4orf6, the helper vector can be propagated in a cell line that expresses Ad5 E1. In one exemplary cell type for adenoviral vector production, HEK293 cells express Ad5 E1b55k, which is known to form a complex with Ad5 E4 protein ORF6.

In various embodiments, an Ad3, 7, 11, 14, 16, 21, 34, 37, or 50 genome differs from a reference Ad sequence at least in that the E1 gene (E1a and E1b) and one or more of non-structural genes E2, E3 and/or E4 are deleted (“second generation” modifications). Second generation Ads have greater payload capacity than first generation Ads and are more deficient for replication than first generation viruses. In some embodiments, second-generation adenoviral vectors, in addition to E1/E3 removal, are engineered to remove non-structural genes E2 and E4, resulting in increased capacity and reduced immunogenicity. Retained portions of the reference genome can be identical in sequence to a reference genome or can have less than 100% identity with a reference genome, e.g., at least 99%, 98%, 97%, 96%, 95%, 90%, 85%, 80%, or 75% identity.

In various embodiments, an Ad3, 7, 11, 14, 16, 21, 34, 37, or 50 genome differs from a reference Ad sequence at least in that they are engineered to remove all viral coding sequences from the Ad genome, and retain only the ITRs of the genome and the packaging sequence of the genome or a functional fragment thereof (“third generation” modifications). Third generation adenoviral vectors can also be referred to as gutless, high capacity adenoviral vectors, or helper-dependent adenoviral vectors (HdAds). Retained portions of the reference genome can be identical in sequence to a reference genome or can have less than 100% identity with a reference genome, e.g., at least 99%, 98%, 97%, 96%, 95%, 90%, 85%, 80%, or 75% identity.

Because third generation Ad genomes do not encode the proteins necessary for viral production, they are helper-dependent: a helper-dependent genome can only be packaged into a vector if they are present in a cell that includes a nucleic acid sequence that provides viral proteins in trans. These helper-dependent vectors are also characterized by still greater capacity than first and second generation vectors and decreased immunogenicity. Because HDAd vectors do not express viral genes when used as a vector, the risk of cytotoxicity or interferon response in recipients is reduced.

Helper-dependent adenoviral vectors (HDAd) engineered to lack all viral coding sequences can efficiently transduce a wide variety of cell types, and can mediate long-term transgene expression with negligible chronic toxicity. By deleting the viral coding sequences and leaving only the cis-acting elements necessary for genome replication (ITRs) and packaging (w), cellular immune response against the Ad vector is reduced. HDAd vectors have a large cloning capacity of up to allowing for the delivery of large payloads. These payloads can include large therapeutic genes or even multiple transgenes and large regulatory components to enhance, prolong, and regulate transgene expression. It has also been observed that the certain HDAd vector genomes can be most efficiently packaged when the genome has at least a minimum a total length, e.g., a minimum to total length of at least 20 kb (e.g., 20, 21, 22, 23, 24, 25, 26, 27, 28, 29, 30, 31, 32, 33, 34, or 35 kb) which length can include, e.g., a therapeutic payload and/or a “stuffer” sequence. Where a payload does not utilize a number of nucleotides that causes the adenoviral genome to have at least a target length, a stuffer sequence can be used to achieve or surpass the target length. The present disclosure includes that a minimum length for efficient packaging is not required for beneficial use of vectors provided herein, such that meeting any target length may be advantageous but not required for use of compositions and methods provided herein. Like other adenoviral vectors, typical HDAd genomes generally remain episomal and do not integrate with a host genome.

Because HDAd vectors do not encode the viral proteins required to produce viral particles, viral proteins must be provided in trans, e.g., expressed in and/or by cells in which the HDAd genome is present. In some HDAd vector systems, one viral genome (a helper genome) encodes all of the proteins (e.g., all of the structural viral proteins) required for replication but has a conditional defect in the packaging sequence, making it less likely to be packaged into a vector under certain vector production conditions (e.g., in the presence of an agent that reduces function of the conditionally defective packaging sequence). Thus, the HDAd donor viral genome includes (e.g., only includes) Ad ITRs, a payload (e.g., a therapeutic payload), and a functional packaging sequence (e.g., a wild-type packaging sequence or a functional fragment thereof), which allows the HDAd donor viral genome to be selectively packaged into HDAd viral vectors produced from structural components expressed from the helper vector genome. In other words, Ad3, 7, 11, 14, 16, 21, 34, 37, or 50 helper vectors can be used for production of Ad3, 7, 11, 14, 16, 21, 34, 37, or 50 donor vectors. Production of HD Ad3, 7, 11, 14, 16, 21, 34, 37, or 50 vectors can include co-transfection of a plasmid containing the HDAd vector genome and a packaging-defective helper virus that provides structural and non-structural viral proteins. The helper virus genome can rescue propagation of the Ad3, 7, 11, 14, 16, 21, 34, 37, or 50 donor vector and Ad3, 7, 11, 14, 16, 21, 34, 37, or 50 donor vector can be produced, e.g., at a large scale, and isolated. Various protocols are known in the art, e.g., at Palmer et al., 2009 Gene Therapy Protocols. Methods in Molecular Biology, Volume 433. Humana Press; Totowa, NJ: 2009. pp. 33-53. In some embodiments, a helper genome is E1-deficient.

In some HDAd vector systems, a helper genome utilizes a recombinase system (e.g., a Cre/loxP system) for conditional packaging. In certain such HDAd vector systems, a helper genome can include a packaging sequence or functional fragment thereof (e.g., a fragment of the packaging sequence that is sufficient for packaging, required for packaging, or required for efficient packaging of the Ad genome into the capsid) flanked by recombinase (e.g., loxP) sites so that contact with a corresponding recombinase (e.g., Cre recombinase) excises the packaging sequence or functional fragment thereof from the helper genome by recombinase-mediated (e.g., Cre-mediated) site-specific recombination between the recombinase sites (e.g., loxP sites). The present disclosure includes, among other things, Ad3, 7, 11, 14, 16, 21, 34, 37, or 50 helper vectors and genomes that include two recombination sites that flank a packaging sequence or functional fragment thereof, where the two recombination sites are sites corresponding to (i.e., for, or acted upon by) the same recombinase.

In various embodiments, a helper genome can include deletion of E1, e.g., where the helper genome includes all of the viral genes except for E1, as E1 expression products can be supplied by complementary expression from the genome of a producer cell line. In some embodiments, to prevent generation of replication competent Ad (RCA) as a consequence of homologous recombination between the helper and HDAd donor genomes present in producer cells, a “stuffer” sequence can be inserted into the E3 region to render any recombinants too large to be packaged and/or efficiently packaged.

For production of HDAd vectors, an HDAd donor genome can be delivered to cells that express a recombinase for excision of the conditional packaging sequence of a helper vector (e.g., 293 cells (HEK293) that expresses Cre recombinase), optionally where the HDAd donor genome is delivered to the cells in a non-viral vector form, such as a bacterial plasmid form (e.g., where the HDAd donor genome is present in a bacterial plasmid (pHDAd) and/or is liberated by restriction enzyme digestion). The same cells can be transduced with the helper genome including a packaging sequence or functional fragment thereof flanked by recombinase sites (e.g., loxP sites). Thus, producer cells can be transfected with the HDAd donor genome and transduced with a helper genome bearing a packaging sequence or a functional fragment thereof flanked by recombinase sites (e.g., loxP sites), where the cells express a recombinase (e.g., Cre) corresponding to the recombinase sites such that excision of the packaging sequence or functional fragment thereof renders the helper virus genome deficient for packaging (e.g., unpackageable), but still able to provide all of the necessary trans-acting factors for production of HDAd donor vector including the HDAd donor genome.

Similar HDAd production systems have been developed using FLP (e.g., FLPe)/frt site-specific recombination, where FLP-mediated recombination between frt sites flanking the packaging sequence or functional fragment thereof of the helper genome reduces or eliminates packaging of helper genomes in producer cells that express FLP.

HDAd vectors including the donor vector genome including the payload can be isolated from the producer cells. HDAd donor vectors can be further purified from helper vectors by physical means. In general, some contamination of helper vectors and/or helper genomes in HDAd viral vectors and HDAd viral vector formulations can occur and can be tolerated.

HDAd3, 7, 11, 14, 16, 21, 34, 37, and 50 donor vectors, donor genomes, helper vectors, and helper genomes are also exemplary of compositions provided herein and can be used in various methods of the present disclosure. An HDAd3, 7, 11, 14, 16, 21, 34, 37, or 50 vector or genome is a helper-dependent Ad3, 7, 11, 14, 16, 21, 34, 37, or 50 vector or genome. An Ad3, 7, 11, 14, 16, 21, 34, 37, or 50 helper vector is a vector that includes a helper genome that includes a conditionally expressed (e.g., frt-site or loxP-site flanked) packaging sequence or fragment thereof and encodes all of the necessary trans-acting factors for production of Ad3, 7, 11, 14, 16, 21, 34, 37, or 50 virions into which the donor genome can be packaged.

The present disclosure further includes an HDAd3, 7, 11, 14, 16, 21, 34, 37, or 50 donor vector production system including a cell including an HDAd3, 7, 11, 14, 16, 21, 34, 37, or 50 donor genome and an Ad3, 7, 11, 14, 16, 21, 34, 37, or 50 helper genome. In certain such cells, viral proteins encoded and expressed by the helper genome can be utilized in production of HDAd3, 7, 11, 14, 16, 21, 34, 37, or 50 donor vectors in which the HDAd3, 7, 11, 14, 16, 21, 34, 37, or 50 donor genome is packaged. Accordingly, the present disclosure includes methods of production of HDAd3, 7, 11, 14, 16, 21, 34, 37, or 50 donor vectors by culturing cells that include an HDAd3, 7, 11, 14, 16, 21, 34, 37, or 50 donor genome and an Ad3, 7, 11, 14, 16, 21, 34, 37, or 50 helper genome. In some embodiments the cells encode and express a recombinase that corresponds to recombinase direct repeats that flank a packaging sequence of the Ad3, 7, 11, 14, 16, 21, 34, 37, or 50 helper vector. In some embodiments, the flanked packaging sequence of the Ad3, 7, 11, 14, 16, 21, 34, 37, or 50 helper genome has been excised.

In some embodiments the Ad3, 7, 11, 14, 16, 21, 34, 37, or 50 helper genome encodes all Ad3, 7, 11, 14, 16, 21, 34, 37, or 50 coding sequences. In some embodiments the Ad3, 7, 11, 14, 16, 21, 34, 37, or 50 helper genome encodes and/or expresses all Ad3, 7, 11, 14, 16, 21, 34, 37, or 50 coding sequences except for one or more coding sequences of E1 and/or an E3 coding sequence and/or an E4 coding sequence. In various embodiments, a helper genome that does not encode and/or express an Ad3, 7, 11, 14, 16, 21, 34, 37, or 50 E1 gene does not encode and/or express an Ad3, 7, 11, 14, 16, 21, 34, 37, or 50 E4 gene. In various embodiments, as will be appreciate by those of skill in the art, cells of compositions and methods for production of HDAd donor vectors can be cells that express an E1 expression product.

The present disclosure includes, among other things, HDAd3, 7, 11, 14, 16, 21, 34, 37, or 50 donor vectors and genomes that include Ad3, 7, 11, 14, 16, 21, 34, 37, or 50 ITRs (a 5′ Ad3, 7, 11, 14, 16, 21, 34, 37, or 50 ITR and a 3′ ITR of the same serotype), e.g., where two Ad3, 7, 11, 14, 16, 21, 34, 37, or 50 ITRs flank a packaging sequence and a payload. The present disclosure includes, among other things, HDAd3, 7, 11, 14, 16, 21, 34, 37, or 50 donor vectors and genomes in which E1 or a fragment thereof is deleted. The present disclosure includes, among other things, HDAd3, 7, 11, 14, 16, 21, 34, 37, or 50 vectors and genomes in which E3 or a fragment thereof is deleted.

In various embodiments, excision of a packaging sequence or functional fragment thereof from an Ad3, 7, 11, 14, 16, 21, 34, 37, or 50 helper genome reduces propagation of the vector by, e.g., at least 20%, 30%, 40%, 50%, 60%, 70%, 80%, 90%, 91%, 92%, 93%, 94%, 95%, 96%, 97%, 98%, 99%, 99.5%, 99.9%, or 100% (e.g., reduces propagation of the vector by a percentage having a lower bound of 20%, 30%, 40%, 50%, 60%, 70%, and an upper bound of 60%, 70%, 80%, 90%, 91%, 92%, 93%, 94%, 95%, 96%, 97%, 98%, 99%, 99.5%, 99.9% or 100%), optionally where percent propagation is measured as the number of viral particles produced by propagation of excised vector (vector from which the recombinase site-flanked sequence has been excised) as compared to complete vector (vector from which the recombinase site-flanked sequence has not been excised) or as compared to wild-type Ad3, 7, 11, 14, 16, 21, 34, 37, or 50 vector under comparable conditions.

An additional optional engineering consideration can be engineering of a helper genome having a size that permits separation of helper vector from HDAd3, 7, 11, 14, 16, 21, 34, 37, or 50 donor vector by centrifugation, e.g., by CsCl ultracentrifugation. One means of achieving this result is to increase the size of the helper genome as compared to a typical Ad3, 7, 11, 14, 16, 21, 34, 37, or 50 genome. In particular, adenoviral genomes can be increased by engineering to at least 104% of wild-type length. Certain helper vectors of the present disclosure can accommodate a payload and/or stuffer sequence.

The present disclosure includes that in various embodiments a vector or genome of the present disclosure can include a selection of components each selected from, or having at least 75% sequence identity (e.g., at least 75%, 80%, 85%, 90%, 95%, 96%, 97%, 98%, 99%, or 100% identity) to, a corresponding sequence of a single particular serotype. To provide an illustrative example, all components can correspond to (e.g., have at least 75% sequence identity to sequences of) Ad34, excepting sequences otherwise indicated (e.g., a payload, e.g., a heterologous payload).

### I(C). Ad3, 7, 11, 14, 16, 21, 34, 37, and 50 Gene Therapy Vector Payloads

Ad3, 7, 11, 14, 16, 21, 34, 37, or 50 donor vectors and genomes of the present disclosure can include a variety of heterologous nucleic acid payloads that can include any of one or more coding sequences that encode one or more expression products, one or more regulatory sequences operably linked to a coding sequence, one or more stuffer sequences, and the like. In various embodiments, the payload is engineered in order to achieve a desired result such as a therapeutic effect in a host cell or system, e.g., expression of a protein of therapeutic interest or of expression of a gene editing system, e.g., a CRISPR/Cas system or base editing system, to generate a sequence modification of therapeutic interest.

In some embodiments, a payload can include a gene. A gene can include not only coding sequences but also regulatory regions such as promoters, enhancers, termination regions, locus control regions (LCRs), termination and polyadenylation signal elements, splicing signal elements, silencers, insulators, and the like. A gene can include introns and other DNA sequences spliced from an expressed mRNA transcript, along with variants resulting from alternative splice sites. Coding sequences can also include alternative synonymous codon usage as compared to a reference sequence, e.g., codon usage modified as compared to a reference in accordance with codon preference of a specific organism or target cell type.

A payload can include a single gene or multiple genes. A payload can include a single coding sequence or a plurality of coding sequences. A payload can include a single regulatory sequence or a plurality of regulatory sequences. A payload can include a plurality of coding sequences where the individual expression products of the coding sequences function together, e.g., as in the case of an endonuclease and a guide RNA, or independently, e.g., as two separate proteins that do not directly or indirectly bind. As will be appreciated by those of skill in the art, any payload or payload component (e.g., a payload-encoded expression product or regulatory sequence) that is not encoded by the reference wild-type Ad3, 7, 11, 14, 16, 21, 34, 37, or 50 genome can be referred to herein as a heterologous expression product.

For the avoidance of doubt, the present disclosure includes variants of amino acid and nucleic acid sequences provided herein. Variants include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein and nucleic acid sequences described or disclosed herein wherein the variant exhibits substantially similar or improved biological function.

### I(C)(i). Payload Expression Products

A payload of an adenoviral donor vector or adenoviral donor genome of the present disclosure can include one or more coding sequences that encode any of a variety of expression products. Exemplary expression products include proteins, including without limitation replacement therapy proteins for treatment of diseases or conditions characterized by low expression or activity of a biologically active protein as compared to a reference level. Exemplary expression products include CRISPR/Cas, base editor, and prime editor systems. Exemplary expression products include antibodies, CARs, and TCRs. Exemplary expression products include small RNAs. In various embodiments, integration of all or a portion of a donor vector payload into a host cell genome is not required in order for delivery to the target cell of a donor vector or genome to produce an intended or target effect, e.g., in certain instances in which the intended or target effect includes editing of the host cell genome by a CRISPR, base editor, or prime editor system. In various embodiments, integration of all or a portion of a donor vector payload is required or preferred in order for delivery to the target cell of a donor vector or genome to produce an intended or target effect, e.g., where expression of a payload-encoded expression product is desired in progeny cells of a transduced target cell. In various embodiments, a payload can include a nucleic acid sequence engineered for integration into a host cell genome (an “integration element”), e.g., by recombination or transposition.

A gene sequence encoding one or more therapeutic proteins can be readily prepared by synthetic or recombinant methods from the relevant amino acid sequence. In particular embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In particular embodiments, the gene sequence encoding the sequences can be codon optimized for expression in mammalian cells.

Particular examples of therapeutic genes and/or expression products include γ-globin, Factor VIII, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, a FANC family gene (e.g., FancA, FancB, FancC, FancD1 (BRCA2), FancD2, FancE, FancF, FancG, Fancl, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)), soluble CD40, CTLA, Fas L, an antibody (e.g., that specifically binds CD4, CD5, CD7, CD52, IL1, IL2, IL6, TNF, P53, PTPN22, or DRB1*1501/DQB1*0602), an antibody to TCR specifically present on autoreactive T cells, IL4, IL10, IL12, IL13, IL1Ra, sIL1RI, sIL1RII, sTNFRI, sTNFRII, globin family genes, WAS, phox, dystrophin, pyruvate kinase, CLN3, ABCD1, arylsulfatase A, SFTPB, SFTPC, NLX2.1, ABCA3, GATA1, ribosomal protein genes, TERT, TERC, DKC1, TINF2, CFTR, LRRK2, PARK2, PARK7, PINK1, SNCA, PSEN1, PSEN2, APP, SOD1, TDP43, FUS, ubiquilin 2, C9ORF72, and other therapeutic genes and/or expression products described herein.

A therapeutic gene can be selected to provide a therapeutically effective response against diseases related to red blood cells and clotting. In particular embodiments, the disease is a hemoglobinopathy like thalassemia, or a sickle cell disease/trait. The therapeutic gene may be, for example, a gene that induces or increases production of hemoglobin; induces or increases production of β-globin, γ-globin, or α-globin; or increases the availability of oxygen to cells in the body. The therapeutic gene may be, for example, HBB or CYB5R3. Exemplary effective treatments may, for example, increase blood cell counts, improve blood cell function, or increase oxygenation of cells in patients. In another particular embodiment, the disease is hemophilia. The therapeutic gene may be, for example, a gene that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary therapeutic genes include F8 and F9. Exemplary effective treatments may, for example, increase or induce the production of coagulation/clotting factors VIII and IX; improve the functioning of coagulation/clotting factors VIII and IX, or reduce clotting time in subjects.

In various embodiments of the present disclosure, a donor vector encodes a globin gene, wherein the globin protein encoded by the globin gene is selected from a γ-globin, a β-globin, and/or an α-globin. Globin genes of the present disclosure can include, e.g., one or more regulatory sequences such as a promoter operably linked to a nucleic acid sequence encoding a globin protein. As those of skill in the art will appreciate, each of γ-globin, β-globin, and/or α-globin is a component of fetal and/or adult hemoglobin and is therefore useful in various vectors disclosed herein.

In various embodiments, increasing expression of a globin protein can refer to any of one or more of (i) increasing the amount, concentration, or expression (e.g., transcription or translation of nucleic acids encoding) in a cell or system of globin protein having a particular sequence; (ii) increasing the amount, concentration, or expression (e.g., transcription or translation of nucleic acids encoding) in a cell or system of globin protein of a particular type (e.g., the total amount of all proteins that would be identified as γ-globin (or alternatively β-globin or α-globin) by those of skill in the art or as set forth in the present specification) without respect to the sequences of the proteins relative to each other; and/or (iii) expressing in a cell or system a heterologous globin protein, e.g., a globin protein not encoded by a host cell prior to gene therapy.

The following references describe particular exemplary sequences of functional globin genes. References 1-4 relate to α-type globin sequences and references 4-12 relate to β-type globin sequences (including β and γ globin sequences), which sequences are hereby incorporated by reference: (1) GenBank Accession No. Z84721 (Mar. 19, 1997); (2) GenBank Accession No. NM 000517 (Oct. 31, 2000); (3) Hardison et al., J. Mol. Biol. (1991) 222(2):233-249; (4) A Syllabus of Human Hemoglobin Variants (1996), by Titus et al., published by The Sickle Cell Anemia Foundation in Augusta, Ga. (available online at globin.cse.psu.edu); (5) GenBank Accession No. J00179 (Aug. 26, 1993) or U01317.1; (6) Tagle et al., Genomics (1992) 13(3):741-760; (7) Grovsfeld et al., Cell (1987) 51(6):975-985; (8) Li et al., Blood (1999) 93(7):2208-2216; (9) Gorman et al., J. Biol. Chem. (2000) 275(46):35914-35919; (10) Slightom et al., Cell (1980) 21(3):627-638; (11) Fritsch et al., Cell (1980) 19(4): 959-972; (12) Marotta et al., J. Biol. Chem. (1977) 252(14):5040-5053. For additional coding and non-coding regions of genes encoding globins see, for example, by Marotta et al., Prog. Nucleic Acid Res. Mol. Biol. 19, 165-175, 1976, Lawn et al., Cell 21 (3), 647-651, 1980, and Sadelain et al., PNAS.; 92:6728-6732, 1995. In some embodiments a globin gene encodes a G16D gamma globin variant.

An exemplary amino acid sequence of hemoglobin subunit R is provided, for example, at NCBI Accession No. P68871. An exemplary amino acid sequence for β-globin is provided, for example, at NCBI Accession No. NP_000509.

In addition to therapeutic genes and/or gene products, the transgene can also encode for therapeutic molecules, such as checkpoint inhibitor reagents, chimeric antigen receptor molecules specific to one or more cancer antigens, and/or T-cell receptors specific to one or more cancer antigens.

As another example, a therapeutic gene can be selected to provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; α-mannosidosis; β-mannosidosis; glycogen storage disease type I, also known as GSDI, von Gierke disease, or Tay Sachs; Pompe disease; Gaucher disease; or Fabry disease. The therapeutic gene may be, for example a gene encoding or inducing production of an enzyme, or that otherwise causes the degradation of mucopolysaccharides in lysosomes. Exemplary therapeutic genes include IDUA or iduronidase, IDS, GNS, HGSNAT, SGSH, NAGLU, GUSB, GALNS, GLB1, ARSB, and HYAL1. Exemplary effective genetic therapies for lysosomal storage disorders may, for example, encode or induce the production of enzymes responsible for the degradation of various substances in lysosomes; reduce, eliminate, prevent, or delay the swelling in various organs, including the head (exp. Macrosephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a therapeutic gene can be selected to provide a therapeutically effective response against a hyperproliferative disease. In particular embodiments, the hyperproliferative disease is cancer. The therapeutic gene may be, for example, a tumor suppressor gene, a gene that induces apoptosis, a gene encoding an enzyme, a gene encoding an antibody, or a gene encoding a hormone. Exemplary therapeutic genes and gene products include (in addition to those listed elsewhere herein) 101F6, 123F2 (RASSF1), 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CNTF, COX-1, CSFIR, CTS-1, cytosine deaminase, DBCCR-1, DCC, Dp, DPC-4, ElA, E2F, EBRB2, erb, ERBA, ERBB, ETS1, ETS2, ETV6, Fab, FCC, FGF, FGR, FHIT, fms, FOX, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-5, IL-6, IL-7, IL-8, IL-9, IL-11, ING1, interferon α, interferon β, interferon γ, IRF-1, JUN, KRAS, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p2′7, p5′7, p′73, p300, PGS, PIM1, PL6, PML, PTEN, raf, RaplA, ras, Rb, RB1, RET, rks-3, ScFv, scFV ras, SEM A3, SRC, TALI, TCL3, TFPI, thrombospondin, thymidine kinase, TNF, TP53, trk, T-VEC, VEGF, VHL, WT1, WT-1, YES, and zacl. Exemplary effective genetic therapies may suppress or eliminate tumors, result in a decreased number of cancer cells, reduced tumor size, slow or eliminate tumor growth, or alleviate symptoms caused by tumors.

As another example, a therapeutic gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

### I(C)(i)(a). Binding Domain, Antibody, CAR, and TCR Payload Expression Products

The present disclosure includes payloads that can include sequences that encode any of a variety of binding domains. Sequences that encode binding domains can encode, for example, antibodies, chimeric antigen receptors, TCRs, or other binding polypeptides.

Antibodies and antibody fragments are exemplary of binding domains. The term “antibody” can refer to a polypeptide that includes one or more canonical immunoglobulin sequence elements sufficient to confer specific binding to a particular antigen (e.g., a heavy chain variable domain, a light chain variable domain, and/or one or more CDRs). Thus, the term antibody includes, without limitation, human antibodies, non-human antibodies, synthetic and/or engineered antibodies, fragments thereof, and agents including the same. Antibodies can be naturally occurring immunoglobulins (e.g., generated by an organism reacting to an antigen). Synthetic, non-naturally occurring, or engineered antibodies can be produced by recombinant engineering, chemical synthesis, or other artificial systems or methodologies known to those of skill in the art.

As is well known in the art, typical human immunoglobulins are approximately 150 kD tetrameric agents that include two identical heavy (H) chain polypeptides (about 50 kD each) and two identical light (L) chain polypeptides (about 25 kD each) that associate with each other to form a structure commonly referred to as a “Y-shaped” structure. Typically, each heavy chain includes a heavy chain variable domain (VH) and a heavy chain constant domain (CH). The heavy chain constant domain includes three CH domains: CH1, CH2 and CH3. A short region, known as the “switch”, connects the heavy chain variable and constant regions. The “hinge” connects CH2 and CH3 domains to the rest of the immunoglobulin. Each light chain includes a light chain variable domain (VL) and a light chain constant domain (CL), separated from one another by another “switch.” Each variable domain contains three hypervariable loops known as “complement determining regions” (CDR1, CDR2, and CDR3) and four somewhat invariant “framework” regions (FR1, FR2, FR3, and FR4). In each VH and VL, the three CDRs and four FRs are arranged from amino-terminus to carboxy-terminus in the following order: FR1, CDR1, FR2, CDR2, FR3, CDR3, and FR4. The variable regions of a heavy and/or a light chain are typically understood to provide a binding moiety that can interact with an antigen. Constant domains can mediate binding of an antibody to various immune system cells (e.g., effector cells and/or cells that mediate cytotoxicity), receptors, and elements of the complement system. Heavy and light chains are linked to one another by a single disulfide bond, and two other disulfide bonds connect the heavy chain hinge regions to one another, so that the dimers are connected to one another and the tetramer is formed. When natural immunoglobulins fold, the FR regions form the beta sheets that provide the structural framework for the domains, and the CDR loop regions from both the heavy and light chains are brought together in three-dimensional space so that they create a single hypervariable antigen binding site located at the tip of the Y structure.

In some embodiments, an antibody is polyclonal, monoclonal, monospecific, or multispecific antibodies (including bispecific antibodies). In some embodiments, an antibody includes at least one light chain monomer or dimer, at least one heavy chain monomer or dimer, at least one heavy chain-light chain dimer, or a tetramer that includes two heavy chain monomers and two light chain monomers. Moreover, the term “antibody” can include (unless otherwise stated or clear from context) any art-known constructs or formats utilizing antibody structural and/or functional features including without limitation intrabodies, domain antibodies, antibody mimetics, Zybodies®, Fab fragments, Fab′ fragments, F(ab′)2 fragments, Fd′ fragments, Fd fragments, isolated CDRs or sets thereof, single chain antibodies, single-chain Fvs (scFvs), disulfide-linked Fvs (sdFv), polypeptide-Fc fusions, single domain antibodies (e.g., shark single domain antibodies such as IgNAR or fragments thereof), cameloid antibodies, camelized antibodies, masked antibodies (e.g., Probodies®), affybodies, anti-idiotypic (anti-Id) antibodies (including, e.g., anti-anti-Id antibodies), Small Modular ImmunoPharmaceuticals (“SMIPs™”), single chain or Tandem diabodies (TandAb®), VHHs, Anticalins®, Nanobodies® minibodies, BiTE®s, ankyrin repeat proteins or DARPINs®, Avimers®, DARTs, TCR-like antibodies, Adnectins®, Affilins®, Trans-bodies®, Affibodies®, TrimerX®, MicroProteins, Fynomers®, Centyrins®, and KALBITOR®s, CARs, engineered TCRs, and antigen-binding fragments of any of the above.

In various embodiments, an antibody includes one or more structural elements recognized by those skilled in the art as a complementarity determining region (CDR) or variable domain. In some embodiments, an antibody can be a covalently modified (“conjugated”) antibody (e.g., an antibody that includes a polypeptide including one or more canonical immunoglobulin sequence elements sufficient to confer specific binding to a particular antigen, where the polypeptide is covalently linked with one or more of a therapeutic agent, a detectable moiety, another polypeptide, a glycan, or a polyethylene glycol molecule). In some embodiments, antibody sequence elements are humanized, primatized, chimeric, etc, as is known in the art.

An antibody including a heavy chain constant domain can be, without limitation, an antibody of any known class, including but not limited to, IgA, secretory IgA, IgG, IgE and IgM, based on heavy chain constant domain amino acid sequence (e.g., alpha (α), delta (δ), epsilon (ε), gamma (γ) and mu (μ)). IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. As used herein, a “light chain” can be of a distinct type, e.g., kappa (κ) or lambda (λ), based on the amino acid sequence of the light chain constant domain. In some embodiments, an antibody has constant region sequences that are characteristic of mouse, rabbit, primate, or human immunoglobulins. Naturally-produced immunoglobulins are glycosylated, typically on the CH2 domain. As is known in the art, affinity and/or other binding attributes of Fc regions for Fc receptors can be modulated through glycosylation or other modification. In some embodiments, an antibody may lack a covalent modification (e.g., attachment of a glycan) that it would have if produced naturally. In some embodiments, antibodies produced and/or utilized in accordance with the present invention include glycosylated Fc domains, including Fc domains with modified or engineered such glycosylation.

The term “antibody fragment” can refer to a portion of an antibody or antibody agent as described herein, and typically refers to a portion that includes an antigen-binding portion or variable region thereof. An antibody fragment can be produced by any means. For example, in some embodiments, an antibody fragment can be enzymatically or chemically produced by fragmentation of an intact antibody or antibody agent. Alternatively, in some embodiments, an antibody fragment can be recombinantly produced (i.e., by expression of an engineered nucleic acid sequence. In some embodiments, an antibody fragment can be wholly or partially synthetically produced. In some embodiments, an antibody fragment (particularly an antigen-binding antibody fragment) can have a length of at least about 50, 60, 70, 80, 90, 100, 110, 120, 130, 140, 150, 160, 170, 180, 190 amino acids or more, in some embodiments at least about 200 amino acids.

In some instances, it is beneficial for the binding domain to be derived from the same species it will ultimately be used in. For example, for use in humans, it may be beneficial for the antigen binding domain to include a human antibody, humanized antibody, or a fragment or engineered form thereof. Antibodies from human origin or humanized antibodies have lowered or no immunogenicity in humans and have a lower number of non-immunogenic epitopes compared to non-human antibodies. Antibodies and their engineered fragments will generally be selected to have a reduced level or no antigenicity in human subjects.

In various embodiments, a payload can encode a binding agent that is a checkpoint inhibitor such as an antibody that specifically binds an immune checkpoint protein. A number of immune checkpoint inhibitors are known. Immune checkpoint inhibitors can include peptides, antibodies, nucleic acid molecules and small molecules. Examples of immune checkpoints include PD-1, PD-L1, lymphocyte activation gene-3 (LAG-3), and T cell immunoglobulin and mucin domain-containing molecule 3 (TIM-3).

The present disclosure further includes antibodies and other binding domains that bind CD4, CD5, CD7, CD52, etc.; antibodies; antibodies to ILL IL2, IL6; an antibody to TCR specifically present on autoreactive T cells; IL4; IL10; IL12; IL13; IL1Ra; sIL1RI; sIL1RII; antibodies to TNF; ABCA3; ABCD1; ADA; AK2; APP; arginase; arylsulfatase A; A1AT; CD3D; CD3E; CD3G; CD3Z; CFTR; CHD7; chimeric antigen receptor (CAR); CIITA; CLN3; complement factor, CORO1A; CTLA; C1 inhibitor; C90RF72; DCLRE1B; DCLRE1C; decoy receptors; DKC1; DRB1*1501/DQB1*0602; dystrophin; enzymes; Factor VIII, FANC family genes (FancA, FancB, FancC, FancD1 (BRCA2), FancD2, FancE, FancF, FancG, Fancl, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)); Fas L; FUS; GATA1; globin family genes (ie. γ-globin); F8; glutaminase; HBA1; HBA2; HBB; IL7RA; JAK3; LCK; LIG4; LRRK2; NHEJ1; NLX2.1; neutralizing antibodies; ORAI1; PARK2; PARK7; phox; PINK1; PNP; PRKDC; PSEN1; PSEN2; PTPN22; PTPRC; P53; pyruvate kinase; RAG1; RAG2; RFXANK; RFXAP; RFX5; RMRP; ribosomal protein genes; SFTPB; SFTPC; SOD1; soluble CD40; STIM1; sTNFRI; sTNFRII; SLC46A1; SNCA; TDP43; TERT; TERC; TINF2; ubiquilin 2; WAS; WHN; ZAP70; yC; and other therapeutic genes described herein.

HSCs can be engineered to encode and/or express chimeric antigen receptor (CAR) constructs. CARs can include several distinct subcomponents that can cause cells to recognize and kill target cells such as cancer cells. The subcomponents include at least an extracellular component and an intracellular component.

An extracellular CAR component can include a binding domain that specifically binds a marker that is preferentially present on the surface of unwanted cells. When the binding domain binds such markers, the intracellular component directs a cell to destroy the bound cancer cell. The binding domain is typically a single-chain variable fragment (scFv) derived from a monoclonal antibody (mAb), but it can be based on other formats which include an antibody-like antigen binding site.

Intracellular CAR components provide activation signals based on the inclusion of an effector domain. First generation CARs utilized the cytoplasmic region of CD3 as an effector domain. Second generation CARs utilized CD3 in combination with cluster of differentiation 28 (CD28) or 4-1BB (CD137), while third generation CARs have utilized CD3 in combination with CD28 and 401BB within intracellular effector domains.

Intracellular or otherwise the cytoplasmic signaling components of a CAR are responsible for activation of the cell in which the CAR is expressed. The term “intracellular signaling components” or “intracellular components” is thus meant to include any portion of the intracellular domain sufficient to transduce an activation signal. Intracellular components of expressed CAR can include effector domains. An effector domain is an intracellular portion of a fusion protein or receptor that can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from a co-receptor or co-stimulatory molecule.

An effector domain can include one, two, three or more receptor signaling domains, intracellular signaling components (e.g., cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8a, CD80, IL2Rf3, IL2Ry, IL7Ra, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRlla, FeRβ (Fcε Rib), DAP10, and DAP12. In particular embodiments, variants of CD3 retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al. Blood. 2012; 119(3):696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

In particular embodiments, the amino acid sequence of the intracellular signaling component includes a variant of CD3ζ and a portion of the 4-1BB intracellular signaling component.

In particular embodiments, the intracellular signaling component includes (i) all or a portion of the signaling domain of CD3ζ, (ii) all or a portion of the signaling domain of 4-1BB, or (iii) all or a portion of the signaling domain of CD3ζ and 4-1BB.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

CAR generally also include one or more linker sequences that are used for a variety of purposes within the molecule. For example, a transmembrane domain can be used to link the extracellular component of the CAR to the intracellular component. A flexible linker sequence often referred to as a spacer region that is membrane-proximal to the binding domain can be used to create additional distance between a binding domain and the cellular membrane. This can be beneficial to reduce steric hindrance to binding based on proximity to the membrane. A common spacer region used for this purpose is the IgG4 linker. More compact spacers or longer spacers can be used, depending on the targeted cell marker. Other potential CAR subcomponents are described in more detail elsewhere herein. Components of CAR are now described in additional detail as follows: (a) Binding Domains; (b) Intracellular Signalling Components; (c) Linkers; (d) Transmembrane Domains; (e) Junction Amino Acids; and (f) Control Features Including Tag Cassettes.

Transmembrane domains within a CAR molecule, often serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge.

TCRs refer to naturally occurring T cell receptors. Payloads of the present disclosure can encode a TCR or a CAR/TCR hybrids that includes an element of a TCR and an element of a CAR. For example, a CAR/TCR hybrid could have a naturally occurring TCR binding domain with an effector domain that the TCR binding domain is not naturally associated with. A CAR/TCR hybrid could have a mutated TCR binding domain and an ITAM signaling domain. A CAR/TCR hybrid could have a naturally occurring TCR with an inserted non-naturally occurring spacer region or transmembrane domain.

### I(C)(i)(b). Gene Editing Systems and Components

In various embodiments, a payload of the present disclosure encodes at least one component, or all components, of a gene editing system. Gene editing systems of the present disclosure include CRISPR systems, base editing, and prime editing systems. Broadly, gene editing systems can include a plurality of components including a gene editing enzyme selected from a CRISPR-associated RNA-guided endonuclease, a base editing enzyme, and a prime editing enzyme and at least one gRNA. Accordingly, gene editing systems of the present disclosure can include either (i) in the case of a CRISPR system, a CRISPR enzyme that is a CRISPR-associated RNA-guided endonuclease and at least one guide RNA (gRNA), (ii) in the case of a base editing system, a base editing enzyme and at least one gRNA, or (iii) in the case of a prime editing system and at least one prime editing gRNA. Nucleotide sequences encoding gene editing systems as disclosed herein are typically too large for inclusion in many limited-capacity vector systems, but the large capacity of adenoviral vectors permits inclusion of such sequences in adenoviral vectors and genomes of the present disclosure. An additional advantage of adenoviral vectors and genomes with payloads encoding gene editing systems or components of the present disclosure is that adenoviral genomes do not naturally integrate into host cell genomes, which facilitates transient expression of gene editing systems and components, which can be desirable, e.g., to avoid immunogenicity and/or genotoxicity.

In other embodiments, a gene editing system can include engineered zing finger nucleases (ZFN). For instance, a ZFN is an artificial endonuclease that consists of a designed zinc finger protein (ZFP) fused to the cleavage domain of the FokI restriction enzyme. A ZFN may be redesigned to cleave new targets by developing ZFPs with new sequence specificities. For genome engineering, a ZFN is targeted to cleave a chosen genomic sequence. The cleavage event induced by the ZFN provokes cellular repair processes that in turn mediate efficient modification of the targeted locus. If the ZFN-induced cleavage event is resolved via non-homologous end joining, this can result in small deletions or insertions, effectively leading to gene knockout. If the break is resolved via a homology-based process in the presence of an investigator-provided donor, small changes or entire transgenes can be transferred, often without selection, into the chromosome; this is referred to as ‘gene correction’ and ‘gene addition’, respectively.

In some embodiments a gene editing system (e.g., a CRISPR system, base editing system, or prime editing system) is engineered to modify a nucleic acid sequence that encodes γ-globin, e.g., to increase expression of γ-globin. The main fetal form of hemoglobin, hemoglobin F (HbF) is formed by pairing of γ-globin polypeptide subunits with α-globin polypeptide subunits. Human fetal γ-globin genes (HBG1 and HBG2; two highly homologous genes produced by evolutionary duplication) are ordinarily silenced around birth, while expression of adult β-globin gene expression (HBB and HBD) increases. Mutations that cause or permit persistent expression of fetal γ-globin throughout life can ameliorate phenotypes of β-globin deficiencies. Thus, reactivation of fetal γ-globin genes can be therapeutically beneficial, particularly in subjects with β-globin deficiency. A variety of mutations that cause increased expression of γ-globin are known in the art (see, e.g., Wienert, Trends in Genetics 34(12): 927-940, 2018, which is incorporated herein by reference in its entirety and with respect to mutations that increase expression of γ-globin). Certain such mutations are found in the HBG1 promoter or HBG2 promoter.

In various embodiments, a gene editing system designed to increase expression of γ-globin includes an HBG1/2 promoter-targeted gRNA that is designed to increase expression of γ-globin coding by modification and/or inactivation of a BCL11A repressor protein binding site. In various embodiments, a gene editing system designed to increase expression of γ-globin includes a bcl11a-targeted gRNA that is designed to increase expression of γ-globin by modification and/or inactivation of the erythroid bcl11a enhancer to reduce BCL11A repressor protein expression in erythroid cells. In various embodiments, a gene editing system designed to increase expression of γ-globin includes a gRNA targeted to cause a loss of function mutation in the gene encoding BCL11A.

### I(C)(i)(b)(1). CRISPR Payload Expression Products

The CRISPR (Clustered Regularly Interspaced Short Palindromic Repeats)/Cas (CRISPR-associated protein) nuclease system is an engineered nuclease system used for genetic engineering that is based on a bacterial system. It is based in part on the adaptive immune response of many bacteria and archaea. When a virus or plasmid invades a bacterium, segments of the invader's DNA are converted into CRISPR RNAs (crRNA) by the bacteria's “immune” response. The crRNA then associates, through a region of partial complementarity, with another type of RNA called tracrRNA to guide a Cas nuclease to a region homologous to the crRNA in the target DNA called a “protospacer.” The Cas nuclease cleaves the DNA to generate blunt ends at the double-strand break at sites specified by a 20-nucleotide complementary strand sequence contained within the crRNA transcript. In some instances, the Cas nuclease requires both the crRNA and the tracrRNA for site-specific DNA recognition and cleavage.

Guide RNA (gRNA) is one example of a targeting element. In its simplest form, gRNA provides a sequence that targets a site within a genome based on complementarity (e.g., crRNA). As explained below, however, gRNA can also include additional components. For example, in particular embodiments, gRNA can include a targeting sequence (e.g., crRNA) and a component to link the targeting sequence to a cutting element. This linking component can be tracrRNA. In particular embodiments, gRNA including crRNA and tracrRNA can be expressed as a single molecule referred to as single gRNA (sgRNA). gRNA can also be linked to a cutting element through other mechanisms such as through a nanoparticle or through expression or construction of a dual or multi-purpose molecule. Those of skill in the art will appreciate that gRNA or other targeting elements to generate a selected nucleic acid sequence correction or modification, e.g., in a host cell of an adenoviral donor vector or genome of the present disclosure, can be readily designed and implemented, e.g., based on available sequence information.

In particular embodiments, targeting elements (e.g., gRNA) can include one or more modifications (e.g., a base modification, a backbone modification), to provide the nucleic acid with a new or enhanced feature (e.g., improved stability). Modified backbones may include those that retain a phosphorus atom in the backbone and those that do not have a phosphorus atom in the backbone. Suitable modified backbones containing a phosphorus atom may include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable targeting elements having inverted polarity can include a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

Examples of cutting elements include nucleases. CRISPR-Cas loci have more than 50 gene families and there are no strictly universal genes, indicating fast evolution and extreme diversity of loci architecture. Exemplary Cas nucleases include Cas1, Cas1B, Cas2, Cas3, Cas4, Cas5, Cash, Cas7, Cas8, Cas9 (also known as Csn1 and Csx12), Cas10, Cpf1, C2c3, C2c2 and C2c1Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, and Csf4.

There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NCBI Ref. Seq. No. NP_269215, and the amino acid sequence of Streptococcus thermophilus wild-type Cas9 polypeptide is set forth, e.g., in NCBI Ref. Seq. No. WP_011681470.

In particular embodiments, Cas9 refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme, in some embodiments, includes one or more catalytic domains of a Cas9 protein derived from bacteria such as Corynebacter, Sutterella, Legionella, Treponema, Filif actor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g. the two catalytic domains are derived from different bacterial species.

In some embodiments, crRNA and tracrRNA can be combined into one molecule called a single gRNA (sgRNA). In this engineered approach, the sgRNA guides Cas to target any desired sequence (see, e.g., Jinek et al., Science 337:816-821, 2012; Jinek et al., eLife 2:e00471, 2013; Segal, eLife 2:e00563, 2013). Thus, the CRISPR/Cas system can be engineered to create a double-strand break at a desired target in a genome of a cell, and harness the cell's endogenous mechanisms to repair the induced break by HDR, or NHEJ. Particular embodiments described herein utilize homology arms to promote HDR at defined integration sites.

In various embodiments, variants of the Cas9 nuclease include a single inactive catalytic domain, such as a RuvC″ or HNH″ enzyme or a nickase. A Cas9 nickase has only one active functional domain and, in some embodiments, cuts only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include N854A and N863 A. A double-strand break is introduced using a Cas9 nickase if at least two DNA-targeting RNAs that target opposite DNA strands are used. A double-nicked induced double-strand break is repaired by HDR or NHEJ. This gene editing strategy generally favors HDR and decreases the frequency of indel mutations at off-target DNA sites. The Cas9 nuclease or nickase, in some embodiments, is codon-optimized for the target cell or target organism.

### I(C)(i)(b)(2). Base Editor Payload Expression Products

The present disclosure includes, among other things, base editing agents and nucleic acids encoding the same, e.g., where the base editing agent or nucleic acid encoding the same is present in an adenoviral vector or genome. A base editing system can include a base editing enzyme and/or at least one gRNA as components thereof. In certain particular embodiments, a base editing agent and/or a base editing system of the present disclosure is present in an Ad3, 7, 11, 14, 16, 21, 34, 37, or 50 adenoviral vector

Base editing refers to the selective modification of a nucleic acid sequence by converting a base or base pair within genomic DNA or cellular RNA to a different base or base pair (Rees & Liu, Nature Reviews Genetics, 19:770-788, 2018). There are two general classes of DNA base editors: (i) cytosine base editors (CBEs) that convert guanine-cytosine base pairs into thymine-adenine base pairs, and (ii) adenine base editors (ABEs) that convert adenine-thymine base pairs to guanine cytosine base pairs. In particular embodiments, components from the CRISPR system are combined with other enzymes or biologically active fragments thereof to directly install, cause, or generate mutations such as point mutations in nucleic acids, e.g., into DNA or RNA, e.g., without making, causing, or generating one or more double-stranded breaks in the mutated nucleic acid. Certain such combinations of components are known as base editors.

DNA base editors can include a catalytically disabled nuclease fused to a nucleobase deaminase enzyme and, in some cases, a DNA glycosylase inhibitor. RNA base editors achieve analogous changes using components that base modify RNA.

Upon binding to its target locus in DNA, base pairing between the guide RNA and target DNA strand leads to displacement of a small segment of single-stranded DNA. DNA bases within this single-stranded DNA bubble can be modified by the deaminase enzyme. In certain embodiments, to improve efficiency in eukaryotic cells, a catalytically disabled nuclease also generates a nick in the non-edited DNA strand, inducing cells to repair the non-edited strand using the edited strand as a template.

For CBEs, CRISPR-based editors can be produced by linking a cytosine deaminase with a Cas nickase, e.g., Cas9 nickase (nCas9). To provide one example, nCas9 can create a nick in target DNA by cutting a single strand, reducing the likelihood of detrimental indel formation as compared to methods that require a double-stranded break. After binding with DNA, the CBE deaminates a target cytosine (C) into a uracil (U) base. Later the resultant U-G pair is either repaired by cellular mismatch repair machinery making an original C-G pair converted to T-A or reverted to the original C-G by base excision repair mediated by uracil glycosylase. In various embodiments, expression of uracil glycosylase inhibitor (UGI), e.g., a UGI present in a payload, reduces the occurrence of the second outcome and increases the generation of T-A base pair formation.

For adenosine base editors (ABEs), exemplary adenosine deaminases that can act on DNA for adenine base editing include a mutant TadA adenosine deaminases (TadA*) that accepts DNA as its substrate. E. coli TadA typically acts as a homodimer to deaminate adenosine in transfer RNA (tRNA). TadA* deaminase catalyzes the conversion of a target ‘A’ to ‘I’ (inosine), which is treated as ‘G’ by cellular polymerases. Subsequently, an original genomic A-T base pair can be converted to a G-C pair. As the cellular inosine excision repair is not as active as uracil excision, ABE does not require any additional inhibitor protein like UGI in CBE. In some embodiments, a typical ABE can include three components including a wild-type E. coli tRNA-specific adenosine deaminase (TadA) monomer, which can play a structural role during base editing, a TadA* mutant TadA monomer that catalyzes deoxyadenosine deamination, and a Cas nickase such as Cas9(D10A). In certain embodiments, there is a linker positioned between TadA and TadA*, and in certain embodiments there is a linker positioned between TadA* and the Cas nickase. In various embodiments, one or both linkers includes at least 6 amino acids, e.g., at least 5, 6, 7, 8, 9, 10, 15, 20, 25, 30, 35, 40, 45, or 50 amino acids (e.g., having a lower bound of 5, 6, 7, 8, 9, 10, or 15, amino acids and an upper bound of 20, 25, 30, 35, 40, 45, or 50 amino acids). In various embodiments, one or both linkers include 32 amino acids. In some embodiments, one or both linkers has a sequence according to (SGGS)₂-XTEN-(SGGS)₂, or a sequence otherwise known to those of skill in the art.

Base editors can directly convert one base or base pair into another, enabling the efficient installation of point mutations in non-dividing cells without generating excess undesired editing by-products, such as insertions and deletions (indels). For example, base editors can generate less than 10%, 9%, 8%, 7%, 6%, 5.5%, 5%, 4.5%, 4%, 3.5%, 3%, 2.5%, 2%, 1.5%, 1%, 0.5%, or 0.1% indels.

DNA base editors can insert such point mutations in non-dividing cells without generating double-strand breaks. Due to the lack of double-strand breaks, base editors do not result in excess undesired editing by-products, such as insertions and deletions (indels). For example, base editors can generate fewer than 10%, 9%, 8%, 7%, 6%, 5.5%, 5%, 4.5%, 4%, 3.5%, 3%, 2.5%, 2%, 1.5%, 1%, 0.5%, or 0.1% indels as compared to technologies that do rely on double-strand breaks.

Components of most base-editing systems include (1) a targeted DNA binding protein, (2) a nucleobase deaminase enzyme, and (3) a DNA glycosylase inhibitor.

Any nuclease of the CRISPR system can be disabled and used within a base editing system. Exemplary Cas nucleases include Cas1, Cas1B, Cas2, Cas3, Cas4, Cas5, Cash, Cas7, Cas8, Cas9 (also known as Csn1 and Csx12), Cas10, Cpf1, C2c3, C2c2 and C2c1Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4 and mutations thereof.

Particular embodiments utilize a nuclease-inactive Cas9 (dCas9) as the catalytically disabled nuclease. However, any nuclease of the CRISPR system (many of which are described above) can be disabled and used within a base editing system. In particular embodiments, a Cas9 domain with high fidelity is selected wherein the Cas9 domain displays decreased electrostatic interactions between the Cas9 domain and a sugar-phosphate backbone of a DNA, as compared to a wild-type Cas9 domain. In some embodiments, a Cas9 domain (e.g., a wild type Cas9 domain) includes one or more mutations that decrease the association between the Cas9 domain and a sugar-phosphate backbone of a DNA. Cas9 domains with high fidelity are known to those skilled in the art. For example, Cas9 domains with high fidelity have been described in Kleinstiver, et al., Nature 529, 490-495, 2016; and Slaymaker et al., Science 351, 84-88, 2015.

Nucleases from other gene-editing systems may also be used. For example, base-editing systems can utilize zinc finger nucleases (ZFNs) (Urnov et al., Nat Rev Genet., 11(9):636-46, 2010) and transcription activator like effector nucleases (TALENs) (Joung et al., Nat Rev Mol Cell Biol. 14(1):49-55, 2013). For additional information regarding DNA-binding nucleases, see US2018/0312825A1.

In particular embodiments, the nucleobase deaminase enzyme includes a cytidine deaminase domain or an adenine deaminase domain.

Particular embodiments utilize a cytidine deaminase domain as the nucleobase deaminase enzyme. Particular embodiments utilize an adenine deaminase domain as the nucleobase deaminase enzyme. Further, particular embodiments utilize a uracil glycosylase inhibitor (UGI) as a glycosylase inhibitor. For example, in particular embodiments, dCas9 or a Cas9 nickase can be fused to a cytidine deaminase domain. The dCas9 or a Cas9 nickase fused to the cytidine deaminase domain can be fused to one or more UGI domains. Base editors with more than one UGI domain can generate less indels and more efficiently deaminates target nucleic acids.

In particular embodiments, a deaminase domain (cytidine and/or adenine) is fused to the N-terminus of the catalytically disabled nuclease. This is because a cytidine deaminase domain fused to the N-terminus of Cas9 can have improved base-editing efficiency when compared to other configurations. In these embodiments, a glycosylase inhibitor (e.g., UGI domain) can be fused to the C-terminus of the catalytically disabled nuclease. When multiple glycosylase inhibitors are used, each can be fused to the C-terminus of the catalytically disabled nuclease.

In particular embodiments, CBE utilizing a cytidine deaminase domain convert guanine-cytosine base pairs into thymine-adenine base pairs by deaminating the exocyclic amine of the cytosine to generate uracil. Examples of cytosine deaminase enzymes include APOBEC1, APOBEC3A, APOBEC3G, CDA1, and AID. APOBEC1 particularly accepts single stranded (ss)DNA as a substrate but is incapable of acting on double stranded (ds)DNA.

Most base-editing systems also include a DNA glycosylase inhibitor that serves to override natural DNA repair mechanisms that might otherwise repair the intended base editing. In particular embodiments, the DNA glycosylase inhibitor includes an uracil glycosylase inhibitor, such as the uracil DNA glycosylase inhibitor protein (UGI) described in Wang et al. (Gene 99, 31-37, 1991).

Components of base editors can be fused directly (e.g., by direct covalent bond) or via linkers. For example, the catalytically disabled nuclease can be fused via a linker to the deaminase enzyme and/or a glycosylase inhibitor. Multiple glycosylase inhibitors can also be fused via linkers. As will be understood by one of ordinary skill in the art, linkers can be used to link any peptides or portions thereof.

Exemplary linkers include polymeric linkers (e.g., polyethylene, polyethylene glycol, polyamide, polyester); amino acid linkers; carbon-nitrogen bond amide linkers; cyclic or acyclic, substituted or unsubstituted, branched or unbranched aliphatic or heteroaliphatic linkers; monomeric, dimeric, or polymeric aminoalkanoic acid linkers; aminoalkanoic acid (e.g., glycine, ethanoic acid, alanine, β-alanine, 3-aminopropanoic acid, 4-aminobutanoic acid, 5-pentanoic acid) linkers; monomeric, dimeric, or polymeric aminohexanoic acid (Ahx) linkers; carbocyclic moiety (e.g., cyclopentane, cyclohexane) linkers; aryl or heteroaryl moiety linkers; and phenyl ring linkers.

Linkers can also include functionalized moieties to facilitate attachment of a nucleophile (e.g., thiol, amino) from a peptide to the linker. Any electrophile may be used as part of the linker. Exemplary electrophiles include activated esters, activated amides, Michael acceptors, alkyl halides, aryl halides, acyl halides, and isothiocyanates.

In particular embodiments, linkers range from 4-100 amino acids in length. In particular embodiments, linkers are 4 amino acids, 9 amino acids, 14 amino acids, 16 amino acids, 32 amino acids, or 100 amino acids.

Numerous base-editing (BE) systems formed by linking targeted DNA binding proteins with cytidine deaminase enzymes and DNA glycosylase inhibitors (e.g., UGI) have been described. These complexes include for example, BE1 ([APOBEC1-16 amino acid (aa) linker-Sp dCas9 (D10A, H840A)] Komer et al., Nature, 533, 420-424, 2016), BE2 ([APOBEC1-16aa linker-Sp dCas9 (D10A, H840A)-4aa linker-UGI] Komer et al., 2016 supra), BE3 ([APOBEC1-16aa linker-Sp nCas9 (D10A)-4aa linker-UGI] Komer et al., supra), HF-BE3 ([APOBEC1-16aa linker-HF nCas9 (D10A)-4aa linker-UGI] Rees et al., Nat. Commun. 8, 15790, 2017), BE4, BE4max ([APOBEC1-32aa linker-Sp nCas9 (D10A)-9aa linker-UGI-9aa linker-UGI] Koblan et al., Nat. Biotechnol 10.1038/nbt.4172, 2018; Komer et al., Sci. Adv., 3, eaao4774, 2017), BE4-GAM ([Gam-16aa linker-APOBEC1-32aa linker-Sp nCas9 (D10A)-9aa linker-UGI-9aa linker-UGI] Komer et al., 2017 supra), YE1-BE3 ([APOBEC1 (W90Y, R126E)-16aa linker-Sp nCas9 (D10A)-4aa linker-UGI] Kim et al., Nat. Biotechnol. 35, 475-480, 2017), EE-BE3 ([APOBEC1 (R126E, R132E)-16aa linker-Sp nCas9 (D10A)-4aa linker-UGI] Kim et al., 2017 supra), YE2-BE3 ([APOBEC1 (W90Y, R132E)-16aa linker-Sp nCas9 (D10A)-4aa linker-UGI]Kim et al., 2017 supra), YEE-BE3 ([APOBEC1 (W90Y, R126E, R132E)-16aa linker-Sp nCas9 (D10A)-4aa linker-UGI] Kim et al., 2017 supra), VQR-BE3 ([APOBEC1-16aa linker-Sp VQR nCas9 (D10A)-4aa linker-UGI] Kim et al., 2017 supra), VRER-BE3 ([APOBEC1-16aa linker-Sp VRER nCas9 (D10A)-4aa linker-UGI] Kim et al., Nat. Biotechnol. 35, 475-480, 2017), Sa-BE3 ([APOBEC1-16aa linker-Sa nCas9 (D10A)-4aa linker-UGI] Kim et al., 2017 supra), SA-BE4 ([APOBEC1-32aa linker-Sa nCas9 (D10A)-9aa linker-UGI-9aa linker-UGI] Komer et al., 2017 supra), SaBE4-Gam ([Gam-16aa linker-APOBEC1-32aa linker-Sa nCas9 (D10A)-9aa linker-UGI-9aa linker-UGI] Komer et al., 2017 supra), SaKKH-BE3 ([APOBEC1-16aa linker-Sa KKH nCas9 (D10A)-4aa linker-UGI] Kim et al., 2017 supra), Cas12a-BE ([APOBEC1-16aa linker-dCas12a-14aa linker-UGI], Li et al., Nat. Biotechnol. 36, 324-327, 2018), Target-AID ([Sp nCas9 (D10A)-100aa linker-CDA1-9aa linker-UGI] Nishida et al., Science, 353, 10.1126/science.aafi3729, 2016), Target-AID-NG ([Sp nCas9 (D10A)-NG-100aa linker-CDA1-9aa linker-UGI] Nishimasu et al., Science, 361(6408): 1259-1262, 2018), xBE3 ([APOBEC1-16aa linker-xCas9(D10A)-4aa linker-UGI] Hu et al., Nature, 556, 57-63, 2018), eA3A-BE3 ([APOBEC3A (N37G)-16aa linker-Sp nCas9(D10A)-4aa linker-UGI] Gerkhe et al., Nat. Biotechnol., 10.1038/nbt.4199, 2018), A3A-BE3 ([hAPOBEC3A-16aa linker-Sp nCas9(D10A)-4aa linker-UGI] Wang et al., Nat. Biotechnol. 10.1038/nbt.4198, 2018), and BE-PLUS ([10X GCN4-Sp nCas9(D10A)/ScFv-rAPOBEC1-UGI] Jiang et al., Cell. Res, 10.1038/s41422-018-0052-4, 2018). For additional examples of BE complexes, including adenine deaminase base editors, see Rees & Liu Nat. Rev Genet. 19(12): 770-788, 2018.

For additional information regarding base editors, see US2018/0312825A1, WO02018/165629A, Urnov et al., Nat Rev Genet. 11(9):636-46, 2010; Joung et al., Nat Rev Mol Cell Biol. 14(1):49-55, 2013; Charpentier et al., Nature.; 495(7439):50-1, 2013; Seo & Kim, Nature Medicine, 24, 1493-1495, 2018, and Rees & Liu, Nature Reviews Genetics, 19, 770-78, 2018 each of which is incorporated herein by reference in its entirety and with specific respect to base editors. Certain base editor constructs that can be used in various embodiments of the present disclosure are described in Zafra et al., Nat Biotech, 36(9):888-893, 2018, and Koblan et al., Nat. Biotech 36(9). 843-846, 2018, each of which is incorporated herein by reference in its entirety and with specific respect to base editor constructs.

### I(C)(i)(b)(3). Prime Editor Payload Expression Products

Prime editing can introduce all possible types of point mutations, small insertions, and small deletions in a precise and targeted manner. Prime editors are fusion proteins including a Cas9 nickase domain (e.g., an inactivated HNH nuclease) and an engineered reverse transcriptase domain. The prime editor enzyme is targeted to the editing site by an engineered prime editing gRNA (pegRNA), which not only specifies the target site in its spacer sequence, but also encodes the desired edit in an extension that is typically at the 3′ end of the pegRNA.

At least three prime editor system have been characterized. PE1 includes a fusion of Cas9 nickase with wild-type Moloney murine leukemia virus (M-MLV) reverse transcriptase (RT). PE2 is similar to PE1 but includes an engineered pentamutant M-MLV RT that increases editing efficiency by about threefold. PE3 combines the PE2 fusion protein and pegRNA with an additional sgRNA that targets the non-edited strand for nicking. A variant of the PE3 system called PE3b includes a nicking sgRNA that targets only the edited sequence, resulting in decreased levels of indel products by preventing nicking of the non-edited DNA strand until the other strand has been converted to the edited sequence.

### I(C)(i)(c). Small RNA Payload Expression Products

Small RNAs are short, non-coding RNA molecules that play a role in regulating gene expression. In particular embodiments, small RNAs are less than 200 nucleotides in length. In particular embodiments, small RNAs are less than 100 nucleotides in length. In particular embodiments, small RNAs are less than 50, 45, 40, 35, 30, 25, or 20 nucleotides in length. In particular embodiments, small RNAs are less than 20 nucleotides in length. In various embodiments a small RNA has a length having a lower bound of 5, 10, 15, 20, 25, or 30 nucleotides and an upper bound of 20, 25, 30, 35, 40, 45, 50, 75, or 100 nucleotides. Small RNAs include but are not limited to microRNAs (miRNAs, Piwi-interacting RNAs (piRNAs), small interfering RNAs (siRNAs), small nucleolar RNAs (snoRNAs), tRNA-derived small RNAs (tsRNAs) small rDNA-derived RNAs (srRNAs), and small nuclear RNAs. Additional classes of small RNAs continue to be discovered.

In particular embodiments, interfering RNA molecules that are homologous to a target mRNA or to which the interfering RNA can hybridize can lead to degradation of the target mRNA molecule or reduced translation of the target mRNA, a process referred to as RNA interference (RNAi) (Carthew, Curr. Opin. Cell. Biol. 13: 244-248, 2001). RNAi occurs in cells naturally to remove foreign RNAs (e.g., viral RNAs). In some instances, natural RNAi proceeds via fragments cleaved from free double-strand RNA (dsRNA) which direct the degradative mechanism to other similar RNA sequences. Alternatively, RNAi can be manufactured, for example, to silence the expression of target genes. Exemplary RNAi molecules include small hairpin RNA (shRNA, also referred to as short hairpin RNA) and small interfering RNA (siRNA).

Without limiting the disclosure, and without being bound by theory, RNA interference in nature and/or in some embodiments is typically a two-step process. In the first step, the initiation step, input dsRNA is digested into 21-23 nucleotide (nt) siRNA, probably by the action of Dicer, a member of the ribonuclease (RNase) III family of dsRNA-specific ribonucleases, which processes (cleaves) dsRNA (introduced directly or via a transgene or a virus) in an ATP-dependent manner. Successive cleavage events degrade the RNA to 19-21 base pair (bp) duplexes (siRNA), each with 2-nucleotide 3′ overhangs.

In a second step, an effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and typically cleaves the mRNA into 12 nucleotide fragments from the 3′ terminus of the siRNA. Research indicates that each RISC contains a single siRNA and an RNase.

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC.

ShRNAs are single-stranded polynucleotides with a hairpin loop structure. The single-stranded polynucleotide has a loop segment linking the 3′ end of one strand in the double-stranded region and the 5′ end of the other strand in the double-stranded region. The double-stranded region is formed from a first sequence that is hybridizable to a target sequence, such as a polynucleotide encoding transgene, and a second sequence that is complementary to the first sequence, thus the first and second sequence form a double stranded region to which the linking sequence connects the ends of to form the hairpin loop structure. The first sequence can be hybridizable to any portion of a polynucleotide encoding transgene. The double-stranded stem domain of the shRNA can include a restriction endonuclease site.

Transcription of shRNAs is initiated at a polymerase III (Pol III) promoter and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of 21-23 nucleotides.

The stem-loop structure of shRNAs can have optional nucleotide overhangs, such as 2-bp overhangs, for example, 3′ UU overhangs. While there may be variation, stems typically range from 15 to 49, 15 to 35, 19 to 35, 21 to 31 bp, or 21 to 29 bp, and the loops can range from 4 to 30 bp, for example, 4 to 23 bp. In particular embodiments, shRNA sequences include 45-65 bp; 50-60 bp; or 51, 52, 53, 54, 55, 56, 57, 58, or 59 bp. In particular embodiments, shRNA sequences include 52 or 55 bp. In particular embodiments siRNAs have 15-25 bp. In particular embodiments siRNAs have 16, 17, 18, 19, 20, 21, 22, 23, or 24 bp. In particular embodiments siRNAs have 19 bp. The skilled artisan will appreciate, however, that siRNAs having a length of less than 16 nucleotides or greater than 24 nucleotides can also function to mediate RNAi. Longer RNAi agents have been demonstrated to elicit an interferon or Protein kinase R (PKR) response in certain mammalian cells which may be undesirable. Preferably the RNAi agents do not elicit a PKR response (i.e., are of a sufficiently short length). However, longer RNAi agents may be useful, for example, in situations where the PKR response has been downregulated or dampened by alternative means.

In certain illustrative embodiments, the present disclosure includes an adenoviral vector payload that encodes an shRNA targeted to the gene encoding BCL11A, where the shRNA causes decreased translation of BCL11A.

### I(C)(ii). Payload Regulatory Sequences

### I(C)(ii)(a). Promoter Regulatory Sequences

A promoter can be a non-coding genomic DNA sequence, usually upstream (5′) to the relevant coding sequence, to which RNA polymerase binds before initiating transcription. This binding aligns the RNA polymerase so that transcription will initiate at a specific transcription initiation site. The nucleotide sequence of the promoter determines the nature of the enzyme and other related protein factors that attach to it and the rate of RNA synthesis. The RNA is processed to produce messenger RNA On RNA) which serves as a template for translation of the RNA sequence into the amino acid sequence of the encoded polypeptide. The 5′ non-translated leader sequence is a region of the mRNA upstream of the coding region that may play a role in initiation and translation of the mRNA. The 3′ transcription termination/polyadenylation signal is a non-translated region downstream of the coding region that functions in the plant cell to cause termination of the RNA synthesis and the addition of polyadenylate nucleotides to the 3′ end.

Promoters can include general promoters, tissue-specific promoters, cell-specific promoters, and/or promoters specific for the cytoplasm. Promoters may include strong promoters, weak promoters, constitutive expression promoters, and/or inducible (conditional) promoters. Inducible promoters direct or control expression in response to certain conditions, signals, or cellular events. For example, the promoter may be an inducible promoter that requires a particular ligand, small molecule, transcription factor, hormone, or hormone protein in order to effect transcription from the promoter. Particular examples of promoters include the AFP (α-fetoprotein) promoter, amylase 1C promoter, aquaporin-5 (AP5) promoter, α1-antitrypsin promoter, β-act promoter, β-globin promoter, β-Kin promoter, B29 promoter, CCKAR promoter, CD14 promoter, CD43 promoter, CD45 promoter, CD68 promoter, CEA promoter, c-erbB2 promoter, COX-2 promoter, CXCR4 promoter, desmin promoter, E2F-1 promoter, human elongation factor 1α promoter (EF1α), CMV (cytomegalovirus viral) promoter, minCMV promoter, SV40 (simian virus 40) immediately early promoter, EGR1 promoter, eIF4A1 promoter, elastase-1 promoter, endoglin promoter, FerH promoter, FerL promoter, fibronectin promoter, Flt-1 promoter, GAPDH promoter, GFAP promoter, GPIIb promoter, GRP78 promoter, GRP94 promoter, HE4 promoter, hGR1/1 promoter, hNIS promoter, Hsp68 promoter, the Hsp68 minimal promoter (proHSP68), HSP70 promoter, HSV-1 virus TK gene promoter, hTERT promoter, ICAM-2 promoter, kallikrein promoter, LP promoter, major late promoter (MLP), Mb promoter, Rho promoter, MT (metallothionein) promoter, MUC1 promoter, NphsI promoter, OG-2 promoter, PGK (Phospho Glycerate kinase) promoters, PGK-1 promoter, polymerase III (Pol III) promoter, PSA promoter, ROSA promoter, SP-B promoter, Survivn promoter, SYN1 promoter, SYT8 gene promoter, TRP1 promoter, Tyr promoter, ubiquitin B promoter, WASP promoter, and the Rous Sarcoma Virus (RSV) long-terminal repeat (LTR) promoter

Promoters may be obtained as native promoters or composite promoters. Native promoters, or minimal promoters, refer to promoters that include a nucleotide sequence from the 5′ region of a given gene. A native promoter includes a core promoter and its natural 5′UTR. In particular embodiments, the 5′UTR includes an intron. Composite promoters refer to promoters that are derived by combining promoter elements of different origins or by combining a distal enhancer with a minimal promoter of the same or different origin.

In particular embodiments, promoters include wild type promoter sequences and sequences with optional changes (including insertions, point mutations or deletions) at certain positions relative to the wild-type promoter. In particular embodiments, promoters vary from naturally occurring promoters by having 1 change per 20 nucleotide stretch, 2 changes per 20 nucleotide stretch, 3 changes per 20 nucleotide stretch, 4 changes per 20 nucleotide stretch, or 5 changes per 20 nucleotide stretch. In particular embodiments, the natural sequence will be altered in 1, 2, 3, 4, 5, 6, 7, 8, 9, or 10 bases. The promoter may vary in length, including from 50 nucleotides of LTR sequence to 100, 200, 250 or 350 nucleotides of LTR sequence, with or without other viral sequence.

Some promoters are specific to a tissue or cell and some promoters are non-specific to a tissue or cell. Each gene in mammalian cells has its own promoter and some promoters can only be activated in certain cell types. A non-specific promoter, or ubiquitous promoter, aids in initiation of transcription of a gene or nucleotide sequence that is operably linked to the promoter sequence in a wide range of cells, tissues and cell cycles. In particular embodiments, the promoter is a non-specific promoter. In particular embodiments, a non-specific promoter includes CMV promoter, RSV promoter, SV40 promoter, mammalian elongation factor 1α (EF1α) promoter, β-act promoter, EGR1 promoter, eIF4A1 promoter, FerH promoter, FerL promoter, GAPDH promoter, GRP78 promoter, GRP94 promoter, HSP70 promoter, β-Kin promoter, PGK-1 promoter, ROSA promoter, and/or ubiquitin B promoter.

A specific promoter aids in cell specific expression of a nucleotide sequence that is operably linked to the promoter sequence.

### I(C)(ii)(b). Micro RNA Site Regulatory Sequences

In various embodiments, a microRNA (or miRNA) control system can refer to a method or composition in which expression of a gene is regulated by the presence of microRNA sites (e.g., nucleic acid sequences with which a microRNA can interact). In various embodiments, the present disclosure includes an adenoviral donor vector that includes a payload in which a nucleic acid sequence encoding an expression product is operably linked to an miRNA target site such that expression of the expression product is controlled by presence, level, activity, and/or contact with a corresponding miRNA. For the avoidance of doubt the present disclosure contemplates that a nucleic acid sequence operably linked with an miRNA site, e.g., as disclosed herein can be a nucleic acid sequence that encodes, e.g., any of one or more expression products provided herein.

### I(C)(iii). Selection Sequences

In particular embodiments vectors include a selection element including a selection cassette. In particular embodiments, a selection cassette includes a promoter, a cDNA that adds or confers resistance to a selection agent, and a poly A sequence that enables stopping the transcription of this independent transcriptional element.

A selection cassette can encode one or more proteins that (a) confer resistance to antibiotics or other toxins, (b) complement auxotrophic deficiencies, or (c) supply critical nutrients not available from complex media, e.g., the gene encoding D-alanine racemase for Bacilli. Any number of selection systems may be used to recover transformed cell lines. In particular embodiments, a positive selection cassette includes resistance genes to neomycin, hygromycin, ampicillin, puromycin, phleomycin, zeomycin, blasticidin, or viomycin. In particular embodiments, a positive selection cassette includes the DHFR (dihydrofolate reductase) gene providing resistance to methotrexate, the MGMT^{P140K} gene responsible for the resistance to O⁶BG/BCNU, the HPRT (Hypoxanthine phosphoribosyl transferase) gene responsible for the transformation of specific bases present in the HAT selection medium (aminopterin, hypoxanthine, thymidine) and other genes for detoxification with respect to some drugs. In particular embodiments, the selection agent includes neomycin, hygromycin, puromycin, phleomycin, zeomycin, blasticidin, viomycin, ampicillin, O⁶BG/BCNU, methotrexate, tetracycline, aminopterin, hypoxanthine, thymidine kinase, DHFR, Gln synthetase, or ADA.

In particular embodiments, negative selection cassettes include a gene for transformation of a substrate present in the culture medium into a toxic substance for the cell that expresses the gene. These molecules include detoxification genes of diptheria toxin (DTA) (Yagi et al., Anal Biochem. 214(1):77-86, 1993; Yanagawa et al., Transgenic Res. 8(3):215-221, 1999), the kinase thymidine gene of the Herpes virus (HSV TK) sensitive to the presence of ganciclovir or FIAU. The HPRT gene may also be used as a negative selection by addition of 6-thioguanine (6TG) into the medium. and for all positive and negative selections, a poly A transcription termination sequence from different origins, the most classical being derived from SV40 poly A, or a eukaryotic gene poly A (bovine growth hormone, rabbit β-globin, etc.).

In particular embodiments, the selection cassette includes MGMT^{P140K} as described in Olszko et al. (Gene Therapy 22: 591-595, 2015). In particular elements, the selection agent includes O⁶BG/BCNU.

The drug resistant gene MGMT encoding human alkyl guanine transferase (hAGT) is a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG but retain their ability to repair DNA damage (Maze et al., J. Pharmacol. Exp. Ther. 290: 1467-1474, 1999). MGMT^{P140K}-based drug resistant gene therapy has been shown to confer chemoprotection to mouse, canine, rhesus macaques, and human cells, specifically hematopoietic cells (Zielske et al., J. Clin. Invest. 112: 1561-1570, 2003; Pollok et al., Hum. Gene Ther. 14: 1703-1714, 2003; Gerull et al., Hum. Gene Ther. 18: 451-456, 2007; Neff et al., Blood 105: 997-1002, 2005; Larochelle et al., J. Clin. Invest. 119: 1952-1963, 2009; Sawai et al., Mol. Ther. 3: 78-87, 2001).

In particular embodiments, combination with an in vivo selection cassette will be a critical component for diseases without a selective advantage of gene-corrected cells. For example, in SCID and some other immunodeficiencies and FA, corrected cells have an advantage and only transducing the therapeutic gene into a “few” HSPCs is sufficient for therapeutic efficacy. For other diseases like hemoglobinopathies (i.e., sickle cell disease and thalassemia) in which therapeutically modified cells do not demonstrate a competitive advantage, in vivo selection of the modified cells, e.g., for expression of an in vivo selection cassette such as MGMT^{P140K}, will select for the few transduced HSPCs, allowing an increase in the gene corrected cells and in order to achieve therapeutic efficacy. This approach can also be applied to HIV by making HSPCs resistant to HIV in vivo rather than ex vivo genetic modification.

### I(C)(iv). Stuffer Sequences

In particular embodiments, the vector includes a stuffer sequence. In particular embodiments, the stuffer sequence may be added to render the genome at a size near that of wild-type length. Stuffer is a term generally recognized in the art intended to define functionally inert sequence intended to extend the length of the genome.

The stuffer sequence is used to achieve efficient packaging and stability of the vector. In particular embodiments, the stuffer sequence is used to render the genome size between 70% and 110% of that of the wild type virus.

The stuffer sequences can be any DNA, preferably of mammalian origin. In a preferred embodiment of the invention, stuffer sequences are non-coding sequences of mammalian origin, for example intronic fragments.

The stuffer sequence, when used to keep the size of the vector a predetermined size, can be any non-coding sequence or sequence that allows the genome to remain stable in dividing or nondividing cells. These sequences can be derived from other viral genomes (e.g. Epstein bar virus) or organism (e.g. yeast). For example, these sequences could be a functional part of centromeres and/or telomeres.

### I(C)(v). Payload Integration and Support Vectors

Gene therapy often requires integration of a desired nucleic acid payload into the genome of a target cell. A variety of systems can be designed and/or used for integration of a payload into a host or target cell genome. Various such systems can include one or more of certain payload sequence features and support vectors and support genomes (support genomes).

One means of engineering adenoviral vectors that integrate a payload into a host cell genome has been to produce integrating viral hybrid vectors. Integrating viral hybrid vectors combine genetic elements of a vector that efficiently transduces target cells with genetic elements of a vector that stably integrates its vector payload. Integration elements of interest, e.g., for use in combination with adenoviral vectors, have included those of bacteriophage integrase PHiC31, retrotransposons, retrovirus (e.g., LTR-mediated or retrovirus integrate-mediated), zinc-finger nuclease, DNA-binding domain-retroviral integrase fusion proteins, AAV (e.g., AAV-ITR or AAV-Rep protein-mediated), and Sleeping Beauty (SB) transposase.

Ad3, 7, 11, 14, 16, 21, 34, 37, or 50 vectors described herein can optionally include transposable elements including transposases and transposons. Transposases can include integrases from retrotransposons or of retroviral origin, as well as an enzyme that is a component of a functional nucleic acid-protein complex capable of transposition and which is mediating transposition. A transposition reaction includes a transposon and a transposase or an integrase enzyme. In particular embodiments, the efficiency of integration, the size of the DNA sequence that can be integrated, and the number of copies of a DNA sequence that can be integrated into a genome can be improved by using such transposable elements. Transposons include a short nucleic acid sequence with terminal repeat sequences upstream and downstream of a larger segment of DNA. Transposases bind the terminal repeat sequences and catalyze the movement of the transposon to another portion of the genome.

A number of transposases have been described in the art that facilitate insertion of nucleic acids into the genome of vertebrates, including humans. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himarl, Passport, Minos, Ac/Ds, PIF, Harbinger, Harbinger3-DR, HSmarl, and spinON.

The PiggyBac (PB) transposase is a compact functional transposase protein that is described in, for example, Fraser et al., Insect Mol. Biol., 1996, 5, 141-51; Mitra et al., EMBO J., 2008, 27, 1097-1109; Ding et al., Cell, 2005, 122, 473-83; and U.S. Pat. Nos. 6,218,185; 6,551,825; 6,962,810; 7,105,343; and 7,932,088. Hyperactive piggyBac transposases are described in U.S. Pat. No. 10,131,885.

Additional information on DNA transposons can be found, for instance, in Muñoz-Lopez & Garcia Perez, Curr Genomics, 11(2):115-128, 2010.

Sleeping Beauty is described in Ivies et al. Cell 91, 501-510, 1997; Izsvak et al., J. Mol. Biol., 302(1):93-102, 2000; Geurts et al., Molecular Therapy, 8(1): 108-117, 2003; Mates et al. Nature Genetics 41:753-761, 2009; and U.S. Pat. Nos. 6,489,458; 7,148,203; and 7,160,682; US Publication Nos. 2011/117072; 2004/077572; and 2006/252140. In certain embodiments, the Sleeping Beauty transposase enzyme is a Hyperactive Sleeping Beauty SB100x transposase enzyme. SB transposons are most efficiently transposed when present in circularized nucleic acid molecules (Yant et al., Nature Biotechnology, 20: 999-1005, 2002).

Systematic mutagenesis studies have been undertaken to increase the activity of the SB transposase. For example, Yant et al., undertook the systematic exchange of the N-terminal 95 AA of the SB transposase for alanine (Mol. Cell Biol. 24: 9239-9247, 2004). Ten of these substitutions caused hyperactivity between 200-400% as compared to SB10 as a reference. SB16, described in Baus et al. (Mol. Therapy 12: 1148-1156, 2005) was reported to have a 16-fold activity increase as compared to SB10. Additional hyperactive SB variants are described in Zayed et al. (Molecular Therapy 9(2):292-304, 2004) and U.S. Pat. No. 9,840,696.

SB transposases transpose nucleic acid transposon payloads that are positioned between SB ITRs. Various SB ITRs are known in the art. In some embodiments, an SB ITR is a 230 bp sequence including imperfect direct repeats of 32 bp in length that serve as recognition signals for the transposase.

In various embodiments, an Ad3, 7, 11, 14, 16, 21, 34, 37, or 50 donor vector or genome includes a payload that includes SB100x transposon inverted repeats that flank an integration element that includes at least one coding sequence that encodes a 0-globin expression product or a γ-globin expression product.

In various embodiments, an adenoviral transposition system includes an Ad3, 7, 11, 14, 16, 21, 34, 37, or 50 donor vector or genome that includes an integration element flanked by transposon inverted repeats, and can further include an adenoviral support vector or support genome. In various embodiments, a support vector includes (i) the adenoviral capsid; and (ii) an adenoviral support genome including a nucleic acid sequence encoding a transposase that corresponds to the inverted repeats that flank the integration element. Accordingly, in various embodiments, at least one function of a support vector or support genome can be to encode, express, and/or deliver to a target cell a transposase for transposition of an integration element present in a donor vector administered to the target cell. For instance, in some embodiments, an Ad3, 7, 11, 14, 16, 21, 34, 37, or 50 donor vector or genome includes SB100x transposon inverted repeats that flank an integration element that includes at least one coding sequence that encodes a β-globin expression product or a γ-globin expression product, and a support vector or support genome includes a coding sequence that encodes SB100x transposase. In certain embodiments, an integration element is flanked by recombinase direct repeats, e.g., where the integration element is flanked by transposon inverted repeats and the transposon inverted repeats are flanked by recombinase direct repeats. In certain such embodiments, at least one function of a support vector or support genome can be to encode, express, and/or deliver to a target cell a recombinase for recombination of recombinase sites present in a donor vector administered to the target cell. In various embodiments, a support vector or support genome can encode, express, and/or deliver to a target cell a recombinase for recombination of recombinase sites present in a donor vector administered to the target cell and also encode, express, and/or deliver to a target cell a transposase for transposition of an integration element present in a donor vector administered to the target cell.

Particular embodiments disclosed herein also use site-specific recombinase systems. In these embodiments, in addition to at least one therapeutic gene, the transposon including transposase-recognized inverted repeats also includes at least one recombinase-recognized site. Thus, in particular embodiments, The present disclosure also provides methods of integrating a therapeutic gene into the genome including administering: (a) a transposon including the therapeutic gene, wherein the therapeutic gene is flanked by (i) an inverted repeat sequence recognized by a transposase and (ii) a recombinase-recognized site; and b) a transposase and recombinase that serve to excise the therapeutic gene from a plasmid, episome, or transgene and integrate the therapeutic gene into the genome. In some embodiments, the protein(s) of (b) are administered as a nucleic acid encoding the protein(s). In some embodiments, the transposon and the nucleic acids encoding the protein(s) of (b) are present on separate vectors. In some embodiments, the transposon and nucleic acid encoding the protein(s) of (b) are present on the same vector. When present on the same vector, the portion of the vector encoding the protein(s) of (b) are located outside the portion carrying the transposon of (a). In other words, the transposase and/or recombinase encoding region is located external to the region flanked by the inverted repeats and/or recombinase-recognition site. In the aforementioned methods, the transposase protein recognizes the inverted repeats that flank an inserted nucleic acid, such as a nucleic acid that is to be inserted into a target cell genome. The use of recombinases and recombinase-recognized sites can increase the size of a transposon that can be integrated into a genome further.

Examples of recombinase systems include the Flp/Frt system, the Cre/loxP system, the Dre/rox system, the Vika/vox system, and the PhiC31 system. The Flp/Frt DNA recombinase system was isolated from Saccharomyces cerevisiae. The Flp/Frt system includes the recombinase Flp (flippase) that catalyzes DNA-recombination on its Frt recognition sites. Variants of the Flp protein include GenBank: ABD57356.1) and GenBank: ANW61888.1.

The Cre/loxP system is described in, for example, EP 02200009B1. Cre is a site-specific DNA recombinase isolated from bacteriophage P1. The recognition site of the Cre protein is a nucleotide sequence of 34 base pairs, the loxP site. Cre recombines the 34 bp loxP DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine. Variants of the lox recognition site that can also be used include: lox2272; lox511; lox66; lox71; loxM2; and 1ox5171. The VCre/VloxP recombinase system was isolated from Vibrio plasmid p0908. The sCre/SloxP system is described in WO 2010/143606. The Dre/rox system is described in U.S. Pat. Nos. 7,422,889 and 7,915,037B2. It generally includes a Dre recombinase isolated from Enterobacteria phage D6 and the rox recognition site. The Vika/vox system is described in U.S. Pat. No. 10,253,332. Additionally, the PhiC31 recombinase recognizes the AttB/AttP binding sites.

The amount of vector nucleic acid including the transposon (including inverted repeats and/or recombinase recognition sites), and in various embodiments the amount of vector nucleic acid encoding the transposase and/or recombinase, introduced into the cell is/are sufficient to provide for the desired excision and insertion of the transposon nucleic acid into the target cell genome. As such, the amount of vector nucleic acid introduced should provide for a sufficient amount of transposase activity and/or recombinase activity and a sufficient copy number of the transposon that is desired to be inserted into the target cell genome. Particular embodiments include a 1:1; 1:2; or 1:3 ratio of transposon to transposase/recombinase.

The subject methods result in stable integration of the nucleic acid into the target cell genome. By stable integration is meant that the nucleic acid remains present in the target cell genome for more than a transient period of time and passes on a part of the chromosomal genetic material to the progeny of the target cell.

As indicated previously, particular embodiments utilize homology arms to facilitate targeted insertion of genetic constructs utilizing homology directed repair. Homology arms can be any length with sufficient homology to a genomic sequence at a cleavage site, e.g. 70%, 80%, 85%, 90%, 95%, or 100% homology with the nucleotide sequences flanking the cleavage site, e.g., within 50 bases or less of the cleavage site, e.g., within 30 bases, within 15 bases, within 10 bases, within 5 bases, or immediately flanking the cleavage site, to support HDR between it and the genomic sequence to which it bears homology. Homology arms are generally identical to the genomic sequence, for example, to the genomic region in which the double stranded break (DSB) occurs. However, as indicated, absolute identity is not required.

Particular embodiments can utilize homology arms with 25, 50, 100, or 200 nucleotides (nt), or more than 200 nt of sequence homology between a homology-directed repair template and a targeted genomic sequence (or any integral value between 10 and 200 nucleotides, or more). In particular embodiments, homology arms are 40-1000 nt in length. In particular embodiments, homology arms are 500-2500 base pairs, 700-2000 base pairs, or 800-1800 base pairs. In particular embodiments, homology arms include at least 800 base pairs or at least 850 base pairs. The length of homology arms can also be symmetric or asymmetric.

Particular embodiment can utilize first and/or second homology arms each including at least 25, 50, 100, 200, 400, 600, 800, 1,000, 1,200, 1,400, 1,600, 1,800, 2,000, 2,500, or 3,000 nucleotides or more, having sequence identity or homology with a corresponding fragment of a target genome. In some embodiments, first and/or second homology arms each include a number of nucleotides having sequence identity or homology with a corresponding fragment of a target genome that has a lower bound of 25, 50, 100, 200, 400, 600, 800, 1,000, 1,200, 1,400, 1,600, or 1,800 nucleotides and an upper bound of 1,000, 1,200, 1,400, 1,600, 1,800, 2,000, 2,500, or 3,000 nucleotides. In some embodiments, first and/or second homology arms each include a number of nucleotides having sequence identity or homology with a corresponding fragment of a target genome that is between 40 and 1,000 nucleotides, between 500 and 2,500 nucleotides, between 700 and 2,000 nucleotides, or between 800 and 1800 nucleotides, or that has a length of at least 800 nucleotides or at least 850 nucleotides. First and second homology arms can have same, similar, or different lengths.

For additional information regarding homology arms, see Richardson et al., Nat Biotechnol. 34(3):339-44, 2016.

In particular embodiments, genetic constructs (e.g., genes leading to expression of a therapeutic product within a cell) are precisely inserted within genomic safe harbors. Genomic safe harbor sites are intragenic or extragenic regions of the genome that are able to accommodate the predictable expression of newly integrated DNA without adverse effects on the host cell. A useful safe harbor must permit sufficient transgene expression to yield desired levels of the encoded protein. A genomic safe harbor site also must not alter cellular functions. Methods for identifying genomic safe harbor sites are described in Sadelain et al., Nature Reviews 12:51-58, 2012; and Papapetrou et al., Nat Biotechnol. 29(1):73-8, 2011. In particular embodiments, a genomic safe harbor site meets one or more (one, two, three, four, or five) of the following criteria: (i) distance of at least 50 kb from the 5′ end of any gene, (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the genome.

In particular embodiments, to meet the criteria of a genomic safe harbor, chromatin sites must be >150 kb away from a known oncogene, >30 kb away from a known transcription start site; and have no overlap with coding mRNA. In particular embodiments, to meet the criteria of a genomic safe harbor, chromatin sites must be >200 kb away from a known oncogene, >40 kb away from a known transcription start site; and have no overlap with coding mRNA. In particular embodiments, to meet the criteria of a genomic safe harbor, chromatin sites must be >300 kb away from a known oncogene, >50 kb away from a known transcription start site; and have no overlap with coding mRNA. In particular embodiments, a genomic safe harbor meets the preceding criteria (>150 kb, >200 kb or >300 kb away from a known transcription start site; and have no overlap with coding mRNA >40 kb, or >50 kb away from a known transcription start site with no overlap with coding mRNA) and additionally is 100% homologous between an animal of a relevant animal model and the human genome to permit rapid clinical translation of relevant findings.

In particular embodiments, a genomic safe harbor meets criteria described herein and also demonstrates a 1:1 ratio of forward:reverse orientations of lentiviral integration further demonstrating the locus does not impact surrounding genetic material.

Particular genomic safe harbors sites include CCR5, HPRT, AAVS1, Rosa and albumin. See also, e.g., U.S. Pat. Nos. 7,951,925 and 8,110,379; U.S. Publication Nos. 2008/0159996; 2010/00218264; 2012/0017290; 2011/0265198; 2013/0137104; 2013/0122591; 2013/0177983 and 2013/0177960 for additional information and options for appropriate genomic safe harbor integration sites.

Various technologies known in the art can be used to direct integration of an integration element at specific genomic loci such as genomic safe harbors. For example AAV-mediated gene targeting, as well as homologous recombination enhanced by the introduction of DNA double-strand breaks using site-specific endonucleases (zinc-finger nucleases, meganucleases, transcription activator-like effector (TALE) nucleases), and CRISPR/Cas systems are all tools that can mediate targeted insertion of foreign DNA at predetermined genomic loci such as genomic safe harbors.

In certain embodiments, integration of an integration element at specific genomic loci such as genomic safe harbors can include homology-directed integration using CRISPR enzyme-mediated cleavage of a target genome. CRISPR enzyme (e.g., Cas9) cleaves double stranded DNA at a site specified by a guide RNA (gRNA). The double strand break can be repaired by homology-directed repair (HDR) when a donor template (such as an Ad3, 7, 11, 14, 16, 21, 34, 37, or 50 payload integration element including left and right homology arms) is present. In various such methods, an integration element is a “repair template” in that it includes left and right homology arms (e.g., of 500-3,000 bp) for insertion into a cleaved target genome. CRISPR-mediated gene insertion can be several orders of magnitude more efficient compared with spontaneous recombination of DNA template, demonstrating that CRISPR-mediated gene insertion can be an effective tool for genome editing. Exemplary methods of homology-directed integration of a nucleic acid sequence into a specified genomic locus are known in the art, e.g., in Richardson et al. (Nat Biotechnol. 34(3):339-44, 2016).

### II. Target Cell Populations

In various embodiments, donor vectors and genomes of the present disclosure can transduce hematopoietic stem cells (HSCs). HSCs can be targeted for in vivo genetic modification by binding CD46. HSCs or subsets thereof can also be identified by any of the following marker profiles: CD34+; Lin−/CD34+/CD38−/CD45RA−/CD90+/CD49f+(HSC1); CD34+/CD38−/CD45RA−/CD90−/CD49f+/(HSC2). In various embodiments, human HSC1 can be identified by any of the following profiles: CD34+/CD38−/CD45RA−/CD90+ or CD34+/CD45RA−/CD90+ and mouse LT-HSC can be identified by Lin−/Scal+/ckit+/CD150+/CD48−/Flt3−/CD34− (where Lin represents the absence of expression of any marker of mature cells including CD3, Cd4, CD8, CD11b, CD11c, NK1.1, Gr1, and TER119). In particular embodiments, HSC are identified by a CD164+ profile. In particular embodiments, HSC are identified by a CD34+/CD164+ profile. For additional information regarding HSC marker profiles, see WO2017/218948.

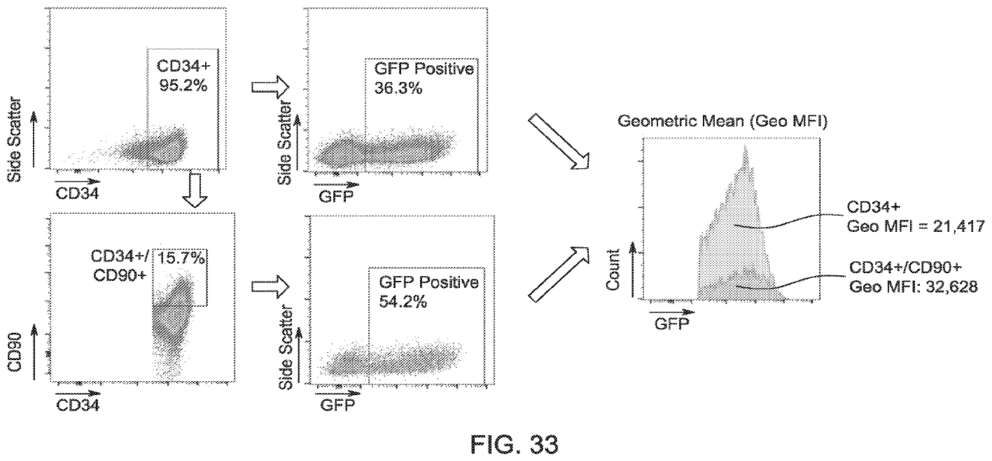

For the avoidance of doubt, in various embodiments, donor vectors and genomes of the present disclosure can infect and/or transduce CD34+ hematopoietic cells. In various embodiments, donor vectors and genomes of the present disclosure can infect and/or transduce CD34+/CD90+ cells. In various embodiments, CD34+ cells and/or a CD34+ phenotype can refer to cells found to express CD34+, e.g., based on binding of cells with a labelled anti-CD34 antibody, e.g., as set forth in Example 6 and/or FIG. 33. In various embodiments, CD90+ cells and/or a CD90+ phenotype can refer to cells found to express CD90+, e.g., based on binding of cells with a labelled anti-CD90 antibody, e.g., as set forth in Example 6 and/or FIG. 33. In various embodiments, CD34+ cells and/or a CD34+ phenotype can refer to cells in sample or population that are most robustly labeled by a label directed to CD34+(e.g., most robustly labeled by a labelled anti-CD34 antibody). For example, in various embodiments in which a sample or population includes cells labeled by a label directed to CD34+, CD34+ cells and/or a CD34+ phenotype can refer to (i) all the cells that are labeled by a label directed to CD34%, or can refer to (ii) the 99%, 98%, 97%, 96%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, or 1% of cells that are most robustly labeled by a label directed to CD34, which CD34+ cells can optionally be referred to as CD34+high cells. In various embodiments, labeling and/or robustness of labeling can be determined by any of a variety of methods known in the art, including without limitation by relative presence of a label, such as fluorescence of a fluorescence label. In various embodiments, labeling and/or robustness of labeling can be measured by techniques including methods such as fluorescence-activated cell sorting (FACS). Accordingly, in various embodiments, CD34+/CD90+ cells can refer to a population of cells that are (i) CD34+ cells and/or determined to have a CD34+ phenotype and are (ii) CD90+ cells and/or determined to have a CD90+ phenotype. In various embodiments, CD34+/CD90+ cells can refer to a population of CD34+high/CD90+ cells that are (i) CD34+high cells and/or determined to have a CD34+high phenotype and are (ii) CD90+ cells and/or determined to have a CD90+ phenotype. In various such embodiments, the cells can be hematopoietic cells. In various embodiments, the cells can be CD45RA−. In various embodiments, the cells can be CD45RA+.

Without wishing to be bound by any particular scientific theory, the present disclosure includes that expression of CD34+(e.g., labeling and/or robustness of labeling of CD34) can correlate with CD46 expression and/or with susceptibility to infection and/or transduction by vectors of the present disclosure, e.g., in hematopoietic cells. Without wishing to be bound by any particular scientific theory, the present disclosure includes that vectors disclosed herein are particularly advantageous in infecting and/or transducing CD34+ cells, CD34+high cells, CD34+/CD90+ cells, and/or CD34+high/CD90+ cells (e.g., can selectively infect and/or transduce CD34+ cells, CD34+high cells, CD34+/CD90+ cells, and/or CD34+high/CD90+ cells), e.g., where the cells are hematopoietic cells.

HSCs can be beneficially caused to encode and/or express various payloads provided herein, including without limitation TCRs and CARs (see, e.g., Gschweng et al. Immunol Rev. 2014 January; 257(1): 237-249).

### III. Dosages, Formulations, and Administration

A vector can be formulated such that it is pharmaceutically acceptable for administration to cells or animals, e.g., to humans. A vector may be administered in vitro, ex vivo, or in vivo. The adenoviral vectors described herein can be formulated for administration to a subject. Formulations include an adenoviral vector encoding a therapeutic agent and one or more pharmaceutically acceptable carriers.

As disclosed herein, a vector can be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, a vector can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intracisternal injection and infusion. A parenteral route of administration can be, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a vector of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A vector can be administered parenterally in the form of an injectable formulation including a sterile solution or suspension in water or another pharmaceutically acceptable liquid. For example, the vector can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of vector included in the pharmaceutical preparations is such that a suitable dose within the designated range is provided. Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

In some embodiments, a vector described herein can be therapeutically delivered to a subject by way of local administration. As used herein, “local administration” or “local delivery,” can refer to delivery that does not rely upon transport of the vector or vector to its intended target tissue or site via the vascular system. For example, the vector may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

Pharmaceutical forms of vector formulations suitable for injection can include sterile aqueous solutions or dispersions. A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Preferably, isotonic agents, for example, sugars or sodium chloride can be used in the formulations.

A suitable dose of a vector described herein can depend on a variety of factors including, e.g., the age, sex, and weight of a subject to be treated, the condition or disease to be treated, and the particular vector used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. A suitable means of administration of a vector can be selected based on the condition or disease to be treated and upon the age and condition of a subject. Dose and method of administration can vary depending on the weight, age, condition, and the like of a patient, and can be suitably selected as needed by those skilled in the art. A specific dosage and treatment regimen for any particular subject can be adjusted based on the judgment of a medical practitioner.

In various instances, a vector can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

In various embodiments, a composition including a vector as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

The formulations disclosed herein can be formulated for administration by, for example, injection. For injection, formulation can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Any formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by US FDA Office of Biological Standards and/or other relevant foreign regulatory agencies.

Therapeutically effective amounts of adenoviral vector associated with a therapeutic gene can include doses ranging from, for example, 1×10⁷ to 50×10⁸ infection units (IU) or from 5×10⁷ to 20×10⁸ IU. In other examples, a dose can include 5×10⁷ IU, 6×10⁷ IU, 7×10⁷ IU, 8×10⁷ IU, 9×10⁷ IU, 1×10⁸ IU, 2×10⁸ IU, 3×10⁸ IU, 4×10⁸ IU, 5×10⁸ IU, 6×10⁸ IU, 7×10⁸ IU, 8×10⁸ IU, 9×10⁸ IU, 10×10⁸ IU, or more. In particular embodiments, a therapeutically effective amount of an adenoviral vector associated with a therapeutic gene includes 4×10⁸ IU. In particular embodiments, a therapeutically effective amount of an adenoviral vector associated with a therapeutic gene can be administered subcutaneously or intravenously. In particular embodiments, a therapeutically effective amount of an adenoviral vector associated with a therapeutic gene can be administered following administration with one or more mobilization factors.

In various embodiments of the present disclosure, an in vivo gene therapy includes administration of at least one viral gene therapy vector to a subject in combination with at least one immune suppression regimen. In an in vivo gene therapy including more than one vector species, such as a first vector that is a supported viral gene therapy vector in combination with a second vector that is a support vector, the first vector and the second vector can be administered in a single formulation or dosage form or in two separate formulations or dosage forms. In various embodiments, the first and second vectors can be administered at the same time or at different times, e.g., during the same one-hour period or during non-overlapping one-hour periods. In various embodiments, the first and second vectors can be administered at the same time or at different times, e.g., on the same day or on different days. In various embodiments, the first and second vectors can be administered at the same dosage or at different dosages, e.g., where the dosage is measured as the total number of viral particles or as a number of viral particles per kilogram of the subject. In various embodiments, the first and second vectors can be administered in a pre-defined ratio. In various embodiments, the ratio is in the range of 2:1 to 1:2, e.g., 1:1.

In various embodiments, a vector is administered to a subject in a single total dose on a single day. In various embodiments a vector is administered in two, three, four, or more unit doses that together constitute a total dose. In various embodiments, one unit dose of a vector is administered to a subject per day on each of one, two, three, four, or more consecutive days. In various embodiments, two unit doses of a vector are administered to a subject per day on each of one, two, three, four, or more consecutive days. Accordingly, in various embodiments, a daily dose can refer to the dose of vector received by a subject over the course of a day. In various embodiments, the term day refers to a twenty-four-hour period, such as a twenty-four-hour period from midnight of a first calendar date to midnight of the next calendar date.

In various embodiments, a unit dose, daily dose, or total dose of a vector, such as a viral gene therapy vector or support vector, or the total combined dose of a viral gene therapy vector and a support vector, can be at least 1E8, 5E8, 1E9, 5E9, 1E10, 5E10, 1E11, 5E11, 1E12, 5E12, 1E13, 5E13, 1E14, or 1E15 viral particles per kilogram (vp/kg). In various embodiments, a unit dose, daily dose, or total dose of a vector, such as a viral gene therapy vector or support vector, or the total combined dose of a viral gene therapy vector and a support vector, can fall within a range having a lower bound selected from 1E8, 5E8, 1E9, 5E9, 1E10, 5E10, 1E11, 5E11, 1E12, 5E12, 1E13, 5E13, 1E14, or 1E15 vp/kg and an upper bound selected from 1E8, 5E8, 1E9, 5E9, 1E10, 5E10, 1E11, 5E11, 1E12, 5E12, 1E13, 5E13, 1E14, or 1E15 vp/kg.

In various embodiments, a viral gene therapy vector is administered at a unit dose, daily dose, or total dose of at least 1E10, 5E10, 1E11, 5E11, 1E12, 5E12, 1E13, 5E13, 1E14, or 1E15 vp/kg and a support vector is administered at a unit dose, daily dose, or total dose of at least 1E8, 5E8, 1E9, 5E9, 1E10, 5E10, 1E11, and 5E11 vp/kg, optionally where the unit dose, daily dose, or total dose of the viral gene therapy vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the support vector is within a range having a lower bound selected from 1E8, 5E8, 1E9, 5E9, 1E10, and 5E10 vp/kg and an upper bound selected from 1E9, 5E9, 1E10, 5E10, 1E11, and 5E11 vp/kg.

In various embodiments, a support vector is administered at a unit dose, daily dose, or total dose of at least 1E10, 5E10, 1E11, 5E11, 1E12, 5E12, 1E13, 5E13, 1E14, or 1E15 vp/kg and a supported viral gene therapy vector is administered at a unit dose, daily dose, or total dose of at least 1E8, 5E8, 1E9, 5E9, 1E10, 5E10, 1E11, and 5E11 vp/kg, optionally where the unit dose, daily dose, or total dose of the support vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the supported viral gene therapy vector is within a range having a lower bound selected from 1E8, 5E8, 1E9, 5E9, 1E10, and 5E10 vp/kg and an upper bound selected from 1E9, 5E9, 1E10, 5E10, 1E11, and 5E11 vp/kg. In various embodiments, a supported viral gene therapy vector and a support vector are administered in a pre-defined ratio. In various embodiments, the ratio is in the range of 2:1 to 1:2, e.g., 1:1.

### IV. Applications

Methods and compositions provided herein are disclosed at least in part for use in in vivo gene therapy. However, for the avoidance of doubt, the present disclosure expressly includes the use of compositions and methods provided herein for ex-vivo engineering of cells and/or tissues, as well as in vitro uses including the engineering of cells and/or tissues for research purposes. Gene therapy includes use of a vector, genome, or system of the present disclosure in a method of introducing exogenous DNA into a host cell (such as a target cell) and/or a nucleic acid (such as a target nucleic acid, such as a target genome, e.g., the genome of a target cell). The present disclosure includes description and exemplification of compositions and methods relating to in vivo, in vitro, and ex vivo therapy and those of skill in the art will appreciate that various methods and compositions provided herein are generally applicable to introduction of a nucleic acid payload into a subject, e.g., a host or target cell. Because such compositions and methods are of general utility, e.g., in gene therapy, they are useful both as tools in gene therapy in general and in various particular conditions, including those provided herein.

### IV(A). In Vivo Gene Therapy

Treatments using in VIVO gene therapy, which includes the direct delivery of a viral vector to a patient, have been explored. In vivo gene therapy is an attractive approach because it may not require any genotoxic conditioning (or could require less genotoxic conditioning) nor ex vivo cell processing and thus could be adopted at many institutions worldwide, including those in developing countries, as the therapy could be administered through an injection, similar to what is already done worldwide for the delivery of vaccines. In various embodiments methods of in vivo gene therapy with adenoviral vectors of the present disclosure can include one or more steps of (i) target cell mobilization, (ii) immunosuppression, (iii) administration of a vector, genome, system or formulation provided herein, and/or (iv) selection of transduced cells and/or cells that have integrated an integration element of a payload of an adenoviral vector or genome.

The adenoviral vector formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts of one or more vectors, genomes, or systems of the present disclosure. Therapeutically effective amounts include those that provide effective amounts, prophylactic treatments, and/or therapeutic treatments.

Vectors described herein can be administered in coordination with mobilization factors. In certain embodiments, adenoviral vector formulations described herein can be administered in concert with HSPC mobilization. In particular embodiments, administration of adenoviral donor vector occurs concurrently with administration of one or more mobilization factors. In particular embodiments, administration of adenoviral donor vector follows administration of one or more mobilization factors. In particular embodiments, administration of adenoviral donor vector follows administration of a first one or more mobilization factors and occurs concurrently with administration of a second one or more mobilization factors. Agents for HSPC mobilization include, for example, granulocyte-colony stimulating factor (G-CSF), granulocyte macrophage colony stimulating factor (GM-CSF), AMD3100, SCF, S-CSF, a CXCR4 antagonist, a CXCR2 agonist, and Gro-Beta (GRO-β). In various embodiments, a CXCR4 antagonist is AMD3100 and/or a CXCR2 agonist is GRO-β.

G-CSF is a cytokine whose functions in HSPC mobilization can include the promotion of granulocyte expansion and both protease-dependent and independent attenuation of adhesion molecules and disruption of the SDF-1/CXCR4 axis. In particular embodiments, any commercially available form of G-CSF known to one of ordinary skill in the art can be used in the methods and formulations as disclosed herein, for example, Filgrastim (Neupogen®, Amgen Inc., Thousand Oaks, CA) and PEGylated Filgrastim (Pegfilgrastim, NEULASTA®, Amgen Inc., Thousand Oaks, CA).

GM-CSF is a monomeric glycoprotein also known as colony-stimulating factor 2 (CSF2) that functions as a cytokine and is naturally secreted by macrophages, T cells, mast cells, natural killer cells, endothelial cells, and fibroblasts. In particular embodiments, any commercially available form of GM-CSF known to one of ordinary skill in the art can be used in the methods and formulations as disclosed herein, for example, Sargramostim (Leukine, Bayer Healthcare Pharmaceuticals, Seattle, WA) and molgramostim (Schering-Plough, Kenilworth, NJ).

AMD3100 (MOZOBIL™, PLERIXAFOR™; Sanofi-Aventis, Paris, France), a synthetic organic molecule of the bicyclam class, is a chemokine receptor antagonist and reversibly inhibits SDF-1 binding to CXCR4, promoting HSPC mobilization. AMD3100 is approved to be used in combination with G-CSF for HSPC mobilization in patients with myeloma and lymphoma. The structure of AMD3100 is:

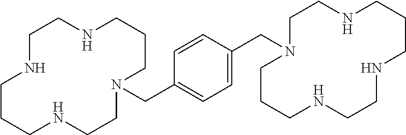 [SMILES: c1cc(CN2CCCNCCNCCCNCC2)ccc1CN1CCCNCCNCCCNCC1]

SCF, also known as KIT ligand, KL, or steel factor, is a cytokine that binds to the c-kit receptor (CD117). SCF can exist both as a transmembrane protein and a soluble protein. This cytokine plays an important role in hematopoiesis, spermatogenesis, and melanogenesis. In particular embodiments, any commercially available form of SCF known to one of ordinary skill in the art can be used in the methods and formulations as disclosed herein, for example, recombinant human SCF (Ancestim, STEMGEN®, Amgen Inc., Thousand Oaks, CA).

Chemotherapy used in intensive myelosuppressive treatments also mobilizes HSPCs to the peripheral blood as a result of compensatory neutrophil production following chemotherapy-induced aplasia. In particular embodiments, chemotherapeutic agents that can be used for mobilization of HSPCs include cyclophosphamide, etoposide, ifosfamide, cisplatin, and cytarabine.

Additional agents that can be used for cell mobilization include: CXCL12/CXCR4 modulators (e.g., CXCR4 antagonists: POL6326 (Polyphor, Allschwil, Switzerland), a synthetic cyclic peptide which reversibly inhibits CXCR4; BKT-140 (4F-benzoyl-TN14003; Biokine Therapeutics, Rehovit, Israel); TG-0054 (Taigen Biotechnology, Taipei, Taiwan); CXCL12 neutralizer NOX-A12 (NOXXON Pharma, Berlin, Germany) which binds to SDF-1, inhibiting its binding to CXCR4); Sphingosine-1-phosphate (SIP) agonists (e.g., SEW2871, Juarez et al. Blood 119: 707-716, 2012); vascular cell adhesion molecule-1 (VCAM) or very late antigen 4 (VLA-4) inhibitors (e.g., Natalizumab, a recombinant humanized monoclonal antibody against α4 subunit of VLA-4 (Zohren et al. Blood 111: 3893-3895, 2008); BI05192, a small molecule inhibitor of VLA-4 (Ramirez et al. Blood 114: 1340-1343, 2009)); parathyroid hormone (Brunner et al. Exp Hematol. 36: 1157-1166, 2008); proteasome inhibitors (e.g., Bortezomib, Ghobadi et al. ASH Annual Meeting Abstracts. p. 583, 2012); Groβ, a member of CXC chemokine family which stimulates chemotaxis and activation of neutrophils by binding to the CXCR2 receptor (e.g., SB-251353, King et al. Blood 97: 1534-1542, 2001); stabilization of hypoxia inducible factor (HIF) (e.g., FG-4497, Forristal et al. ASH Annual Meeting Abstracts. p. 216, 2012); Firategrast, an α4β1 and α4β7 integrin inhibitor (α4β1/7) (Kim et al. Blood 128: 2457-2461, 2016); Vedolizumab, a humanized monoclonal antibody against the α4β7 integrin (Rosario et al. Clin Drug Investig 36: 913-923, 2016); and BOP (N-(benzenesulfonyl)-L-prolyl-L-O-(1-pyrrolidinylcarbonyl) tyrosine) which targets integrins α9β1/α4β1 (Cao et al. Nat Commun 7: 11007, 2016). Additional agents that can be used for HSPC mobilization are described in, for example, Richter R et al. Transfus Med Hemother 44:151-164, 2017, Bendall & Bradstock, Cytokine & Growth Factor Reviews 25: 355-367, 2014, WO 2003043651, WO 2005017160, WO 2011069336, U.S. Pat. Nos. 5,637,323, 7,288,521, 9,782,429, US 2002/0142462, and US 2010/02268.

In particular embodiments, a therapeutically effective amount of G-CSF includes 0.1 μg/kg to 100 μg/kg. In particular embodiments, a therapeutically effective amount of G-CSF includes 0.5 μg/kg to 50 μg/kg. In particular embodiments, a therapeutically effective amount of G-CSF includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, a therapeutically effective amount of G-CSF includes 5 μg/kg. In particular embodiments, G-CSF can be administered subcutaneously or intravenously. In particular embodiments, G-CSF can be administered for 1 day, 2 consecutive days, 3 consecutive days, 4 consecutive days, 5 consecutive days, or more. In particular embodiments, G-CSF can be administered for 4 consecutive days. In particular embodiments, G-CSF can be administered for 5 consecutive days. In particular embodiments, as a single agent, G-CSF can be used at a dose of 10 μg/kg subcutaneously daily, initiated 3, 4, 5, 6, 7, or 8 days before adenoviral delivery. In particular embodiments, G-CSF can be administered as a single agent followed by concurrent administration with another mobilization factor. In particular embodiments, G-CSF can be administered as a single agent followed by concurrent administration with AMD3100. In particular embodiments, a treatment protocol includes a 5 day treatment where G-CSF can be administered on day 1, day 2, day 3, and day 4 and on day 5, G-CSF and AMD3100 are administered 6 to 8 hours prior to adenoviral administration.

Therapeutically effective amounts of GM-CSF to administer can include doses ranging from, for example, 0.1 to 50 μg/kg or from 0.5 to 30 μg/kg. In particular embodiments, a dose at which GM-CSF can be administered includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, GM-CSF can be administered subcutaneously for 1 day, 2 consecutive days, 3 consecutive days, 4 consecutive days, 5 consecutive days, or more. In particular embodiments, GM-CSF can be administered subcutaneously or intravenously. In particular embodiments, GM-CSF can be administered at a dose of 10 μg/kg subcutaneously daily initiated 3, 4, 5, 6, 7, or 8 days before adenoviral delivery. In particular embodiments, GM-CSF can be administered as a single agent followed by concurrent administration with another mobilization factor. In particular embodiments, GM-CSF can be administered as a single agent followed by concurrent administration with AMD3100. In particular embodiments, a treatment protocol includes a 5 day treatment where GM-CSF can be administered on day 1, day 2, day 3, and day 4 and on day 5, GM-CSF and AMD3100 are administered 6 to 8 hours prior to adenoviral administration. A dosing regimen for Sargramostim can include 200 μg/m², 210 μg/m², 220 μg/m², 230 μg/m², 240 μg/m², 250 μg/m², 260 μg/m², 270 μg/m², 280 μg/m², 290 μg/m², 300 μg/m², or more. In particular embodiments, Sargramostim can be administered for 1 day, 2 consecutive days, 3 consecutive days, 4 consecutive days, 5 consecutive days, or more. In particular embodiments, Sargramostim can be administered subcutaneously or intravenously. In particular embodiments, a dosing regimen for Sargramostim can include 250 μg/m²/day intravenous or subcutaneous and can be continued until a targeted cell amount is reached in the peripheral blood or can be continued for 5 days. In particular embodiments, Sargramostim can be administered as a single agent followed by concurrent administration with another mobilization factor. In particular embodiments, Sargramostim can be administered as a single agent followed by concurrent administration with AMD3100. In particular embodiments, a treatment protocol includes a 5 day treatment where Sargramostim can be administered on day 1, day 2, day 3, and day 4 and on day 5, Sargramostim and AMD3100 are administered 6 to 8 hours prior to adenoviral administration.

In particular embodiments, a therapeutically effective amount of AMD3100 includes 0.1 mg/kg to 100 mg/kg. In particular embodiments, a therapeutically effective amount of AMD3100 includes 0.5 mg/kg to 50 mg/kg. In particular embodiments, a therapeutically effective amount of AMD3100 includes 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or more. In particular embodiments, a therapeutically effective amount of AMD3100 includes 4 mg/kg. In particular embodiments, a therapeutically effective amount of AMD3100 includes 5 mg/kg. In particular embodiments, a therapeutically effective amount of AMD3100 includes 10 μg/kg to 500 μg/kg or from 50 μg/kg to 400 μg/kg. In particular embodiments, a therapeutically effective amount of AMD3100 includes 100 μg/kg, 150 μg/kg, 200 μg/kg, 250 μg/kg, 300 μg/kg, 350 μg/kg, or more. In particular embodiments, AMD3100 can be administered subcutaneously or intravenously. In particular embodiments, AMD3100 can be administered subcutaneously at 160-240 μg/kg 6 to 11 hours prior to adenoviral delivery. In particular embodiments, a therapeutically effective amount of AMD3100 can be administered concurrently with administration of another mobilization factor. In particular embodiments, a therapeutically effective amount of AMD3100 can be administered following administration of another mobilization factor. In particular embodiments, a therapeutically effective amount of AMD3100 can be administered following administration of G-CSF. In particular embodiments, a treatment protocol includes a 5-day treatment where G-CSF is administered on day 1, day 2, day 3, and day 4 and on day 5, G-CSF and AMD3100 are administered 6 to 8 hours prior to adenoviral injection.

Therapeutically effective amounts of SCF to administer can include doses ranging from, for example, 0.1 to 100 μg/kg/day or from 0.5 to 50 μg/kg/day. In particular embodiments, a dose at which SCF can be administered includes 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day, 10 μg/kg/day, 11 μg/kg/day, 12 μg/kg/day, 13 μg/kg/day, 14 μg/kg/day, 15 μg/kg/day, 16 μg/kg/day, 17 μg/kg/day, 18 μg/kg/day, 19 μg/kg/day, 20 μg/kg/day, 21 μg/kg/day, 22 μg/kg/day, 23 μg/kg/day, 24 μg/kg/day, 25 μg/kg/day, 26 μg/kg/day, 27 μg/kg/day, 28 μg/kg/day, 29 μg/kg/day, 30 μg/kg/day, or more. In particular embodiments, SCF can be administered for 1 day, 2 consecutive days, 3 consecutive days, 4 consecutive days, 5 consecutive days, or more. In particular embodiments, SCF can be administered subcutaneously or intravenously. In particular embodiments, SCF can be injected subcutaneously at 20 μg/kg/day. In particular embodiments, SCF can be administered as a single agent followed by concurrent administration with another mobilization factor. In particular embodiments, SCF can be administered as a single agent followed by concurrent administration with AMD3100. In particular embodiments, a treatment protocol includes a 5 day treatment where SCF can be administered on day 1, day 2, day 3, and day 4 and on day 5, SCF and AMD3100 are administered 6 to 8 hours prior to adenoviral administration.

In particular embodiments, growth factors GM-CSF and G-CSF can be administered to mobilize HSPC in the bone marrow niches to the peripheral circulating blood to increase the fraction of HSPCs circulating in the blood. In particular embodiments, mobilization can be achieved with administration of G-CSF/Filgrastim (Amgen) and/or AMD3100 (Sigma). In particular embodiments, mobilization can be achieved with administration of GM-CSF/Sargramostim (Amgen) and/or AMD3100 (Sigma). In particular embodiments, mobilization can be achieved with administration of SCF/Ancestim (Amgen) and/or AMD3100 (Sigma). In particular embodiments, administration of G-CSF/Filgrastim precedes administration of AMD3100. In particular embodiments, administration of G-CSF/Filgrastim occurs concurrently with administration of AMD3100. In particular embodiments, administration of G-CSF/Filgrastim precedes administration of AMD3100, followed by concurrent administration of G-CSF/Filgrastim and AMD3100. US 20140193376 describes mobilization protocols utilizing a CXCR4 antagonist with a S1P receptor 1 (S1PR1) modulator agent. US 20110044997 describes mobilization protocols utilizing a CXCR4 antagonist with a vascular endothelial growth factor receptor (VEGFR) agonist.

Adenoviral vectors (e.g. Ad3, 7, 11, 14, 16, 21, 34, 37, or 50 vectors) are exemplary of vectors that can be administered in concert with HSPC mobilization. In particular embodiments, administration of an adenoviral vector occurs concurrently with administration of one or more mobilization factors. In particular embodiments, administration of an Adenoviral vector follows administration of one or more mobilization factors. In particular embodiments, administration of an Adenoviral vector follows administration of a first one or more mobilization factors and occurs concurrently with administration of a second one or more mobilization factors.

In particular embodiments, an HSC enriching agent, such as a CD19 immunotoxin or 5-FU can be administered to enrich for HSPCs. CD19 immunotoxin can be used to deplete all CD19 lineage cells, which accounts for 30% of bone marrow cells. Depletion encourages exit from the bone marrow. By forcing HSPCs to proliferate (whether via, e.g., CD19 immunotoxin of 5-FU), this stimulates their differentiation and exit from the bone marrow and increases transgene marking in peripheral blood cells.

Therapeutically effective amounts of HSC mobilization factors and/or HSC enriching agents can be administered through any appropriate administration route such as by, injection, infusion, perfusion, and more particularly by administration by one or more of bone marrow, intravenous, intradermal, intraarterial, intranodal, intralymphatic, intraperitoneal injection, infusion, or perfusion).

In particular embodiments, methods of the present disclosure can include selection for cells modified to express a selection marker (e.g., a mutant form of MGMT that is resistant to inactivation by 6-BG, but retains the ability to repair DNA damage). For example, particular embodiments include regimens that combine mobilization (e.g., a mobilization protocol described herein) with administration of an adenoviral vector described herein and administration BCNU or benzylguanine and temozolomide in the case of an adenoviral vector including a MGMT^{P140K} selection marker. In particular embodiments, the in vivo selection marker can include MGMT^{P140K} as described in Olszko et al., Gene Therapy 22: 591-595, 2015. Thus, selection for cells that express MGMT^{P140K} can select for transduced cells and/or contribute to therapeutic efficacy.

Adenoviral vectors can be administered concurrently with or following administration of one or more immunosuppression agents or immunosuppression regimens.

### IV(B). In Vitro and Ex Vivo Gene Therapy

In vitro gene therapy includes use of a vector, genome, or system of the present disclosure in a method of introducing exogenous DNA into a host cell (such as a target cell) and/or a nucleic acid (such as a target nucleic acid, such as a target genome), where the host cell or nucleic acid is not present in a multicellular organism (e.g., in a laboratory). In some embodiments, a target cell or nucleic acid is derived from a multicellular organism, such as a mammal (e.g., a mouse, rat, human, or non-human primate). In vitro engineering of a cell derived from a multicellular organism can be referred to as ex vivo engineering, and can be used in ex vivo therapy. In various embodiments, methods and compositions of the present disclosure are utilized, e.g., as disclosed herein, to modify a target cell or nucleic acid derived from a first multicellular organism and the engineered target cell or nucleic acid is then administered to a second multicellular organism, such as a mammal (e.g., a mouse, rat, human, or non-human primate), e.g., in a method of adoptive cell therapy. In some instances, the first and second organisms are the same single subject organism. Return of in vitro engineered material to a subject from which the material was derived can be an autologous therapy. In some instances, the first and second organisms are different organisms (e.g., two organisms of the same species, e.g., two mice, two rats, two humans, or two non-human primates of the same species). Transfer of engineered material derived from a first subject to a second different subject can be an allogeneic therapy.

Ex vivo cell therapies can include isolation of stem, progenitor or differentiated cells from a patient or a normal donor, expansion of isolated cells ex vivo—with or without genetic engineering—and administration of the cells to a subject to establish a transient or stable graft of the infused cells and/or their progeny. Such ex vivo approaches can be used, for example, to treat an inherited, infectious or neoplastic disease, to regenerate a tissue or to deliver a therapeutic agent to a disease site. In various ex vivo therapies there is no direct exposure of the subject to the gene transfer vector, and the target cells of transduction can be selected, expanded and/or differentiated, before or after any genetic engineering, to improve efficacy and safety.

Ex vivo therapies include haematopoietic stem cell (HSC) transplantation (HCT). Autologous HSC gene therapy represents a therapeutic option for several monogenic diseases of the blood and the immune system as well as for storage disorders, and it may become a first-line treatment option for selected disease conditions.

Applications of ex-vivo therapy include reconstituting dysfunctional cell lineages. For inherited diseases characterized by a defective or absent cell lineage, the lineage can be regenerated by functional progenitor cells, derived either from normal donors or from autologous cells that have been subjected to ex vivo gene transfer to correct the deficiency. An example is provided by SCIDs, in which a deficiency in any one of several genes blocks the development of mature lymphoid cells. Transplantation of non-manipulated normal donor HSCs, which can allow generation of donor-derived functional haematopoietic cells of various lineages in the host, represents a therapeutic option for SCIDs, as well as many other diseases that affect the blood and immune system. Autologous HSC gene therapy, which can include replacing a functional copy of a defective gene in transplanted haematopoietic stem/progenitor cells (HSPCs) and, similarly to HCT, can provide a steady supply of functional progeny, may have several advantages, including reduced risk of graft versus host disease (GvHD), reduced risk of graft rejection, and reduced need for post-transplant immunosuppression.

Applications of ex-vivo therapy include augmenting therapeutic gene dosage. In some applications, HSC gene therapy may augment the therapeutic efficacy of allogenic HCT. Therapeutic gene dosage can be engineered to supra-normal levels in transplanted cells.

Applications of ex-vivo therapy include introducing novel function and targeting gene therapy. Ex vivo gene therapy can confer a novel function to HSCs or their progeny, such as establishing drug resistance to allow administration of a high-dose antitumor chemotherapy regime or establishing resistance to a pre-established infection with a virus, such as HIV, or other pathogen by expressing RNA-based agents (for example, ribozymes, RNA decoys, antisense RNA, RNA aptamers and small interfering RNA) and protein-based agents (for example, dominant-negative mutant viral proteins, fusion inhibitors and engineered nucleases that target the pathogen's genome).

### IV(C). Conditions Treatable by Gene Therapy

At least in part because adenoviral vectors of the present disclosure (e.g. Ad3, 7, 11, 14, 16, 21, 34, 37, or 50 vectors) can be used in vivo, in vitro, or ex vivo for modification of host and/or target cells, and further because an adenoviral vector can include payloads encoding a wide variety of expression products, it will be clear from the present specification that various technologies provided herein have broad applicability and can be used to treat a wide variety of conditions. Examples of conditions treatable by administration of an adenoviral vector, genome, or system of the present disclosure include, without limitation, hemoglobinopathies, immunodeficiencies, point mutation conditions, cancers, protein deficiencies, infectious diseases, and inflammatory conditions.

In certain embodiments, vectors, genomes, systems and formulations disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts. Therapeutically effective amounts include those that provide effective amounts, prophylactic treatments, and/or therapeutic treatments.

In particular embodiments, methods and formulations disclosed herein can be used to treat blood disorders. In particular embodiments, formulations are administered to subjects to treat hemophilia, β-thalassemia major, Diamond Blackfan anemia (DBA), paroxysmal nocturnal hemoglobinuria (PNH), pure red cell aplasia (PRCA), refractory anemia, severe aplastic anemia, and/or blood cancers such as leukemia, lymphoma, and myeloma.

Hemoglobinopathies represent a global health burden with disproportionate outcomes. Defects in hemoglobin proteins or in the expression of globin genes can result in diseases termed hemoglobinopathies. Hemoglobinopathies are amongst the most common genetic disorders world-wide.

Every year, 1.1 million births worldwide are at risk for hemoglobinopathies, affecting as many as 25 in every 1,000 births in geographic regions where malaria falciparum is prevalent, owing to a natural resistance to malaria infection conferred by hemoglobin (Hb) genetic variance. In developed regions, patients are at risk of iron overload from chronic transfusions. In underdeveloped regions, survival is significantly lower. For example, in Africa, childhood mortality is 40% in patients with hemoglobinopathies, compared to 16% in all children.

Mutations in the globin genes may generate an abnormal form of hemoglobin, as in sickle cell disease (SCD) and hemoglobin C, D, and E disease, or result in reduced production of the α or β polypeptides and thus an imbalance of the globin chains in the cell. These latter conditions are termed α- or β-thalassemias, depending on which globin chain is impaired. 5% of the world population carries a significant hemoglobin variant with the sickle cell mutation in the b-globin (HBB) gene (a glutamate to valine conversion; historically E6V, contemporaneously E7V) being by far the most common (40% of carriers). The high prevalence and severity of hemoglobin disorders presents a substantial burden, impacting not only the lives of those affected but also health-care systems, since lifelong patient care is costly.

There are two forms of hemoglobin, fetal (HbF), which includes two alpha (α) and two gamma (γ) chains, and adult (HbA), which includes two a and two beta (β) chains. The natural switch from HbF to HbA occurs shortly after birth and is regulated by transcriptional repression of γ globin genes by factors including a master regulator, bcl11a. Critically, a variety of clinical observations demonstrate that the severity of β-hemoglobinopathies such as sickle cell disease and β-thalassemia are ameliorated by increased production of HbF.

In particular embodiments, a therapeutically effective treatment induces or increases expression of HbF, induces or increases production of hemoglobin and/or induces or increases production of β-globin. In particular embodiments, a therapeutically effective treatment improves blood cell function, and/or increases oxygenation of cells.

In various embodiments, the present disclosure includes treatment of a blood disorder using an adenoviral donor vector of the present disclosure that includes a coding nucleic acid sequence that encodes a protein or agent for treatment of the blood disorder. In various embodiments, the blood disorder is thalassemia and the protein is a β-globin or γ-globin protein, or a protein that otherwise partially or completely functionally replaces β-globin or γ-globin. In various embodiments, the blood disorder is hemophilia and the protein is ET3 or a protein that otherwise partially or completely functionally replaces Factor VIII. In various embodiments, the blood disorder is a point mutation disease such as sickle cell anemia, and the agent is a gene editing protein.

ET3 can have or include the following amino acid sequence: SEQ ID NO 154. In various embodiments, a Factor VIII replacement protein can have an amino acid sequence having at least 70%, 75%, 80%, 85%, 90%, 91%, 92%, 93%, 94%, 95%, 96%, 97%, 98%, 99%, or 100% identity to the SEQ ID NO: 154

(MQLELSTCVFLCLLPLGFSAIRRYYLGAVELSWDYRQSELLRELHVDTR
FPATAPGALPLGPSVLYKKTVFVEFTDQLFSVARPRPPWMGLLGPTIQAE
VYDTVVVTLKNMASHPVSLHAVGVSFWKSSEGAEYEDHTSQREKEDDKVL
PGKSQTYVWQVLKENGPTASDPPCLTYSYLSHVDLVKDLNSGLIGALLVC
REGSLTRERTQNLHEFVLLFAVFDEGKSWHSARNDSWTRAMDPAPARAQP
AMHTVNGYVNRSLPGLIGCHKKSVYWHVIGMGTSPEVHSIFLEGHTFLVR
HHRQASLEISPLTFLTAQTFLMDLGQFLLFCHISSHHHGGMEAHVRVESC
AEEPQLRRKADEEEDYDDNLYDSDMDVVRLDGDDVSPFIQIRSVAKKHPK
TWVHYIAAEEEDWDYAPLVLAPDDRSYKSQYLNNGPQRIGRKYKKVRFMA
YTDETFKTREAIQHESGILGPLLYGEVGDTLLIIFKNQASRPYNIYPHGI
TDVRPLYSRRLPKGVKHLKDFPILPGEIFKYKWTVTVEDGPTKSDPRCLT
RYYSSFVNMERDLASGLIGPLLICYKESVDQRGNQIMSDKRNVILFSVFD
ENRSWYLTENIQRFLPNPAGVQLEDPEFQASNIMHSINGYVFDSLQLSVC
LHEVAYWYILSIGAQTDFLSVFFSGYTFKHKMVYEDTLTLFPFSGETVFM
SMENPGLWILGCHNSDFRNRGMTALLKVSSCDKNTGDYYEDSYEDISAYL
LSKNNAIEPRSFAQNSRPPSASAPKPPVLRRHQRDISLPTFQPEEDKMDY
DDIFSTETKGEDFDIYGEDENQDPRSFQKRTRHYFIAAVEQLWDYGMSES
PRALRNRAQNGEVPRFKKVVFREFADGSFTQPSYRGELNKHLGLLGPYIR
AEVEDNIMVTFKNQASRPYSFYSSLISYPDDQEQGAEPRHNFVQPNETRT
YFWKVQHHMAPTEDEFDCKAWAYFSDVDLEKDVHSGLIGPLLICRANTLN
AAHGRQVTVQEFALFFTIFDETKSWYFTENVERNCRAPCHLQMEDPTLKE
NYRFHAINGYVMDTLPGLVMAQNQRIRWYLLSMGSNENIHSIHFSGHVFS
VRKKEEYKMAVYNLYPGVFETVEMLPSKVGIWRIECLIGEHLQAGMSTTF
LVYSKKCQTPLGMASGHIRDFQITASGQYGQWAPKLARLHYSGSINAWST
KEPFSWIKVDLLAPMIIHGIKTQGARQKFSSLYISQFIIMYSLDGKKWQT
YRGNSTGTLMVFFGNVDSSGIKHNIFNPPIIARYIRLHPTHYSIRSTLRM
ELMGCDLNSCSMPLGMESKAISDAQITASSYFTNMFATWSPSKARLHLQG
RSNAWRPQVNNPKEWLQVDFQKTMKVTGVTTQGVKSLLTSMYVKEFLISS
SQDGHQWTLFFQNGKVKVFQGNQDSFTPVVNSLDPPLLTRYLRIHPQSWV
HQIALRMEVLGCEAQDLYV).

β-globin can have or include the following amino acid sequence: SEQ ID NO 155. In various embodiments, a β-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 155

(MVHLTPEEKSAVTALWGKVNVDEVGGEALGRLLVVYPWTQRFFESFGDL
STPDAVMGNPKVKAHGKKVLGAFSDGLAHLDNLKGTFATLSELHCDKLHV
DPENFRLLGNVLVCVLAHHFGKEFTPPVQAAYQKVVAGVANALAHKYH).

γ-globin can have or include the following amino acid sequence: SEQ ID NO 156. In various embodiments, a γ-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 156

(MGHFTEEDKATITSLWGKVNVEDAGGETLGRLLVVYPWTQRFFDSFGNL
SSASAIMGNPKVKAHGKKVLTSLGDATKHLDDLKGTFAQLSELHCDKLHV
DPENFKLLGNVLVTVLAIHFGKEFTPEVQASWQKMVTAVASALSSRYH).

More than 80 primary immune deficiency diseases are recognized by the World Health Organization. These diseases are characterized by an intrinsic defect in the immune system in which, in some cases, the body is unable to produce any or enough antibodies against infection. In other cases, cellular defenses to fight infection fail to work properly. Typically, primary immune deficiencies are inherited disorders.

Secondary, or acquired, immune deficiencies are not the result of inherited genetic abnormalities, but rather occur in individuals in which the immune system is compromised by factors outside the immune system. Examples include trauma, viruses, chemotherapy, toxins, and pollution. Acquired immunodeficiency syndrome (AIDS) is an example of a secondary immune deficiency disorder caused by a virus, the human immunodeficiency virus (HIV), in which a depletion of T lymphocytes renders the body unable to fight infection.

X-linked severe combined immunodeficiency (SCID-X1) is both a cellular and humoral immune depletion caused by mutations in the common gamma chain gene (γC), which result in the absence of T and natural killer (NK) lymphocytes and the presence of nonfunctional B lymphocytes. SCID-X1 is fatal in the first two years of life unless the immune system is reconstituted, for example, through bone marrow transplant (BMT) or gene therapy.

Because most individuals lack a matched donor for BMT or non-autologous gene therapy, haploidentical parental bone marrow depleted of mature T cells is often used; however, complications include graft versus host disease (GVHD), failure to make adequate antibodies hence requiring long-term immunoglobulin replacement, late loss of T cells due to failure to engraft hematopoietic stem and progenitor cells (HSPCs), chronic warts, and lymphocyte dysregulation.

Fanconi anemia (FA) is an inherited blood disorder that leads to bone marrow failure. It is characterized, in part, by a deficient DNA-repair mechanism. At least 20% of patients with FA develop cancers such as acute myeloid leukemias, and cancers of the skin, liver, gastrointestinal tract, and gynecological systems. The skin and gastrointestinal tumors are usually squamous cell carcinomas. The average age of patients who develop cancer is 15 years for leukemia, 16 years for liver tumors, and 23 years for other tumors.

A therapeutic gene can be selected to provide a therapeutically effective response against a condition that, in particular embodiments, is inherited. In particular embodiments, the condition can be Grave's Disease, rheumatoid arthritis, pernicious anemia, Multiple Sclerosis (MS), inflammatory bowel disease, systemic lupus erythematosus (SLE), adenosine deaminase deficiency (ADA-SCID) or severe combined immunodeficiency disease (SCID), Wiskott-Aldrich syndrome (WAS), chronic granulomatous disease (CGD), Fanconi anemia (FA), Battens disease, adrenoleukodystrophy (ALD) or metachromatic leukodystrophy (MLD), muscular dystrophy, pulmonary alveolar proteinosis (PAP), pyruvate kinase deficiency, Schwachman-Diamond-Blackfan anemia, dyskeratosis congenita, cystic fibrosis, Parkinson's disease, Alzheimer's disease, or amyotrophic lateral sclerosis (Lou Gehrig's disease). In particular embodiments, depending on the condition, the therapeutic gene may be a gene that encodes a protein and/or a gene whose function has been interrupted.

In particular embodiments, methods and formulations disclosed herein can be used to treat cancer. In particular embodiments, formulations are administered to subjects to treat acute lymphoblastic leukemia (ALL), acute myelogenous leukemia (AML), chronic lymphocytic leukemia (CLL), chronic myelogenous leukemia (CML), chronic myelomonocytic leukemia, diffuse large B-cell lymphoma, follicular lymphoma, Hodgkin's lymphoma, juvenile myelomonocytic leukemia, multiple myeloma, myelodysplasia, and/or non-Hodgkin's lymphoma.

Additional exemplary cancers that may be treated include astrocytoma, atypical teratoid rhabdoid tumor, brain and central nervous system (CNS) cancer, breast cancer, carcinosarcoma, chondrosarcoma, chordoma, choroid plexus carcinoma, choroid plexus papilloma, clear cell sarcoma of soft tissue, diffuse large B-cell lymphoma, ependymoma, epithelioid sarcoma, extragonadal germ cell tumor, extrarenal rhabdoid tumor, Ewing sarcoma, gastrointestinal stromal tumor, glioblastoma, HBV-induced hepatocellular carcinoma, head and neck cancer, kidney cancer, lung cancer, malignant rhabdoid tumor, medulloblastoma, melanoma, meningioma, mesothelioma, multiple myeloma, neuroglial tumor, not otherwise specified (NOS) sarcoma, oligoastrocytoma, oligodendroglioma, osteosarcoma, ovarian cancer, ovarian clear cell adenocarcinoma, ovarian endometrioid adenocarcinoma, ovarian serous adenocarcinoma, pancreatic cancer, pancreatic ductal adenocarcinoma, pancreatic endocrine tumor, pineoblastoma, prostate cancer, renal cell carcinoma, renal medullary carcinoma, rhabdomyosarcoma, sarcoma, schwannoma, skin squamous cell carcinoma, and stem cell cancer. In various particular embodiments, the cancer is ovarian cancer. In various particular embodiments the cancer is breast cancer. Particular embodiments, formulations are administered to subjects to prevent or delay cancer reoccurrence or prevent or delay cancer onset in carriers of high-risk germ line mutations.

In the context of cancers, therapeutically effective amounts can decrease the number of tumor cells, decrease the number of metastases, decrease tumor volume, increase life expectancy, induce apoptosis of cancer cells, induce cancer cell death, induce chemo- or radiosensitivity in cancer cells, inhibit angiogenesis near cancer cells, inhibit cancer cell proliferation, inhibit tumor growth, prevent metastasis, prolong a subject's life, reduce cancer-associated pain, reduce the number of metastases, and/or reduce relapse or re-occurrence of the cancer following treatment.

In particular embodiments, methods and formulations disclosed herein can be used to treat point mutation conditions. In particular embodiments, formulations are administered to subjects to treat sickle cell disease, cystic fibrosis, Tay-Sachs disease, and/or phenylketonuria. In various embodiments, a transposon payload of the present disclosure encodes a CRISPR-Cas for corrective editing of a nucleic acid lesion. In various embodiments, a transposon payload of the present disclosure encodes a base editor for corrective editing of a nucleic acid lesion. In various embodiments, a transposon payload of the present disclosure encodes a prime editor for corrective editing of a nucleic acid lesion.

In particular embodiments, methods and formulations disclosed herein can be used to treat particular enzyme deficiency. In particular embodiments, formulations are administered to subjects to treat Hurler's syndrome, selective IgA deficiency, hyper IgM, IgG subclass deficiency, Niemann-Pick disease, Tay-Sachs disease, Gaucher disease, Fabry disease, Krabbe disease, glucosemia, maple syrup urine disease, phenylketonuria, glycogen storage disease, Friedreich ataxia, Zellweger syndrome, adrenoleukodystrophy, complement disorders, and/or mucopolysaccharidoses.

Therapeutically effective amounts may provide function to immune and other blood cells and/or microglial cells or may alternatively—depending on the treated condition—inhibit lymphocyte activation, induce apoptosis in lymphocytes, eliminate various subsets of lymphocytes, inhibit T cell activation, eliminate or inhibit autoreactive T cells, inhibit Th-2 or Th-1 lymphocyte activity, antagonize IL-1 or TNF, reduce inflammation, induce selective tolerance to an inciting agent, reduce or eliminate an immune-mediated condition; and/or reduce or eliminate a symptom of the immune-mediated condition. Therapeutically effective amounts may also provide functional DNA repair mechanisms; surfactant protein expression; telomere maintenance; lysosomal function; breakdown of lipids or other proteins such as amyloids; permit ribosomal function; and/or permit development of mature blood cell lineages which would otherwise not develop such as macrophages other white blood cell types.

In particular embodiments, methods of the present disclosure can restore T-cell mediated immune responses in a subject in need thereof. Restoration of T-cell mediated immune responses can include restoring thymic output and/or restoring normal T lymphocyte development.

In particular embodiments, restoring thymic output can include restoring the frequency of CD3+ T cells expressing CD45RA in peripheral blood to a level comparable to that of a reference level derived from a control population. In particular embodiments, restoring thymic output can include restoring the number of T cell receptor excision circles (TRECs) per 106 maturing T cells to a level comparable to that of a reference level derived from a control population. The number of TRECs per 106 maturing T cells can be determined as described in Kennedy et al., Vet Immunol Immunopathol 142: 36-48, 2011.

In particular embodiments, restoring normal T lymphocyte development includes restoring the ratio of CD4+ cells: CD8+ cells to 2. In particular embodiments, restoring normal T lymphocyte development includes detecting the presence of αβ TCR in circulating T-lymphocytes. The presence of αβ TCR in circulating T-lymphocytes can be detected, for example, by flow cytometry using antibodies that bind an α and/or β chain of a TCR. In particular embodiments, restoring normal T lymphocyte development includes detecting the presence of a diverse TCR repertoire comparable to that of a reference level derived from a control population. TCR diversity can be assessed by TCRVβ spectratyping, which analyzes genetic rearrangement of the variable region of the TCRβ gene. Robust, normal spectratype profiles can be characterized by a Gaussian distribution of fragments sized across 17 families of TCRVβ segments. In particular embodiments, restoring normal T lymphocyte development includes restoring T-cell specific signaling pathways. Restoration of T-cell specific signaling pathways can be assessed by lymphocyte proliferation following exposure to the T cell mitogen phytohemagglutinin (PHA). In particular embodiments, restoring normal T lymphocyte development includes restoring white blood cell count, neutrophil cell count, monocyte cell count, lymphocyte cell count, and/or platelet cell count to a level comparable to a reference level derived from a control population.

In particular embodiments, methods of the present disclosure can improve the kinetics and/or clonal diversity of lymphocyte reconstitution in a subject in need thereof. In particular embodiments, improving the kinetics of lymphocyte reconstitution can include increasing the number of circulating T lymphocytes to within a range of a reference level derived from a control population. In particular embodiments, improving the kinetics of lymphocyte reconstitution can include increasing the absolute CD3+ lymphocyte count to within a range of a reference level derived from a control population. A range of can be a range of values observed in or exhibited by normal (i.e., non-immuno-compromised) subjects for a given parameter. In particular embodiments, improving the kinetics of lymphocyte reconstitution can include reducing the time required to reach normal lymphocyte counts as compared to a subject in need thereof not administered a therapy described herein. In particular embodiments, improving the kinetics of lymphocyte reconstitution can include increasing the frequency of gene corrected lymphocytes as compared to a subject in need thereof not administered a therapy described herein. In particular embodiments, improving the kinetics of lymphocyte reconstitution can include increasing diversity of clonal repertoire of gene corrected lymphocytes in the subject as compared to a subject in need thereof not administered a gene therapy described herein. Increasing diversity of clonal repertoire of gene corrected lymphocytes can include increasing the number of unique retroviral integration site (RIS) clones as measured by a RIS analysis.

In particular embodiments, methods of the present disclosure can restore bone marrow function in a subject in need thereof. In particular embodiments, restoring bone marrow function can include improving bone marrow repopulation with gene corrected cells as compared to a subject in need thereof not administered a therapy described herein. Improving bone marrow repopulation with gene corrected cells can include increasing the percentage of cells that are gene corrected. In particular embodiments, the cells are selected from white blood cells and bone marrow derived cells. In particular embodiments, the percentage of cells that are gene corrected can be measured using an assay selected from quantitative real time PCR and flow cytometry.

In particular embodiments, methods of the present disclosure can normalize primary and secondary antibody responses to immunization in a subject in need thereof. Normalizing primary and secondary antibody responses to immunization can include restoring B-cell and/or T-cell cytokine signaling programs functioning in class switching and memory response to an antigen. Normalizing primary and secondary antibody responses to immunization can be measured by a bacteriophage immunization assay. In particular embodiments, restoration of B-cell and/or T-cell cytokine signaling programs can be assayed after immunization with the T-cell dependent neoantigen bacteriophage ψX174. In particular embodiments, normalizing primary and secondary antibody responses to immunization can include increasing the level of IgA, IgM, and/or IgG in a subject in need thereof to a level comparable to a reference level derived from a control population. In particular embodiments, normalizing primary and secondary antibody responses to immunization can include increasing the level of IgA, IgM, and/or IgG in a subject in need thereof to a level greater than that of a subject in need thereof not administered a gene therapy described herein. The level of IgA, IgM, and/or IgG can be measured by, for example, an immunoglobulin test. In particular embodiments, the immunoglobulin test includes antibodies binding IgG, IgA, IgM, kappa light chain, lambda light chain, and/or heavy chain. In particular embodiments, the immunoglobulin test includes serum protein electrophoresis, immunoelectrophoresis, radial immunodiffusion, nephelometry and turbidimetry. Commercially available immunoglobulin test kits include MININEPH™ (Binding site, Birmingham, UK), and immunoglobulin test systems from Dako (Denmark) and Dade Behring (Marburg, Germany). In particular embodiments, a sample that can be used to measure immunoglobulin levels includes a blood sample, a plasma sample, a cerebrospinal fluid sample, and a urine sample.

In particular embodiments, methods of the present disclosure can be used to treat SCID-X1. In particular embodiments, methods of the present disclosure can be used to treat SCID (e.g., JAK 3 kinase deficiency SCID, purine nucleoside phosphorylase (PNP) deficiency SCID, adenosine deaminase (ADA) deficiency SCID, MEW class II deficiency or recombinase activating gene (RAG) deficiency SCID). In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating SCIDX-1 with methods of the present disclosure include restoring functionality to the γC-dependent signaling pathway. The functionality of the γC-dependent signaling pathway can be assayed by measuring tyrosine phosphorylation of effector molecules STAT3 and/or STAT5 following in vitro stimulation with IL-21 and/or IL-2, respectively. Tyrosine phosphorylation of STAT3 and/or STAT5 can be measured by intracellular antibody staining.

In particular embodiments, methods of the present disclosure can be used to treat FA. In particular embodiments, therapeutic efficacy can be observed through lymphocyte reconstitution, improved clonal diversity and thymopoiesis, reduced infections, and/or improved patient outcome. Therapeutic efficacy can also be observed through one or more of weight gain and growth, improved gastrointestinal function (e.g., reduced diarrhea), reduced upper respiratory symptoms, reduced fungal infections of the mouth (thrush), reduced incidences and severity of pneumonia, reduced meningitis and blood stream infections, and reduced ear infections. In particular embodiments, treating FA with methods of the present disclosure include increasing resistance of bone marrow derived cells to mitomycin C (MMC). In particular embodiments, the resistance of bone marrow derived cells to MMC can be measured by a cell survival assay in methylcellulose and MMC.

In particular embodiments, methods of the present disclosure can be used to treat hypogammaglobulinemia. Hypogammaglobulinemia is caused by a lack of B-lymphocytes and is characterized by low levels of antibodies in the blood. Hypogammaglobulinemia can occur in patients with chronic lymphocytic leukemia (CLL), multiple myeloma (MM), non-Hodgkin's lymphoma (NHL) and other relevant malignancies as a result of both leukemia-related immune dysfunction and therapy-related immunosuppression. Patients with acquired hypogammaglobulinemia secondary to such hematological malignancies, and those patients receiving post-HSPC transplantation are susceptible to bacterial infections. The deficiency in humoral immunity is largely responsible for the increased risk of infection-related morbidity and mortality in these patients, especially by encapsulated microorganisms. For example, Streptococcus pneumoniae, Haemophilus influenzae, and Staphylococcus aureus, as well as Legionella and Nocardia spp. are frequent bacterial pathogens that cause pneumonia in patients with CLL. Opportunistic infections such as Pneumocystis carinii, fungi, viruses, and mycobacteria also have been observed. The number and severity of infections in these patients can be significantly reduced by administration of immune globulin (Griffiths et al. Blood 73: 366-368, 1989; Chapel et al. Lancet 343: 1059-1063, 1994).

In particular embodiments, formulations are administered to subjects to treat acute lymphoblastic leukemia (ALL), acute myelogenous leukemia (AML), adrenoleukodystrophy, agnogenic myeloid metaplasia, amegakaryocytosic/congenital thrombocytopenia, ataxia telangiectasia, β-thalassemia, chronic granulomatous disease, chronic lymphocytic leukemia (CLL), chronic myelogenous leukemia (CML), chronic myelomonocytic leukemia, common variable immune deficiency (CVID), complement disorders, congenital agammaglobulinemia, Diamond Blackfan anemia (DBA), diffuse large B-cell lymphoma, familial erythrophagocytic lymphohistiocytosis, follicular lymphoma, Hodgkin's lymphoma, Hurler's syndrome, hyper IgM, IgG subclass deficiency, juvenile myelomonocytic leukemia, metachromatic leukodystrophy, mucopolysaccharidoses, multiple myeloma, myelodysplasia, non-Hodgkin's lymphoma, paroxysmal nocturnal hemoglobinuria (PNH), primary immunodeficiency diseases, pure red cell aplasia, refractory anemia, Shwachman-Diamond syndrome, selective IgA deficiency, severe aplastic anemia, sickle cell disease, specific antibody deficiency, Wiskott-Aldrich syndrome, and/or X-linked agammaglobulinemia (XLA).

Particular embodiments include treatment of secondary, or acquired, immune deficiencies such as immune deficiencies caused by trauma, viruses, chemotherapy, toxins, and pollution. As previously indicated, acquired immunodeficiency syndrome (AIDS) is an example of a secondary immune deficiency disorder caused by a virus, the human immunodeficiency virus (HIV), in which a depletion of T lymphocytes renders the body unable to fight infection. Thus, as another example, a gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

Patients with MGMT expressing tumors would benefit from administration of adenoviral vector (e.g. Ad3, 7, 11, 14, 16, 21, 34, 37, or 50 vectors) with a therapeutic payload (such as a CAR, TCR, or checkpoint inhibitor) combined with the MGMT^{P140K} in vivo selection cassette. Ex vivo approaches have shown the applicability of this approach. In particular embodiments, therapeutic amounts of TMZ and benzylguanine or BCNU are administered to reduce the tumor burden or volume.

In particular embodiments, therapeutically effective amounts may provide function to immune and other blood cells, reduce or eliminate an immune-mediated condition; and/or reduce or eliminate a symptom of the immune-mediated condition.

The Exemplary Embodiments and Example(s) provided herein are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXAMPLES

The present Examples demonstrate that certain adenoviral serotypes are particularly effective for infection of CD34+ cells such as HSCs. Because HSCs are a therapeutically important target for gene therapy, identification of vectors effective for transduction of CD34+ cells is of substantial clinical importance. Certain tested adenoviral serotypes were similarly or more effective for infection of CD34+ cells than others commonly associated with gene therapy trials and research, such as Ad5 and Ad5/35++.

### Example 1: Analysis of Adenoviral Vector Infection of CD34+ Cells by Anti-Hexon Staining

The present example utilizes anti-hexon staining to measure the infection of CD34+ cells by various adenoviral vectors. Serotypes used in experiments of this Example included Ad3, Ad5, Ad7, Ad11, Ad14, Ad16, Ad21, Ad26, Ad34, Ad35, Ad37, Ad48, Ad50, and Ad52, as well as an Ad5/35++ vector including E1 deletion (“F35”). Vectors were wild type human adenoviral vectors except as otherwise noted.

Human CD34+ cells (REF: 4Y-101C, LOT: 3038009, Donor ID: 15846) were infected with wild type human adenoviruses (identified by Ad type number) with 5,000 or 2,000 viral particles per cell (vp/c). Three hours post-incubation, cells were first washed with phosphate buffered saline (PBS), quickly trypsinized to remove all extracellular viral particles, and washed with PBS. Washed cells were then split into two aliquots utilized in the present Example for analysis of intra-cellular adenovirus particles by anti-hexon staining and in Example 2 for analysis of adenoviral DNA internalization by qPCR, respectively. A replicate trial was additionally conducted in which CD34+ cells were infected at 2,000, 10,000, and 20,000 viral particles per cell (vp/c).

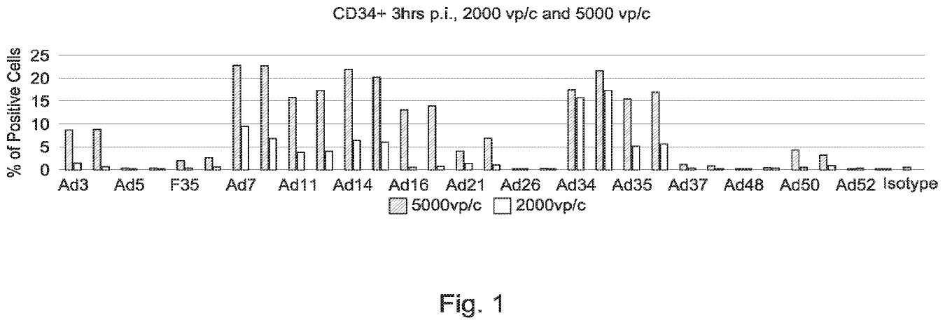

In the present Example, cells were first fixed with fixation medium (Thermofisher) for 15 minutes at room temperature. After a PBS washing step, cells were resuspended in permeabilization medium (Thermofisher). Anti-adenovirus hexon antibody (clone 20/11, MAB8052, Sigma) was added to the permeabilization medium and incubated at 4° C. overnight. On the second day, cells were washed twice with PBS and stained with the Alexa Fluor 488-labeled secondary antibody (Catalog #A-21121, Thermofisher) in permeabilization medium. Staining was stopped with two PBS washing steps, and the cells were analyzed on a Beckman Coulter Gallios Flow Cytometer. Background signal was obtained by analyzing the isotype control, which refers to staining using mouse IgG1 Isotype Control antibody (Sigma, REF: M5284-.1MG, Clone: MOPC 21). The percentage of FITC positive cells is displayed in the FIG. 1. For each virus two samples are shown for each virus dose.

Results of anti-hexon staining are provided in FIG. 1. Reference serotypes in this Example, as shown in FIG. 1, include Ad5 and Ad5/35++ (F35) serotypes that are often used, e.g., that have been used in gene therapy research or adenoviral vector constructs. Unexpectedly, several adenoviral vector serotypes consistently outperformed these reference serotypes for internalization into CD34+ cells. These included Ad3, 7, 11, 14, 16, 21, 34, 35, and 50. By contrast, serotypes Ad26, Ad37, Ad48, and Ad52 consistently did not outperform reference serotypes for internalization into CD34+ cells. These data demonstrate that Ad3, 7, 11, 14, 16, 21, 34, 35, and 50 are particularly and unexpectedly useful for engineering of vectors for transduction of CD34+ cells such as HSCs.

### Example 2: Analysis of the Internalization of Adenovirus Particles into CD34+ Cells by qPCR

The present example utilizes qPCR to measure the internalization of adenovirus particles into CD34+ cells by various adenoviral serotypes. Serotypes used in experiments of this Example included Ad3, Ad5, Ad7, Ad11, Ad14, Ad16, Ad21, Ad26, Ad34, Ad37, Ad35, Ad48, Ad50, and Ad52, as well as Ad5/35++ vector including an E1 deletion (“F35”). The viruses used were purified wild type human adenoviruses except as otherwise noted. Cells were prepared as described in Example 1.

In the present Example, total genomic DNA was isolated using the Monarch® Genomic DNA Purification Kit (NEB). For qPCR analyses, samples were split into two experiments: Ad3, 7, 11, 14, 16, 21, 34, 35, and 50 in a first experiment; and Ad26, Ad37, Ad48, Ad52, Ad5, and F35 in a second experiment. For the first experiment, primers and probe targeting DNA polymerase were used for amplification and purified plasmid containing the Ad35 genome (pAd35) was used to generate a standard curve. For the second experiment, primers and probe targeting hexon were used for amplification and purified plasmid containing the Ad5 genome (pAd5) was used to generate a standard curve. For normalization, primers that amplify the gene hB2M were applied.

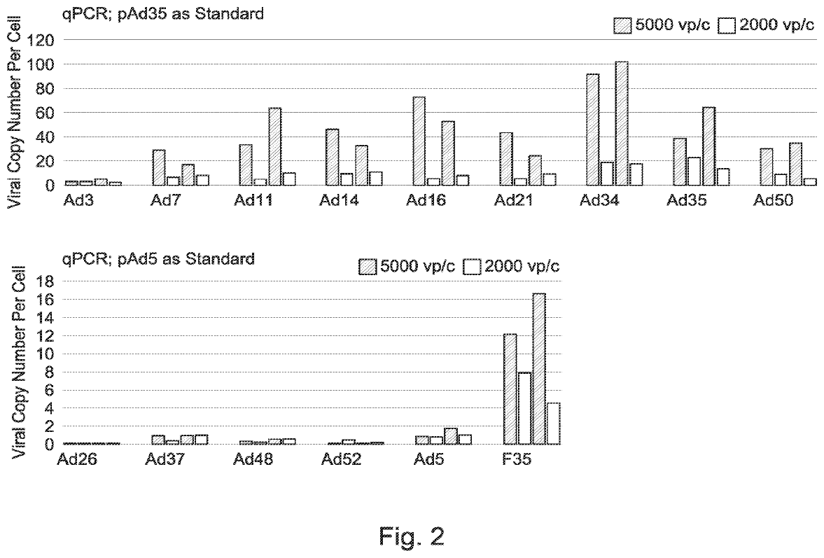

Results of the qPCR analyses of this Example are provided in FIG. 2. Broadly, viral copy number per cell was highest using Ad7, Ad11, Ad14, Ad16, Ad21, Ad34, Ad35, Ad50, and F35. Viral copies per cell were also detected for Ad3, Ad37, Ad48, Ad52, and Ad5. Viral copy number per cell was lowest for Ad26.

### Example 3: Analysis of Adenoviral Vector Infection of CD34+ Cells by Anti-Hexon Staining

The present example utilizes anti-hexon staining to measure the infection of CD34+ cells by various adenoviral vectors. Serotypes used in experiments of this Example included Ad3, Ad5, Ad7, Ad11, Ad14, Ad16, Ad21, Ad26, Ad34, Ad35, Ad37, Ad48, Ad50, and Ad52, as well as an Ad5/35++ vector including E1 deletion (“F35”). Vectors were wild type human adenoviral vectors except as otherwise noted.

Human CD34+ cells from three donors were infected with wild type human adenoviruses (identified by Ad type number) with 5,000 or 2,000 viral particles per cell (vp/c). Donor 1 cells (Lonza, REF: 4Y-101C, LOT: 3038009, Donor ID: 15846) and Donor 2 cells (Lonza, REF: 4Y-101E, LOT: 3046829, Donor ID: 14538) were from donors subjected to mobilization of hematopoietic stem cells (HSCs) by G-CSF; while Donor 3 cells (Hemacare, REF: M34C-MOZ-1, LOT: 20063998) were from donors subjected to HSC mobilization by plerixafor. Three or six hours post-incubation, cells were first washed with phosphate buffered saline (PBS), quickly trypsinized to remove all extracellular viral particles, and washed with PBS. Washed cells were then split into two aliquots utilized in the present Example for analysis of intra-cellular adenovirus particles by anti-hexon staining (this Example), and for analysis of adenoviral DNA internalization by qPCR (Example 4), respectively.

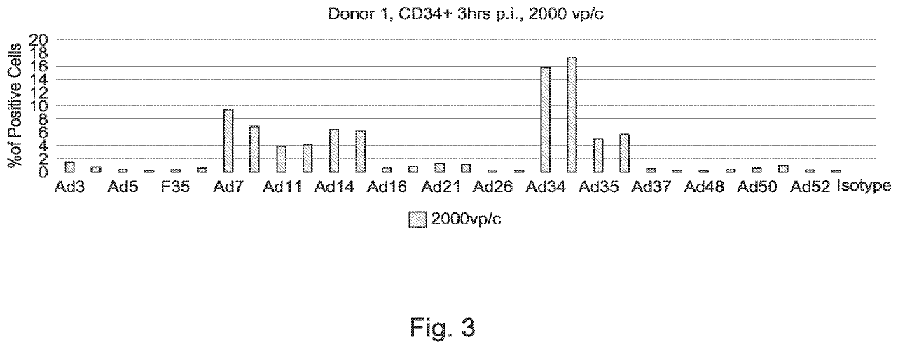

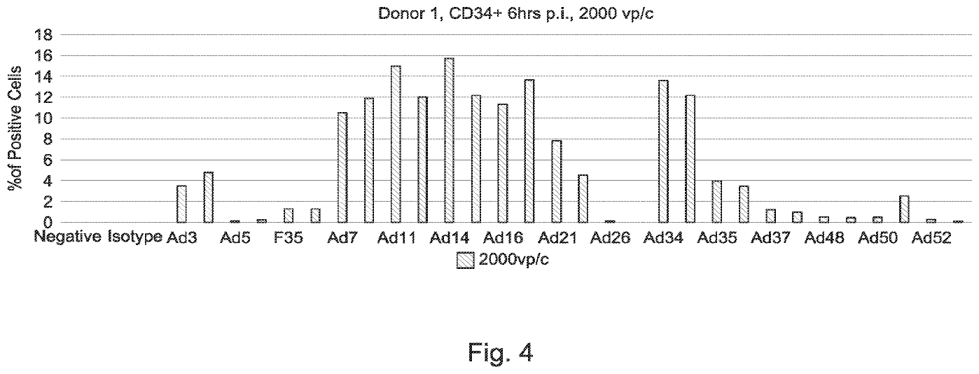

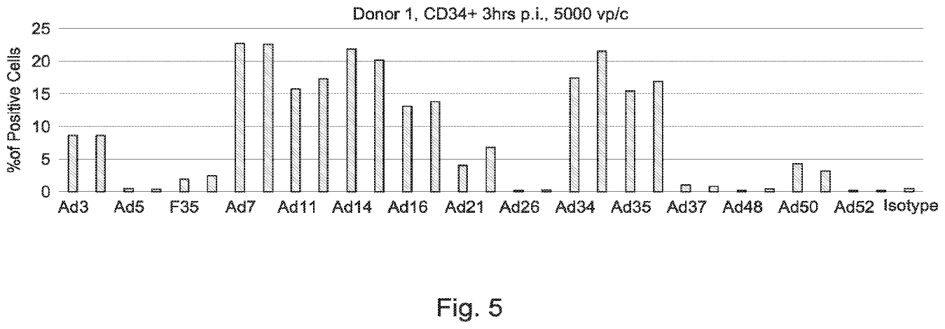

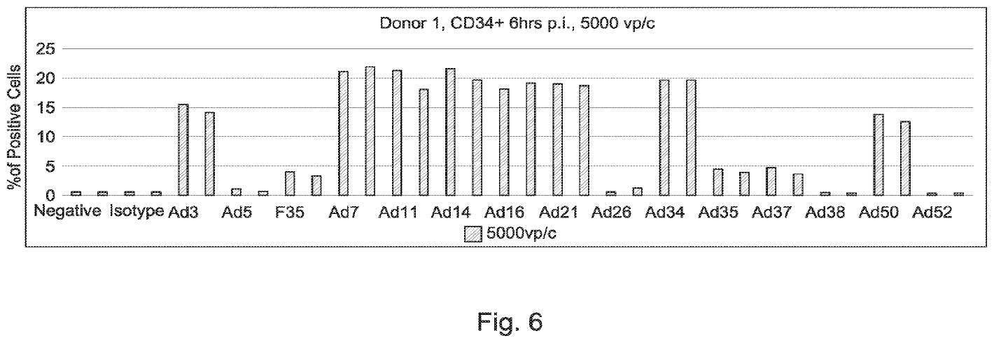

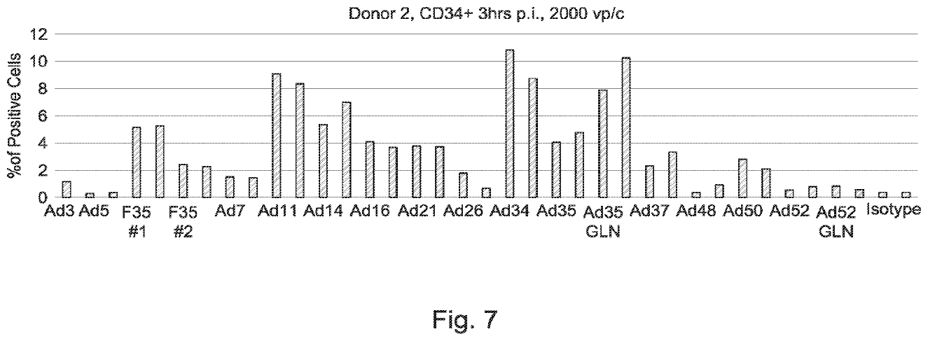

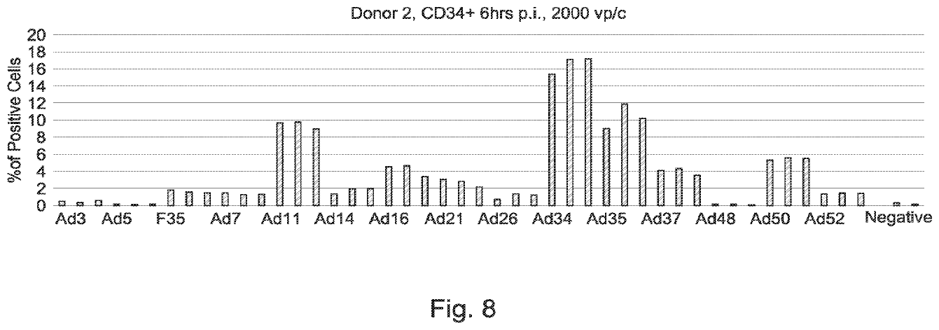

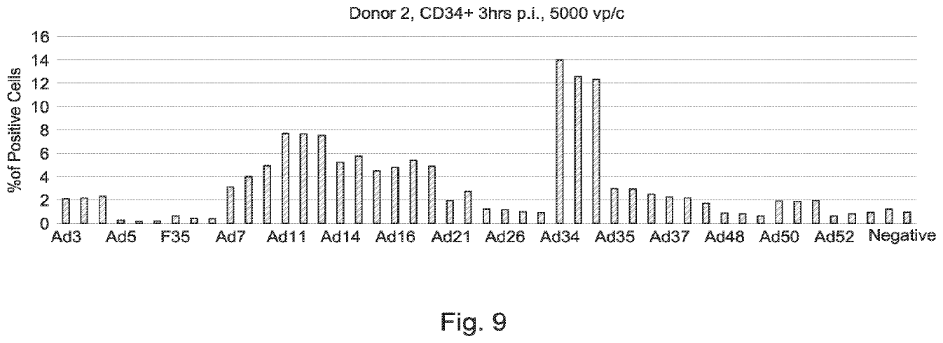

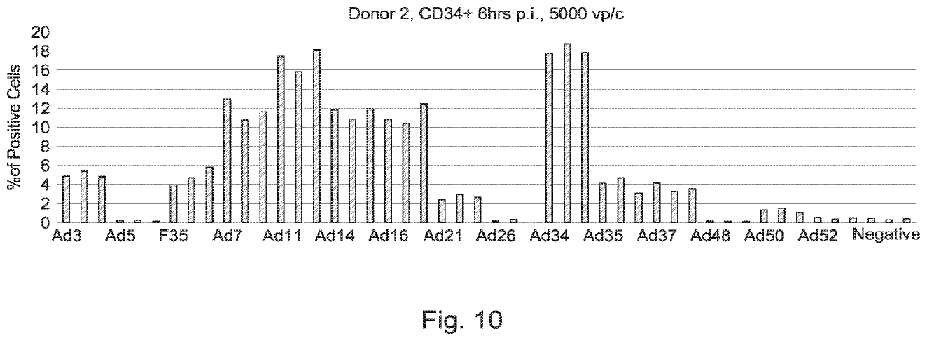

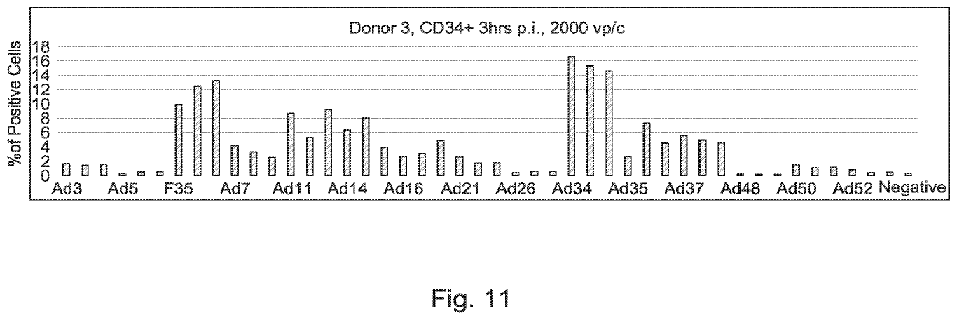

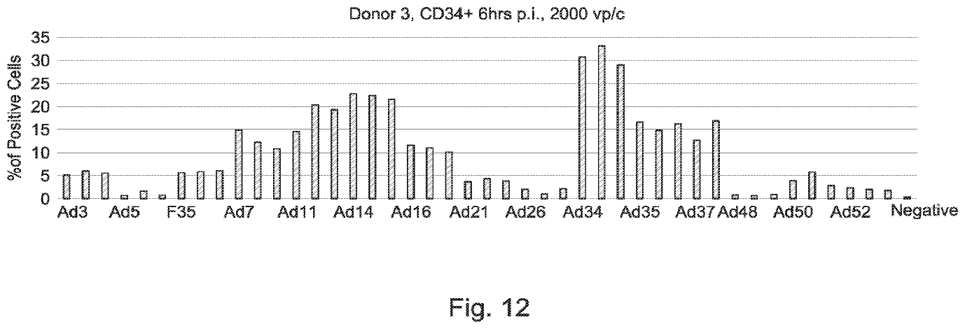

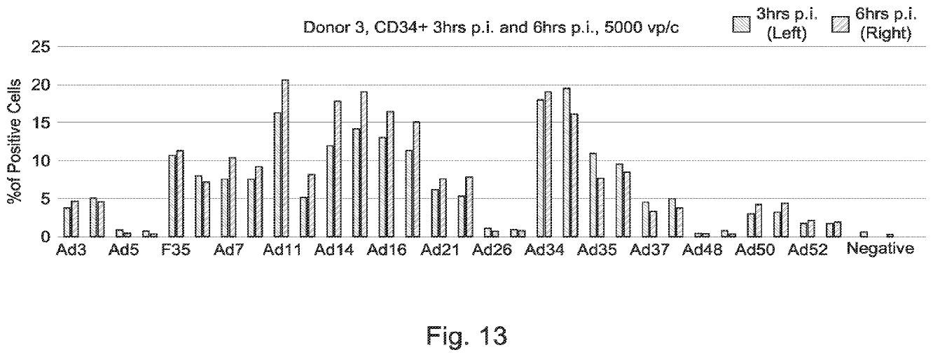

In the present Example, cells were first fixed with fixation medium (Thermofisher) for 15 minutes at room temperature. After a PBS washing step, cells were resuspended in permeabilization medium (Thermofisher). Anti-adenovirus hexon antibody (clone 20/11, MAB8052, Sigma) was added to the permeabilization medium and incubated at 4° C. overnight. On the second day, cells were washed twice with PBS and stained with the Alexa Fluor 488-labeled secondary antibody (Catalog #A-21121, Thermofisher) in permeabilization medium. Staining was stopped with two PBS washing steps, and the cells were analyzed on a Beckman Coulter Gallios Flow Cytometer. Background signal was obtained by analyzing the negative control, which refers to uninfected cells stained with the same antibodies as the sample, and/or isotype control, which refers to staining using mouse IgG1 Isotype Control antibody (Sigma, REF: M5284-.1MG, Clone: MOPC 21). The percentage of FITC positive cells is displayed in FIGS. 3-13. For each virus two or three samples are shown for each virus dose.

Results of anti-hexon staining are provided in FIG. 3-13. Reference serotypes in this Example, as shown in FIGS. 3-13, include Ad5 and Ad5/35++(F35) serotypes that are often used, e.g., that have been used in gene therapy research or adenoviral vector constructs. Unexpectedly, several adenoviral serotypes consistently outperformed the reference Ad5 serotype, and in some instances also outperformed the reference F35 serotype, for internalization into CD34+ cells. These included Ad3, 7, 11, 14, 16, 21, 34, 35, 37, and 50. Serotype Ad37 outperformed reference serotype Ad5 for internalization into CD34+ cells from Donors 2 and 3, but not Donor 1. By contrast, serotypes Ad26, Ad48, and Ad52 consistently did not outperform reference serotypes for internalization into CD34+ cells. These data demonstrate that Ad3, 7, 11, 14, 16, 21, 34, 35, 37, and 50 are particularly and unexpectedly useful for engineering of vectors for transduction of CD34+ cells such as HSCs.

### Example 4: Analysis of the Internalization of Adenovirus Particles into CD34+ Cells by qPCR

The present example utilizes qPCR to measure the internalization of adenovirus particles into CD34+ cells by various adenoviral serotypes. Serotypes used in experiments of this Example included Ad3, Ad5, Ad7, Ad11, Ad14, Ad16, Ad21, Ad26, Ad34, Ad37, Ad35, Ad48, Ad50, and Ad52, as well as Ad5/35++ vector including an E1 deletion (“F35”). The viruses used were purified wild type human adenoviruses except as otherwise noted. Cells were prepared as described in Example 3.

In the present Example, total genomic DNA was isolated using the Monarch® Genomic DNA Purification Kit (NEB). For qPCR analyses, samples were split into two experiments: Ad3, 7, 11, 14, 16, 21, 34, 35, and 50 in a first experiment; and Ad26, Ad37, Ad48, Ad52, Ad5, and F35 in a second experiment. For the first experiment, primers and probe targeting DNA polymerase were used for amplification and purified plasmid containing the Ad35 genome (pAd35) was used to generate a standard curve. For the second experiment, primers and probe targeting hexon were used for amplification and purified plasmid containing the Ad5 genome (pAd5) was used to generate a standard curve. For normalization, primers that amplify the gene hB2M were applied. Where examined, background signal was obtained by analyzing the negative control, which refers to genomic DNA isolated from non-infected cells, and/or water (H₂O) control, which refers to using water instead of genomic DNA in the qPCR reaction.

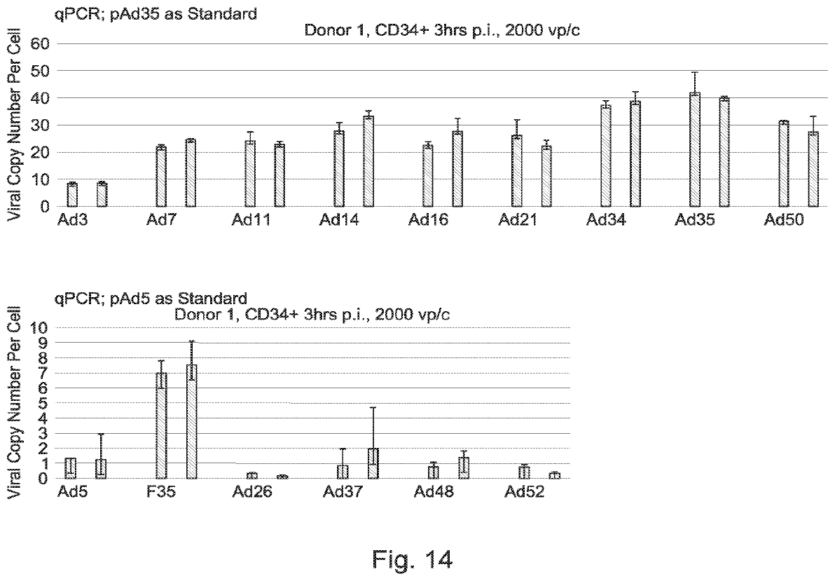

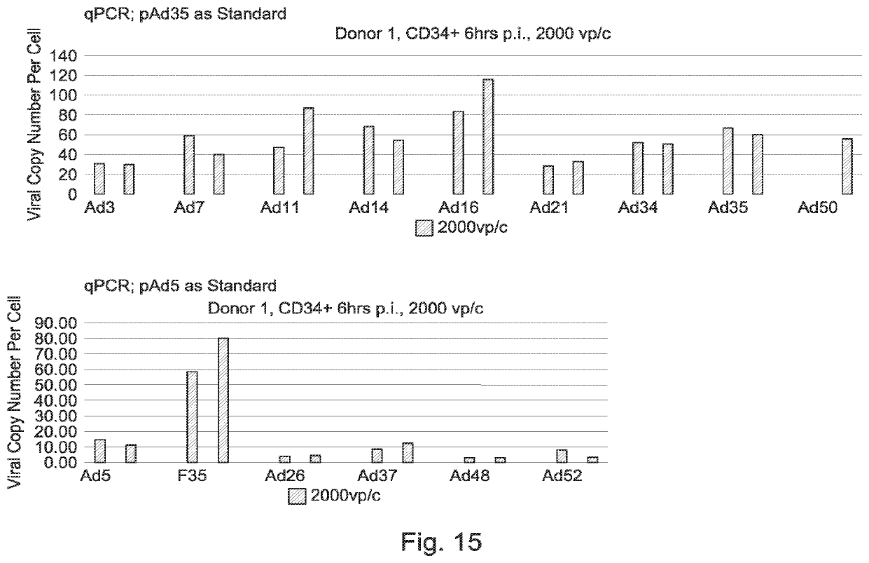

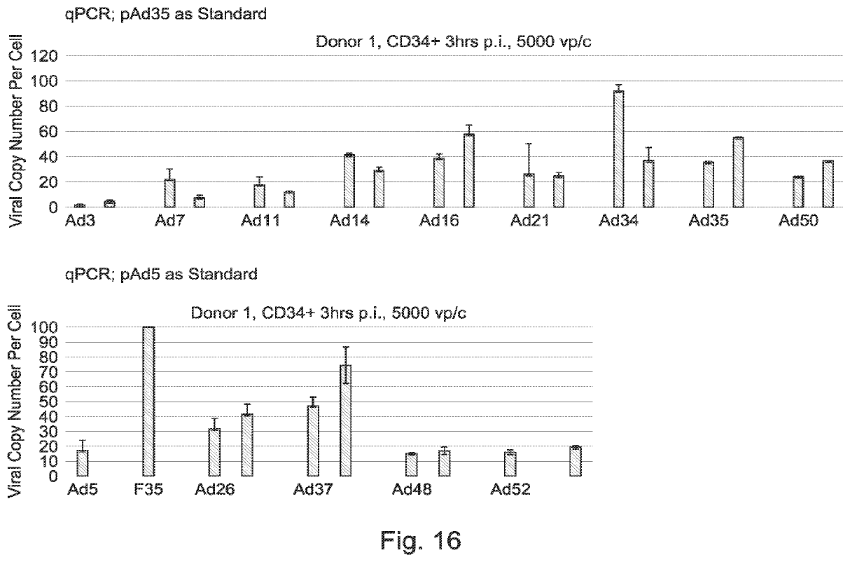

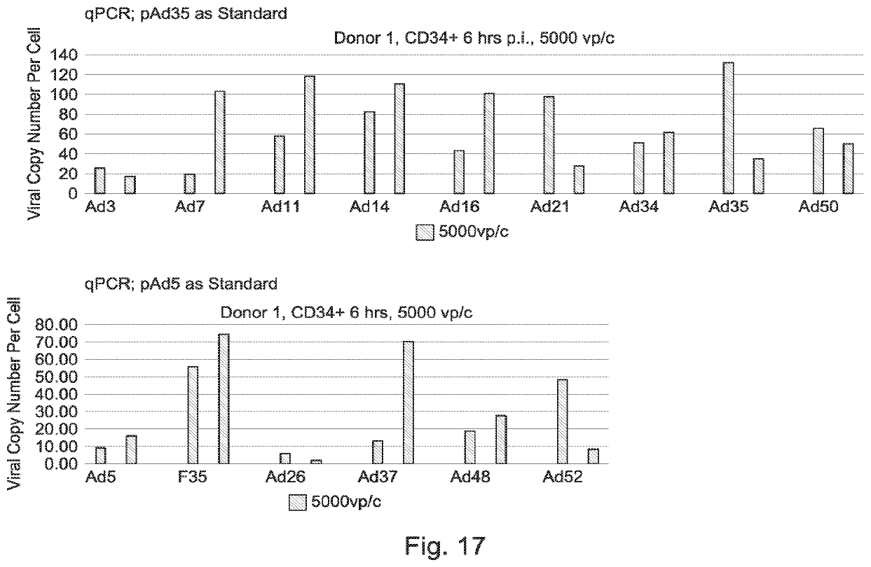

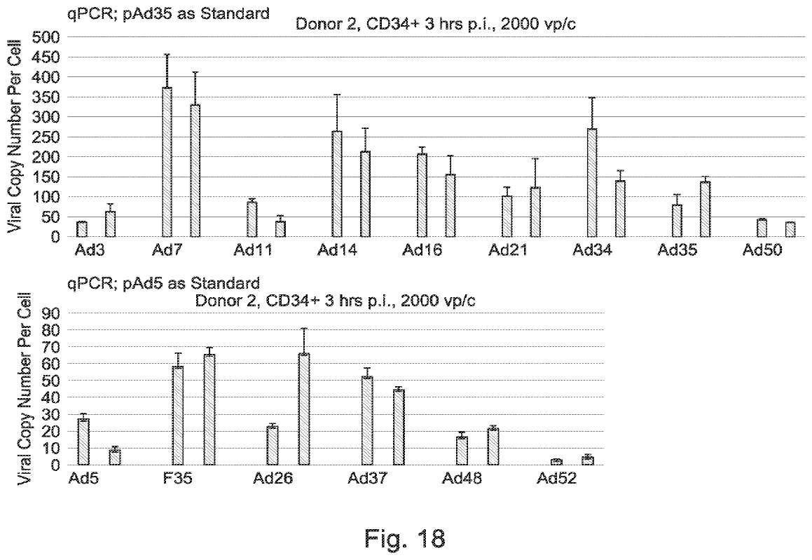

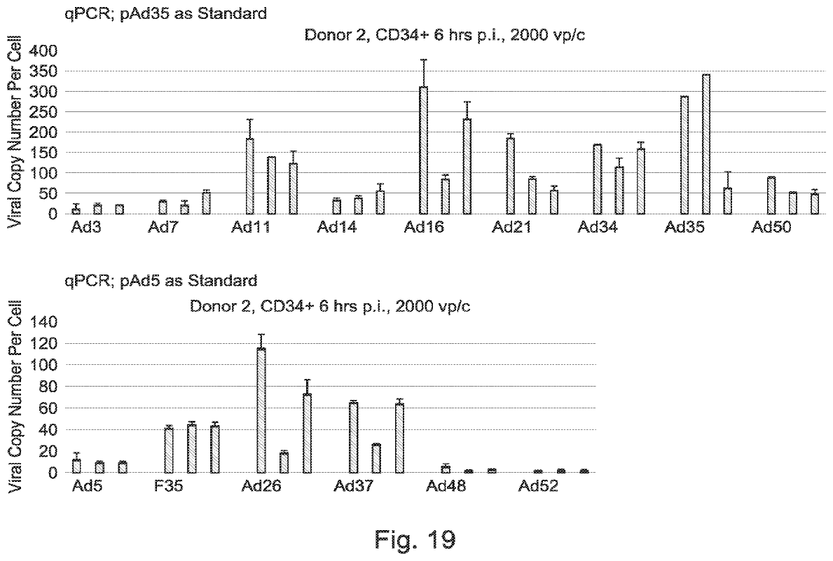

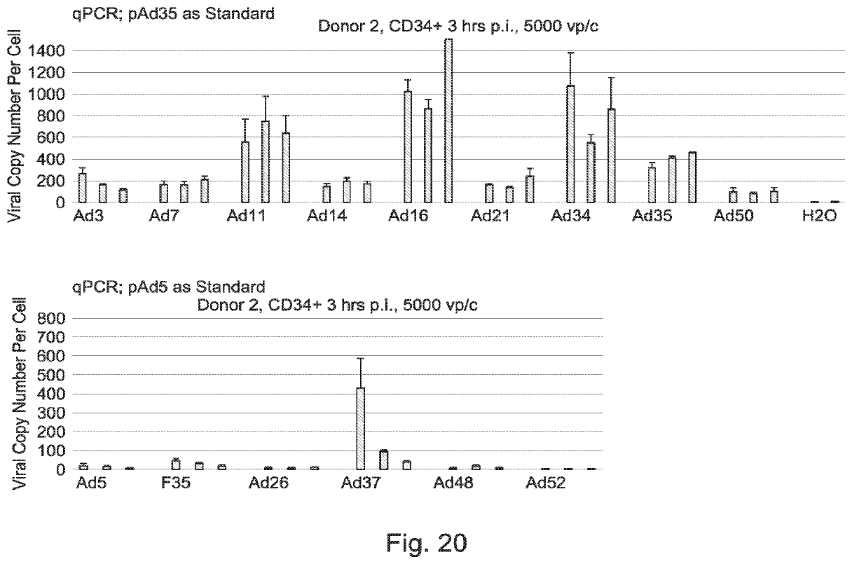

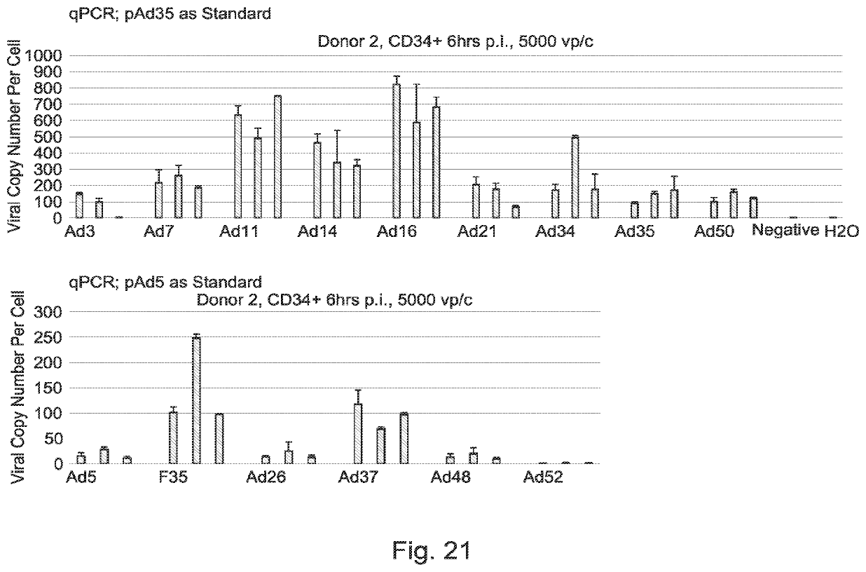

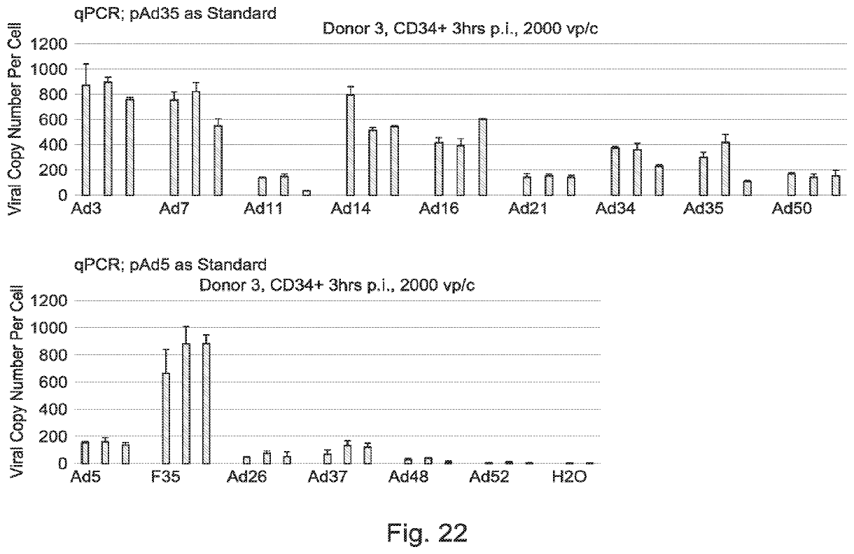

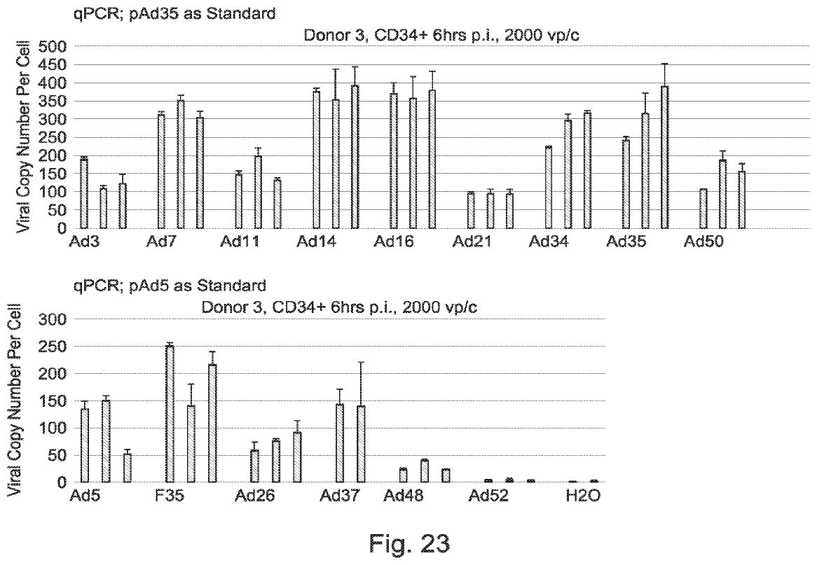

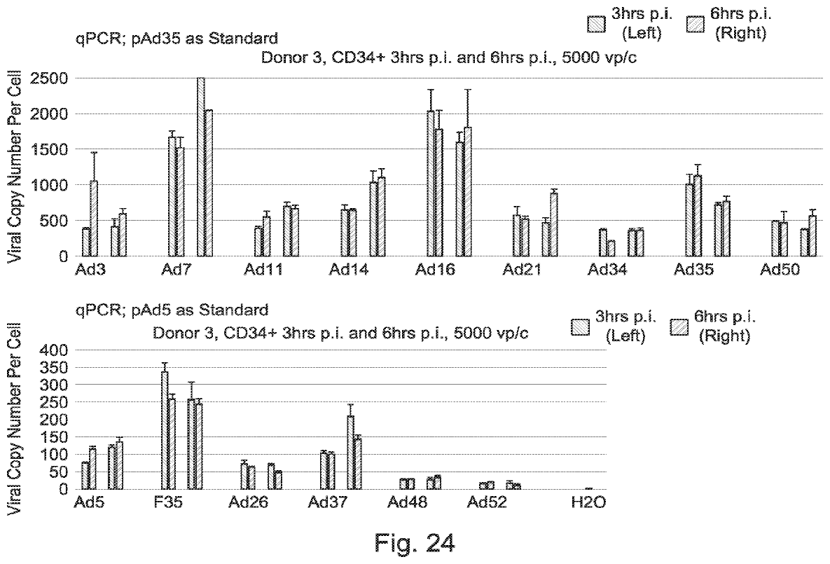

Results of the qPCR analyses of this Example are provided in FIGS. 14-24. Broadly, viral copy number per cell was highest using Ad3, Ad7, Ad11, Ad14, Ad16, Ad21, Ad34, Ad35, Ad37, Ad50, and F35. Viral copies per cell were also detected for Ad5, Ad26, Ad48, and Ad52.

### Example 5: Production of First Generation Adenoviral Vectors

The present example includes the production of first generation adenoviral vectors from various adenoviral serotypes. Serotypes used in experiments of this Example included Ad11, Ad34, and Ad35. First generation adenoviral genomes were produced with the regulatory E1 gene (E1a and E1b) removed from the Ad genome. Additionally, first generation Ad genomes were engineered to replace the endogenous E4orf6 gene, if the endogenous E4orf6 gene was not of Ad5 serotype, with an Ad5 E4orf6 gene. The first generation Ad35 genome further included a mutant Ad35++ fiber knob, which fiber knob is described elsewhere herein, and which first generation Ad35 genome is referred to in the present Examples as a first generation Ad35++ genome. First generation Ad genomes of the present Example were also engineered to include a nucleic acid payload expressing green fluorescence protein (GFP) from a coding sequence under the control of an EF1-alpha promoter and operably linked with a bovine growth hormone (BGH) polyadenylation signal. Those of ordinary skill in the art will appreciate from the present Examples and disclosure that other adenoviral serotypes (e.g., Ad3, Ad5, Ad7, Ad14, Ad16, Ad21, Ad26, Ad37, Ad48, Ad50, and Ad52) can also be used to produce adenoviral vector genomes such as first generation adenoviral vector genomes and other forms or generations disclosed herein.

Plasmids encoding first-generation Ad genomes were transfected into HEK293 cells and propagated to determine whether viable Ad vectors could be rescued. Rescued Ad vectors were purified using standard methods (see, e.g., Su et al. doi:10.1101/pdb.prot095547 Cold Spring Harb Protoc 2019).

Purified Ad vectors were characterized using several approaches. The physical titer or yield of the purified virus preparations was determined by spectrophotometry and can be expressed as the total number of purified viral particles (vp) or the number of viral particles per transfected HEK293 cell (vp/cell). Table 19 shows the results from experiments to characterize the purified first generation Ad preparations.

TABLE 19
Characterization of Purified First Generation Ad Preparations

| | Yield | Yield |
|---|---|---|
| Vector | (vp) | (vp/cell) |
| First generation Ad11 | 1.2e12 | 3.1e3 |
| First generation Ad34 | 2e12 | 4.3e3 |
| First generation Ad35++ | 8e12 | 3e4 |

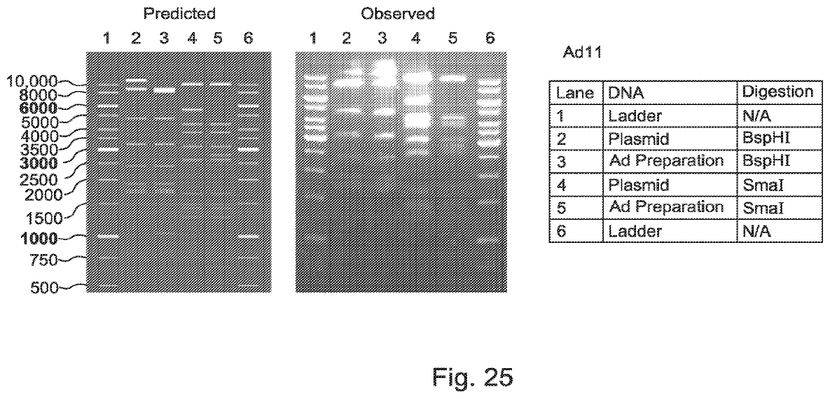

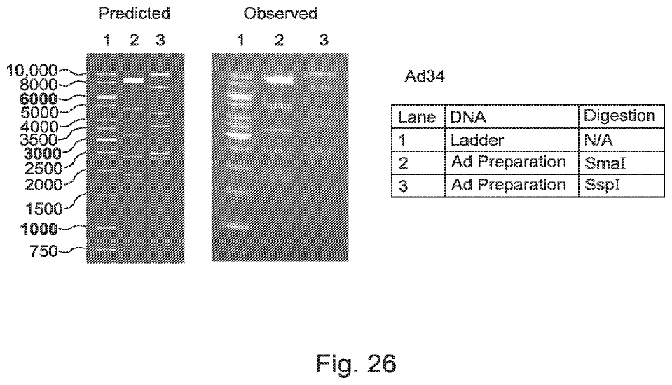

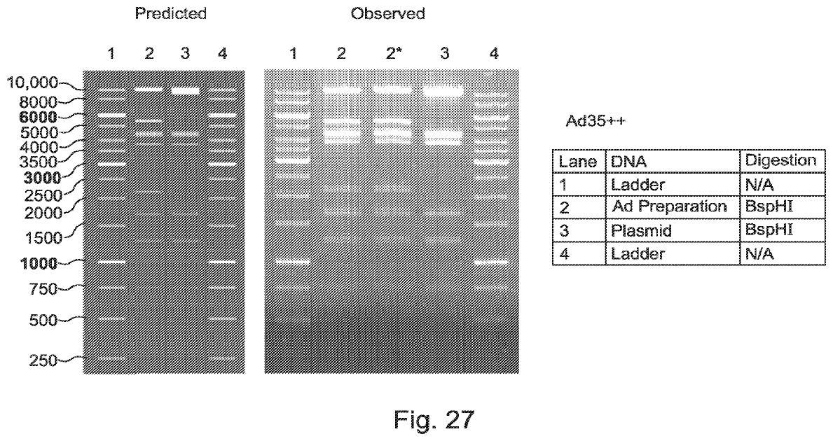

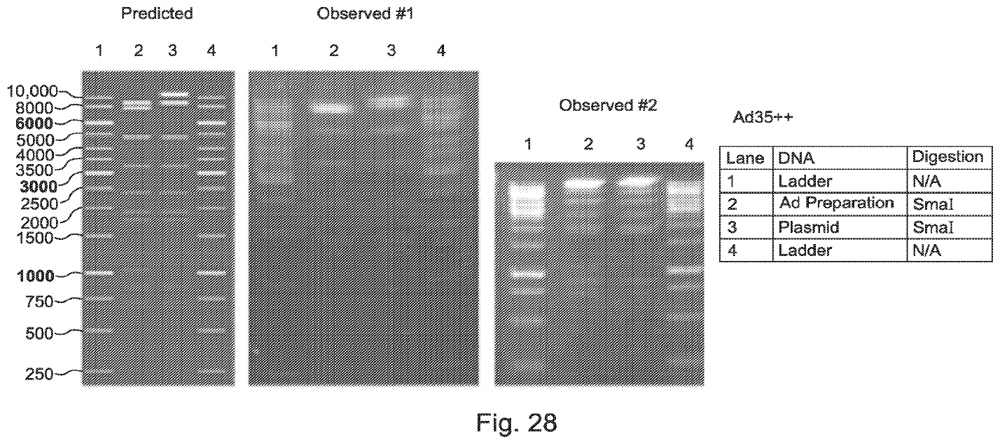

Purified Ad vectors were additionally characterized by restriction enzyme digestion of DNA isolated from the purified Ad preparations. Isolated DNA was digested using restriction enzymes (Sural, SspI, or BspHI), and the restriction pattern was compared to the restriction pattern obtained by digestion using the same restriction enzyme of the starting plasmid encoding the first generation Ad genome and/or the predicted restriction pattern based on the sequence of the Ad genome. Analysis of the restriction patterns on a gel showed the expected banding pattern and expected band sizes (FIGS. 25-28), demonstrating successful production of first generation Ad11, Ad34, and Ad35++ vectors.

### Example 6: Analysis of First Generation Adenoviral Vector Infection of Cells

The present example utilizes analysis of GFP payload expression to measure the infection of cells by various first generation adenoviral vectors. Serotypes used in experiments of this Example included Ad11, Ad34, Ad35, and Ad35++(Ad35 with mutant Ad35 fiber knob as described elsewhere herein). Vectors were first generation adenoviral vectors and included a nucleic acid payload encoding GFP, as described in Example 5.

Human cell lines (HEK293 and K562) and CD34+ cells (from Donors 1, 2, and 3 cells as set forth in Example 3) were infected with first generation adenoviral vectors (identified by Ad type number) with between 100 to 5,000 viral particles per cell (vp/c). At 3, 24, 25, or 48 hours post-incubation, cells were first washed with phosphate buffered saline (PBS), quickly trypsinized to remove all extracellular viral particles, and washed with PBS. Washed cells were then split into two aliquots utilized in the present Example for analysis of intra-cellular adenovirus particles by analysis of GFP payload expression (this Example) and for analysis of adenoviral DNA internalization by qPCR (in Example 7), respectively.

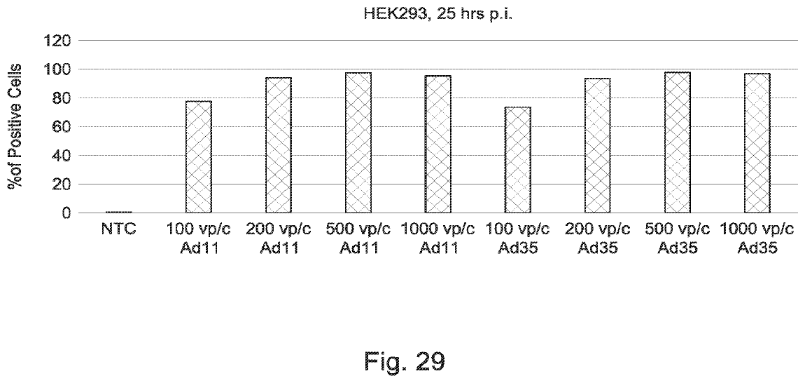

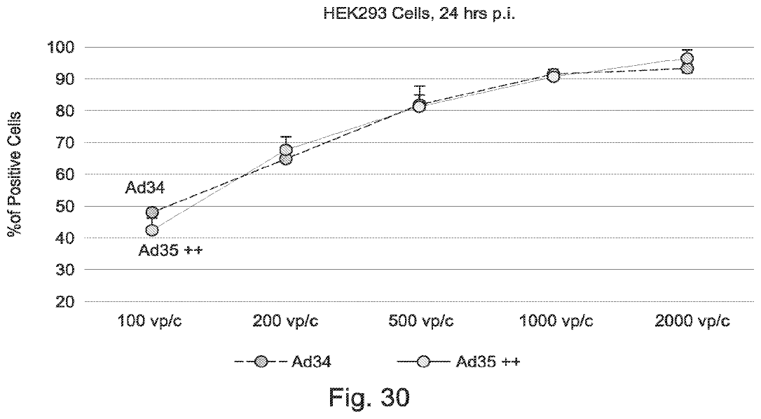

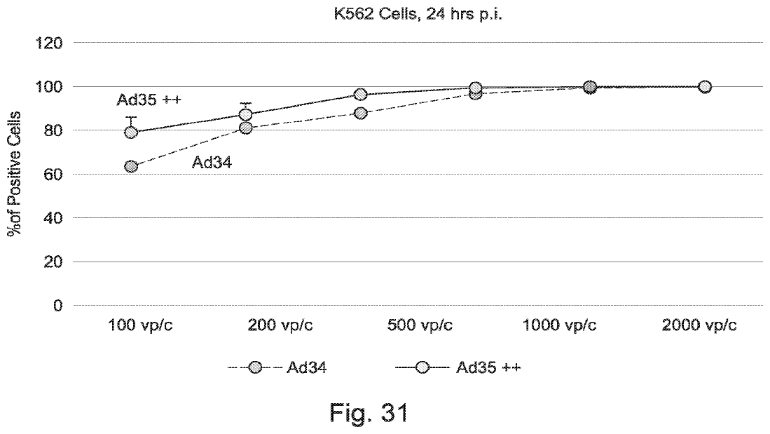

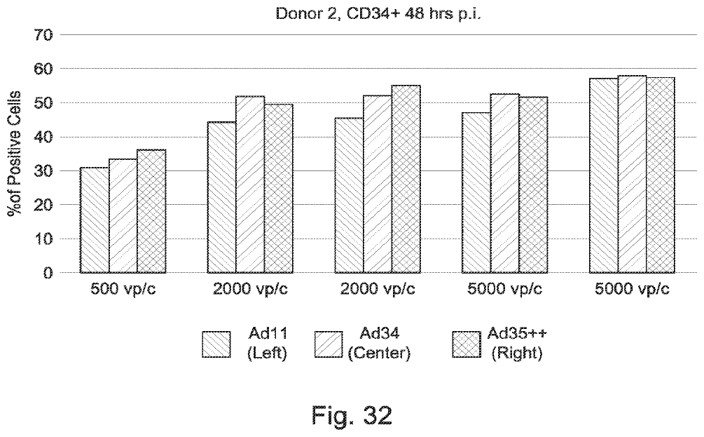

In the present Example, cells were analyzed on a Beckman Coulter Gallios Flow Cytometer by detecting GFP payload expression. Results of analysis of GFP payload expression are provided as the percentage of GFP positive cells in FIGS. 29-32. First generation adenoviral vectors of serotypes Ad11, Ad34, Ad35, and Ad35++ showed substantial performance for internalization into HEK293 cells (FIGS. 29 and 30). First generation adenoviral vectors of serotypes Ad34 and Ad35++ showed substantial performance for internalization into K562 cells (FIG. 31). First generation adenoviral vectors of serotypes Ad11, Ad34, and Ad35++ showed substantial performance for internalization into CD34+ cells (FIG. 32). These data demonstrate that the tested serotypes can be engineered into vectors for transduction of human cells, and further demonstrate that serotypes Ad11, Ad34, and Ad35++ can be engineered into vectors for transduction of CD34+ cells, such as HSCs.

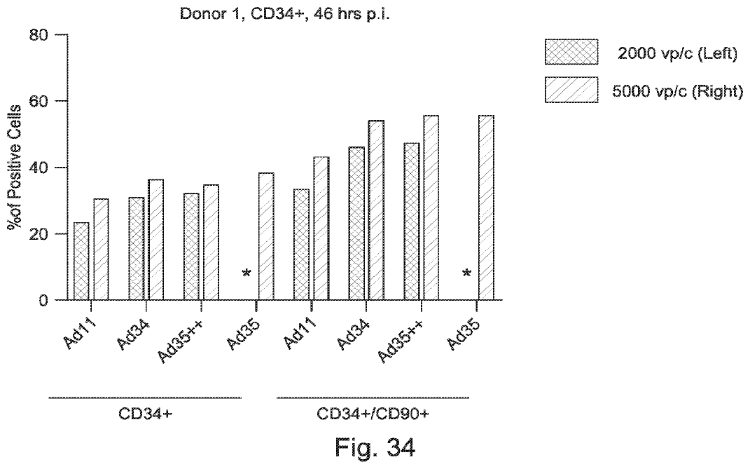

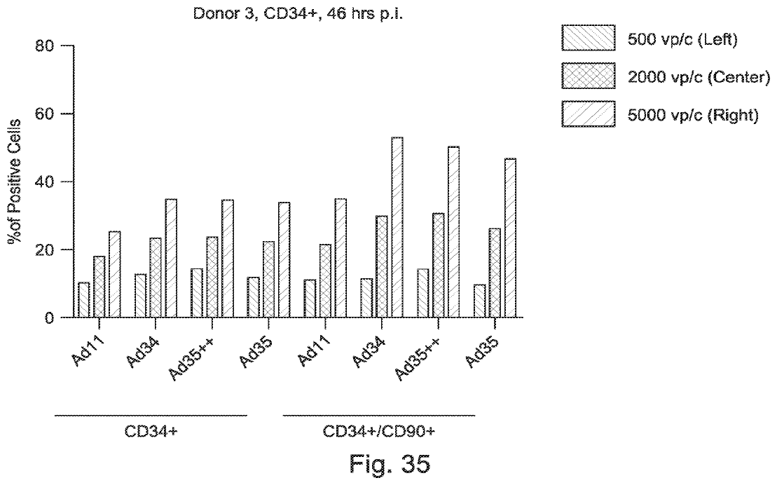

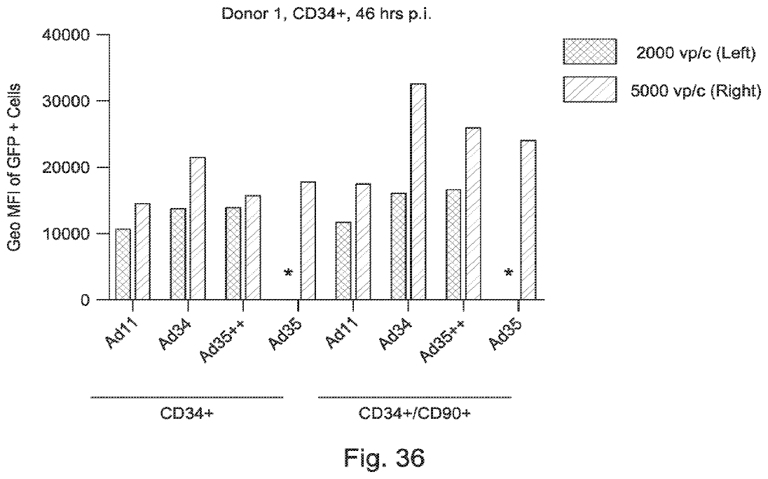

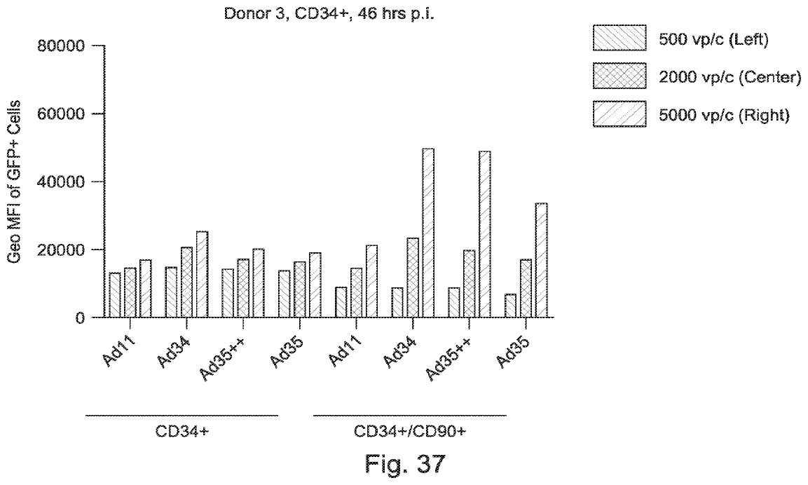

Further characterization of infection of cells using first generation adenoviral vectors from serotypes Ad11, Ad34, Ad35 (first generation Ad35 and first generation Ad35++) was performed by examining GFP payload expression in CD34+/CD90+ subpopulation of CD34+ cells from Donor 1 and Donor 3. The CD34+/CD90+ subpopulation defines a more primitive subpopulation of HSCs. To distinguish the CD34+/CD90+ subpopulation, 46 hours after transduction cells were resuspended in staining buffer (0.5% BSA in PBS) with a Fc receptor blocking solution (BioLegend, Human TruStain FcX) at 4° C. for 15 minutes. Next, the cells were incubated at 4° C. for 20 minutes with anti-CD34 antibody conjugated to APC (BD Biosciences, REF: 340441, clone 8G12) and anti-CD90 antibody conjugated to BV421 (BD Biosciences, REF: 562556, clone 5E10). Cells were washed once with 0.5% BSA in PBS, and then analyzed by flow cytometry. The flow cytometry data were to identify CD34+ cells and CD34+/CD90+ cells. Within each populations of cells, the GFP positive cells were identified in order to determine the percentage of GFP positive cells and the geometric mean fluorescence intensity (MFI) of GFP in the GFP positive cells. An exemplary gating is shown in FIG. 33. Results of analysis of GFP payload expression in the CD34+/CD90+ subpopulation compared to the CD34+ population are provided as the percentage of GFP positive cells in FIGS. 34 and 35 and the geometric MFI of GFP in the GFP positive cells in FIGS. 36 and 37. First generation adenoviral vectors of serotypes Ad11, Ad34, Ad35, and Ad35++ showed greater infectivity of the CD34+/CD90+ subpopulation of cells compared to the general CD34+ population at 2,000 and 5,000 viral particles per cell. The tested serotypes also showed greater expression of payload encoded GFP in the CD34+/CD90+ subpopulation of cells compared to the general CD34+ population at 5,000 viral particles per cell. These data demonstrate that the tested serotypes can be engineered into vectors for transduction of human CD34+ cells, and can be particularly effective in transducing CD34+/CD90+ primitive HSCs.

### Example 7: Analysis of First Generation Adenoviral Vector Infection of Cells by qPCR

The present example utilizes qPCR to measure the internalization of adenovirus particles into HEK293 cells and CD34+ cells (from Donor 2) by various adenoviral serotypes. Serotypes used in experiments of this Example included Ad11, Ad34, and Ad35++. The viruses used were purified first generation adenoviral vectors and included a nucleic acid payload encoding GFP, as described in Example 5. Cells were prepared as described in Example 6.

In the present Example, total genomic DNA was isolated using the Monarch® Genomic DNA Purification Kit (NEB). For qPCR analyses, primers and probe targeting DNA polymerase were used for amplification and purified plasmid containing the Ad35 genome (pAd35) was used to generate a standard curve. For normalization, primers that amplify the gene hB2M were applied.

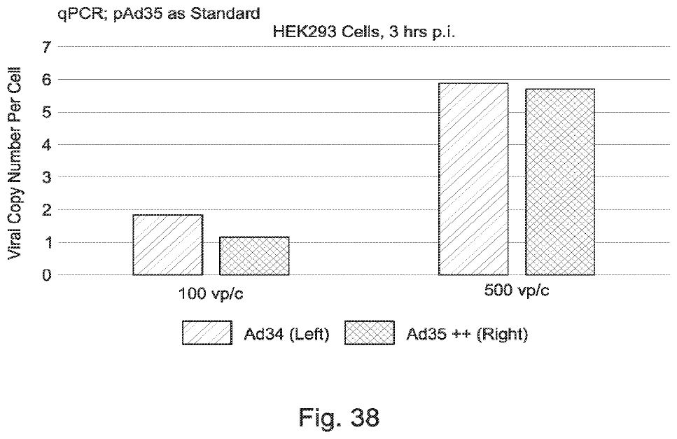

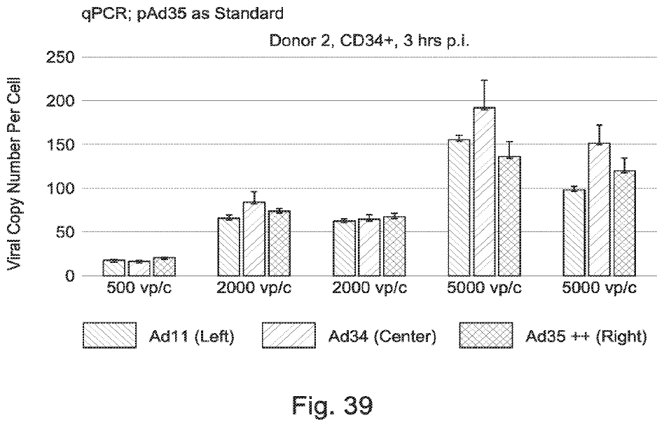

Results of the qPCR analyses of this Example are provided in FIGS. 38 and 39. Broadly, viral copy number per cell was detected and comparable for Ad11, Ad34, and Ad35++.

### OTHER EMBODIMENTS

While we have described a number of embodiments, it is apparent that our disclosure and examples also provide other embodiments that utilize or are encompassed by the compositions and methods described herein. Therefore, it will be appreciated that the scope of disclosure is to be defined by that which may be understood from the disclosure rather than by the specific embodiments that have been represented by way of example. Limitations described with respect to one aspect of the disclosure, in certain embodiments, be practiced with respect to other aspects of the disclosure. For example, limitations of claims that depend directly or indirectly from a certain independent claim presented herein serve as support for those limitations being presented in additional dependent claims of one or more other independent claims.

What is claimed is:

1. A method of in vivo gene therapy in a mammalian subject, the method comprising administering to the subject an adenoviral vector, wherein the adenoviral vector comprises:
(a) a capsid comprising one or more viral polypeptides of an Ad3, Ad7, Ad11, Ad14, Ad16, Ad21, Ad34, Ad37, or Ad50 serotype, wherein the one or more viral polypeptides comprise one or more of a:
(i) fiber knob;
(ii) fiber shaft;
(iii) fiber tail;
(iv) penton; and
(v) hexon; and
(b) a double-stranded DNA genome comprising a heterologous nucleic acid payload.

2. The method of claim 1, wherein the genome further comprises:
(a) a 3′ ITR and a 5′ ITR, wherein each of the 3′ ITR and the 5′ ITR are of the viral polypeptide serotype; and
(b) a packaging sequence, wherein the packing sequence is of the viral polypeptide serotype.

3. The method of claim 1 or 2, wherein the method comprises mobilization of hematopoietic stem cells of the subject prior to administration of the adenoviral vector.

4. The method of any one of claims 1-3, wherein the heterologous nucleic acid payload comprises a selectable marker, optionally wherein the selectable marker is MGMT^{P140K}.

5. The method of claim 4, wherein the method comprises administering a selecting agent to the subject, optionally wherein the selecting agent comprises O⁶BG and/or BCNU.

6. The method of any one of claims 1-5, wherein the method comprises administering one or more immunosuppression agents to the subject, optionally wherein the administration of the one or more immunosuppression agents is prior to the administration of the adenoviral vector.

7. An adenoviral donor vector comprising:
(a) a capsid comprising one or more viral polypeptides of an Ad3, Ad7, Ad11, Ad14, Ad16, Ad21, Ad34, Ad37, or Ad50 serotype, wherein the one or more viral polypeptides comprise one or more of a:
(i) fiber knob;
(ii) fiber shaft;
(iii) fiber tail;
(iv) penton; and
(v) hexon; and
(b) a double-stranded DNA genome comprising a heterologous nucleic acid payload.

8. The vector of claim 7, wherein the genome further comprises:
(a) a 3′ ITR and a 5′ ITR, wherein each of the 3′ ITR and the 5′ ITR are of the viral polypeptide serotype; and
(b) a packaging sequence, wherein the packing sequence is of the viral polypeptide serotype.

9. The vector of claim 7 or 8, wherein the heterologous nucleic acid payload comprises a selectable marker, optionally wherein the selectable marker is MGMT^{P140K}.

10. The method or vector of any one of claims 1-9, wherein the one or more viral polypeptides comprise the:
(a) fiber knob and fiber shaft;
(b) fiber knob and fiber tail;
(c) fiber knob and penton;
(d) fiber knob and hexon;
(e) fiber knob, hexon, and penton;
(f) fiber shaft and fiber tail;
(g) fiber shaft and penton;
(h) fiber shaft and hexon;
(i) fiber shaft, hexon, and penton;
(j) fiber tail and penton;
(k) fiber tail and hexon;
(l) fiber tail, hexon, and penton;
(m) fiber knob, fiber shaft, and fiber tail;
(n) fiber knob, fiber shaft, and penton;
(o) fiber knob, fiber shaft, and hexon;
(p) fiber knob, fiber shaft, hexon, and penton;
(q) fiber knob, fiber shaft, fiber tail, and penton;
(r) fiber knob, fiber shaft, fiber tail, penton, and hexon; or
(s) penton and hexon.

11. The method or vector of any one of claims 1-10, wherein the fiber knob has a sequence that has at least 80% identity to a sequence selected from SEQ ID NOs: 14, 30, 46, 62, 78, 94, 110, 126, and 142.

12. The method or vector of any one of claims 1-11, wherein the fiber shaft has a sequence that has at least 80% identity to a sequence selected from SEQ ID NOs: 13, 29, 45, 61, 77, 93, 109, 125, and 141.

13. The method or vector of any one of claims 1-12, wherein the fiber tail has a sequence that has at least 80% identity to a sequence selected from SEQ ID NOs: 157, 158, 159, 160, 161, 162, 163, 164, and 165.

14. The method or vector of any one of claims 1-13, wherein the penton has a sequence that has at least 80% identity to a sequence selected from SEQ ID NOs: 15, 31, 47, 63, 79, 95, 111, 127, and 143.

15. The method or vector of any one of claims 1-14, wherein the hexon has a sequence that has at least 80% identity to a sequence selected from SEQ ID NOs: 16, 32, 48, 64, 80, 96, 112, 128, and 144.

16. The method or vector of any one of claims 1-15, wherein the adenoviral vector comprises a fiber of the serotype of the viral peptides.

17. The method or vector of any one of claims 1-16, wherein the fiber has a sequence that has at least 80% identity to a sequence selected from SEQ ID NOs: 12, 28, 44, 60, 76, 92, 108, 124, and 140.

18. The method or vector of any one of claims 1-17, wherein the adenoviral vector is a chimeric vector characterized in that the capsid comprises at least one of a fiber knob, fiber shaft, fiber tail, hexon, or penton that is not of the serotype of the viral peptides.

19. The method of any one of claims 1-18, wherein the adenoviral vector is a helper dependent vector.

20. An adenoviral donor vector genome comprising:
(a) a 3′ ITR and a 5′ ITR, wherein the 3′ ITR and the 5′ ITR are each of the same serotype selected from an Ad3, Ad7, Ad11, Ad14, Ad16, Ad21, Ad34, Ad37, or Ad50 serotype;
(b) a packaging sequence, wherein the packing sequence is of the ITR serotype; and
(c) a heterologous nucleic acid payload.

21. The adenoviral donor vector genome of claim 20, wherein the heterologous nucleic acid payload comprises a selectable marker, optionally wherein the selectable marker is MGMT P^{140K}.

22. The method, vector, or genome of any one of claims 1-21, wherein the heterologous nucleic acid payload encodes a protein.

23. The method, vector, or genome of any one of claims 1-21, wherein the heterologous nucleic acid payload encodes a chimeric antigen receptor (CAR), T cell receptor (TCR), or small RNA, optionally wherein the small RNA is an shRNA.

24. The method, vector, or genome of any one of claims 1-21, wherein the heterologous nucleic acid payload encodes a gene editing enzyme or system, wherein the gene editing is selected from CRISPR editing, base editing, prime editing, or zinc finger nuclease editing.

25. The method, vector, or genome of any one of claims 1-24, wherein the heterologous nucleic acid payload encodes an agent for treatment of a condition selected from glioblastoma, hemoglobinopathies, platelet disorders, Fanconi anemia, alpha-1 antitrypsin deficiency, sickle cell anemia, thalassemia, thalassemia intermedia, von Willebrand Disease, hemophilia A, hemophilia B, Factor V Deficiency, Factor VII Deficiency, Factor X Deficiency, Factor XI Deficiency, Factor XII Deficiency, Factor XIII Deficiency, Bernard-Soulier Syndrome, Gray Platelet Syndrome, mucopolysaccharidosis, cystic fibrosis, Tay-Sachs disease, chronic granulomatous disease, Wiskott Aldrich syndrome and phenylketonuria.

26. The method, vector, or genome of any one of claims 1-24, wherein the heterologous nucleic acid payload encodes an agent for treatment of a condition selected from Grave's Disease, rheumatoid arthritis, pernicious anemia, Multiple Sclerosis (MS), inflammatory bowel disease, systemic lupus erythematosus (SLE), adenosine deaminase deficiency (ADA-SCID) or severe combined immunodeficiency disease (SCID), Wiskott-Aldrich syndrome (WAS), chronic granulomatous disease (CGD), Fanconi anemia (FA), Battens disease, adrenoleukodystrophy (ALD) or metachromatic leukodystrophy (MLD), muscular dystrophy, pulmonary alveolar proteinosis (PAP), pyruvate kinase deficiency, Schwachman-Diamond-Blackfan anemia, dyskeratosis congenita, cystic fibrosis, Parkinson's disease, Alzheimer's disease, and amyotrophic lateral sclerosis (Lou Gehrig's disease).

27. The method, vector, or genome of any one of claims 1-26, wherein the serotype of the viral polypeptides is Ad34.

28. A pharmaceutical composition comprising an adenoviral vector of any one of claims 7-27, wherein the pharmaceutical composition is formulated for injection to a subject in need thereof.

29. The method, vector, genome, or pharmaceutical composition of any one of claims 1-28, wherein the adenoviral vector infects and/or transduces CD34+ cells, CD34+high cells, CD34+/CD90+ cells, and/or CD34+high/CD90+ cells, optionally wherein the cells are hematopoietic cells.
